(19) United States
(12) Patent Application Publication
(10) Pub. No.: US 2023/0405157 A1
(43) Pub. Date: Dec. 21, 2023

(54) MELANOCORTIN TYPE 2 RECEPTOR (MC2R) TARGETED THERAPEUTICS AND USES THEREOF

(71) Applicant: Radionetics Oncology, Inc., Menlo Park, CA (US)

(72) Inventors: Jian ZHAO, San Diego, CA (US); Mi CHEN, San Diego, CA (US); Yunfei ZHU, San Diego, CA (US); Sun Hee KIM, San Diego, CA (US)

(21) Appl. No.: 18/313,915

(22) Filed: May 8, 2023

Related U.S. Application Data
(60) Provisional application No. 63/340,380, filed on May 10, 2022.

(51) Int. Cl. A61K 51/04; A61P 35/00; A61P 5/00

(52) U.S. Cl. CPC A61K 51/0485; A61P 35/00; A61P 5/00

(57) ABSTRACT

Described herein are radiotherapeutics that target tumor cells expressing the melanocortin type 2 receptor (MC2R) and their use in the treatment and/or diagnosis of cancer.

### CROSS-REFERENCE TO RELATED APPLICATIONS

This application claims the benefit of U.S. Provisional Patent Application No. 63/340,380, filed May 10, 2022, which is incorporated herein by reference in its entirety.

### FIELD OF THE INVENTION

Described herein are radiotherapeutics that target tumor cells expressing the melanocortin type 2 receptor (MC2R) and methods of using such radiotherapeutics as cancer therapeutics, diagnostics, or both.

### BACKGROUND OF THE INVENTION

Neoplasms are abnormal growth of cells and cause enormous medical burdens, including morbidity and mortality, in humans. Neoplasms include benign or noncancerous neoplasms which do not display malignant features and are generally unlikely to become dangerous (e.g. adenomas); malignant neoplasms display features such as genetic mutations, loss of normal function, rapid division, and ability metastasize (invade) to other tissues; and neoplasms of uncertain or unknown behavior. Malignant neoplasms (i.e. cancerous solid tumors) are the leading cause of death in industrialized countries. Noncancerous neoplasms including benign adenomas can also cause significant morbidity and mortality. Although standard treatments can achieve significant effects in tumor growth inhibition and even tumor elimination, the applied drugs exhibit only minor selectivity for the malignant tissue over healthy tissue and their severe side effects limit their efficacy and use. Specific targeting of neoplastic cells without affecting healthy tissue is a major desire for effective solid tumor therapy.

As one of 3 main classes of cell surface receptors, G protein-coupled receptors (GPCRs) are frequently overexpressed in tumor cells and are considered promising targets for selective tumor therapy. MC2R is a GPCR that is primarily expressed in the adrenal gland. Targeted delivery of radionuclides to adrenal tumors with small molecule MC2R-targeting ligands offers a novel approach to treat and diagnose adrenal cancers, such as adrenocortical carcinoma (ACC).

### SUMMARY OF THE INVENTION

Described herein are radiopharmaceuticals for use in the diagnosis and/or treatment of tumors. The present disclosure provides an alternative and improved method for the treatment of tumors by targeting tumors that overexpress the melanocortin type 2 receptor (MC2R). In some embodiments, the radiopharmaceuticals disclosed herein are useful in the treatment of tumors that overexpress MC2R. In some other embodiments, the radiopharmaceuticals disclosed herein are useful in the identification of tissues or organs in a subject that comprise tumors overexpressing MC2R. Radiopharmaceuticals disclosed herein are also useful in vivo imaging of a subject for the presence of and distribution of tumors that overexpress MC2R in the subject.

In one aspect, described herein is a compound of Formula (I), or a pharmaceutically acceptable salt thereof:

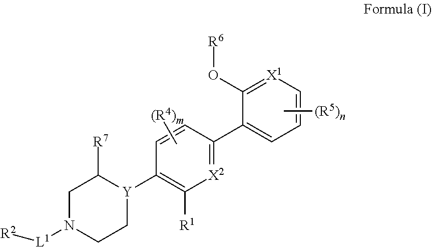

wherein:
R¹ is R^{a}; and Y is N; or R¹ is R⁴; and Y is C—R^{a};
R^{a} is —CH₂NR⁸-L²-R^{b}, or —C(═O)NR⁸-L²-R^{b};
L² is absent, -(unsubstituted or substituted C₁-C₆ alkylene)-, -(unsubstituted or substituted C₁-C₆ alkylene)-N(R⁹)—, -(unsubstituted or substituted C₁-C₆ alkylene)_{q}-(unsubstituted or substituted C₃-C₆cycloalkyl), -(unsubstituted or substituted C₁-C₆ alkylene)_{q}-(unsubstituted or substituted aryl), -(unsubstituted or substituted C₁-C₆ alkylene)_{q}-(unsubstituted or substituted C₂-C₆heterocycloalkyl), or -(unsubstituted or substituted C₁-C₆ alkylene)_{q}-(unsubstituted or substituted heteroaryl); q is 0 or 1;
R^{b} is -L³-Q; L³ is a linker; Q is a chelating moiety or a radionuclide complex thereof;
L¹ is absent or —C(═O)—;
R² is ring that is selected from the group consisting of: C₃-C₈cycloalkyl, C₂-C₈heterocycloalkyl, aryl, or heteroaryl, wherein R² is unsubstituted or is substituted with R^{3a}, R^{3b}, or R^{3c}, or combinations thereof;
each R^{3a}, R^{3b}, R^{3c}, R⁴, and R⁵ is independently hydrogen, halogen, substituted or unsubstituted C₁-C₄alkyl, substituted or unsubstituted C₁-C₄alkenyl, substituted or unsubstituted C₁-C₄alkynyl, substituted or unsubstituted C₁-C₄fluoroalkyl, substituted or unsubstituted C₁-C₄heteroalkyl, —CN, —N(R⁹)₂, or —OR⁹;
R⁶ is C₁-C₄alkyl; R⁷ is hydrogen or C₁-C₄alkyl; R⁸ is hydrogen or C₁-C₄alkyl;
each R⁹ is independently hydrogen, C₁-C₄alkyl, C₁-C₄fluoroalkyl, substituted or unsubstituted C₁-C₄heteroalkyl;
X¹ is CR⁵ or N; X² is CR⁴ or N;
m is 0, 1, or 2; and n is 0, 1, 2, or 3.

In some embodiments, the compound of Formula (I) has the structure of Formula (II), or a pharmaceutically acceptable salt thereof:

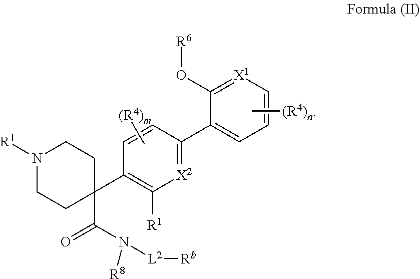

In some embodiments, the compound of Formula (I) has the structure of Formula (III) or Formula (IV), or a pharmaceutically acceptable salt thereof:

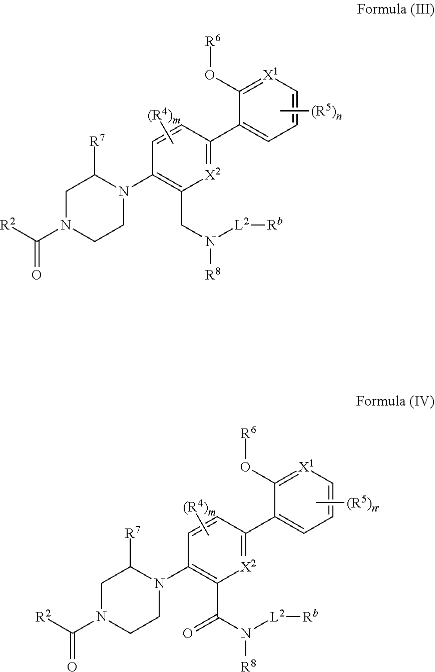

In some embodiments, the compound of Formula (I) has the structure of Formula (V), or a pharmaceutically acceptable salt thereof:

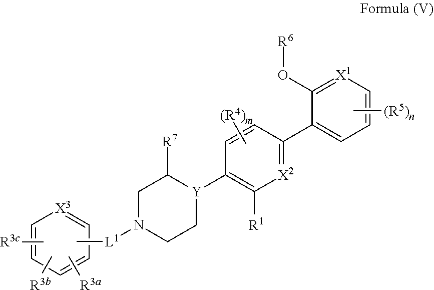

wherein X³ is CH or N.

In some embodiments, the compound of Formula (I) has the structure of Formula (VI), or a pharmaceutically acceptable salt thereof:

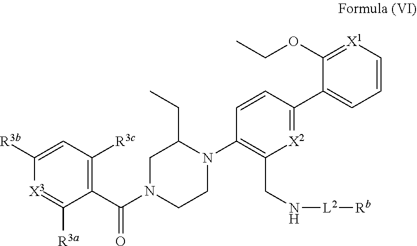

wherein X³ is CH or N.

In some embodiments, the compound of Formula (VI) has the structure of Formula (VIa), Formula (VIb), Formula (VIc), or Formula (VId), or a pharmaceutically acceptable salt thereof:

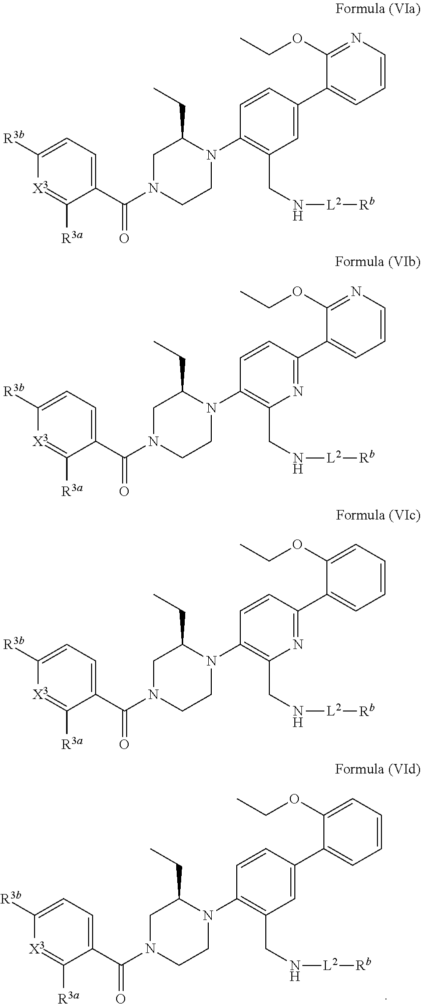

In some embodiments, the compound of Formula (I) has the structure of Formula (VII), or a pharmaceutically acceptable salt thereof:

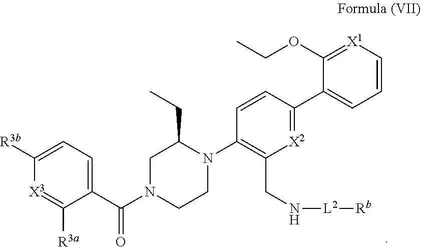

In some embodiments, the compound of Formula (I) has the structure of Formula (VIII), or a pharmaceutically acceptable salt thereof:

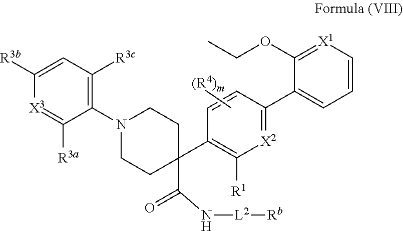

wherein X³ is CH or N.

In some embodiments, the compound of Formula (I) has the structure of Formula (VIIIa), Formula (VIIIb), or Formula (VIIIc), or a pharmaceutically acceptable salt thereof:

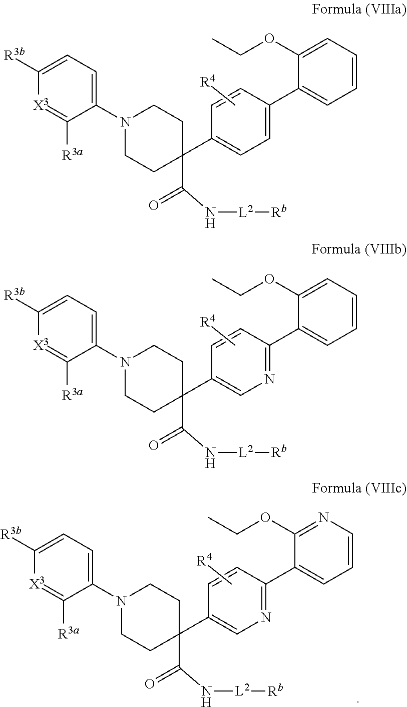

In some embodiments, Q is a chelating moiety selected from the group consisting of: DOTA; DO3A; DO2A; DOTMA; DOTAM; DOTPA; 2,2′,2″-(10-(2-amino-2-oxoethyl)-1,4,7,10-tetraazacyclododecane-1,4,7-triyl)triacetic acid; Bn-DOTA; p-OH-Bn-DOTA; p-SCN-Bn-DOTA; H₄pypa; H₄pypa-benzyl; H₄pypa-benzyl-NCS; H₄py4pa; H₄py4pa-benzyl; H₄py4pa-benzyl-NCS; H₄octapa; H₄octapa-benzyl-NCS; H₄octapa-benzyl; TTHA; or a radionuclide complex thereof. In some embodiments, Q is a chelating moiety selected from the group consisting of: DOTA; and DO3A; or a radionuclide complex thereof.

In some embodiments, the radionuclide of the radionuclide complex is a lanthanide or an actinide. In some embodiments, the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium. In some embodiments, the radionuclide of the radionuclide complex is a diagnostic or therapeutic radionuclide. In some embodiments, the radionuclide of the radionuclide complex is an Auger electron-emitting radionuclide, α-emitting radionuclide, β-emitting radionuclide, or γ-emitting radionuclide. In some embodiments, the radionuclide of the radionuclide complex is 111-indium (¹¹¹In), 115-indium (¹¹⁵In), 67-gallium (⁶⁷Ga), 68-gallium (⁶⁸Ga), 70-gallium (⁷⁰Ga), 225-actinium (²²⁵Ac), 175-lutetium (¹⁷⁵Lu) or 177-lutetium (¹⁷⁷Lu).

In some embodiments, L³ is -L⁴-, -L⁵-, -L⁶-, -L⁷-, -L⁸-, -L⁹-, -L¹⁰-, -L⁴-L⁹-L¹⁰-, -L⁴-L⁵-L⁶-L⁷-L⁸-L⁹-L¹⁰-, or a combination thereof; L⁴ is unsubstituted or substituted C₁-C₂₀alkylene, unsubstituted or substituted C₁-C₂₀heteroalkylene, unsubstituted or substituted C₂-C₂₀alkenylene, unsubstituted or substituted C₂-C₂₀alkynylene, C₄-C₂₀polyethylene glycol, —C(═O)—, —C(═O)NH—, —C(═O)-unsubstituted or substituted C₁-C₂₀alkylene, —C(═O)-unsubstituted or substituted C₁-C₂₀heteroalkylene, —C(═O)—C₄-C₂₀polyethylene glycol, —C(═O)NH-unsubstituted or substituted C₁-C₂₀alkylene, —C(═O)NH-unsubstituted or substituted C₁-C₂₀heteroalkylene, —C(═O)NH—C₄-C₂₀polyethylene glycol, —NHC(═O)-unsubstituted or substituted C₁-C₂₀alkylene, —NHC(═O)-unsubstituted or substituted C₁-C₂₀heteroalkylene, or —NHC(═O)—C₄-C₂₀polyethylene glycol; L⁵ is absent, —S—S—, one or more independently selected natural or unnatural amino acids, wherein when 2 or more independently selected natural or unnatural amino acids are present the peptide that is formed is a linear or branched peptide; L⁶ is absent, —O—, —S—, —S(═O)—, —S(═O)₂—, —NH—, —CH(OH)—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ᵥ—, —C(═O)—(CH₂CH₂X⁴)ᵥ—, or —(CH₂CH₂X⁴)ᵥ—, —C(═O)—(X⁴CH₂CH₂)ᵥ—, or —(X⁴CH₂CH₂)ᵥ—, each instance of v is independently 1, 2, 3, 4, 5, 6, 7, 8, 9, 10, 11, or 12; each X⁴ is independently selected from O and NR^{X}; and each R^{X} is independently selected from hydrogen, C₁-C₄alkyl and —CH₂CO₂H; L⁷ is absent, unsubstituted or substituted C₁-C₆alkylene, unsubstituted or substituted C₁-C₆heteroalkylene, unsubstituted or substituted C₂-C₆alkenylene, unsubstituted or substituted C₂-C₆alkynylene, unsubstituted or substituted cycloalkylene, unsubstituted or substituted heterocycloalkylene, unsubstituted or substituted arylene, unsubstituted or substituted heteroarylene; L⁸ is absent, —[CH(R^{Y})]_{y}—, —(CH₂)_{y}—, —(X⁵CH₂CH₂)_{y}—, or —(CH₂CH₂X⁵)_{y}—, each y is 1, 2, 3, 4, 5, 6, 7, 8, 9, 10, 11, or 12; each R^{y} is independently selected from hydrogen and —OH; each X⁵ is independently selected from O and NR^{X}; and each R^{X} is independently selected from hydrogen, C₁-C₄alkyl and —CH₂CO₂H; L⁹ is absent, —(CH₂)—, —O—, —S—, —S(O)—, —S(O)₂—, —NH—, —CH(OH)—, —C(═O)—, —C(═O)NH—, —NHC(═O)—, —C(═S)NH—, —NHC(═S)—, —C(═O)O—, —OC(═O)—, —OC(═O)NH—, —NHC(═O)NH—, or —NHC(═O)O—; L¹⁰ is absent, unsubstituted or substituted C₁-C₆alkylene, or unsubstituted or substituted C₁-C₆heteroalkylene, or unsubstituted or substituted benzyl.

Also described herein is a pharmaceutical composition comprising a compound described herein (e.g., a compound of Formula (I)), or a pharmaceutically acceptable salt thereof, and at least one pharmaceutically acceptable excipient. In some embodiments, the pharmaceutical composition is formulated for administration to a mammal by intravenous administration or subcutaneous administration. In some embodiments, the pharmaceutical composition is formulated for administration to a mammal by intravenous administration.

In another aspect, described herein is a method for the treatment of cancer comprising administering to a mammal with cancer an effective amount of a compound described herein (e.g., a compound of Formula (I)), or a pharmaceutically acceptable salt thereof, or an effective amount of pharmaceutical composition comprising a compound described herein (e.g., a compound of Formula (I)), or a pharmaceutically acceptable salt thereof. In some embodiments, the cancer comprises tumors and the tumor overexpress the melanocortin subtype-2 receptor (MC2R). In some embodiments, the cancer is an endocrine cancer. In some embodiments, the endocrine cancer comprises adrenal tumors. In some embodiments, the cancer is adrenocortical carcinoma.

In another aspect, described herein is a method for treating adrenal tumors in a mammal with a radionuclide comprising administering to the mammal a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof. In some embodiments, the mammal has been diagnosed with adrenocortical carcinoma. In some embodiments, the endocrine cancer comprises adrenal tumors, neuroendocrine tumors, parathyroid tumors, pituitary tumors, or thyroid tumors.

In another aspect, described herein is a method of targeting delivery of a radionuclide to tumors in a mammal comprising administering to a mammal with tumors a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof; wherein the tumors overexpress the melanocortin subtype-2 receptor (MC2R).

In another aspect, described herein is a method for identifying tissues or organs in a mammal with tumors expressing the melanocortin subtype-2 receptor (MC2R) comprising administering to the mammal a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof; and performing positron emission tomography (PET) analysis, single-photon emission computerized tomography (SPECT), or magnetic resonance imaging (MRI); wherein Q is a chelating moiety-diagnostic radionuclide complex.

In yet another aspect, described herein is a method for the in vivo imaging of tissues or organs in a mammal with tumors expressing the melanocortin subtype-2 receptor (MC2R) comprising administering to the mammal a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof; and performing positron emission tomography (PET) analysis, single-photon emission computerized tomography (SPECT), or magnetic resonance imaging (MRI); wherein Q is a chelating moiety-diagnostic radionuclide complex.

In any of the embodiments disclosed herein, the mammal is a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE FIGURES

FIG. 1 shows the time-activity curves of selected organ activity of ¹¹¹In-Compound 10 in non tumor-bearing male Sprague Dawley Rats.

FIG. 2 shows the uptake of ¹¹¹In-Compound 10 plus/minus excess ¹¹⁵In-Compound 10 at 3 h post-dose in non tumor-bearing male Sprague Dawley Rats.

FIG. 3 shows Time-activity curves of selected organ activity of ¹⁷⁷Lu-Compound 10 in non tumor-bearing male Sprague Dawley Rats.

FIG. 4 shows the uptake of ¹⁷⁷Lu-Compound 10 administered with excess unlabeled/cold-Compound 10 at 3 h post-dose in non tumor-bearing male Sprague Dawley Rats.

FIG. 5 shows the time-activity curves of selected organ activity of ¹¹¹In-Compound 10 in MC2R-positive tumor-bearing female BRGSF mice.

FIG. 6 shows the uptake of ¹¹¹In-Compound 10 plus/minus excess ¹¹⁵In-Compound 10 2 h post-dose in MC2R-positive tumor-bearing female BRGSF mice.

FIG. 7 shows the anti-tumor efficacy of ¹⁷⁷Lu-Compound 10 in MC2R positive mouse tumor model.

### DETAILED DESCRIPTION OF THE INVENTION

Cancer, a disease in which some cells undergo a genetic change in the control of their growth and replication that results in uncontrolled growth and spreading, is one of the leading causes of death worldwide. General types of cancers include solid tumors (cancers that typically originate in organs), carcinomas (cancers that originate in skin or tissues that line organs), sarcomas (cancers of connective tissues such as bones), leukemias (cancers of bone marrow), and lymphomas and myelomas (cancers of the immune system). Neoplasms are abnormal growth of cells that result in solid tumors which may be benign (i.e. do not display malignant features and are generally unlikely to become dangerous such as adenomas), malignant (i.e. display features such as genetic mutations, loss of normal function, rapid division, and ability metastasize (invade) to other tissues), and of uncertain or unknown behavior. State-of-the-art treatment of neoplasms is accomplished by a combination of surgical procedures, chemotherapy, and radiation therapy. Surgical procedures can be curative under some conditions, but often requires multiple interventions as well as combination with radiation and chemotherapy. Chemotherapy proves to be a potent weapon in the fight against cancer in many cases, further optimization is required. Chemotherapy is typically performed by systemic administration of potent cytotoxic drugs, but these compounds lack tumor selectivity and therefore also kill healthy cells in the body. The resulting non-specific toxicity is the cause of severe side effects of chemotherapy which does not target the cancerous cells specifically over other cells. Radiotherapy is the use of high-energy radiation to kill cells. The source of radiation may be external-beam radiation (applied using an external source), internal radiation (placement of a radioactive material near the target cells), or radiotherapy from the systemic administration of a radioactive material. Like chemotherapy, many radiation therapy options also lack tumor cell identification properties needed to achieve the ultimate goal of targeted tumor therapy with drug molecules or radionuclides.

Described herein are radiopharmaceuticals that selectively deliver radionuclides to the malignant cells that overexpress MC2R.

The melanocortin receptors (MCRs) are a family of five G protein-coupled receptors (GPCRs; MC1R, MC2R, MC3R, MC4R, and MC5R) expressed in diverse tissues, which serve discrete physiological functions. Many of the MCRs bind to endogenous peptides beyond the melanocortin peptides, which can act as agonists, antagonists, partial agonists, and even inverse agonists at these receptors (Cone, (2006). Endocr. Rev. 27, 736-749).

GPCRs are generally poorly antigenic making them difficult targets for antibody-based strategies. For many GPCRs, a large proportion of the protein population resides in intracellular compartments at any given time reducing the total number of cell surface binding sites accessible to antibodies or peptides.

Peptides are intrinsically sensitive to proteolytic enzymes and peptidases present in most tissues and are rapidly degraded into multiple fragments which no longer have significant affinity to the intended receptors. In addition, peptides may cause unwanted immunogenic responses complicating later stages of development by masking the therapeutic effect and impacting the safety assessment.

When peptide ligands are linked to radionuclide payloads, the resulting conjugates often degrade apart rapidly in blood plasma and produce cytotoxic or radioactive peptide fragments which may nonspecifically bind to both tumor and normal tissue. Such premature breakdown of peptide radionuclide conjugates and antibody radionuclide conjugates reduce the amount of radionuclide payloads distributed to targeted tumors, lowering treatment efficacy, and possibly increasing toxicity. In addition, peptides are most likely exclusively excreted via kidney, which may limit their applications. Marked kidney uptake of some peptide-based therapeutics has limited their routine use.

High affinity, small molecule ligands that bind GPCRs have been described and are cell permeable and can access populations of receptors in endoplasmic reticulum and endosomes. Owing to the low molecular weight of non-peptide small molecules, vascular permeability and tumor penetration should be improved compared to high molecular weight conjugates based on peptides and antibodies. The binding affinity of small molecule nonpeptide ligands in many cases surpasses that of FDA approved antibodies by orders of magnitude.

### The Melanocortin Receptors (MCRs)

The 5 subtypes of melanocortin receptors (MCRs) expressed in diverse tissues. MC1R is expressed in the melanocytes of the skin and hair follicles. MC2R is predominantly expressed in the adrenal gland where it promotes the expression of steroidogenic enzymes in response to binding plasma ACTH. MC3R is primarily expressed in the central nervous system where it is found in the hypothalamus and the limbic regions (Roselli-Rehfuss et al., (1993). Proc. Natl. Acad. Sci. U.S.A. 90, 8856-8860). MC4R is widely expressed in the central nervous system where it is abundant in several regions including the paraventricular nucleus (PVN) of the hypothalamus (Mountjoy, K. G., et al, (1994). Mol. Endocrinol. 8, 1298-1308). MC5R is widely expressed in peripheral tissues.

There are two adrenal glands. The adrenal glands are small and shaped like a triangle. One adrenal gland sits on top of each kidney. Each adrenal gland has two parts. The outer layer of the adrenal gland is the adrenal cortex. The center of the adrenal gland is the adrenal medulla.

### Adrenal Tumors

Tumors can form in the adrenal glands. Adrenal tumors may be functioning (makes more hormones than normal) or nonfunctioning (does not make more hormones than normal). Most adrenal tumors are functioning. The hormones made by functioning tumors may cause certain signs or symptoms of disease. A functioning adrenal tumor makes too much of one of the following hormones: cortisol, aldosterone, testosterone, and/or estrogen. Tumors that develop in the adrenal glands include adrenal incidentalomas, adenomas (adrenal cortex tumors), adrenocortical carcinoma (ACC), pheochromocytomas, and paragangliomas.

Adrenal incidentalomas are benign and asymptomatic and are typically found during imaging tests. Adenomas (adrenal cortex tumors) are benign but can cause an overproduction of hormones (eg. cortisol, aldosterone).

Adrenocortical carcinoma (ACC) is the most common type of cancerous adrenal tumor and can be aggressive. ACC is a disease in which malignant (cancer) cells form in the outer layer of the adrenal gland. Adrenocortical carcinoma is also called cancer of the adrenal cortex.

The adrenal medulla makes hormones that help the body react to stress. Cancer that forms in the adrenal medulla is called pheochromocytoma. Pheochromocytomas are a rare type of neuroendocrine tumor (NET) that originate in the medulla, cause the over-production of epinephrine and norepinephrine, and are prevalent in certain genetic diseases (e.g. Von Hippel-Lindau disease and multiple endocrine neoplasia (MEN)). Paragangliomas are a type of NET similar to pheochromocytomas but originating outside of the adrenal glands that secrete high levels of epinephrine and norepinephrine.

Although ACCs have heterogenous gene expression, MC2R mRNA is present in tumors originating in the adrenal cortex (Imai et al., Annals of Surgery, Vol. 234, No. 1, 85-91, 2001).

After ACC has been diagnosed, tests are done to find out if cancer cells have spread within the adrenal gland or to other parts of the body. The process used to find out if cancer has spread within the adrenal gland or to other parts of the body is called staging. Tests and procedures used in the staging process include CT scan (CAT scan; also called computed tomography, computerized tomography, or computerized axial tomography), magnetic resonance imaging (MRI; also called nuclear magnetic resonance imaging (NMRI)), positron emission tomography (PET) scan, ultrasound exam, and adrenalectomy (a procedure to remove the affected adrenal gland).

The following stages are used for adrenocortical carcinoma: stage I, stage II, stage III, stage IV, and stage V. In stage I, the tumor is 5 centimeters or smaller and is found in the adrenal gland only. In stage II, the tumor is larger than 5 centimeters and is found in the adrenal gland only. In stage III, the tumor is any size and has spread to nearby lymph nodes; or to nearby tissues or organs (kidney, diaphragm, pancreas, spleen, or liver) or to large blood vessels (renal vein or vena cava) and may have spread to nearby lymph nodes. In stage IV, the tumor is any size, may have spread to nearby lymph nodes, and has spread to other parts of the body, such as the lung, bone, or peritoneum.

Cancer may spread from where it began to other parts of the body, which is called metastasis. Cancer cells break away from where they began (the primary tumor) and travel through the lymph system or blood. The metastatic tumor is the same type of cancer as the primary tumor. For example, if adrenocortical carcinoma spreads to the lung, the cancer cells in the lung are actually adrenocortical carcinoma cells. The disease is metastatic adrenocortical carcinoma, not lung cancer.

Three types of treatment are used to treat adrenal tumors: surgery, radiation therapy, chemotherapy. Surgery to remove the adrenal gland (adrenalectomy) is often used to treat adrenocortical carcinoma. Sometimes surgery is done to remove the nearby lymph nodes and other tissue where the cancer has spread. Radiation therapy uses high-energy x-rays or other types of radiation to kill cancer cells or keep them from growing. There are two types of radiation therapy: external radiation therapy uses a machine outside the body to send radiation toward the area of the body with cancer; internal radiation therapy uses a radioactive substance sealed in needles, seeds, wires, or catheters that are placed directly into or near the cancer. Chemotherapy is a cancer treatment that uses drugs to stop the growth of cancer cells, either by killing the cells or by stopping them from dividing.

Treatment of ACC depends on the stage of the cancer being treated. Treatment of stage I, stage II and stage III ACC may include surgery (adrenalectomy). Nearby lymph nodes may also be removed if they are larger than normal. Treatment of stage IV and recurrent ACC may include the following as palliative therapy to relieve symptoms and improve the quality of life: Chemotherapy, radiation therapy to bones or other sites where cancer has spread, and/or surgery to remove cancer that has spread to tissues near the adrenal cortex.

Despite these options, treatments for adrenal cancers are limited to surgery or use of mitotane. Mitotane, a derivative of DTT insecticide, is a small molecule that has adrenolytic effects, a difficult to titrate dosing regimen, and a suboptimal side effect profile. In addition to these limited options, precision oncology targeting molecular pathways of ACC are used, but typically suboptimal response rate are obtained (see Aymen A Elfiky, “Assessment and Management of Advanced Adrenocortical Carcinoma Using a Precision Oncology Care Model”, Discovery Medicine; ISSN: 1539-6509; Discov Med 21(113):49-56, January 2016). Thus, a need exists for treatment options for adrenal tumors, such as ACC. Described herein are radiopharmaceuticals that target delivery of radionuclides to adrenal tumors, which overexpress the MC2R. Targeted therapies usually cause less harm to normal cells than chemotherapy or radiation therapy do.

Solid Tumors: Benign and/or Malignant Neoplasms (Cancer)

In one aspect, compounds of Formula (I) are used to treat benign and/or malignant neoplasms (solid tumors), wherein the neoplasm comprises cells that overexpress cell surface MC2R.

The term “neoplasm” as used herein, refers to an abnormal growth of cells that may proliferate in an uncontrolled way and may have the ability to metastasize (spread).

Neoplasms include solid tumors, adenomas, carcinomas, sarcomas, leukemias and lymphomas, at any stage of the disease with or without metastases.

A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

Solid tumors are cancers that typically originate in organs, such as the bladder, bowel, brain, breast, endometrium, heart, kidney, lung, liver, uterus, ovaries, pancreas or other endocrine organs (thyroid), and prostate.

In some embodiments, compounds of Formula (I) are used to treat an adenoma. An adenoma is a tumor that is not cancer. It starts in gland-like cells of the epithelial tissue (thin layer of tissue that covers organs, glands, and other structures within the body). An adenoma can grow from many glandular organs, including the adrenal glands, pituitary gland, thyroid, prostate, and others. Over time adenomas may transform to become malignant, at which point they are called adenocarcinomas. Even though benign, they have the potential to cause serious health complications by compressing other structures (mass effect) and by producing large amounts of hormones in an unregulated, non-feedback-dependent manner (causing paraneoplastic syndromes).

Adenomas typically are found in the colon (e.g. adenomatous polyps, which have a tendency to become malignant and to lead to colon cancer), kidneys (e.g. renal adenomas may be precursor lesions to renal carcinomas), adrenal glands (e.g. adrenal adenomas; some secrete hormones such as cortisol, causing Cushing's syndrome, aldosterone causing Conn's syndrome, or androgens causing hyperandrogenism), thyroid (e.g. thyroid adenoma), pituitary (e.g. pituitary adenomas, such as prolactinoma), parathyroid (e.g. an adenoma of a parathyroid gland may secrete inappropriately high amounts of parathyroid hormone and thereby cause primary hyperparathyroidism), liver (e.g. hepatocellular adenoma), breast (e.g. fibroadenomas), appendix (e.g. cystadenoma), bronchial (e.g. bronchial adenomas may cause carcinoid syndrome, a type of paraneoplastic syndrome), prostate (e.g. prostate adenoma), sebaceous gland (e.g. sebaceous adenoma), and salivary glands.

Metastasis is the spread of malignant cells to new areas of the body, often by way of the lymph system or bloodstream. A metastatic tumor is one that has spread from the primary site of origin, or where it started, into different areas of the body. Metastatic tumors comprise malignant cells that express cell surface MC2R.

Tumors formed from cells that have spread are called secondary tumors. Tumors may have spread to areas near the primary site, called regional metastasis, or to parts of the body that are farther away, called distant metastasis.

In some embodiments, the tumor to be treated comprises tumor cells expressing MC2R, wherein the tumor is a primary or metastatic tumor. In some embodiments, the tumor to be treated comprises tumor cells expressing MC2R, wherein the tumor is a primary or metastatic tumor of adrenal origin.

In some embodiments, compounds of Formula (I) are used to treat a carcinoma. Carcinomas include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

In some embodiments, compounds of Formula (I) are used to treat a sarcoma. Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Solid tumors include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Benign solid tumors include adenomas.

Primary and metastatic tumors include, e.g., lung cancer (including, but not limited to, lung adenocarcinoma, squamous cell carcinoma, large cell carcinoma, bronchioloalveolar carcinoma, non-small-cell carcinoma, small cell carcinoma, mesothelioma); breast cancer (including, but not limited to, ductal carcinoma, lobular carcinoma, inflammatory breast cancer, clear cell carcinoma, mucinous carcinoma); colorectal cancer (including, but not limited to, colon cancer, rectal cancer); anal cancer; pancreatic cancer (including, but not limited to, pancreatic adenocarcinoma, islet cell carcinoma, neuroendocrine tumors); prostate cancer; ovarian carcinoma (including, but not limited to, ovarian epithelial carcinoma or surface epithelial-stromal tumor including serous tumor, endometrioid tumor and mucinous cystadenocarcinoma, sex-cord-stromal tumor); liver and bile duct carcinoma (including, but not limited to, hepatocellular carcinoma, cholangiocarcinoma, hemangioma); esophageal carcinoma (including, but not limited to, esophageal adenocarcinoma and squamous cell carcinoma); non-Hodgkin's lymphoma; bladder carcinoma; carcinoma of the uterus (including, but not limited to, endometrial adenocarcinoma, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including, but not limited to, renal cell carcinoma, clear cell carcinoma, Wilm's tumor); cancer of the head and neck (including, but not limited to, squamous cell carcinomas); cancer of the stomach (including, but not limited to, stomach adenocarcinoma, gastrointestinal stromal tumor); multiple myeloma; testicular cancer; germ cell tumor; neuroendocrine tumor; cervical cancer; carcinoids of the gastrointestinal tract, breast, and other organs; and signet ring cell carcinoma.

### Representative Melanocortin Type 2 Receptor (MC2R) Targeting Ligands

In some embodiments, the compound of Formula (I) has a binding affinity to MC2R that is at least 10-fold, at least 50-fold, at least 100-fold, at least 200-fold, at least 500-fold, or at least 1000-fold greater than the binding affinity for other non-target receptors. In some embodiments, the compound of Formula (I) is selective for MC2R as compared to any one of MC1R, MC3R, MC4R, and MC5R. In some embodiments, the compound of Formula (I) has a binding affinity to MC2R that is at least 10-fold, at least 50-fold, at least 100-fold, at least 200-fold, at least 500-fold, or at least 1000-fold greater than the binding affinity for any one of MC1R, MC3R, MC4R, and MC5R.

In some embodiments, the compound of Formula (I) preferentially accumulates in tumor tissues that express the targetted MC2R. In some embodiments, the compound of Formula (I) preferentially accumulates in tissues or organs comprising tumor cells that express MC2R as compared to tissues or organ(s) lacking tumor cells that express MC2R. In some embodiments, the compound of Formula (I) preferentially accumulates at least 1-fold, at least 2-fold, 3-fold, at least 4-fold, at least 5-fold, or greater than 5-fold more in tissues or organ(s) comprising tumor cells that express MC2R as compared to tissues or organs lacking tumor cells that express MC2R. It is understood that the compound may accumulate in certain tissues and organs involved in the metabolism and/or excretion of therapeutics, including but not limited to the kidneys and liver.

In some embodiments, the MC2R targeting ligand is a compound described in U.S. Pat. No. 10,562,884 (U.S. application Ser. No. 16/432,228), which is herein incorporated by reference for such compounds. In some embodiments, the MC2R targeting ligand is a compound of Formula (I), Formula (II), Formula (III), Formula (IV), Formula (V), Formula (VI), Formula (VII), Formula (VIII), Formula (IX), or Formula (IXa), which is described in U.S. Pat. No. 10,562,884. In some embodiments, the MC2R targeting ligand is a compound described in Table 1, Table 2, or Table 3 of U.S. Pat. No. 10,562,884.

In some embodiments, MC2R targeting ligand is a compound described in International Patent Application Number PCT/US2020/058202 (published as International Publication Number WO 2021/091788 A1), which is herein incorporated by reference for such compounds. In some embodiments, the MC2R targeting ligand is a compound of Formula (A), Formula (A2), Formula (A2a), Formula (A2b), Formula (A3), Formula (A3a), Formula (A3b), Formula (A4), Formula (A4a), Formula (A4b), Formula (A5), Formula (A5a), Formula (A5b), Formula (A6), Formula (A6a), Formula (A6b), Formula (I), Formula (II), Formula (IIa), Formula (IIb), Formula (IIc), Formula (IId), Formula (IIe), Formula (IIf), Formula (III), Formula (IIIa), Formula (IIIb), Formula (IIIc), Formula (IIId), Formula (IIIe), Formula (IIIf), Formula (IV), Formula (IVa), Formula (IVb), Formula (IVc), Formula (IVd), Formula (IVe), Formula (IVf), Formula (V), Formula (Va), Formula (Vb), Formula (Vc), Formula (Vd), Formula (Ve), Formula (Vf), Formula (VI), Formula (VIa), Formula (VIb), Formula (VIc), Formula (VId), Formula (VIe), Formula (VIf), Formula (VII), Formula (VIIa), or Formula (VIIb), which is described in International Patent Application Number PCT/US2020/058202. In some embodiments, the MC2R targeting ligand is a compound described in Table 1, Table 2, Table 3, Table 4, or Table 5 of international Patent Application Number PCT/US2020/058202.

In some embodiments, MC2R targeting ligand is a compound described in International Patent Application Number PCT/US2020/064493 (published as International Publication Number WO 2021/126693 A1), which is herein incorporated by reference for such compounds. In some embodiments, the MC2R targeting ligand is a compound of Formula (I), Formula (IIa), Formula (IIb), Formula (III), Formula (IIIa), Formula (IIIb), Formula (IV), Formula (IVa), Formula (IVb), Formula (V), Formula (Va), Formula (Vb), Formula (Vc), Formula (Vd), Formula (Ve), Formula (Vf), Formula (Vg), Formula (Vh), Formula (Vi), Formula (Vj), Formula (Vk), Formula (Vl), Formula (Vm), Formula (Vn), Formula (Vo), Formula (Vp), Formula (VI), Formula (VIa), Formula (VIb), Formula (VIc), Formula (VId), Formula (VIIa), Formula (VIIb), Formula (VIIc), Formula (VIId), Formula (VIIe), Formula (VIIf), Formula (VIII), Formula (IX), Formula (IXa), Formula (X), Formula (Xa), Formula (Xb), Formula (Xc), Formula (Xd), Formula (Xe), Formula (XI), Formula (XIa), Formula (XIb), Formula (XIc), Formula (XId), Formula (XIe), Formula (XII), Formula (XIIa), Formula (XIIb), Formula (XIIc), Formula (XIII), Formula (XIIIa), Formula (XIIIb), Formula (XIIIc), Formula (XIIId), or Formula (XIIIe), which is described in International Patent Application Number PCT/US2020/064493. In some embodiments, the MC2R targeting ligand is a compound described in Table 1 or Table 2 of International Patent Application Number PCT/US2020/064493.

In some embodiments, MC2R targeting ligand is a compound described in International Patent Application Number PCT/US2020/064252 (published as International Publication Number WO 2021/133563 A1), which is herein incorporated by reference for such compounds. In some embodiments, the MC2R targeting ligand is a compound of Formula (I), Formula (II), Formula (IIa), Formula (IIb), Formula (IIc), Formula (IId), Formula (III), Formula (IIIa), Formula (IIIb), Formula (IIIc), Formula (IIId), Formula (IV), Formula (V), Formula (Va), Formula (Vb), Formula (Vc), Formula (Vd), Formula (Ve), Formula (Vf), Formula (VI), Formula (VIa), Formula (VIb), Formula (VIc), Formula (VId), Formula (VII), Formula (VIIa), Formula (VIIb), Formula (VIIc), Formula (VIId), Formula (VIII), Formula (IXa), Formula (IXb), Formula (IXc), Formula (IXd), Formula (IXe), Formula (IXf), Formula (IXg), Formula (X), Formula (Xa), Formula (Xb), Formula (Xc), Formula (Xd), Formula (XI), Formula (XIa), Formula (XIc), Formula (XId), Formula (XIIa), Formula (XIIb), Formula (XIIc), Formula (XIId), Formula (XIIIa), Formula (XIIIb), Formula (XIIIc), Formula (XIIId), Formula (IX), Formula (XV), Formula (XVIa), Formula (XVIb), Formula (XVIc), Formula (XVId), Formula (XVIIa), Formula (XVIIb), Formula (XVIIc), Formula (XVIId), Formula (XVIIIa), Formula (XVIIIb), Formula (XVIIIc), or Formula (XVIIId), which is described in International Patent Application Number PCT/US2020/064252. In some embodiments, the MC2R targeting ligand is a compound described in Table 1 of International Patent Application Number PCT/US2020/064252.

In one aspect, the MC2R targeting ligand is a compound of Formula (I), or a pharmaceutically acceptable salt thereof:

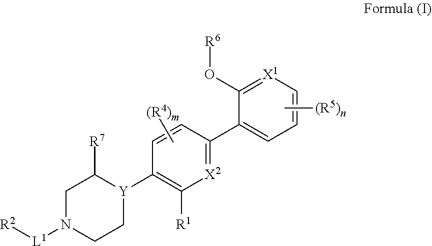

wherein:
R¹ is R^{a}; and Y is N; or R¹ is R⁴; and Y is C—R^{a};
R^{a} is —CH₂NR⁸-L²-R^{b}, or —C(═O)NR⁸-L²-R^{b};
   L² is absent, -(unsubstituted or substituted C₁-C₆ alkylene)-, -(unsubstituted or substituted C₁-C₆ alkylene)-N(R⁹)—, -(unsubstituted or substituted C₁-C₆ alkylene)_{q}-(unsubstituted or substituted C₃-C₆cycloalkyl), -(unsubstituted or substituted C₁-C₆ alkylene)_{q}-(unsubstituted or substituted aryl), -(unsubstituted or substituted C₁-C₆ alkylene)_{q}-(unsubstituted or substituted C₂-C₆heterocycloalkyl), or -(unsubstituted or substituted C₁-C₆ alkylene)_{q}-(unsubstituted or substituted heteroaryl); q is 0 or 1;
R^{b} is -L³-Q; L³ is a linker; Q is a chelating moiety or a radionuclide complex thereof;
L¹ is absent or —C(═O)—;
R² is ring that is selected from the group consisting of C₃-C₈cycloalkyl, C₂-C₈heterocycloalkyl, aryl, or heteroaryl, wherein R² is unsubstituted or is substituted with R^{3a}, R^{3b}, or R^{3c}, or combinations thereof;
each R^{3a}, R^{3b}, R^{3c}, R⁴, and R⁵ is independently hydrogen, halogen, substituted or unsubstituted C₁-C₄alkyl, substituted or unsubstituted C₁-C₄alkenyl, substituted or unsubstituted C₁-C₄alkynyl, substituted or unsubstituted C₁-C₄fluoroalkyl, substituted or unsubstituted C₁-C₄heteroalkyl, —CN, —N(R⁹)₂, or —OR⁹;
R⁶ is C₁-C₄alkyl; R⁷ is hydrogen or C₁-C₄alkyl; R⁸ is hydrogen or C₁-C₄alkyl;
each R⁹ is independently hydrogen, C₁-C₄alkyl, C₁-C₄fluoroalkyl, substituted or unsubstituted C₁-C₄heteroalkyl;
X¹ is CR⁵ or N; X² is CR⁴ or N;
m is 0, 1, or 2; and n is 0, 1, 2, or 3.

In some embodiments, the compound of Formula (I) has the structure of Formula (II), or a pharmaceutically acceptable salt thereof:

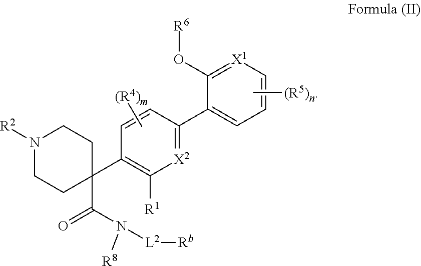

In some embodiments, the compound of Formula (I) has the structure of Formula (III) or Formula (IV), or a pharmaceutically acceptable salt thereof:

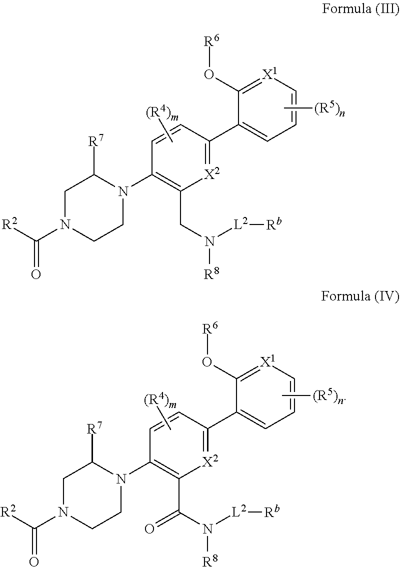

In some embodiments, R² is

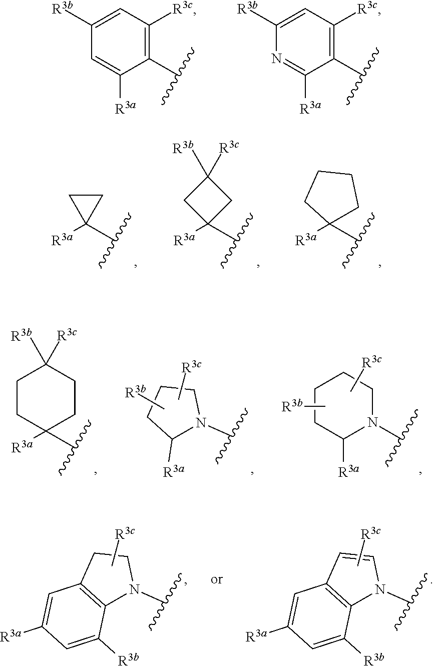

In some embodiments, R² is

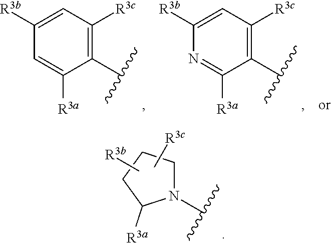

In some embodiments, R² is

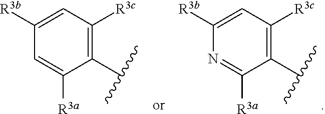

In some embodiments, R² is

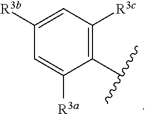

In some embodiments, R² is

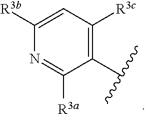

In some embodiments, R² is

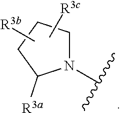

In some embodiments, R² is

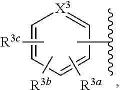

wherein X³ is CH or N.

In some embodiments, the compound of Formula (I) has the structure of Formula (V), or a pharmaceutically acceptable salt thereof:

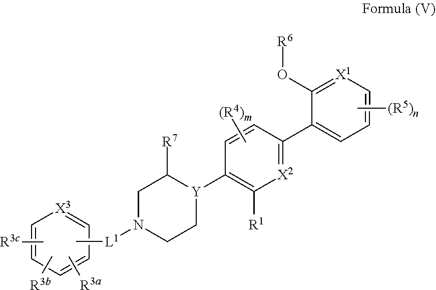

wherein X³ is CH or N.

In some embodiments, each R^{3a}, R^{3b}, R^{3c}, R⁴, and R⁵ is independently hydrogen, F, Cl, Br, —CN, —OH, —OCH₃, —OCH₂CH₃, —CH₃, —CH₂CH₃, —CH₂CH₂CH₃, —CH(CH₃)₂, —C(CH₃)₃, —CH═CH₂, —CH₂OH, —CH₂CN, —CH₂F, —CHF₂, —CF₃, —CH₂CH₂OH, —CH₂CH₂CN, —CH₂CH₂F, —CH₂CHF₂, —CH₂CF₃, —CH₂OCH₃, —CH₂CH₂OCH₃, —CH₂NH₂, —CH₂NHCH₃, —CH₂N(CH₃)₂, —CH₂CH₂NH₂, —CH₂CH₂NHCH₃, or —CH₂CH₂N(CH₃)₂.

In some embodiments, each R^{3a}, R^{3b}, R^{3c}, R⁴, and R⁵ is independently hydrogen, F, Cl, Br, —CN, —OH, —OCH₃, —OCH₂CH₃, —CH₃, —CH₂CH₃, —CH₂F, —CHF₂, or —CF₃.

In some embodiments, R⁶ is —CH₂CH₃; and R⁷ is hydrogen, —CH₃ or —CH₂CH₃.

In some embodiments, the compound of Formula (I) has the structure of Formula (VI), or a pharmaceutically acceptable salt thereof:

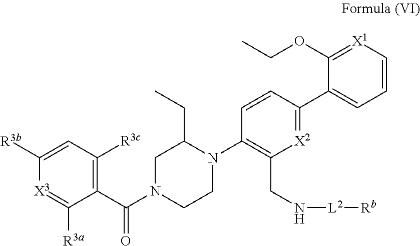

wherein X³ is CH or N.

In some embodiments, the compound of Formula (VI) has the structure of Formula (VIa), Formula (VIb), Formula (VIc), or Formula (VId), or a pharmaceutically acceptable salt thereof:

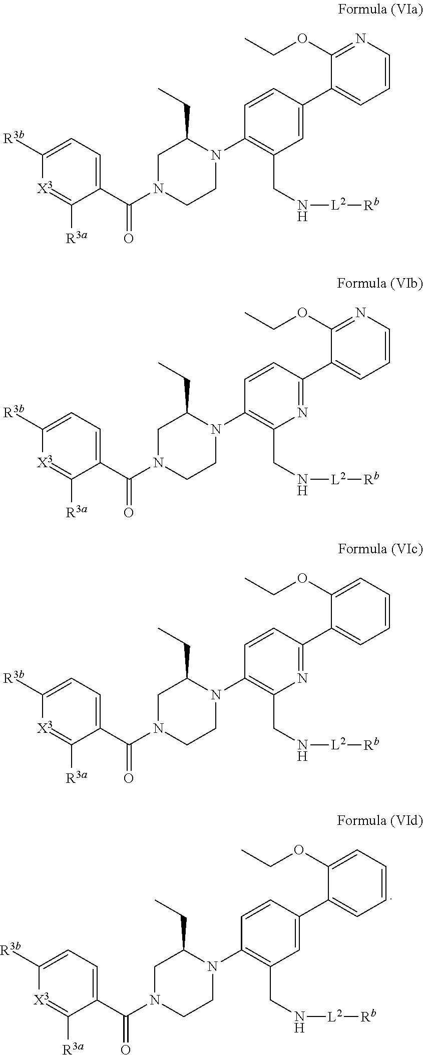

In some embodiments, X³ is CH. In some embodiments, X³ is N.

In some embodiments, each R^{3a}, R^{3b}, R^{3c}, is independently hydrogen, F, Cl, Br, —CN, —OH, —OCH₃, —OCH₂CH₃, —CH₃, —CH₂CH₃, —CH₂CH₂CH₃, —CH(CH₃)₂, —C(CH₃)₃, —CH═CH₂, —CH₂OH, —CH₂CN, —CH₂F, —CHF₂, —CF₃, —CH₂CH₂OH, —CH₂CH₂CN, —CH₂CH₂F, —CH₂CHF₂, —CH₂CF₃, —CH₂OCH₃, —CH₂CH₂OCH₃, —CH₂NH₂, —CH₂NHCH₃, —CH₂N(CH₃)₂, —CH₂CH₂NH₂, —CH₂CH₂NHCH₃, or —CH₂CH₂N(CH₃)₂.

In some embodiments, R^{3a} is hydrogen, F, Cl, —CN, —OH, —OCH₃, —OCH₂CH₃, —CH₃, —CH₂CH₃, —CH₂F, —CHF₂, or —CF₃; and R^{3b} is hydrogen, F, Cl, —CN, —OH, —OCH₃, —OCH₂CH₃, —CH₃, —CH₂CH₃, —CH₂F, —CHF₂, or —CF₃.

In some embodiments, the compound of Formula (I) has the structure of Formula (III) or Formula (VII), or a pharmaceutically acceptable salt thereof:

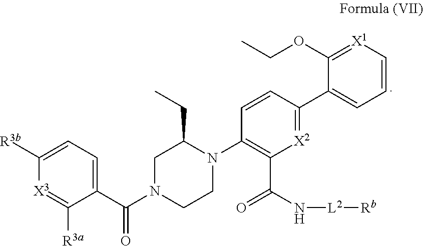

In some embodiments, R^{3a} is hydrogen, F, Cl, —CN, —OCH₃, —OCH₂CH₃, —CH₃, —CH₂CH₃, —CHF₂, or —CF₃; and R^{3b} is hydrogen, F, Cl, —CN, —OCH₃, —OCH₂CH₃, —CH₃, —CH₂CH₃, —CHF₂, or —CF₃.

In some embodiments, R^{3a} is hydrogen, F, Cl, —CN, —OCH₂CH₃, —CH₃, —CHF₂, or —CF₃; and R^{3b} is hydrogen, F, Cl, —CN, —OCH₂CH₃, —CH₃, —CHF₂, or —CF₃.

The compound of claim 1, or a pharmaceutically acceptable salt thereof, wherein the compound of Formula (I) has the structure of Formula (VIII), or a pharmaceutically acceptable salt thereof:

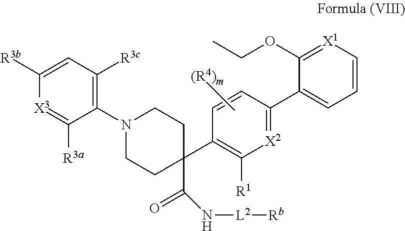

wherein X³ is CH or N.

In some embodiments, the compound of Formula (I) has the structure of Formula (VIIIa), Formula (VIIIb), or Formula (VIIIc), or a pharmaceutically acceptable salt thereof.

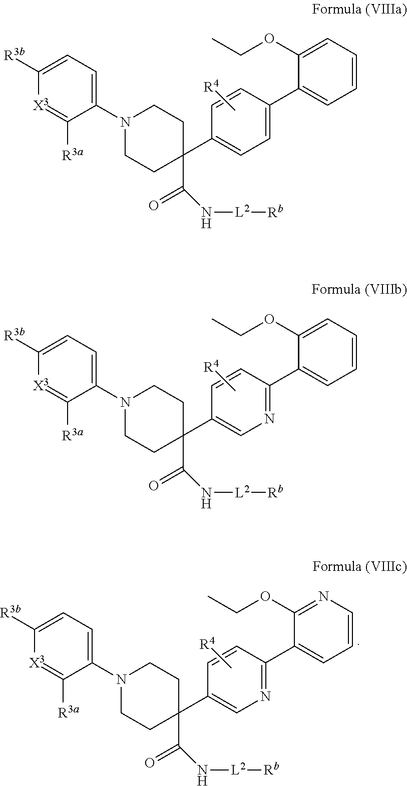

In some embodiments, X³ is CH. In some embodiments, wherein X³ is N.

In some embodiments, each R^{3a} and R^{3b} is independently hydrogen, F, Cl, Br, —CN, —OH, —OCH₃, —OCH₂CH₃, —CH₃, —CH₂CH₃, —CH₂CH₂CH₃, —CH(CH₃)₂, —C(CH₃)₃, —CH═CH₂, —CH₂OH, —CH₂CN, —CH₂F, —CHF₂, —CF₃, —CH₂CH₂OH, —CH₂CH₂CN, —CH₂CH₂F, —CH₂CHF₂, —CH₂CF₃, —CH₂OCH₃, —CH₂CH₂OCH₃, —CH₂NH₂, —CH₂NHCH₃, —CH₂N(CH₃)₂, —CH₂CH₂NH₂, —CH₂CH₂NHCH₃, or —CH₂CH₂N(CH₃)₂; and each R⁴ is independently hydrogen, F, Cl, Br, —CN, —OH, —OCH₃, —CH₃, —CH₂F, —CHF₂, or —CF₃.

In some embodiments, R^{3a} is hydrogen, F, Cl, —CN, —OH, —OCH₃, —OCH₂CH₃, —CH₃, —CH₂CH₃, —CH₂F, —CHF₂, or —CF₃; R^{3b} is hydrogen, F, Cl, —CN, —OH, —OCH₃, —OCH₂CH₃, —CH₃, —CH₂CH₃, —CH₂F, —CHF₂, or —CF₃; and each R⁴ is independently hydrogen or F.

In some embodiments, the compound of Formula (I) has one of the following structures, or a pharmaceutically acceptable salt thereof.

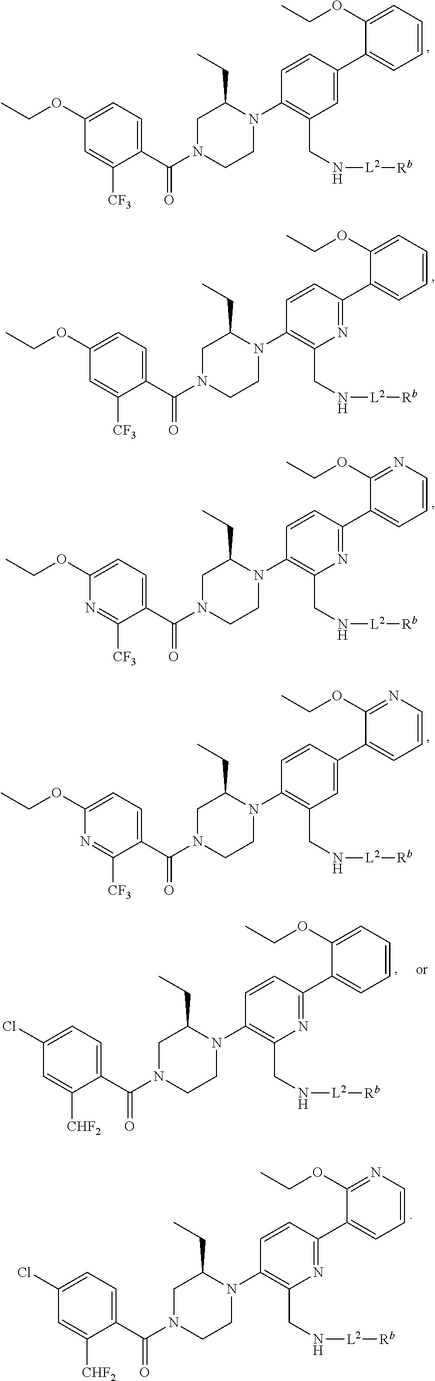

In some embodiments, the compound of Formula (I) has one of the following structures, or a pharmaceutically acceptable salt thereof:

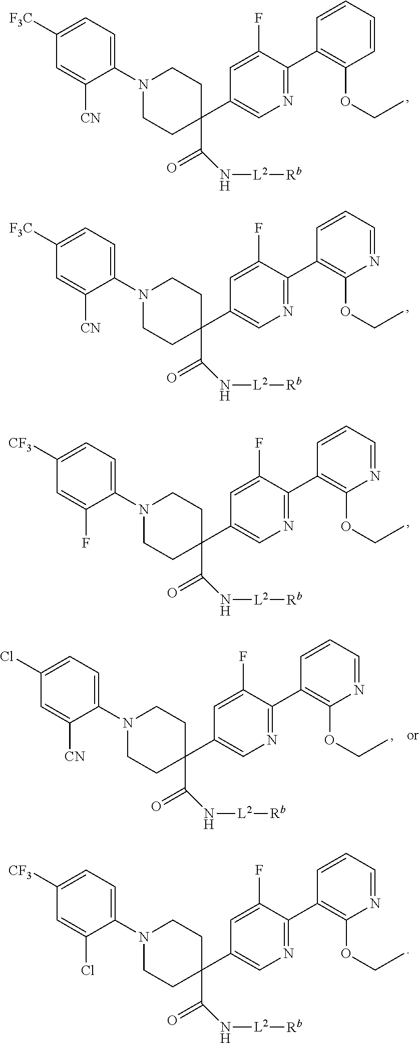

In some embodiments, L² is absent, —CH₂—, —CH₂CH₂—, —CH₂CH₂CH₂—, —CH₂CH₂CH₂CH₂—, —CH₂NH—, —CH₂CH₂NH—, —CH₂CH₂CH₂NH—, —CH₂(CH₂)₂NH—

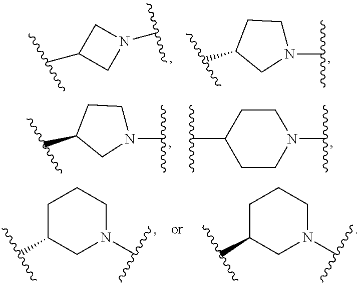

In some embodiments, L² is absent, —CH₂—, —CH₂CH₂—, —CH₂CH₂CH₂—, —CH₂CH₂CH₂CH₂—, —CH₂CH₂NH—, —CH₂CH₂CH₂NH—,

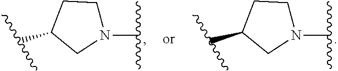 [SMILES: *[C@@H]1CCN(*)C1 ; *[C@H]1CCN(*)C1]

In some embodiments, L² is absent, —CH₂CH₂NH—, —CH₂CH₂CH₂NH—,

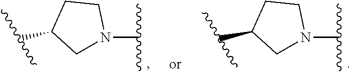 [SMILES: *[C@@H]1CCN(*)C1 ; *[C@H]1CCN(*)C1]

In some embodiments, L² is

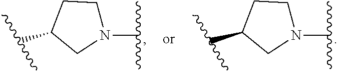 [SMILES: *[C@@H]1CCN(*)C1 ; *[C@H]1CCN(*)C1]

### Radionuclide Complexes

Radiopharmaceuticals have increasingly become very useful tools for physicians to diagnose, stage, treat, and monitor the progression of several diseases, especially cancer. The primary difference between radiopharmaceuticals and other pharmaceutical drugs is that radiopharmaceuticals contain a radionuclide. The nuclear decay properties of the radionuclide determine whether a radiopharmaceutical will be used clinically as a diagnostic agent or as a therapeutic agent. Diagnostic radiopharmaceuticals require radionuclides that emit either gamma (γ) rays or positrons (0+), which subsequently annihilate with nearby electrons to produce two 511 keV annihilation photons emitted approximately 180° away from each other. Gamma ray-emitting radionuclides (e. g. ^{99m}Tc ¹¹¹In, ²⁰¹T1, etc.) are useful for single photon emission computed tomography (SPECT), while positron-emitting radionuclides (e. g. ¹⁸F, ⁸⁹Zr, ⁶⁸Ga, etc.) are useful for positron emission tomography (PET).

In contrast, therapeutic radiopharmaceuticals require radionuclides that emit particulate radiation, such as alpha (α) particles, beta (β-) particles, or Auger electrons. These particles, which strongly interact with target tissues (e. g. cancerous tumor) and lead to extensive localized ionization, can damage chemical bonds in DNA molecules and potentially induce cytotoxicity.

For most nuclear medicine applications, it is desired that a diagnostic radiopharmaceutical is paired with a therapeutic radiopharmaceutical. This concept is commonly known as “theranostics”. As a first step in the theranostic concept, a target molecule labeled with a diagnostic radionuclide is used for quantitative imaging of a tumor imaging biomarker, using positron emission tomography (PET) or single photon emission computed tomography (SPECT).

When it is demonstrated that, with this targeted molecule, a tumoricidal radiation absorbed dose can be delivered to tumor and metastases, as a second step, the administration of the same or a similar target molecule labeled with a therapeutic radionuclide will be conducted.

In some embodiments, the chemical and pharmacokinetic behaviors of both the diagnostic and therapeutic radiopharmaceuticals match. In some embodiments, the diagnostic and therapeutic radionuclides are a chemically identical radioisotope pair (also known as a “matched pair”). One examples of a matched pair for theranostic radiopharmaceutical applications is the ¹²³I/¹³¹I pair, where ¹²³I-labeled compounds are used for diagnosis, while ¹³¹I-labeled compounds are used for therapy. Other theranostic matched pairs include ⁴⁴Sc/⁴⁷Sc, ⁶⁴Cu/⁶⁷Cu, ⁷²As/⁷⁷As, ⁸⁶Y/⁹⁰Y and ²⁰³Pb/²¹²Pb, among others. Alternatively, radionuclide pairs from different elements can be utilized for theranostic radiopharmaceutical development when their chemistry is very similar (e. g. ^{99m}Tc/^{186/188}Re) and there is no significant difference in the pharmacokinetic behavior between the diagnostic and therapeutic analogues. Another example is the ⁶⁸Ga/¹⁷⁷Lu pair, where ⁶⁸Ga is used for diagnosis and ¹⁷⁷Lu is used for therapy. For example, gastroenteropancreatic endocrine tumors express high amounts of sst2 receptor that can be targeted with somatostatin receptor scintigraphy for diagnostic purposes with a ⁶⁸Ga sst2 ligand conjugate ([⁶⁸Ga]Ga-DOTA-TATE (NETSPOT™) or [⁶⁸Ga]Ga-DOTA-TOC (DOTA-(D-Phel,Tyr3)-octreotide, SomaKit TOC®)), followed by treatment with a ¹⁷⁷Lu sst2 ligand conjugate ([¹⁷⁷Lu]Lu-DOTA-TATE) for endoradiotherapy.

Chelating Moieties Used to Generate Metal (Radionuclide) Complexes

The compounds described herein comprise at least one Q group, wherein Q is chelating moiety capable of chelating a radionuclide (Z), or radionuclide complex thereof. In some embodiments, any suitable group or atom(s) of the chelator are used to connect, via an optional linker, to the MC2R targeting ligand.

In some embodiments, the chelator is capable of binding a radioactive atom. In some embodiments, the binding is direct, e.g., the chelator makes hydrogen bonds or electrostatic interactions with a radioactive atom. In some embodiments, the binding is indirect, e.g., the chelator binds to a molecule that comprises a radioactive atom. In some embodiments, the chelator is or comprises a macrocycle.

In some embodiments, the chelator comprises one or more amine groups. In some embodiments, the metal chelator comprises two or more amine groups. In some embodiments, the chelator comprises three or more amine groups. In some embodiments, the chelator comprises four or more amine groups. In some embodiments, the chelator includes 4 or more N atoms, 4 or more carboxylic acid groups, or a combination thereof. In some embodiments, the chelator does not comprise S. In some embodiments, the chelator comprises a ring. In some embodiments, the ring comprises an O and/or a N atom. In some embodiments, the chelator is a ring that includes 3 or more N atoms, 3 or more carboxylic acid groups, or a combination thereof. In some embodiments, the chelator is polydentate ligand, bidentate ligand, or monodentate ligand. Polydentate ligands range in the number of atoms used to bond to a metal atom or ion. EDTA, a hexadentate ligand, is an example of a polydentate ligand that has six donor atoms with electron pairs that can be used to bond to a central metal atom or ion. Bidentate ligands have two donor atoms which allow them to bind to a central metal atom or ion at two points. Ethylenediamine (en) and the oxalate ion (ox) are examples of bidentate ligands.

In some embodiments, a chelator described herein comprises a cyclic chelating agent or an acyclic chelating agent. In some embodiments, a chelator described herein comprises a cyclic chelating agent. In some embodiments, a chelator described herein comprises an acyclic chelating agent.

In some embodiments, a chelator described herein comprises DOTA, DOTAGA, DOTA(GA)2, NOTA, NODAGA, TRITA, TETA, DOTA-MA, DO3A-HP, DOTMA, DOTA-pNB, DOTP, DOTMP, DOTEP, DOTMPE, F-DOTPME, DOTPP, DOTBzP, DOTA-monoamide, p-NCS-DOTA, p-NCS-PADOTA, BAT, DO3TMP-Monoamide, p-NCS-TRITA, and CHX-A″-DTPA. In some embodiments, a chelator described herein comprises DTA, CyEDTA, EDTMP, DTPMP, DTPA, CyDTPA, Cy2DTPA, DTPA-MA, DTPA-BA, and BOPA.

In some embodiments, a chelator described herein comprises DOTA, DOTAGA, DOTA(GA)2, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP.

In some embodiments, a chelator described herein comprises HP-DO3A, BT-DO3A, DO3A-Nprop, DO3AP, DO2A2P, DOA3P, DOTP, DOTPMB, DOTAMAE, DOTAMAP, DO3AM^{Bu}, DOTMA, TCE-DOTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATA^{m}, THP, HEHA, HBED, or HBED-CC TFP.

In some embodiments, a chelator described herein comprises DOTA, NOTA, NODAGA, DOTAGA, HBED, HBED-CC TFP, H2DEPDPA, DFO-B, Deferiprone, CP256, YM103, TETA, CB-TE2A, TE2A, Sar, DiAmSar, TRAPH, TRAP-Pr, TRAP-OH, TRAP-Ph, NOPO, DEADPA, PCTA, EDTA, PEPA, HEHA, DTPA, EDTMP, AAZTA, DO3AP, DO3AP^{PrA}, DO3AP^{ABn} or DOTAM.

In some embodiments, the chelator is or comprises DOTA, HBED-CC, DOTAGA, DOTA(GA)2, NOTA, and DOTAM. In some embodiments, the chelator is or comprises NODAGA, NOTA, DOTAGA, DOTA(GA)2, TRAP, NOPO, NCTA, DFO, DTPA, and HYNIC.

In some embodiments, the chelator comprises a macrocycle, e.g., a macrocycle comprising an O and/or a N atom, DOTA, HBED-CC, DOTAGA, DOTA(GA)2, NOTA, DOTAM, one or more amines, one or more ethers, one or more carboxylic acids, EDTA, DTPA, TETA, DO3A, PCTA, or desferrioxamine.

In some embodiments, a metal chelator described herein comprises one of the following structures.

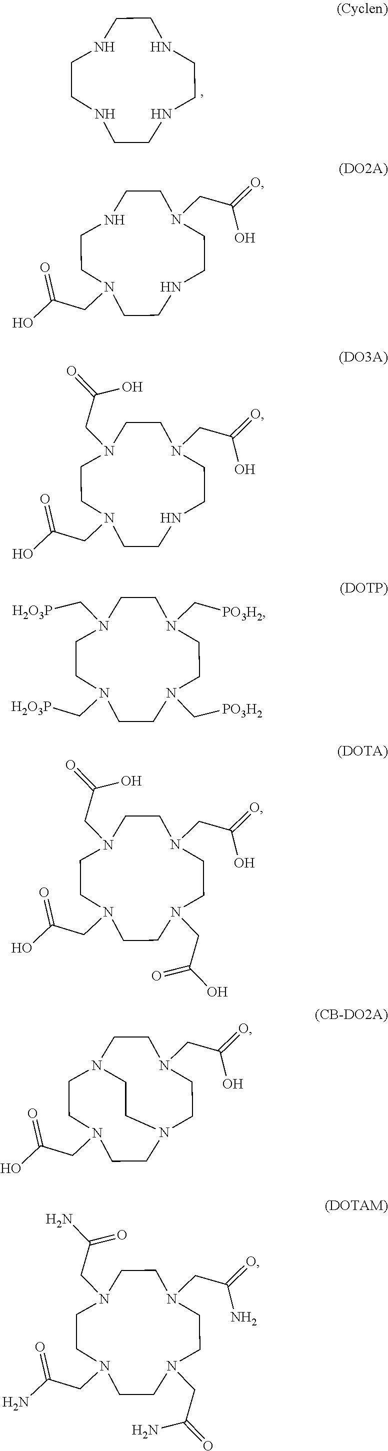

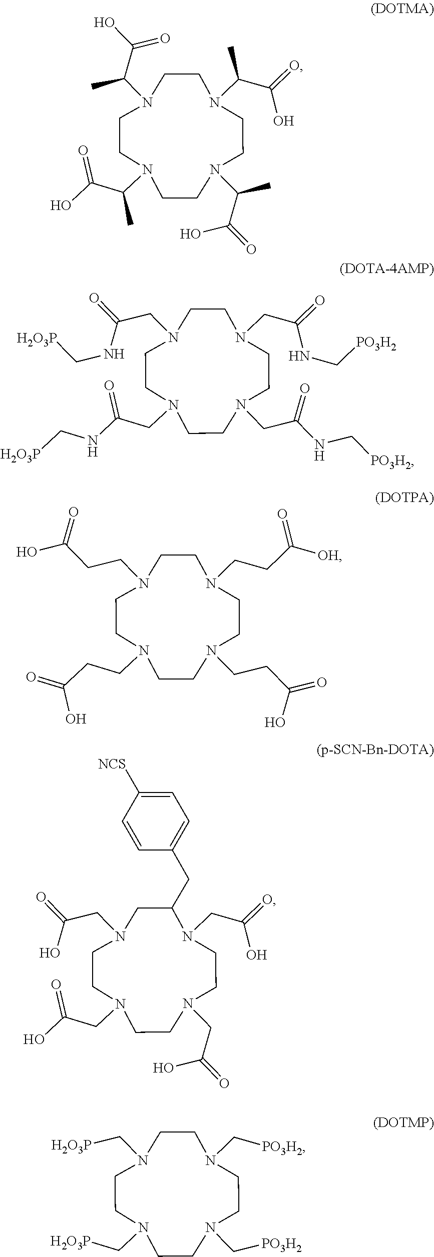

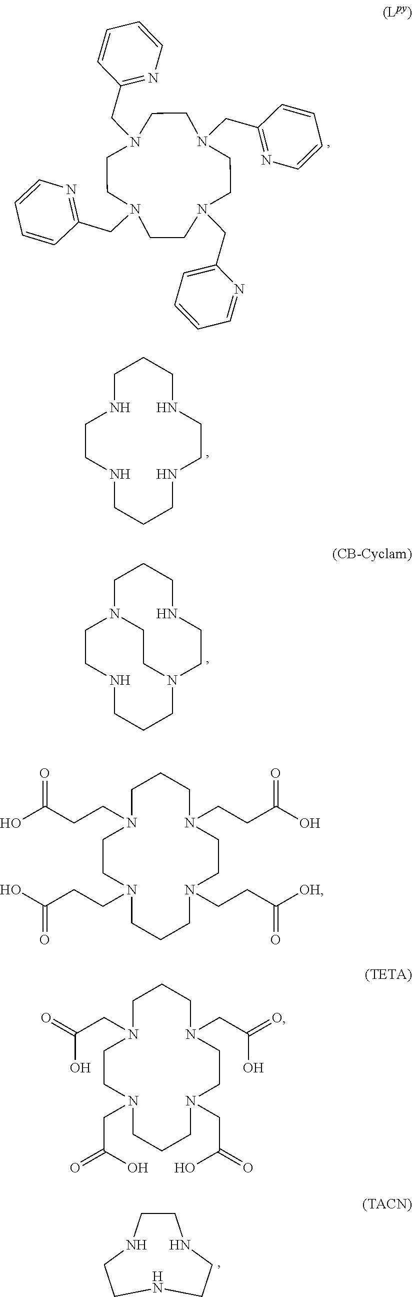

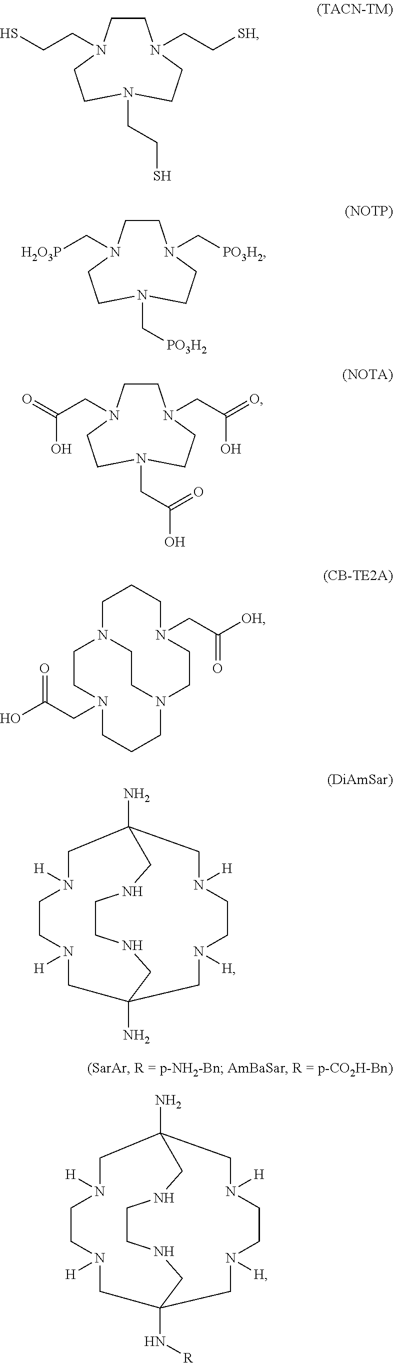

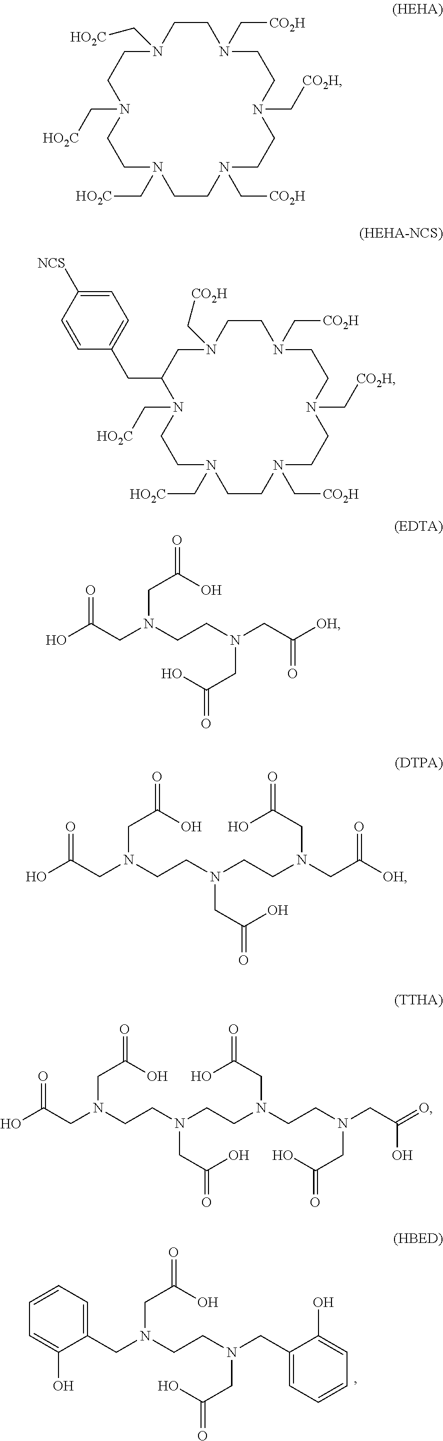

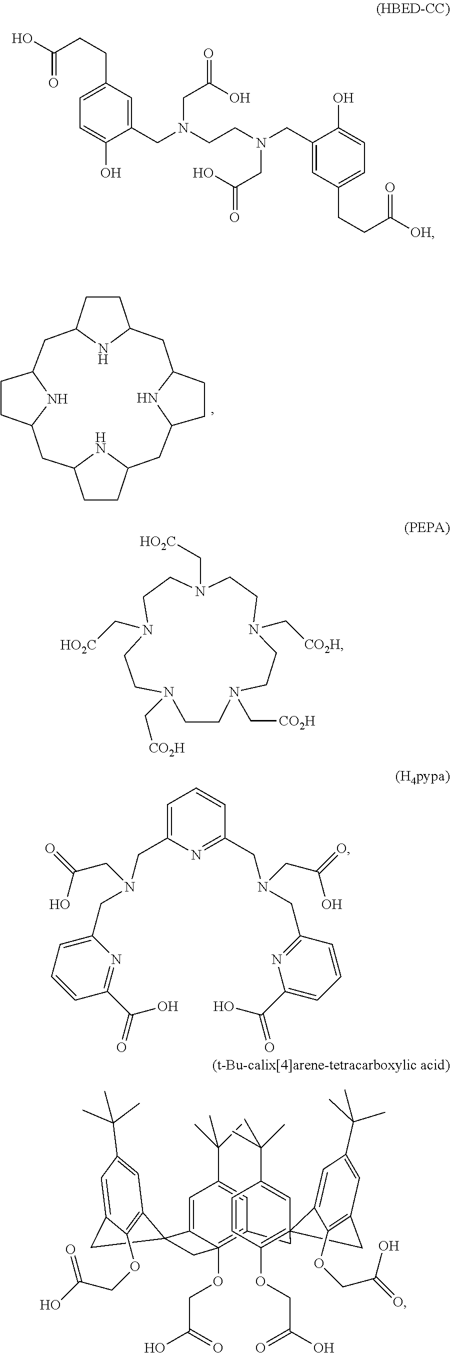

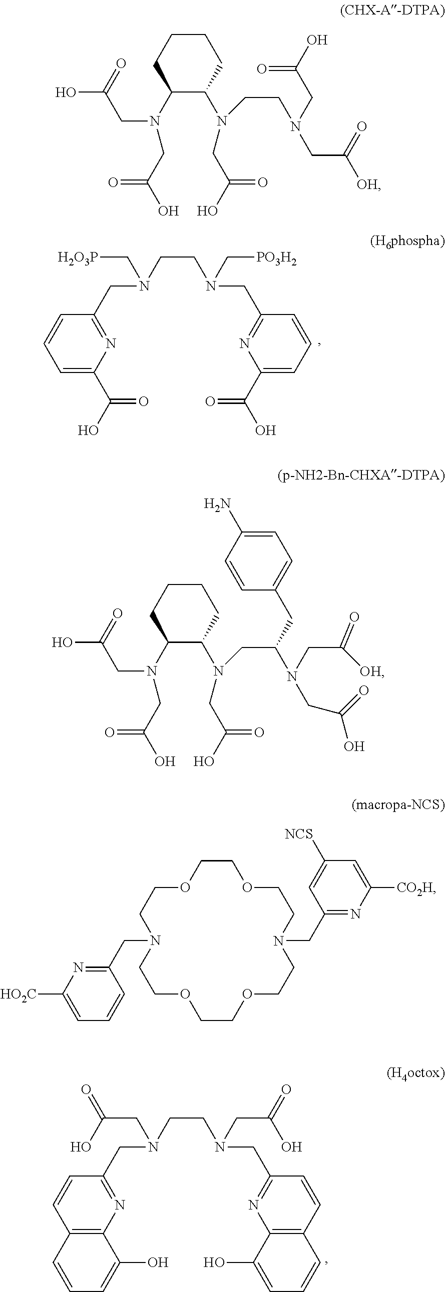

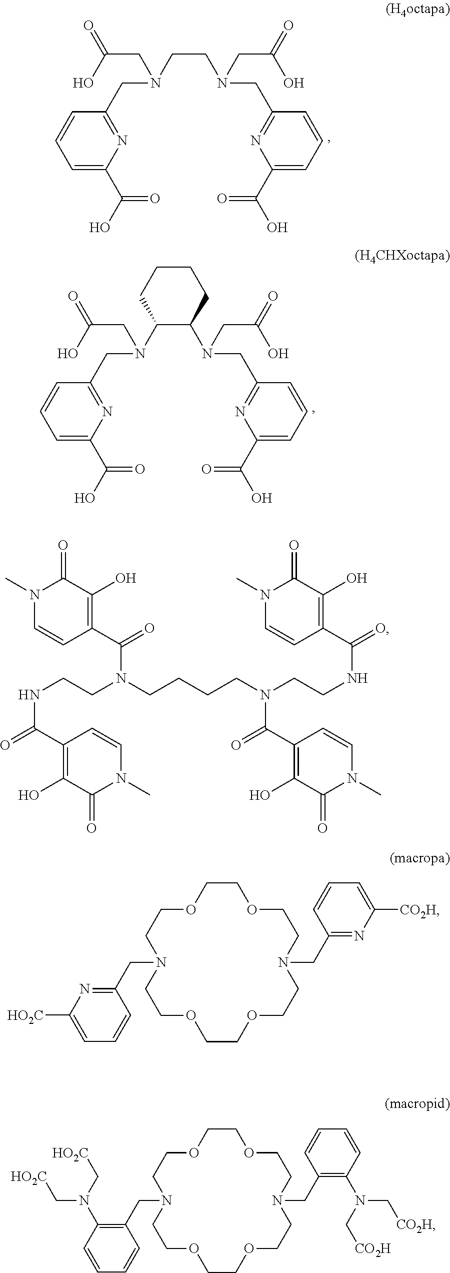 [SMILES: O=C(O)CN(CCN(CC(=O)O)Cc1cccc(C(=O)O)n1)Cc1cccc(C(=O)O)n1 ; O=C(O)CN(Cc1cccc(C(=O)O)n1)[C@@H]1CCCC[C@H]1N(CC(=O)O)Cc1cccc(C(=O)O)n1 ; Cn1ccc(C(=O)NCCN(CCCCN(CCNC(=O)c2ccn(C)c(=O)c2O)C(=O)c2ccn(C)c(=O)c2O)C(=O)c2ccn(C)c(=O)c2O)c(O)c1=O ; O=C(O)c1cccc(CN2CCOCCOCCN(Cc3cccc(C(=O)O)n3)CCOCCOCC2)n1 ; O=C(O)CN(CC(=O)O)c1ccccc1CN1CCOCCOCCN(Cc2ccccc2N(CC(=O)O)CC(=O)O)CCOCCOCC1]

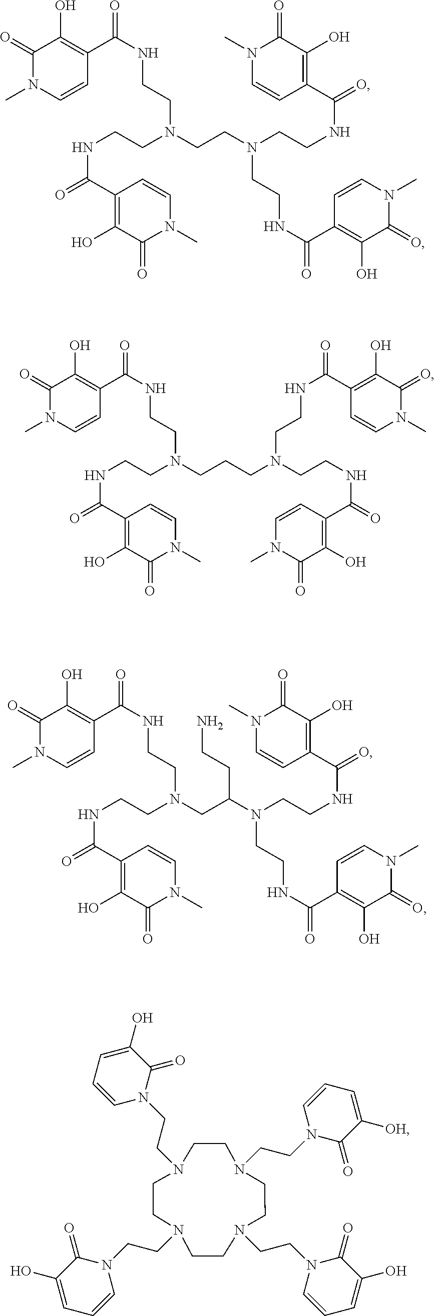 [SMILES: Cn1ccc(C(=O)NCCN(CCNC(=O)c2ccn(C)c(=O)c2O)CCN(CCNC(=O)c2ccn(C)c(=O)c2O)CCNC(=O)c2ccn(C)c(=O)c2O)c(O)c1=O ; Cn1ccc(C(=O)NCCN(CCCN(CCNC(=O)c2ccn(C)c(=O)c2O)CCNC(=O)c2ccn(C)c(=O)c2O)CCNC(=O)c2ccn(C)c(=O)c2O)c(O)c1=O ; Cn1ccc(C(=O)NCCN(CCNC(=O)c2ccn(C)c(=O)c2O)CC(CCN)N(CCNC(=O)c2ccn(C)c(=O)c2O)CCNC(=O)c2ccn(C)c(=O)c2O)c(O)c1=O ; O=c1c(O)cccn1CCN1CCN(CCn2cccc(O)c2=O)CCN(CCn2cccc(O)c2=O)CCN(CCn2cccc(O)c2=O)CC1]

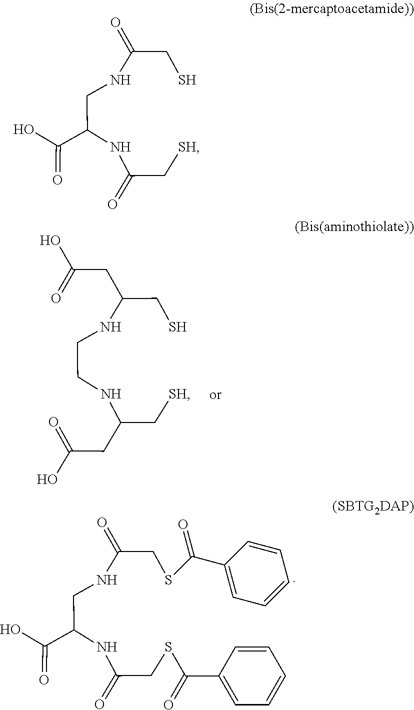 [SMILES: O=C(CS)NCC(NC(=O)CS)C(=O)O ; O=C(O)CC(CS)NCCNC(CS)CC(=O)O ; O=C(CSC(=O)c1ccccc1)NCC(NC(=O)CSC(=O)c1ccccc1)C(=O)O]

In some embodiments, the chelating moiety Q comprises a radionuclide and DOTA. In some embodiments, the chelating moiety Q comprises a radionuclide and a DOTA derivative, such as p-SCN-Bn-DOTA and MeO-DOTA-NCS. In some embodiments, the chelating moiety comprises two independent chelators, and at least one or both are DOTA.

In some embodiments, the chelating moiety comprises a radionuclide and a chelator configured to bind the radionuclide (Z), wherein the chelator comprises DOTA, DOTP, DOTMA, DOTAM, DTPA, NOTA, NODAGA, EDTA, DN3A, DO2A, NOC, TETA, CB-TE2A, DiAmSar, CB-Cyclam, DOTA-4AMP, H₄pypa, H₄octox, H₄octapa, p-NO₂-Bn-neunpa, or NOTP.

In some embodiments, Q is a chelating moiety selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-di acetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); p-SCN-benzyl-1,4,7,10-tetraazacyclododecane-1,4,7,10-tetraacetic acid (p-SCN-Bn-DOTA); 6,6′-(((pyridine-2,6-diylbis(methylene))bis((carboxymethyl)azanediyl))bis(methylene))dipicolinic acid (H₄pypa); H₄pypa-benzyl; H₄pypa-benzyl-NCS; 6,6′,6″,6′″-(((pyridine-2,6-diylbis(methylene))bis(azanetriyl))tetrakis(methylene))-tetrapicolinic acid (H₄py4pa); H₄py4pa-benzyl; H₄py4pa-benzyl-NCS; 6,6′-((ethane-1,2-diylbis((carboxymethyl)azanediyl))bis(methylene))dipicolinic acid (H₄octapa); H₄octapa-benzyl-NCS; H₄octapa-benzyl; 3,6,9,12-tetrakis(carboxymethyl)-3,6,9,12-tetraazatetradecanedioic acid (TTHA); or a radionuclide complex thereof.

In some embodiments, Q is a chelating moiety selected from the group consisting of: 1,4,7,10-tetraazacyclododecane-1,4,7,10-tetraacetic acid (DOTA); or 1,4,7,10-tetraazacyclododecane-1,4,7-triacetic acid (DO3A); or a radionuclide complex thereof.

In some embodiments, Q is a chelating moiety selected from the group consisting of:

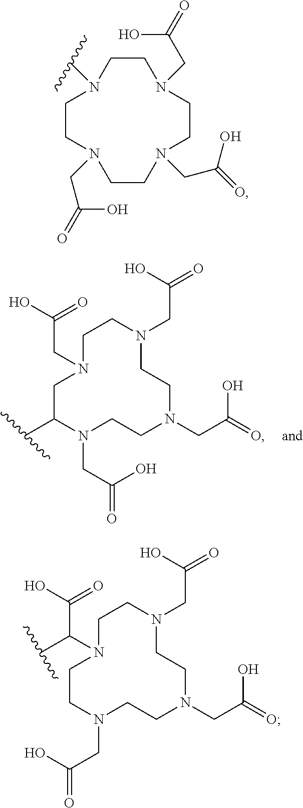 [SMILES: *N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, Q is:

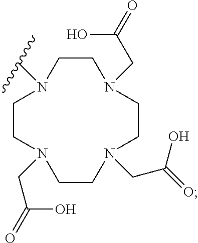 [SMILES: *N1CCN(CC(=O)O)CCN(CC(=O)O)CCN(CC(=O)O)CC1]

or a radionuclide complex thereof.

In some embodiments, Q is:

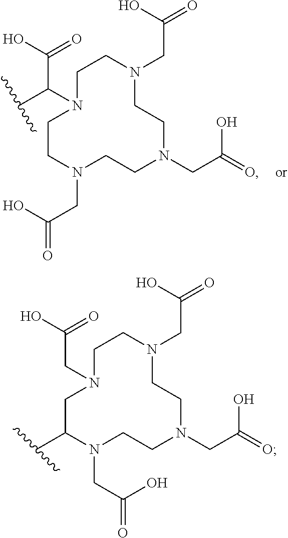 [SMILES: *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O]

or a radionuclide complex thereof.

In some embodiments, Q is:

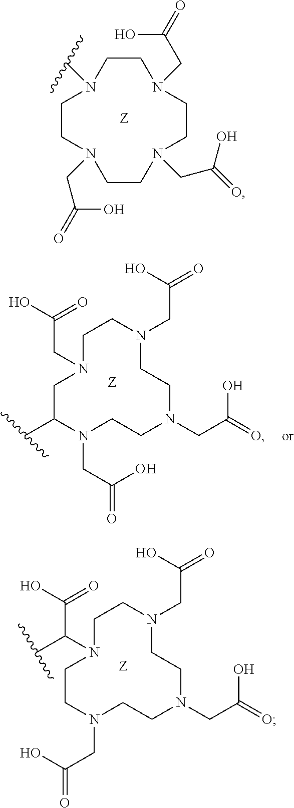

wherein Z is a diagnostic or therapeutic radionuclide.

In some embodiments, Z is an Auger electron-emitting radionuclide, α-emitting radionuclide, β-emitting radionuclide, or γ-emitting radionuclide. In some embodiments, Z is an Auger electron-emitting radionuclide that is 111-indium (¹¹¹In), 67-gallium (⁶⁷Ga), 68 gallium (⁶⁸Ga), 99m-technetium (^{99m}Tc), or 195m-platinum (^{195m}Pt). In some embodiments, Z is an α-emitting radionuclide that is 225-actinium (²²⁵Ac), 213-bismuth (²¹³Bi), 223-Radium (²²³Ra), or 212-lead (²¹²Pb). In some embodiments, Z is a β-emitting radionuclide that is 90-yttrium (⁹⁰Y) 177-lutetium (¹⁷⁷Lu), iodine-131 (¹³¹I), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy) 99m-technetium (^{99m}Tc), 89-zirconium (⁸⁹Zr), or 52-manganese (⁵²Mn). In some embodiments, Z is a γ-emitting radionuclide that is 60-cobalt (⁶⁰Co), 103-pallidum (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Q comprises a radionuclide (Z) and a chelator configured to bind the radionuclide (Z), wherein the radionuclide is suitable for positron emission tomography (PET) analysis, single-photon emission computerized tomography (SPECT), or magnetic resonance imaging (MRI). In some embodiments, the radionuclide is copper-64 (⁶⁴Cu), gallium-68 (⁶⁸Ga), 111-indium (¹¹¹In), or technetium-99m (^{99m}Tc).

Metals (Radionuclides)

In some embodiments, Z is an Auger electron-emitting radionuclide. In some embodiments, Z is an α-emitting radionuclide. In some embodiments, Z is a β-emitting radionuclide. In some embodiments, Z is a γ-emitting radionuclide. In some embodiments, the type of radionuclide used in a non-peptide targeted therapeutic compound can be tailored to the specific type of cancer, the type of targeting moiety (e.g., non-peptide ligand), etc. Radionuclides that undergo α-decay emit α-particles (helium ions with a+2 charge) from their nuclei. As a result of α-decay the daughter nuclide has 2 protons less and 2 neutrons less than the parent nuclide. This means that in α-decay, the proton number is reduced by 2 while the nucleon number is reduced by 4. Radionuclides that undergo β-decay emit β-particles (electrons) from their nuclei. During β-decay, one of the neutrons changes into a proton and an electron. The proton remains in the nucleus while the electron is emitted as a β-particle. This means that in β-decay, the nucleus loses a neutron but gains a proton. In γ-decay, a nucleus in an excited state (higher energy state) emits a γ-ray photon to change to a lower energy state. There is no change in the proton number and nucleon number during the γ-decay. The emission of γ-rays often accompanies the emission of α-particles and β-particles.

Auger electrons (AEs) are very low energy electrons that are emitted by radionuclides that decay by electron capture (EC) (e.g. ¹¹¹In, ⁶⁷Ga, ^{99m}Tc, ^{195m}Pt, ¹²⁵I and ¹²³I). This energy is deposited over nanometre-micrometre distances, resulting in high linear energy transfer that is potent for causing lethal damage in cancer cells. Thus, AE-emitting radiotherapeutic agents have great potential for treatment of cancer.

β-Particles are electrons emitted from the nucleus. They typically have a longer range in tissue (of the order of 1-5 mm) and are the most frequently used.

α-Particles are helium nuclei (two protons and two neutrons) that are emitted from the nucleus of a radioactive atom. Depending on their emission energy, they can travel 50-100 μm in tissue. They are positively charged and are orders of magnitude larger than electrons. The amount of energy deposited per path length travelled (designated ‘linear energy transfer’) of α-particles is approximately 400 times greater than that of electrons. This leads to substantially more damage along their path than that caused by electrons. An α-particle track leads to a preponderance of complex and largely irreparable DNA double-strand breaks. The absorbed dose required to achieve cytotoxicity relates to the number of α-particles traversing the cell nucleus. With use of this as a measure, cytotoxicity may be achieved with a range of 1 to 20 α-particle traversals of the cell nucleus. The resulting high potency, combined with the short range of α-particles (which reduces normal organ toxicity), has led to substantial interest in developing α-particle-emitting agents. The α-particle emitters typically used include bismuth-212, lead-212, bismuth-213, actinium-225, radium-223 and thorium-227.

In some embodiments, Z is a diagnostic or therapeutic radionuclide.

### Representative Radionuclides

| Isotope | Radionuclide t_{1/2} (h) | Decay mode |
|---|---|---|
| ⁶⁰Cu | 0.4 | β+ (93%), EC (7%) |
| ⁶¹Cu | 3.3 | β+ (62%), EC (38%) |
| ⁶²Cu | 0.16 | β+ (98%), EC (2%) |
| ⁶⁴Cu | 12.7 | β+ (19%), EC (41%), |
| | | β− (40%) |
| ⁶⁷Cu | 61.9 | |
| ⁶⁶Ga | 9.5 | β+ (56%), EC (44%) |
| ⁶⁷Ga | 78.2 | EC (100%) |
| ⁶⁸Ga | 1.1 | β+ (90%), EC (10%) |
| ⁴⁴Sc | 3.9 | β+ (94%), EC (6%) |
| ⁴⁷Sc | 80.2 | β− (100%) |
| ¹¹¹In | 67.2 | EC (100%) |
| ^{114m}In | 49.5 d | EC (100%) |
| ¹¹⁴In (daughter) | 73 s | β− (100%) |
| ¹⁷⁷Lu | 159.4 | β− (100%) |
| ⁸⁶Y | 14.7 | β+ (33%), EC (66%) |
| ⁹⁰Y | 64.1 | β− (100%) |
| ⁸⁹Zr | 78.5 | β+ (23%), EC (77%) |
| ²¹²Bi | 1.1 | α (36%), β− (64%) |
| ²¹³Bi | 0.76 | α (2.2%), β− (97.8%) |
| ²¹²Pb (daughter is ²¹²Bi) | 10.6 | β− (100%) |
| ²²⁵Ac | 240 | α (100%) |
| ²²⁷Th | 448.8 | α |
| ²¹¹At | 7.2 | α |

In some embodiments, Z is an Auger electron-emitting radionuclide. In some embodiments, Z is an Auger electron-emitting radionuclide that is 111-indium (¹¹¹In), 67-gallium (⁶⁷Ga), 68 gallium (⁶⁸Ga), 99m-technetium (^{99m}Tc), or 195m-platinum (^{195m}Pt).

In some embodiments, Z is an α-emitting radionuclide. In some embodiments, Z is an α-emitting radionuclide that is 225-actinium (²²⁵Ac), 213-bismuth (²¹³Bi), 223-Radium (²²³Ra), or 212-lead (²¹²Pb).

In some embodiments, Z is an β-emitting radionuclide. In some embodiments, Z is a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵1Dy), 99m-technetium (^{99m}Tc), 89-zirconium (⁸⁹Zr), or 52-manganese (⁵²Mn).

In some embodiments, Z is a γ-emitting radionuclide. In some embodiments, Z is a γ-emitting radionuclide that is 60-cobalt (⁶⁰Co), 103-pallidum (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z is an Auger electron-emitting radionuclide that is 111-indium (¹¹¹In), 67-gallium (⁶⁷Ga), 68 gallium (⁶⁸Ga), 99m-technetium (^{99m}Tc), or 195m-platinum (^{195m}Pt); or Z is an α-emitting radionuclide that is 225-actinium (²²⁵Ac), 213-bismuth (²¹³Bi), 223-Radium (²²³Ra), or 212-lead (²¹²Pb); or Z is a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); Z is a γ-emitting radionuclide that is 60-cobalt (⁶⁰Co), 103-pallidum (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

In some embodiments, Z is ⁹⁴Tc, ⁹⁰In, ¹¹¹In, ⁶⁷Ga, ⁶⁸Ga, ⁸⁶Y, ⁹⁰Y, ¹⁷⁷Lu, ¹⁶¹Tb, ¹⁸⁶Re, ¹⁸⁸Re, 64Cu, ⁶⁷Cu, ⁵⁵Co, ⁵⁷Co, ⁴³Sc, ⁴⁴Sc, ⁴⁷Sc, ²²⁵Ac, ²¹³Bi, ²²⁵Bi, ²¹²Pb, ²²⁷Th, ¹⁵³Sm, ¹⁶⁶Ho, ¹⁵²Gd, ¹⁵³Gd, ¹⁵⁷Gd, and ¹⁶⁶D.

In some embodiments, Z is ⁶⁷Cu, ⁶⁴Cu, ⁹⁰Y, ¹⁰⁹Pd, ¹¹¹Ag, ¹⁴⁹Pm, ¹⁵³Sm, ¹⁶⁶Ho, ^{99m}Tc, ⁶⁷Ga ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ⁴⁴Sc, ⁴⁷Sc, ⁷⁰As, ⁷¹As, ⁷²As, ⁷³As, ⁷⁴As, ⁷⁶As, ⁷⁷As, ²¹²Pb, ²¹²Bi, ²¹³Bi, ²²⁵Ac, ^{117m}Sn, ⁶⁷Ga, ²⁰¹Tl, ¹⁶⁰Gd, ¹⁴⁸Nd, and ⁸⁹Sr.

In some embodiments, Z is ⁶⁸Ga, ⁴³Sc, ⁴⁴Sc, ⁴⁷Sc, ¹⁷⁷Lu, ¹⁶¹Tb, ²²⁵Ac, ²¹³Bi, ²¹²Bi, or ²¹²Pb. In some embodiments, Z is ⁶⁷Ga, ^{99m}Tc, ¹¹¹In, or ²⁰¹Tl.

In some embodiments, compounds described herein comprise a nonradioactive metal. In some embodiments, Q is a chelating moiety comprising a nonradioactive metal. In some embodiments, Q is a chelated nonradioactive metal. In some embodiments, nonradioactive metal comprises nonradioactive gallium, nonradioactive lutetium, or nonradioactive indium. In some embodiments, nonradioactive indium comprises ¹¹⁵In. In some embodiments, nonradioactive lutetium comprises ¹⁷⁵Lu. In some embodiments, nonradioactive gallium comprises ⁶⁹Ga In some embodiments, nonradioactive gallium comprises ⁷¹Ga. In some embodiments, nonradioactive gallium comprises ^{69/71}Ga, wherein ^{69/71}Ga comprises a mixture of ⁶⁹Ga and ⁷¹Ga.

Exemplary Chelator and Radionuclide Complexes

Radionuclides have useful emission properties that can be used for diagnostic imaging techniques, such as single photon emission computed tomography (SPECT, e.g., ⁶⁷Ga, ^{99m}Tc, ¹¹¹In, ¹⁷⁷Lu) and positron emission tomography (PET, e.g. ⁶⁸Ga, ⁶⁴Cu, ⁴⁴Sc, ⁸⁶Y, ⁸⁹Zr), as well as therapeutic applications (e.g. ⁴⁷Sc, ¹¹⁴mIn, ¹⁷⁷Lu, ⁹⁰Y, ^{212/213}Bi, ²¹²Pb, ²²⁵Ac, ^{186/188}Re). A fundamental component of a radiometal-based radiopharmaceutical is the chelator, the ligand system that binds the radiometal ion in a tight stable coordination complex so that it can be properly directed to a desirable molecular target in vivo. Guidance for selecting the optimal match between chelator and radiometal for a particular use is provided in the art (e.g. see Price et al., “Matching chelators to radiometals for radiopharmaceuticals”, Chem. Soc. Rev., 2014, 43, 260-290).

In some embodiments, Q is a chelating moiety selected from the group consisting of: DOTA; DO3A; DO2A; DOTMA; DOTAM; DOTPA; Bn-DOTA; p-OH-Bn-DOTA; p-SCN-Bn-DOTA; H₄pypa; H₄pypa-benzyl; H₄pypa-benzyl-NCS; H₄py4pa; H₄py4pa-benzyl; H₄py4pa-benzyl-NCS; H₄octapa; H₄octapa-benzyl-NCS; H₄octapa-benzyl; TTHA; or a radionuclide complex thereof.

In some embodiments, Q is:

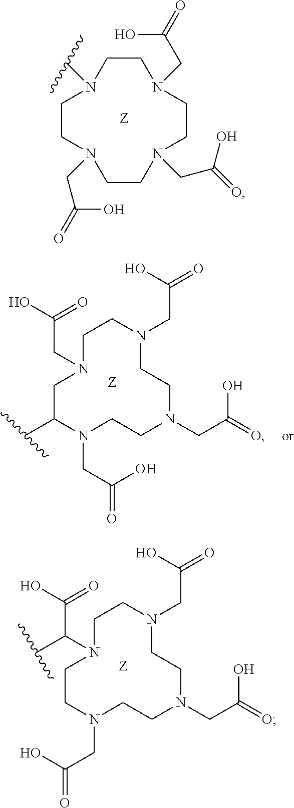

wherein Z is a radionuclide.

In some embodiments, the radionuclide (Z) is 111-indium (¹¹¹In), 115-indium (¹¹⁵In), 67-gallium (⁶⁷Ga), 68-gallium (⁶⁸Ga), 70-gallium (⁷⁰Ga), 225-actinium (²²⁵Ac), 175-lutetium (¹⁷⁵Lu) or 177-lutetium (¹⁷⁷Lu). In some embodiments, the radionuclide (Z) is 90-yttrium (⁹⁰Y), 177-lutetium (¹⁷⁷Lu), 186-rhenium (¹¹⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

Emission Tomography

In some embodiments, Q comprises a chelated radionuclide that is suitable for positron emission tomography (PET) analysis or single-photon emission computerized tomography (SPECT). In some embodiments, Q comprises a chelated radionuclide that is suitable for single-photon emission computerized tomography (SPECT). In some embodiments, Q comprises a chelated radionuclide that is suitable for positron emission tomography (PET) analysis. In some embodiments, Q comprises a chelated radionuclide that is suitable for positron emission tomography imaging, positron emission tomography with computed tomography imaging, or positron emission tomography with magnetic resonance imaging.

In some embodiments, Q is a chelating moiety selected from the group consisting of DOTA; DO3A; DO2A; DOTMA; DOTAM; DOTPA; Bn-DOTA; p-OH-Bn-DOTA; p-SCN-Bn-DOTA; H₄pypa; H₄pypa-benzyl; H₄pypa-benzyl-NCS; H₄py4pa; H₄py4pa-benzyl; H₄py4pa-benzyl-NCS; H₄octapa; H₄octapa-benzyl-NCS; H₄octapa-benzyl; TTHA; or a radionuclide complex thereof. In some embodiments, the radionuclide is copper-64 (⁶⁴Cu), gallium-68 (⁶⁸Ga), or technetium-99m (^{99m}Tc).

In some embodiments, a conjugate described herein is designed to have a prescribed elimination profile. The elimination profile can be designed by adjusting the sequence and length of the non-peptide ligand, the property of the linker, the type of radionuclide, etc. In some embodiments, the conjugate has an elimination half-life of about 5 minutes to about 12 hours. In some embodiments, the conjugate has an elimination half-life of about 10 minutes to about 8 hours. In some embodiments, the conjugate has an elimination half-life of at least about 15 minutes, at least about 30 minutes, at least about 1 hour, at least about 2 hours, at least about 3 hours, at least about 4 hours, at least about 5 hours, at least about 6 hours, at least about 8 hours. In some embodiments, the conjugate has an elimination half-life of at most about 15 minutes, at most about 30 minutes, at most about 1 hour, at most about 2 hours, at most about 3 hours, at most about 4 hours, at most about 5 hours, at most about 6 hours, or at most about 8 hours. In some embodiments, the elimination half-life is determined in rats. In some embodiments, the elimination half-life is determined in humans.

A herein described conjugate can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is about 15 minutes to about 1 day. In some embodiments, the elimination half-life of the conjugate in a tumor is at least 1.1, at least 1.2, at least 1.3, at least 1.4, at least 1.5, at least 2.0, at least 2.5, at least 3.0, at least 4.0, or at least 5.0-fold of the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the pharmaceutical in the living system. In some embodiments, the elimination half-life is measured as effective half-life, which is the half-life of a radiopharmaceutical in a living system taking into account the half-life of the radionuclide.

Response and toxicity prediction is essential for the rational implementation of cancer therapy. The biological effects of radionuclide therapy are mediated by a well-defined physical quantity, the absorbed dose (D), which is defined as the energy absorbed per unit mass of tissue.

Radiation dosimetry is the measurement, calculation and assessment of the ionizing radiation dose absorbed by an object, usually the human body, and may be thought of as the ability to perform the equivalent of a pharmacodynamic study in treated patients in real time. This applies both internally, due to ingested or inhaled radioactive substances, or externally due to irradiation by sources of radiation. Dosimetry analysis may be performed as part of patient treatment to calculate tumour versus normal organ absorbed dose and therefore the likelihood of treatment success.

A conjugate described herein can have a prescribed time-integrated activity coefficient (i.e., ã) in a tumor or non-tumor tissues of a subject. As used herein, ã represents the cumulative number of nuclear transformations occurring in a source tissue over a dose-integration period per unit administered activity. The ã value of a conjugate can be tuned by modifications of the NPDC. The ã value can be determined using a method known in the art. In some embodiments, the ã value of the conjugate in a tumor is from about 10 minutes to about 1 day. The ã value of the conjugate in a tumor can be the same as the ã value of the conjugate in a non-tumor tissue of the subject. The ã value of the conjugate in a tumor can be longer or shorter than the ã value of the conjugate in a non-tumor tissue of the subject. In some embodiments, the ã value of the conjugate in a tumor is at least 1.1, at least 1.2, at least 1.3, at least 1.4, at least 1.5, at least 2.0, at least 2.5, at least 3.0, at least 4.0, or at least 5.0-fold of the ã value of the conjugate in a non-tumor tissue of the subject.

A conjugate described herein can have an ã value in an organ of a subject. In some embodiments, the conjugate has an ã value in a kidney of the subject of at most 24 hours. In some embodiments, the ã value of the conjugate in a kidney of the subject is at most 18 hours, 15 hours, 12 hours, 10 hours, 8 hours, 6 hours, or 5 hours. In some embodiments, the ã value of the conjugate in a kidney of the subject is about 30 minutes to about 24 hours. In some embodiments, the ã value of the conjugate in a kidney of the subject is about 2 to 24 hours. In some embodiments, the ã value of the conjugate in a kidney of the subject is more than 24 hours. In some embodiments, the ã value of the conjugate in a liver of the subject is at most 24 hours. In some embodiments, the ã value of the conjugate in a liver of the subject is at most 18 hours, 15 hours, 12 hours, 10 hours, 8 hours, 6 hours, or 5 hours. In some embodiments, the ã value of the conjugate in a liver of the subject is about 30 minutes to about 24 hours. In some embodiments, the ã value of the conjugate in a liver of the subject is about 2 to 24 hours. In some embodiments, the ã value of the conjugate in a liver of the subject is more than 24 hours.

### Linkers

In some embodiments, the linker has a prescribed length thereby linking the melanocortin type 2 receptor (MC2R) targeting ligand and the chelating moiety or a radionuclide complex thereof (Q) while allowing an appropriate distance therebetween.

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker comprises one or more linear structures, one or more non-linear structures, one or more branched structures, one or more cyclic structures, one or more flexible moieties, one or more rigid moieties, or combinations thereof.

In some embodiments, a linker comprises one or more amino acid residues. In some embodiments, the linker comprises 1 to 3, 1 to 5, 1 to 10, 5 to 10, or 5 to 20 amino acid residues.

In some embodiments, one or more amino acids of the linker are unnatural amino acids.

In some embodiments, the linker comprises a peptide linkage. The peptide linkage comprises L-amino acids and/or D-amino acids. In some embodiments, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a linker has 1 to 10 O atoms, 1 to 5 O atoms, 1 to 3 O atoms, 1 to 20 atoms, 1 to 15 atoms, 1 to 1 O atoms, or 1 to 5 atoms in length. In some embodiments, the linker has 1 to 1 O atoms in length. In some embodiments, the linker has 1 to 2 O atoms in length.

In some embodiments, a linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences, proline-rich sequences, and regions rich in double and/or triple bonds.

In some embodiments, the cleavable linker comprises one or more of unsubstituted or substituted alkylene, unsubstituted or substituted cycloalkylene, unsubstituted or substituted heterocycloalkylene, unsubstituted or substituted arylene, and unsubstituted or substituted heteroarylene.

In some embodiments, the linker comprises a click chemistry residue. In some embodiments, the linker is attached to a non-peptide ligand, to a metal chelator or both via click chemistry. For example, in some embodiments, a non-peptide ligand comprises an azide group that reacts with an alkyne moiety of the linker. For another example, in some embodiments, a non-peptide ligand comprises an alkyne group that reacts with an azide of the linker. The metal chelator and the linker can be attached similarly. In some embodiments, the linker comprises an azide moiety, an alkyne moiety, or both. In some embodiments, the linker comprises a triazole moiety.

In some embodiments, the linker is designated as L³. In some embodiments, L³ is -L⁴-, -L⁵-, -L⁶-, -L⁷-, -L⁸-, -L⁹-, -L¹⁰-, -L⁴-L⁹-L¹⁰-, -L⁴-L⁵-L⁶-L⁷-L⁸-L⁹-L¹⁰-, or a combination thereof; wherein:
L⁴ is unsubstituted or substituted C₁-C₂₀alkylene, unsubstituted or substituted C₁-C₂₀heteroalkylene, unsubstituted or substituted C₂-C₂₀alkenylene, unsubstituted or substituted C₂-C₂₀alkynylene, C₄-C₂₀polyethylene glycol, —C(═O)—, —C(═O)NH—, —C(═O)-unsubstituted or substituted C₁-C₂₀alkylene, —C(═O)-unsubstituted or substituted C₁-C₂₀heteroalkylene, —C(═O)—C₄-C₂₀polyethylene glycol, —C(═O)NH-unsubstituted or substituted C₁-C₂₀alkylene, —C(═O)NH-unsubstituted or substituted C₁-C₂₀heteroalkylene, —C(═O)NH—C₄-C₂₀polyethylene glycol, —NHC(═O)-unsubstituted or substituted C₁-C₂₀alkylene, —NHC(═O)-unsubstituted or substituted C₁-C₂₀heteroalkylene, or —NHC(═O)—C₄-C₂₀polyethylene glycol;
L⁵ is absent, —S—S—, one or more independently selected natural or unnatural amino acids, wherein when 2 or more independently selected natural or unnatural amino acids are present the peptide that is formed is a linear or branched peptide;
L⁶ is absent, —O—, —S—, —S(═O)—, —S(═O)₂—, —NH—, —CH(OH)—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ᵥ—, —C(═O)—(CH₂CH₂X⁴)ᵥ—, or —(CH₂CH₂X⁴)ᵥ—, —C(═O)—(X⁴CH₂CH₂)ᵥ—, or —(X⁴CH₂CH₂)ᵥ—, each instance of v is independently 1, 2, 3, 4, 5, 6, 7, 8, 9, 10, 11, or 12;
each X⁴ is independently selected from O and NR^{X}; and each R^{X} is independently selected from hydrogen, C₁-C₄alkyl and —CH₂CO₂H;
L⁷ is absent, unsubstituted or substituted C₁-C₆alkylene, unsubstituted or substituted C₁-C₆heteroalkylene, unsubstituted or substituted C₂-C₆alkenylene, unsubstituted or substituted C₂-C₆alkynylene, unsubstituted or substituted cycloalkylene, unsubstituted or substituted heterocycloalkylene, unsubstituted or substituted arylene, unsubstituted or substituted heteroarylene,
L⁸ is absent, —[CH(R^{Y})]_{y}—, —(CH₂)_{y}—, —(X⁵CH₂CH₂)_{y}—, or —(CH₂CH₂X⁵)_{y}—, each y is 1, 2, 3, 4, 5, 6, 7, 8, 9, 10, 11, or 12; each R^{Y} is independently selected from hydrogen and —OH; each X⁵ is independently selected from O and NR^{X}; and each R^{X} is independently selected from hydrogen, C₁-C₄alkyl and —CH₂CO₂H;
L⁹ is absent, —(CH₂)—, —O—, —S—, —S(O)—, —S(O)₂—, —NH—, —CH(OH)—, —C(═O)—, —C(═O)NH—, —NHC(═O)—, —C(═S)NH—, —NHC(═S)—, —C(═O)O—, —OC(═O)—, —OC(═O)NH—, —NHC(═O)NH—, or —NHC(═O)O—; and
L¹⁰ is absent, unsubstituted or substituted C₁-C₆alkylene, or unsubstituted or substituted C₁-C₆heteroalkylene, or unsubstituted or substituted benzyl.

In some embodiments, L³ is -L⁴-, -L⁵-, -L⁶-, -L⁷-, -L⁸-, -L⁹-, -L¹⁰-, -L⁴-L⁹-L¹⁰-, -L⁴-L⁵-L⁶-L⁷-L⁸-L⁹-L¹⁰-, -L⁴-L⁶-L⁵-L⁷-L⁸-L⁹-L¹⁰-, -L⁶-L⁵-L⁷-L⁸-L⁹-L¹⁰-, or -L⁵-L⁷-L⁸-L⁹- L¹⁰. In some embodiments, L³ is -L⁴-L⁶-L⁵-L⁷-L⁸-L⁹-L¹⁰-, -L⁶-L⁵-L⁷-L⁸-L⁹-L¹⁰-, or -L⁵-L⁷-L⁸-L⁹-L¹⁰. In some embodiments, L³ is -L⁴-L⁶-L⁵-L⁷-L⁸-L⁹-L¹⁰-. In some embodiments, L³ is -L⁶-L⁵-L⁷-L⁸-L⁹-L¹⁰-. In some embodiments, L³ is or -L⁵-L⁷-L⁸-L⁹-L¹⁰.

In some embodiments, L³-Q is -L⁴-L⁶-L⁵-L⁷-L⁸-L⁹-L¹⁰-Q. In some embodiments, L³-Q is -L⁶-L⁵-L⁷-L-L⁹-L¹⁰-Q. In some embodiments, L³-Q is or -L⁵-L⁷-L⁸-L⁹-L¹⁰-Q. In some embodiments, L⁴ is —CH₂—, —CH₂CH₂—, —CH₂CH₂CH₂—, or —CH₂CH₂CH₂CH₂—.

In some embodiments, L⁵ is absent, aspartic acid, valine-citrulline, phenylalanine-lysine, valine-alanine, or glycine-glycine-phenylalanine-glycine.

In some embodiments, L⁵ is absent, alanine, arginine, asparagine, aspartate, cysteine, glutamine, glutamate, glycine, histidine, isoleucine, leucine, lysine, methionine, phenylalanine, proline, serine, threonine, tryptophan, tyrosine, valine, or combination thereof.

In some embodiments, L⁵ is absent, one or more independently selected natural or unnatural amino acids, wherein any free amine of an amino acid or amide bond linking the 2 or more amino acids is optionally independently substituted with —CH₃, and any peptide that is formed is a linear or branched peptide, and wherein any free amine of the amino acid or peptide is optionally substituted with —C(═O)—(CH₂)₁₋₄-(4-iodophenyl). In some embodiments, L⁵ is absent, glycine, glycine-glycine, glycine-glycine-glycine, alanine, alanine-alanine, alanine-alanine-alanine, sarcosine, sarcosine-sarcosine, sarcosine-sarcosine-sarcosine, aspartic acid, glutamic acid, lysine, glutamic acid-glutamic acid, glutamic acid-lysine, valine-citrulline, phenylalanine-lysine, valine-alanine, or glycine-glycine-phenylalanine-glycine; wherein any free amine of the amino acid or peptide is optionally substituted with —C(═O)—(CH₂)₁₋₄-(4-iodophenyl).

In some embodiments, L⁵ is absent, one or more independently selected natural or unnatural amino acids, wherein any free amine of an amino acid or amide bond linking 2 or more amino acids is optionally independently substituted with —CH₃, and any peptide that is formed is a linear or branched peptide, and wherein any free amine of the amino acid or peptide is optionally substituted with —C(═O)—(CH₂)₁₋₄-(4-iodophenyl); wherein the one or more independently selected natural or unnatural amino acids are independently selected from the group consisting of Glycine (Gly), Alanine (Ala), Valine (Val), Leucine (Leu), Isoleucine (Ile), Homoalanine (HAla), Norvaline (Nva), Norleucine (Nle), tert-Leucine (Tle), Aspartic Acid (Asp), Glutamic acid (Glu), Glutamine (Gln), Asparagine (Asn), Lysine (Lys), Homolysine (HLys), Ornithine (Om), Methionine (Met), Cysteine (Cys), Homocysteine (HCys), Homoserine (HSer), Threonine (Thr), Serine (Ser), Histidine (His), Tryptophan (Trp), 7-aza-Trp, 1-methyltryptophan (1MT), Phenylalanine (Phe), Tyrosine (Tyr), Homophenylalanine (HPhe), 4-cyano phenylalanine (Phe(4-CN), Homotyrosine (HTyr), Arginine (Arg), Arg(Me), Citrulline (Cit), Homoarginine (HArg), Methyl homoarginine (homo-Arg(Me)), Norarginine (AGBA), Canavanine, Methyl Citrulline (Cit(Me)), Methyl Norarginine (AGBA(Me)), Methyl Canavanine, Biphenylalanine (Bip), Phenylglycine (Phg), Cyclohexylalanine (Cha), Cyclohexylglycine (Chg), Azaglycine (AzaGly), 2-Amino-2-indancarboxylic acid (Aic), Sarcosine (Sar), 3-sulfo-alanine (Ala-SO₃H), β-alanine (bAla), β-(2-thienyl)-Ala, β³-homoserine, β³-homolysine, β³-homoglutamic acid.

In some embodiments, L⁷ is absent, unsubstituted or substituted C₁-C₆alkylene, unsubstituted or substituted C₁-C₆heteroalkylene, unsubstituted or substituted cyclohexylene, unsubstituted or substituted piperidinylene, unsubstituted or substituted phenylene, or unsubstituted or substituted pyridinylene.

In some embodiments, L⁸ is absent, —[CH(R^{Y})]_{y}—, —(CH₂)_{y}—, —(NR^{X}CH₂CH₂)—(OCH₂CH₂)_{y}—, each y is 1, 2, 3, 4, 5, 6, 7 or 8; each R^{Y} is independently selected from hydrogen and —OH; R^{X} is selected from hydrogen, C₁-C₄alkyl and —CH₂CO₂H.

In some embodiments, -L⁹-L¹⁰- is —NHC(═O)—C₁-C₂alkylene.

In some embodiments, L⁴ is —CH₂—, —CH₂CH₂—, —CH₂CH₂CH₂—, or —CH₂CH₂CH₂CH₂—, and L⁶ is absent, —O—, —NH—, —CH(OH)—, —NHC(═O)—, —(CH₂)ᵥ—, —C(═O)—(CH₂CH₂O)ᵥ—, —C(═O)—(CH₂CH₂O)ᵥ—(CH₂CH₂NR^{X})—, —(CH₂CH₂O)ᵥ—, —(CH₂CH₂NR^{X})—(CH₂CH₂O)ᵥ—, —(OCH₂CH₂)ᵥ—, —(NR^{X}CH₂CH₂)—(OCH₂CH₂)ᵥ—, wherein each instance of v is independently 1, 2, 3, 4, 5, 6, 7, or 8.

In some embodiments, L³ is -L⁴-L⁹-L¹⁰-; L⁴ is unsubstituted or substituted C₁-C₂₀alkylene, C₄-C₂₀polyethylene glycol, —C(═O)-unsubstituted or substituted C₁-C₂₀alkylene, —C(═O)—C₄-C₂₀polyethylene glycol, —C(═O)NH-unsubstituted or substituted C₁-C₂₀alkylene, —C(═O)NH—C₄-C₂₀polyethylene glycol, —NHC(═O)-unsubstituted or substituted C₁-C₂₀alkylene, or —NHC(═O)—C₄-C₂₀polyethylene glycol; L⁹ is —C(═O)NH—, —NHC(═O)—, —C(═S)NH—, or —NHC(═S)—; and L¹⁰ is C₁-C₂alkylene or benzyl.

In some embodiments, L³ comprises -L⁹-L¹⁰-, and -L⁹-L¹⁰- is —NHC(═O)—C₁-C₂alkylene.

In some embodiments, L³ comprises -L⁹-L¹⁰-, and -L⁹-L¹⁰- is —NHC(═O)—CH₂— or —NHC(═O)—CH₂CH₂—.

In some embodiments, L³ is -L⁵-L⁷-L⁸-L⁹-L¹⁰-, wherein L⁵ is absent, one or more independently selected natural or unnatural amino acids, wherein any free amine of an amino acid or amide bond linking the 2 or more amino acids is optionally independently substituted with —CH₃, and any peptide that is formed is a linear or branched peptide, and wherein any free amine of the amino acid or peptide is optionally substituted with —C(═O)—(CH₂)₁₋₄-(4-iodophenyl); L⁷ is absent, unsubstituted or substituted C₁-C₆alkylene, unsubstituted or substituted C₁-C₆heteroalkylene, unsubstituted or substituted cycloalkylene, unsubstituted or substituted heterocycloalkylene, unsubstituted or substituted phenylene, unsubstituted or substituted heteroarylene, L⁸ is absent, —[CH(R^{Y})]_{y}—, —(CH₂)_{y}—, —(NR^{X}CH₂CH₂)—(OCH₂CH₂)_{y}—, each y is 1, 2, 3, 4, 5, 6, 7 or 8; each R^{Y} is independently selected from hydrogen and —OH; R^{X} is selected from hydrogen, C₁-C₄alkyl and —CH₂CO₂H; and -L⁹-L¹⁰- is —NHC(═O)—C₁-C₂alkylene-.

In some embodiments, L⁵ is

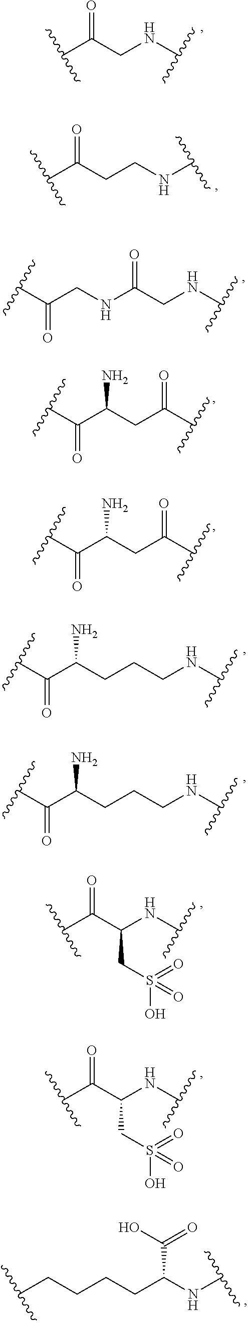

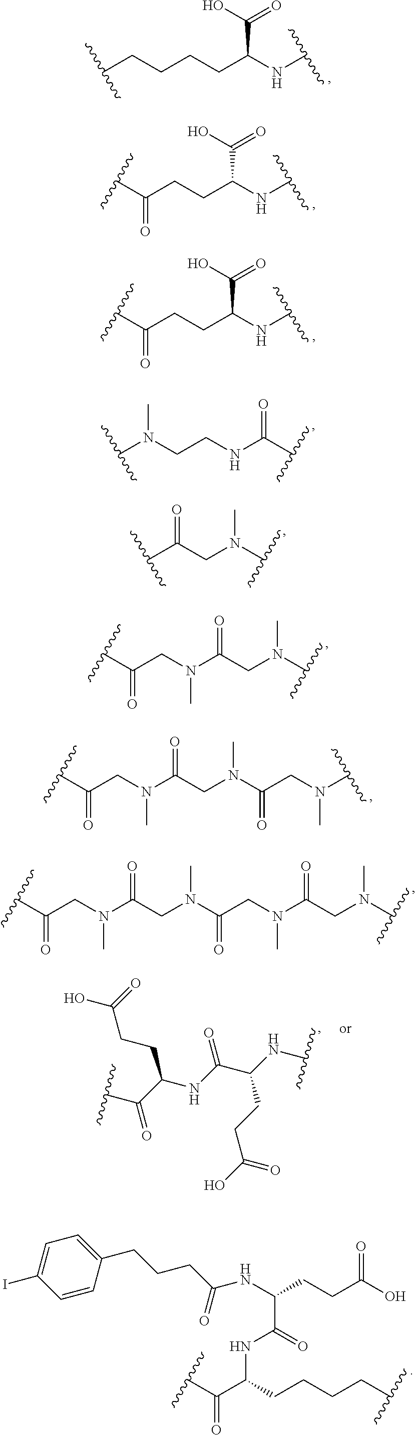

In some embodiments, L³ is:

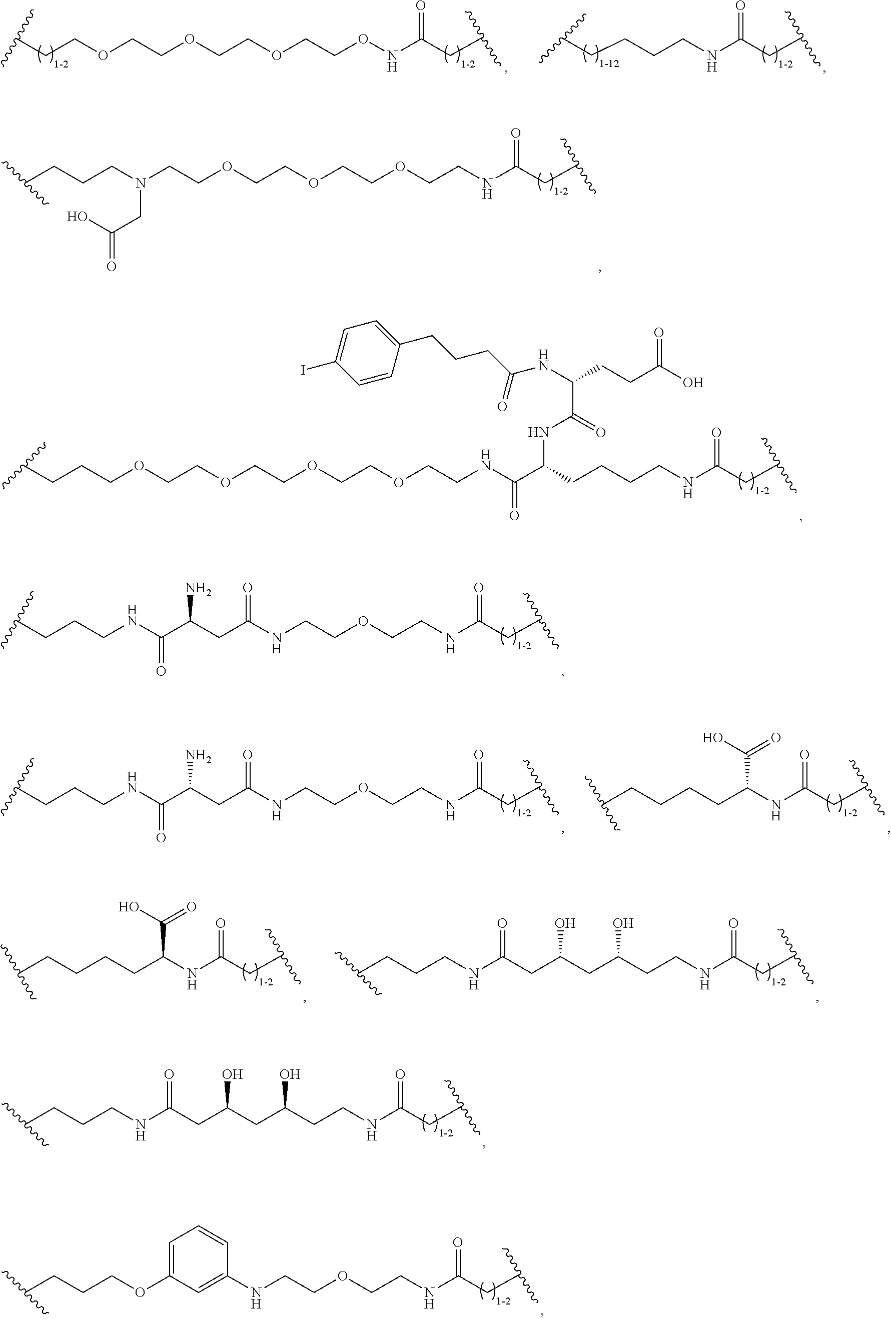

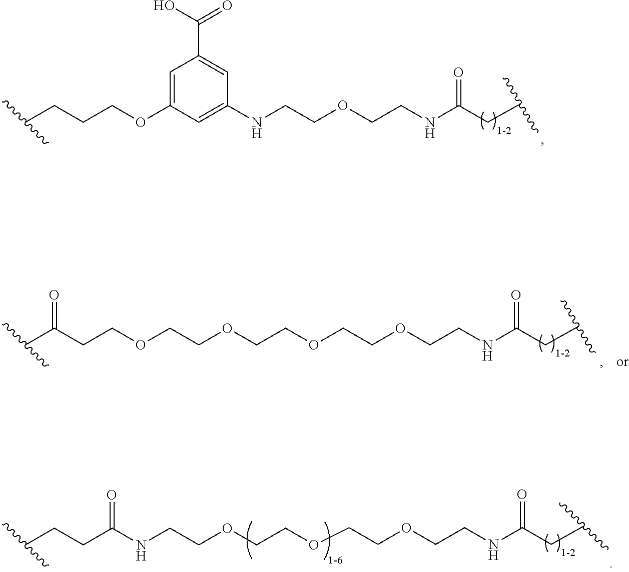

In some embodiments, L³ is

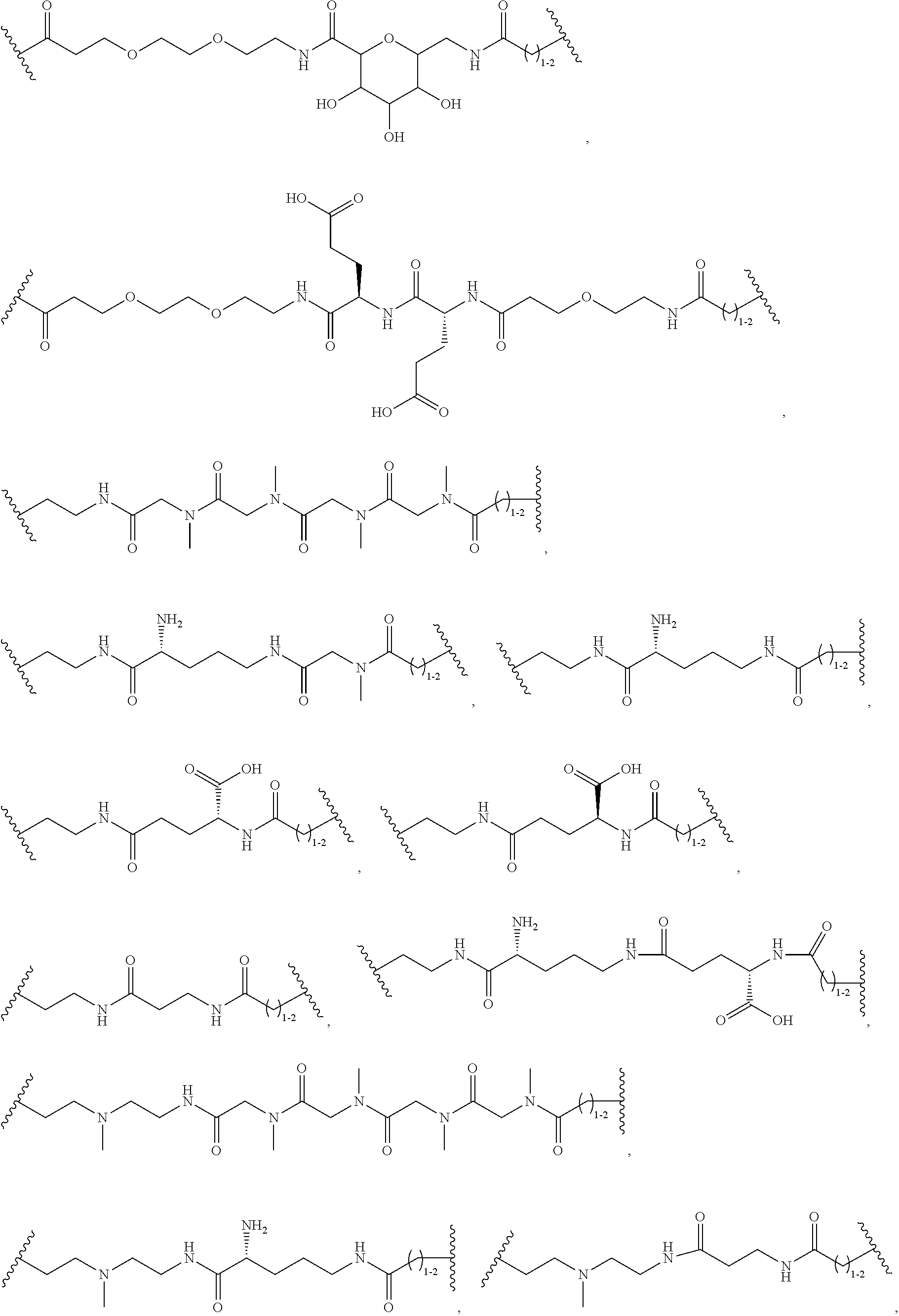

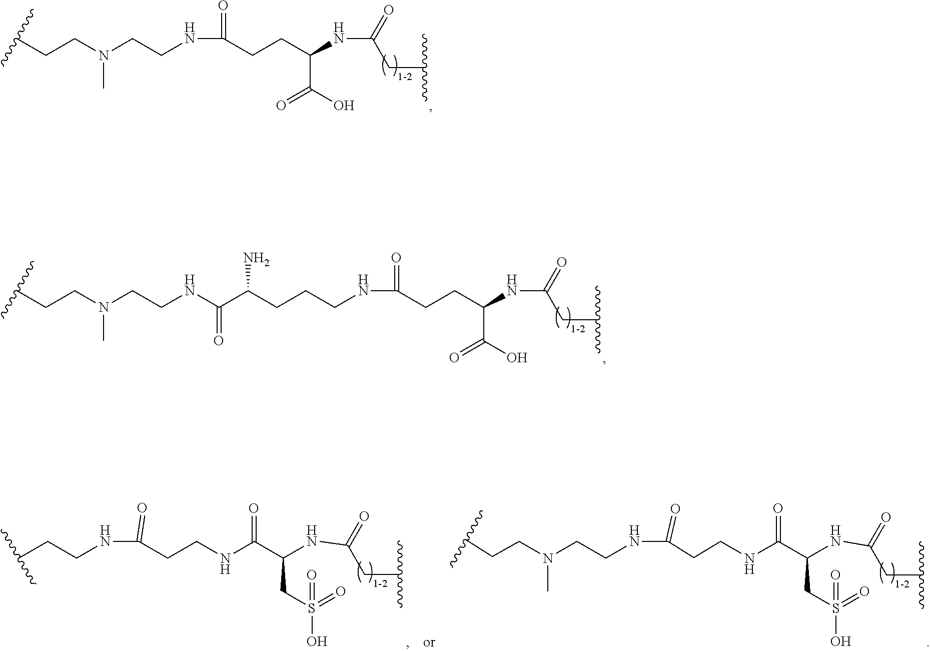

In some embodiments, L³-Q is:

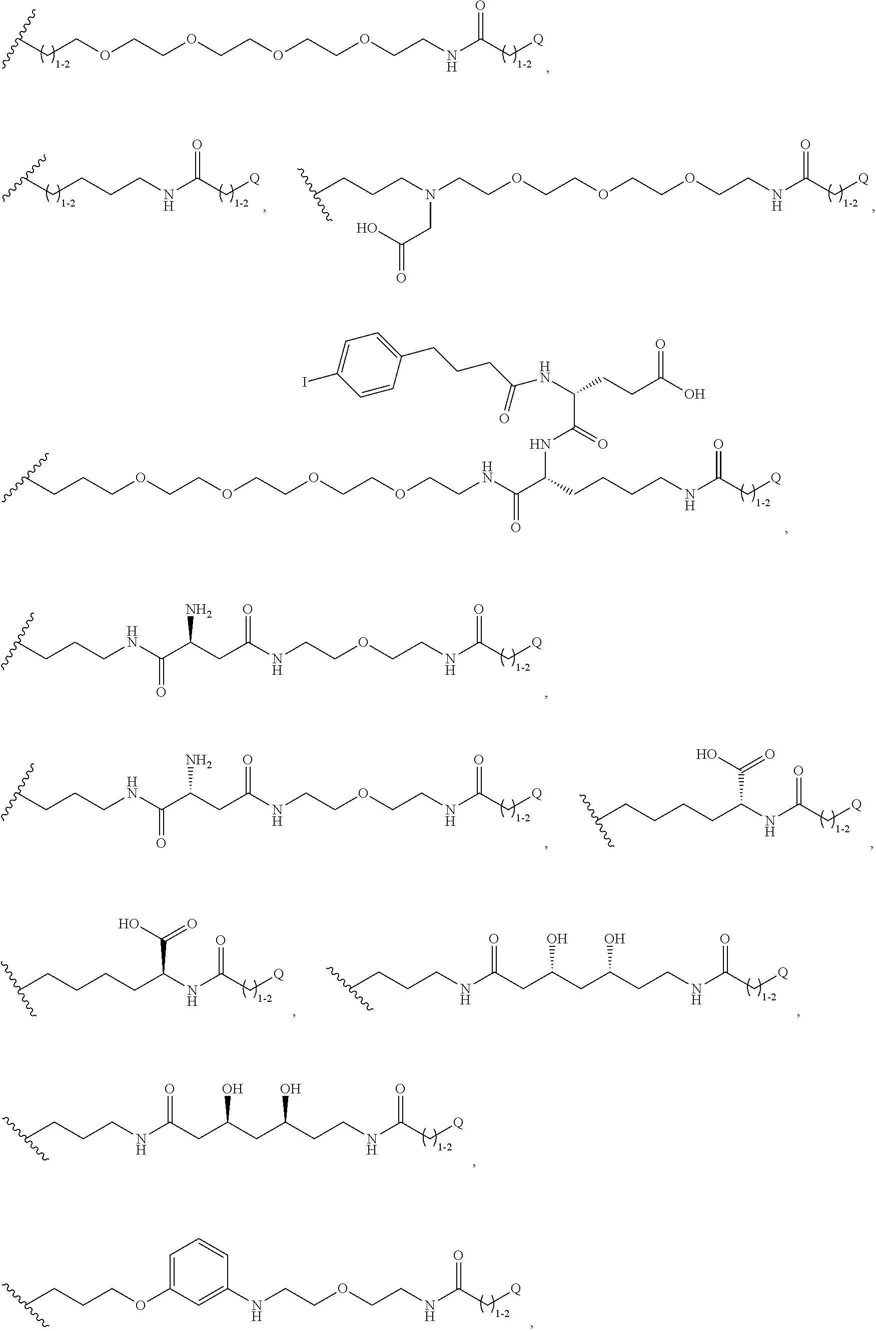

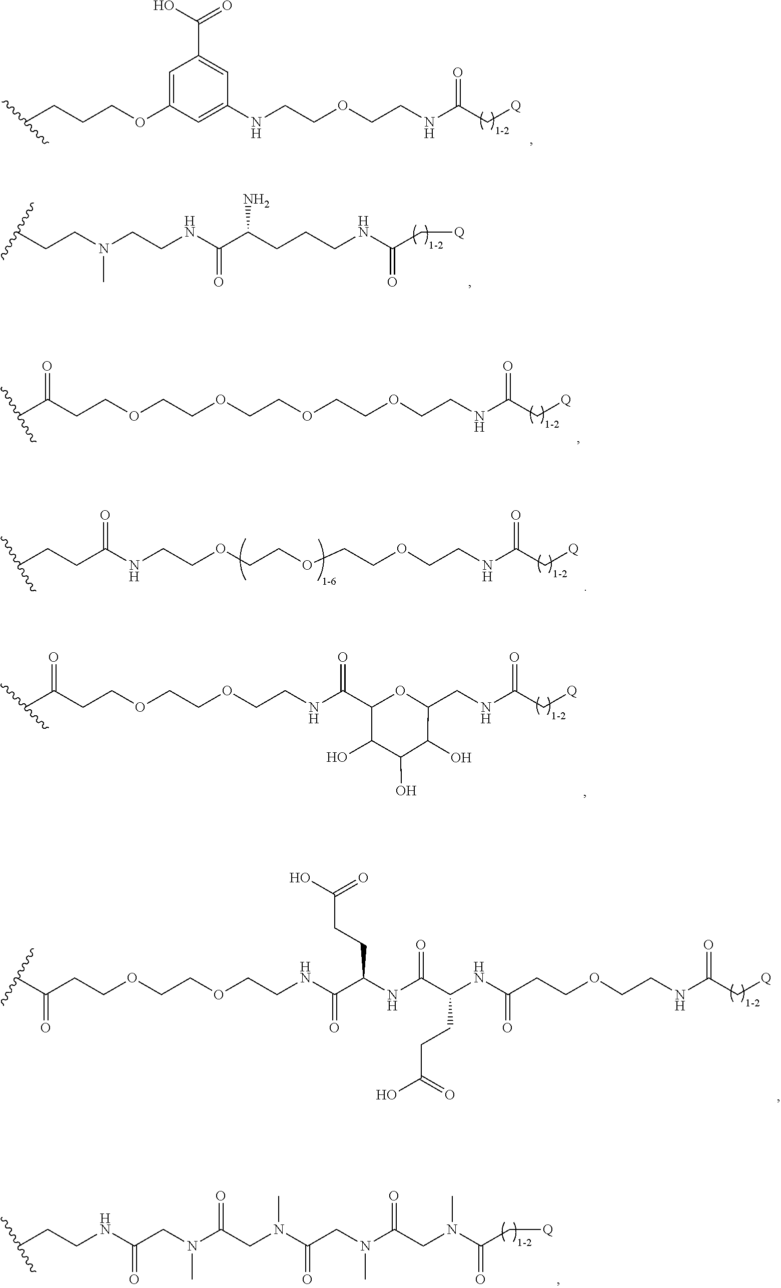

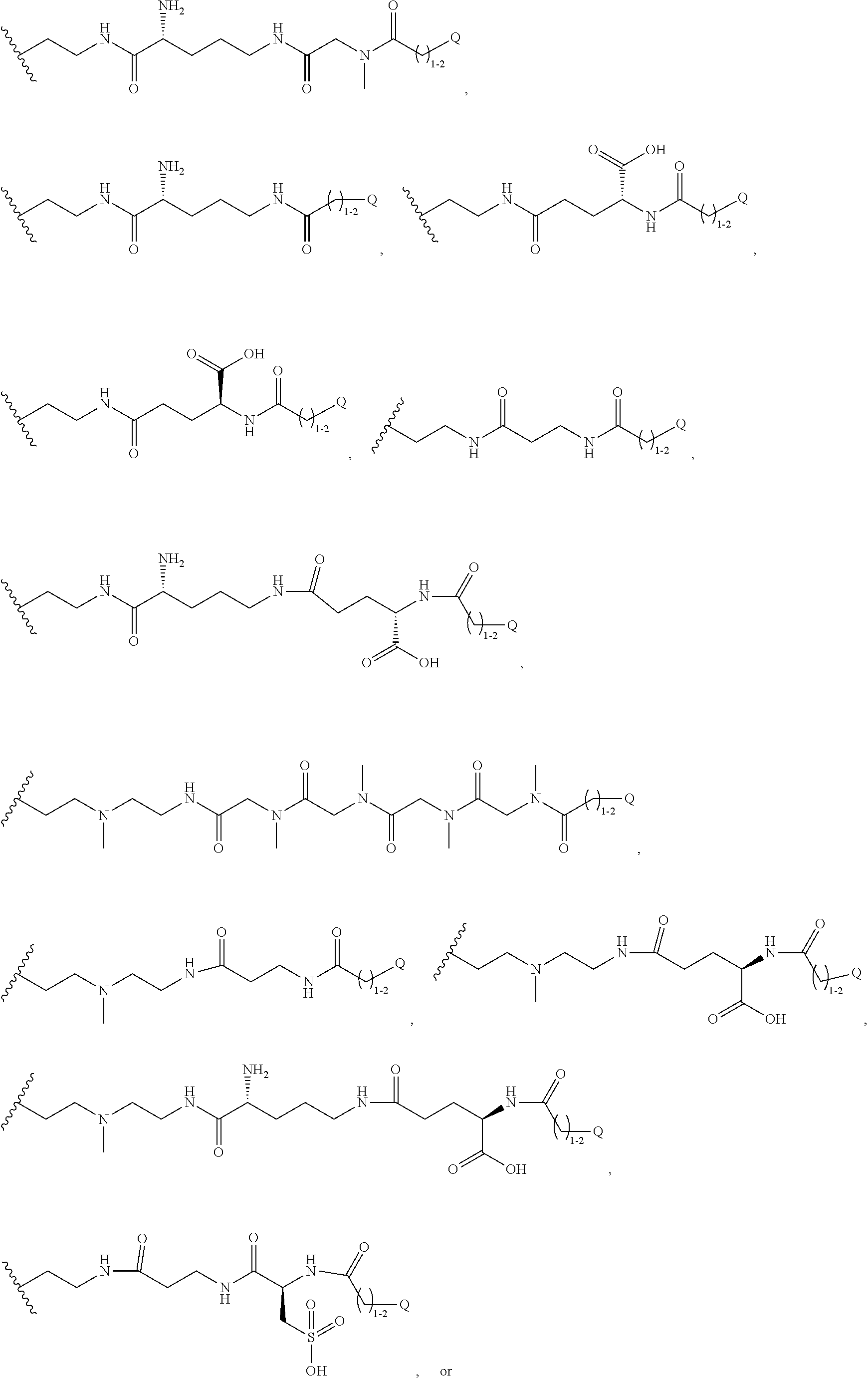

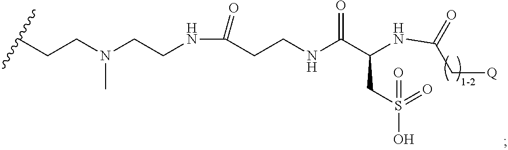

### Q is

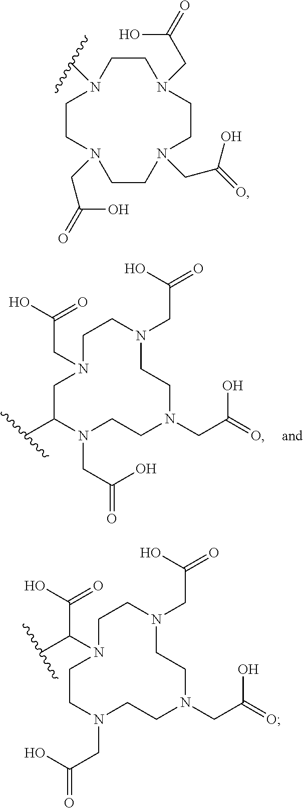 [SMILES: *N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

### Representative Linker and Chelating Moieties

In some embodiments, -L³-Q- is: —(CH₂)ᵥ(CH₂)_{y}NHC(═O)CH₂Q, —(CH₂)ᵥ(OCH₂CH₂)_{y}NHC(═O)CH₂Q, —C(═O)(CH₂)ᵥ(CH₂)_{y}NHC(═O)CH₂Q, —C(═O)(CH₂)ᵥ(OCH₂CH₂)_{y}NHC(═O)CH₂Q, —(CH₂)ᵥNHC(═O)[CH(R^{Y})]_{y}NHC(═O)CH₂Q,
—C(═O)CH(NH₂)CH₂C(═O)NHCH₂CH₂OCH₂CH₂NHC(═O)CH₂-Q, —C(═O)[CH(R^{Y})]_{y}NHC(═O)CH₂Q,
—(CH₂)ᵥ—O-substituted or unsubstituted phenylene-NHCH₂CH₂OCH₂CH₂NHC(═O)CH₂-Q;
—(CH₂)ᵥ(OCH₂CH₂)_{y}NHC(═O)CHR^{z}(CH₂)_{y}NHC(═O)CH₂Q or —(C₂-C₄alkylene)(NR^{X}CH₂CH₂)ᵥ(OCH₂CH₂)_{y}NHC(═O)CH₂Q;
v is 1, 2, 3, 4, 5, 6, 7, 8, 9, 10, 11, or 12; y is 1, 2, 3, 4, 5, or 6;
each R^{Y} is independently selected from hydrogen and —OH; each R^{X} is independently selected from hydrogen, C₁-C₄alkyl and —CH₂CO₂H; R^{Z} is a substituted or unsubstituted branched peptide;
and Q is

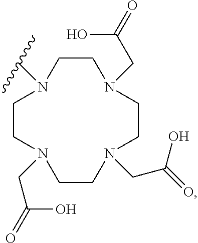 [SMILES: *N1CCN(CC(=O)O)CCN(CC(=O)O)CCN(CC(=O)O)CC1]

or a radionuclide complex thereof.

In some embodiments, -L³-Q- is: —(CH₂)ᵥ(CH₂)₆NHC(═O)CH₂Q, —CH₂CH₂(OCH₂CH₂)₄NHC(═O)CH₂Q, —CH₂CH₂CH₂(OCH₂CH₂)₄NHC(═O)CH₂Q, —C(═O)(CH₂)ᵥ(CH₂)₆NHC(═O)CH₂Q, —C(═O)CH₂CH₂(OCH₂CH₂)₄NHC(═O)CH₂Q, —C(═O)CH(NH₂)CH₂C(═O)NHCH₂CH₂OCH₂CH₂NHC(═O)CH₂-Q, —C(═O)[CH₂CH(OH)]2CH₂CH₂NHC(═O)CH₂Q, —(CH₂)₄CH(CO₂H)NHC(═O)CH₂Q, —(CH₂CH₂CH₂)—O-substituted or unsubstituted phenylene-NHCH₂CH₂OCH₂CH₂NHC(═O)CH₂-Q, —(CH₂CH₂CH₂)(OCH₂CH₂)₄NHC(═O)CHR^{z}(CH₂)₄NHC(═O)CH₂Q, or —(C₂-C₄alkylene)N(CH₂CO₂H)CH₂CH₂(OCH₂CH₂)₃NHC(═O)CH₂Q;
v is 1, 2, 3, 4, 5, 6, 7, 8, 9, 10, 11, or 12; R^{z} is a branched peptide substituted with —C(═O)—(CH₂)₄-(4-iodophenyl); and
Q is

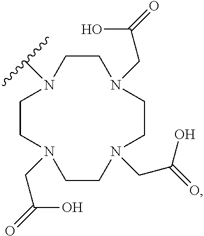 [SMILES: *N1CCN(CC(=O)O)CCN(CC(=O)O)CCN(CC(=O)O)CC1]

or a radionuclide complex thereof.

In some embodiments, -L³-Q- is: —(CH₂)ᵥ(CH₂)_{y}NHC(═O)CH₂CH₂Q, —(CH₂)ᵥ(OCH₂CH₂)_{y}NHC(═O)CH₂CH₂Q, —C(═O)(CH₂)ᵥ(CH₂)_{y}NHC(═O)CH₂CH₂Q, —C(═O)(CH₂)ᵥ(OCH₂CH₂)_{y}NHC(═O)CH₂CH₂Q, —C(═O)CH(NH₂)CH₂C(═O)NHCH₂CH₂OCH₂CH₂NHC(═O)CH₂CH₂-Q, —(CH₂)ᵥNHC(═O)[CH(R^{Y})]_{y}NHC(═O)CH₂CH₂Q, —(CH₂)ᵥ—O-substituted or unsubstituted phenylene-NHCH₂CH₂OCH₂CH₂NHC(═O)CH₂-Q; —(CH₂)ᵥ(OCH₂CH₂)_{y}NHC(═O)CHR^{z}(CH₂)_{y}NHC(═O)CH₂Q or
(C₂-C₄alkylene)(NR^{X}CH₂CH₂)ᵥ(OCH₂CH₂)_{y}NHC(═O)CH₂CH₂Q;
v is 1, 2, 3, 4, 5, 6, 7, 8, 9, 10, 11, or 12; y is 1, 2, 3, or 4;
each R^{Y} is independently selected from hydrogen and —OH; each R^{X} is independently selected from hydrogen, C₁-C₄alkyl and —CH₂CO₂H; R^{Z} is a substituted or unsubstituted branched peptide;

### and Q is

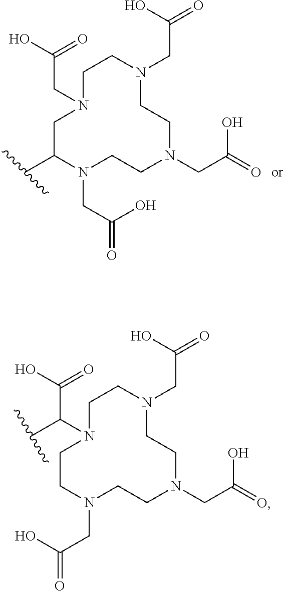 [SMILES: *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, -L³-Q- is: —(CH₂)ᵥ(CH₂)₆NHC(═O)CH₂CH₂Q, —CH₂CH₂(OCH₂CH₂)₄NHC(═O)CH₂CH₂Q, —CH₂CH₂CH₂(OCH₂CH₂)₄NHC(═O)CH₂CH₂Q, —C(═O)(CH₂)ᵥ(CH₂)₆NHC(═O)CH₂CH₂Q, —C(═O)CH₂CH₂(OCH₂CH₂)₄NHC(═O)CH₂CH₂Q, —C(═O)CH(NH₂)CH₂C(═O)NHCH₂CH₂OCH₂CH₂NHC(═O)CH₂CH₂Q, —C(═O)[CH₂CH(OH)]2CH₂CH₂NHC(═O)CH₂CH₂Q, —(CH₂)₄CH(CO₂H)NHC(═O)CH₂CH₂Q, —(CH₂CH₂CH₂)—O-substituted or unsubstituted phenylene-NHCH₂CH₂OCH₂CH₂NHC(═O)CH₂-Q, —(CH₂CH₂CH₂)(OCH₂CH₂)₄NHC(═O)CHR^{z}(CH₂)₄NHC(═O)CH₂Q, or —(C₂-C₄alkylene)N(CH₂CO₂H)CH₂CH₂(OCH₂CH₂)₃NHC(═O)CH₂CH₂Q; v is 1, 2, 3, 4, 5, 6, 7, 8, 9, 10, 11, or 12; R^{z} is a branched peptide substituted with —C(═O)—(CH₂)₄-(4-iodophenyl); and

### Q is

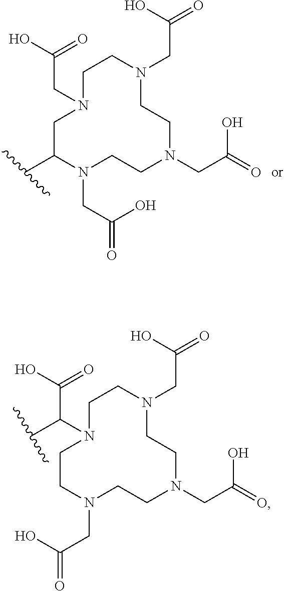 [SMILES: *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, -L³-Q is:

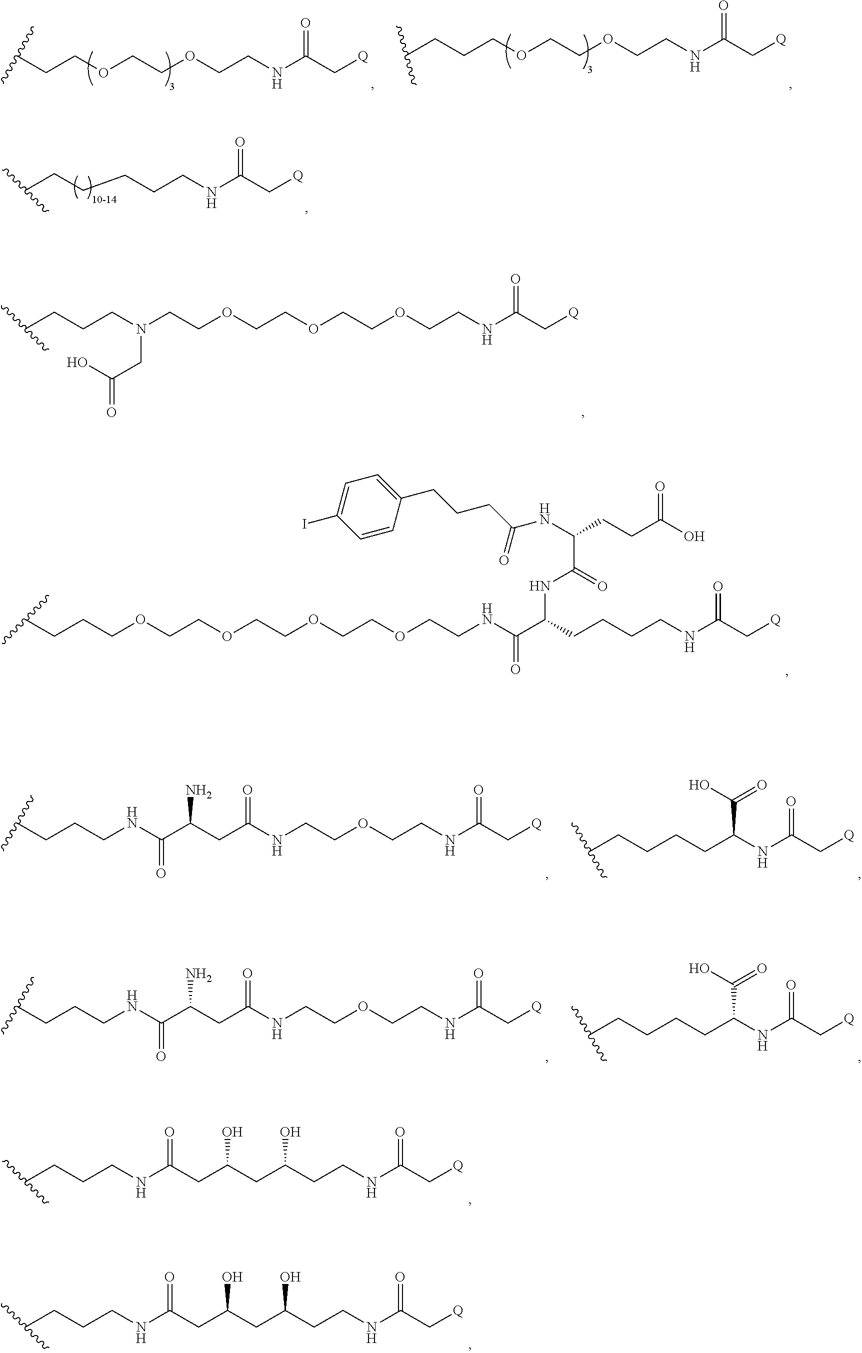

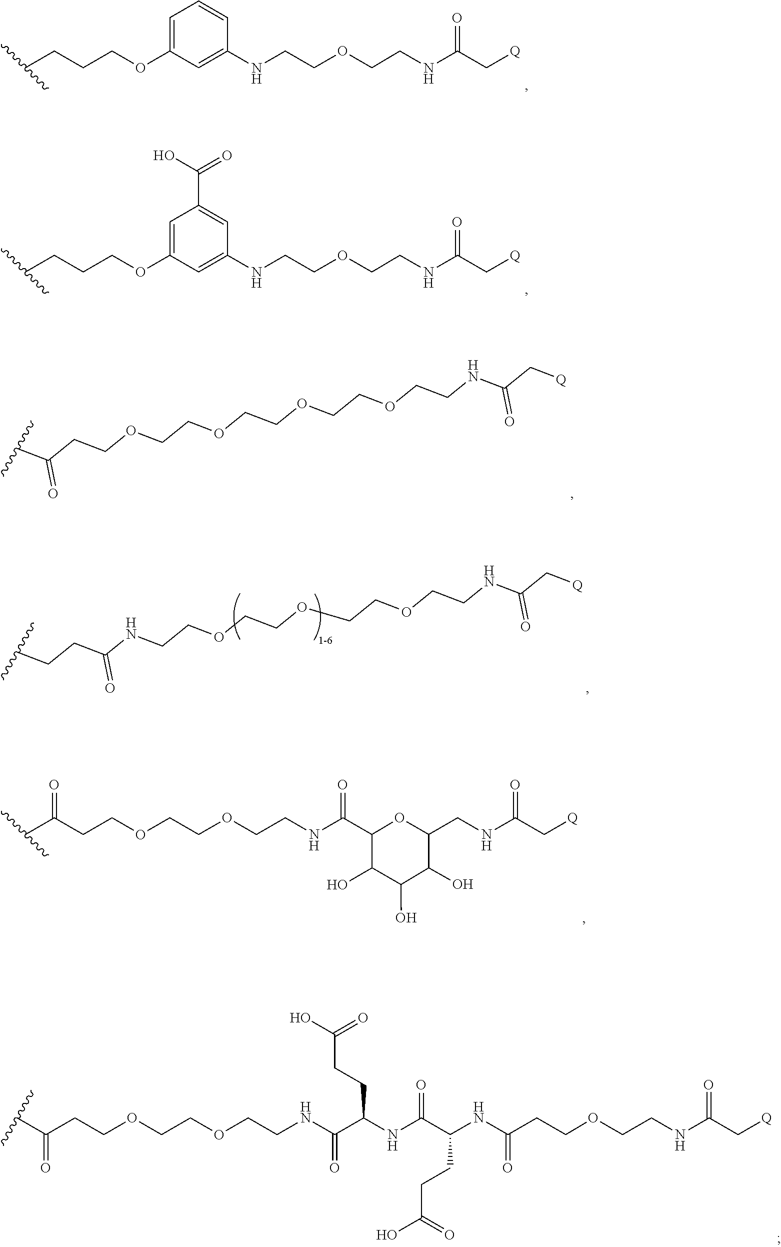

Q is

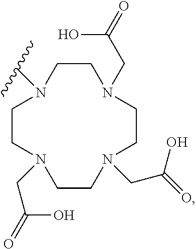 [SMILES: *N1CCN(CC(=O)O)CCN(CC(=O)O)CCN(CC(=O)O)CC1]

or a radionuclide complex thereof.

In some embodiments, L² is absent, —CH₂CH₂NH—, —CH₂CH₂CH₂NH—,

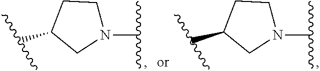 [SMILES: *[C@@H]1CCN(*)C1 ; *[C@H]1CCN(*)C1]

and -L³-Q is as defined in the previous paragraph.

In some embodiments, -L³-Q is

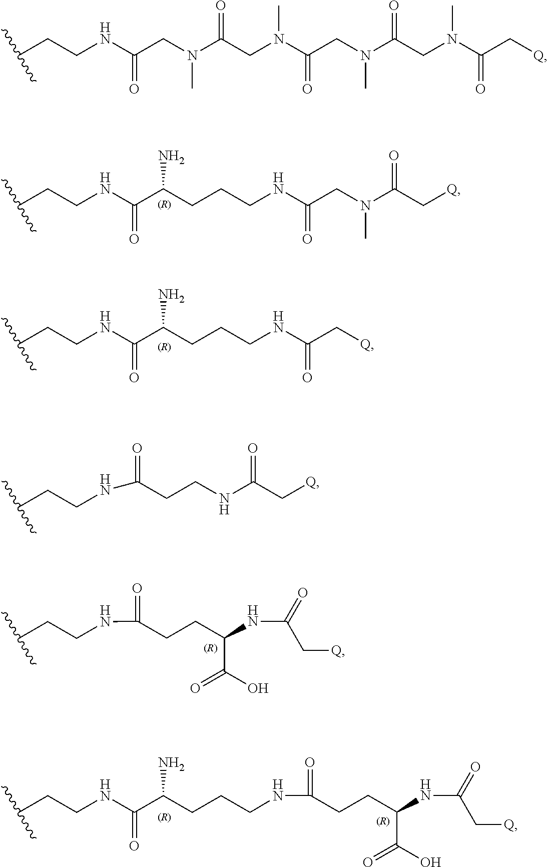

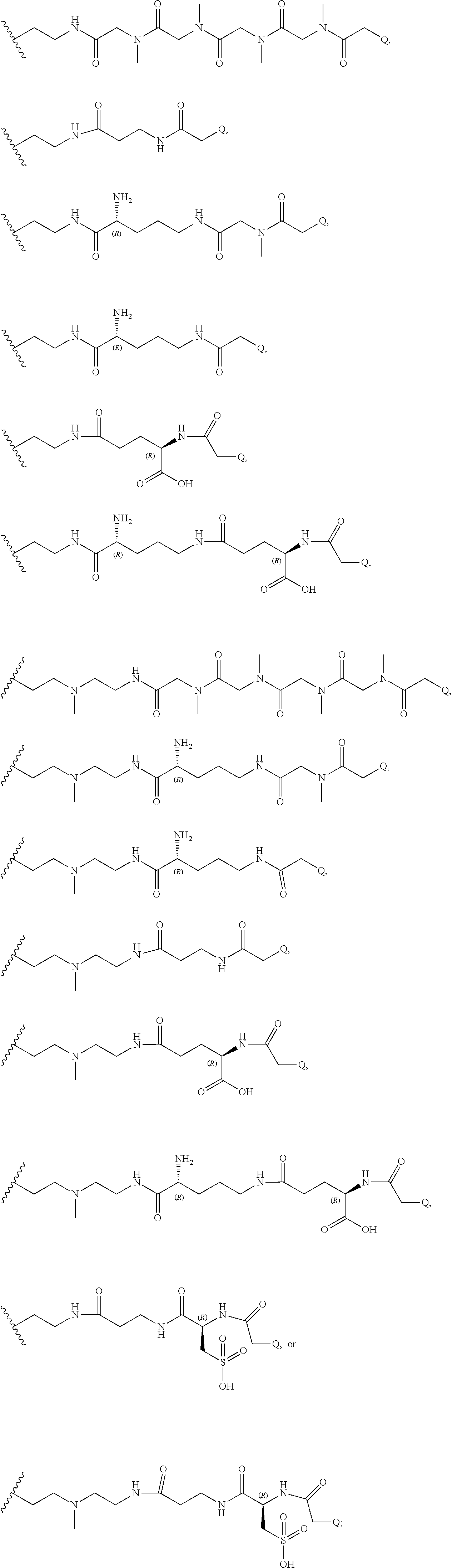

Q is

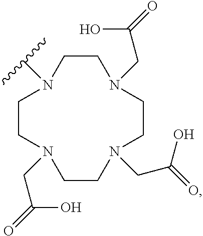 [SMILES: *N1CCN(CC(=O)O)CCN(CC(=O)O)CCN(CC(=O)O)CC1]

or a radionuclide complex thereof.

In some embodiments, L is absent, —CH₂CH₂NH—, —CH₂CH₂CH₂NH—,

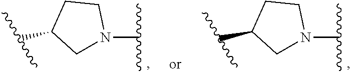 [SMILES: *[C@@H]1CCN(*)C1 ; *[C@H]1CCN(*)C1]

and -L³-Q is as defined in the previous paragraph.

### Representative Compounds

In some embodiments, the compound of Formula (I) has one of the following structures, or a pharmaceutically acceptable salt thereof:

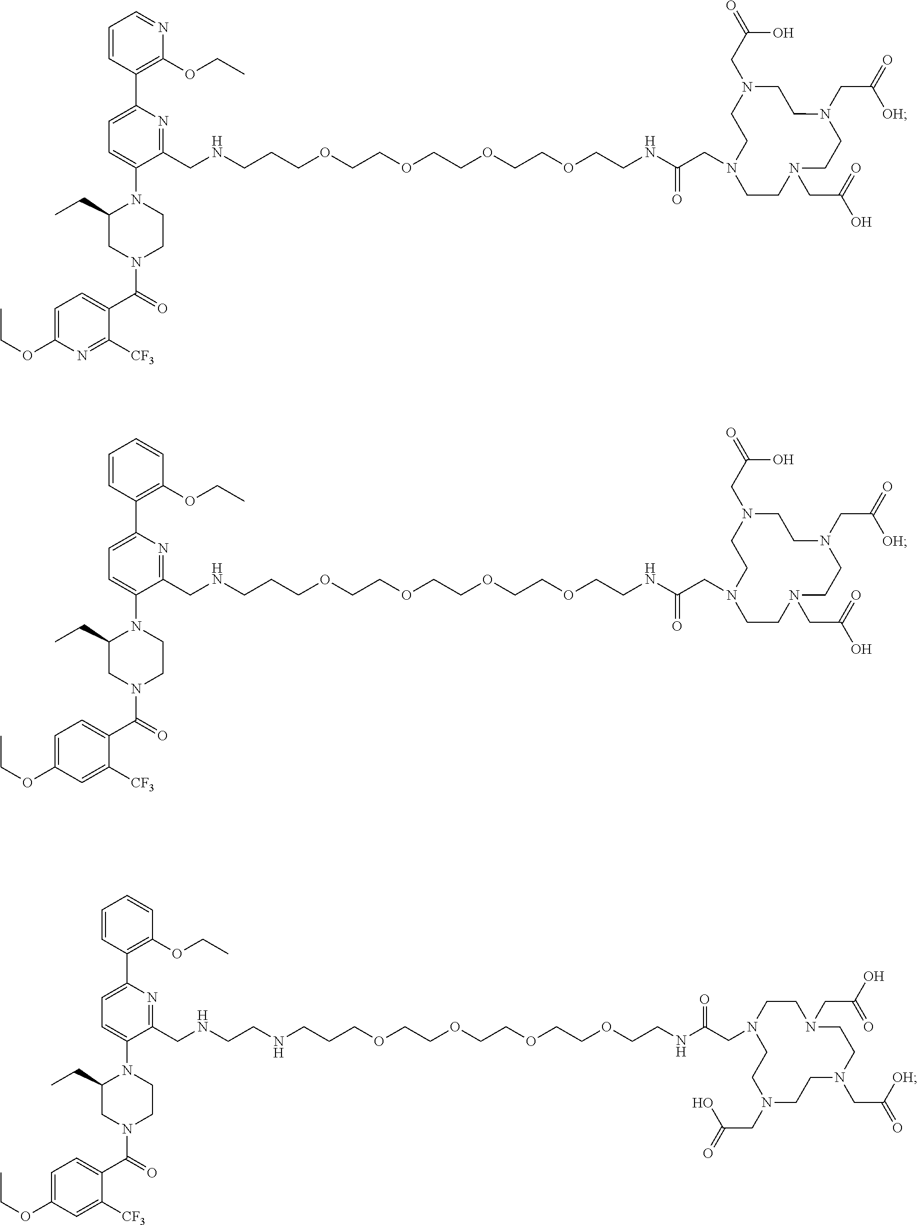 [SMILES: CCOc1ccc(C(=O)N2CCN(c3ccc(-c4cccnc4OCC)nc3CNCCCOCCOCCOCCOCCNC(=O)CN3CCN(CC(=O)O)CCN(CC(=O)O)CCN(CC(=O)O)CC3)[C@H](CC)C2)c(C(F)(F)F)n1 ; CCOc1ccc(C(=O)N2CCN(c3ccc(-c4ccccc4OCC)nc3CNCCCOCCOCCOCCOCCNC(=O)CN3CCN(CC(=O)O)CCN(CC(=O)O)CCN(CC(=O)O)CC3)[C@H](CC)C2)c(C(F)(F)F)c1 ; CCOc1ccc(C(=O)N2CCN(c3ccc(-c4ccccc4OCC)nc3CNCCNCCCOCCOCCOCCOCCNC(=O)CN3CCN(CC(=O)O)CCN(CC(=O)O)CCN(CC(=O)O)CC3)[C@H](CC)C2)c(C(F)(F)F)c1]

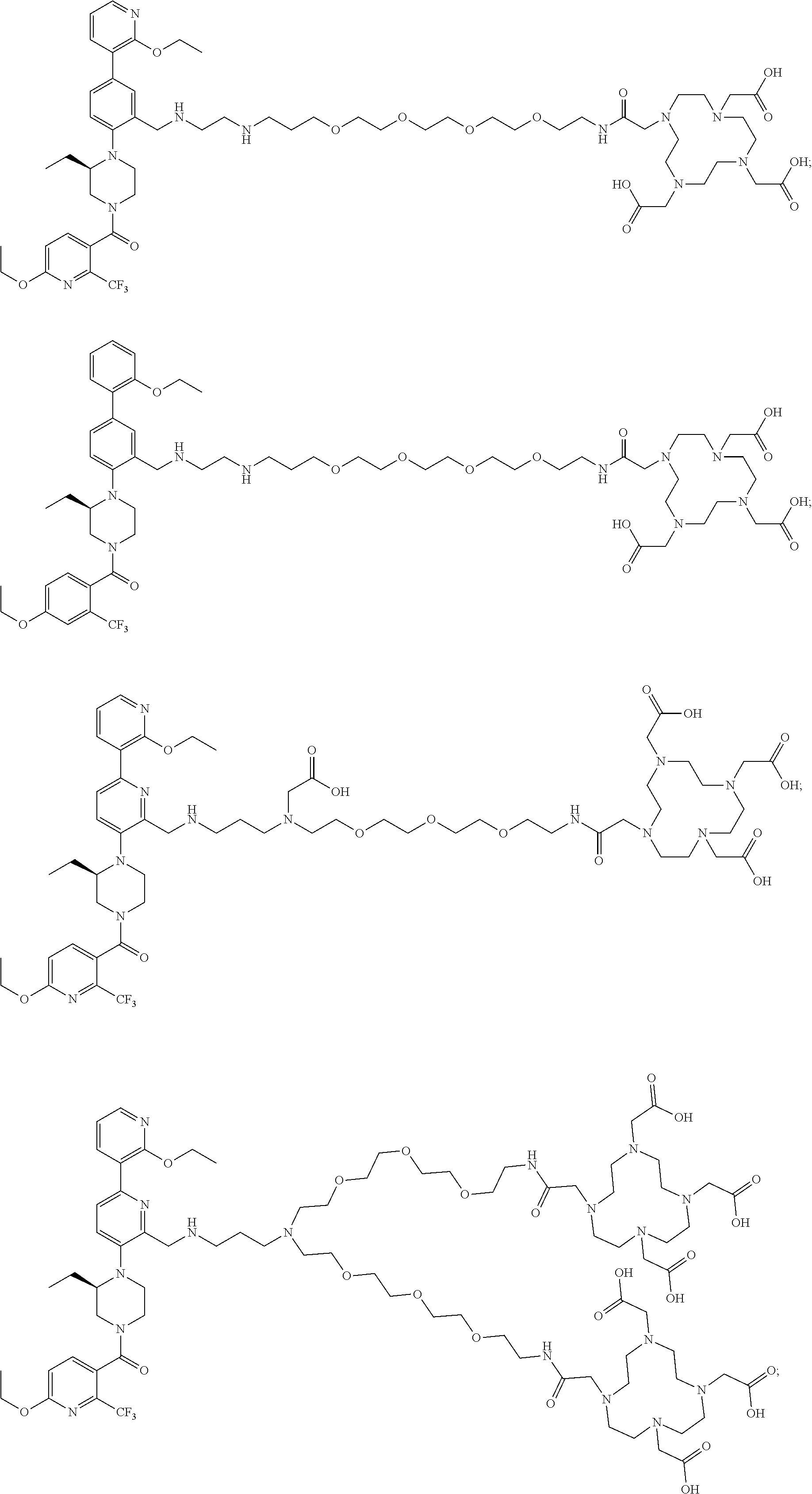

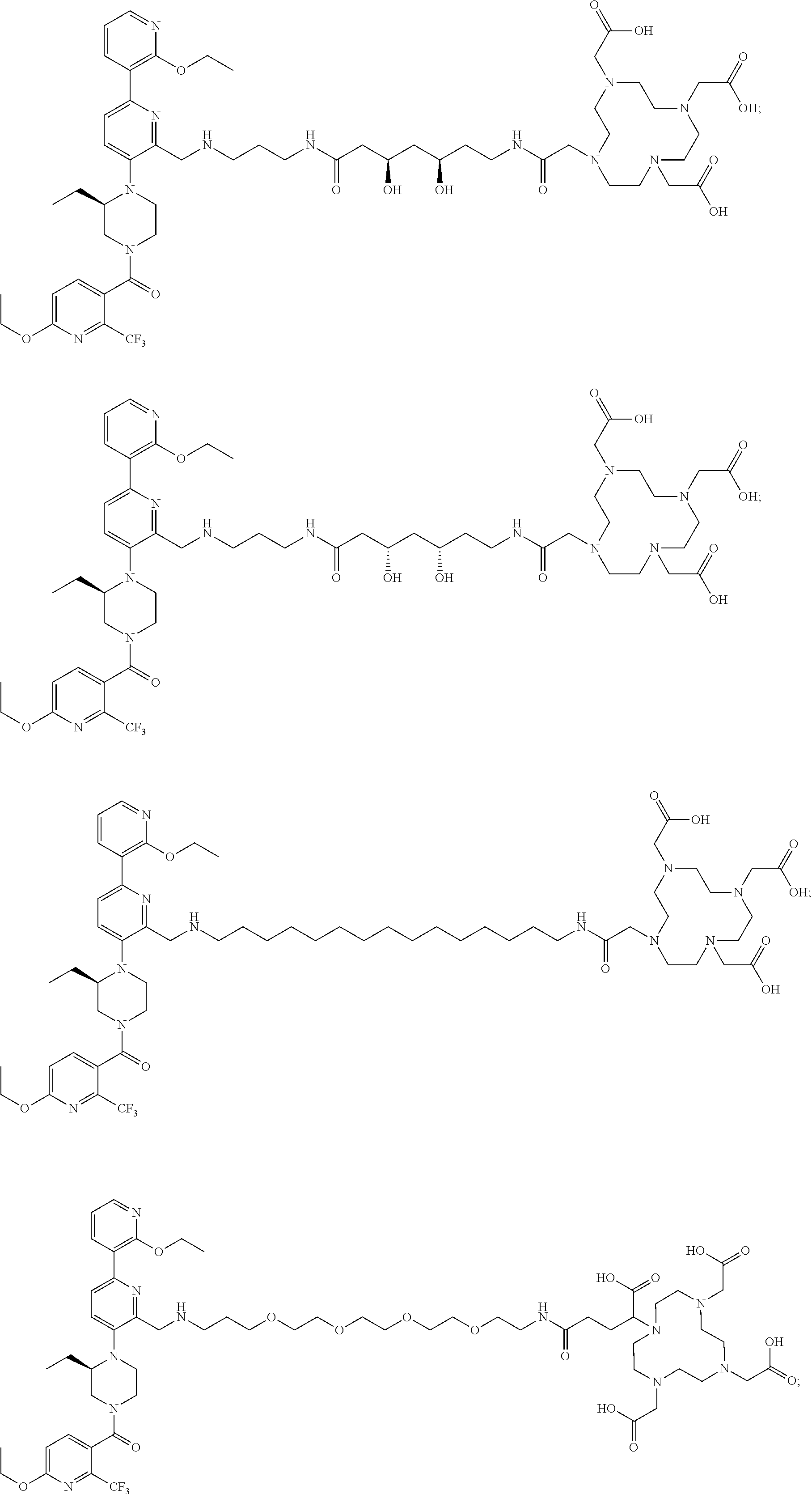

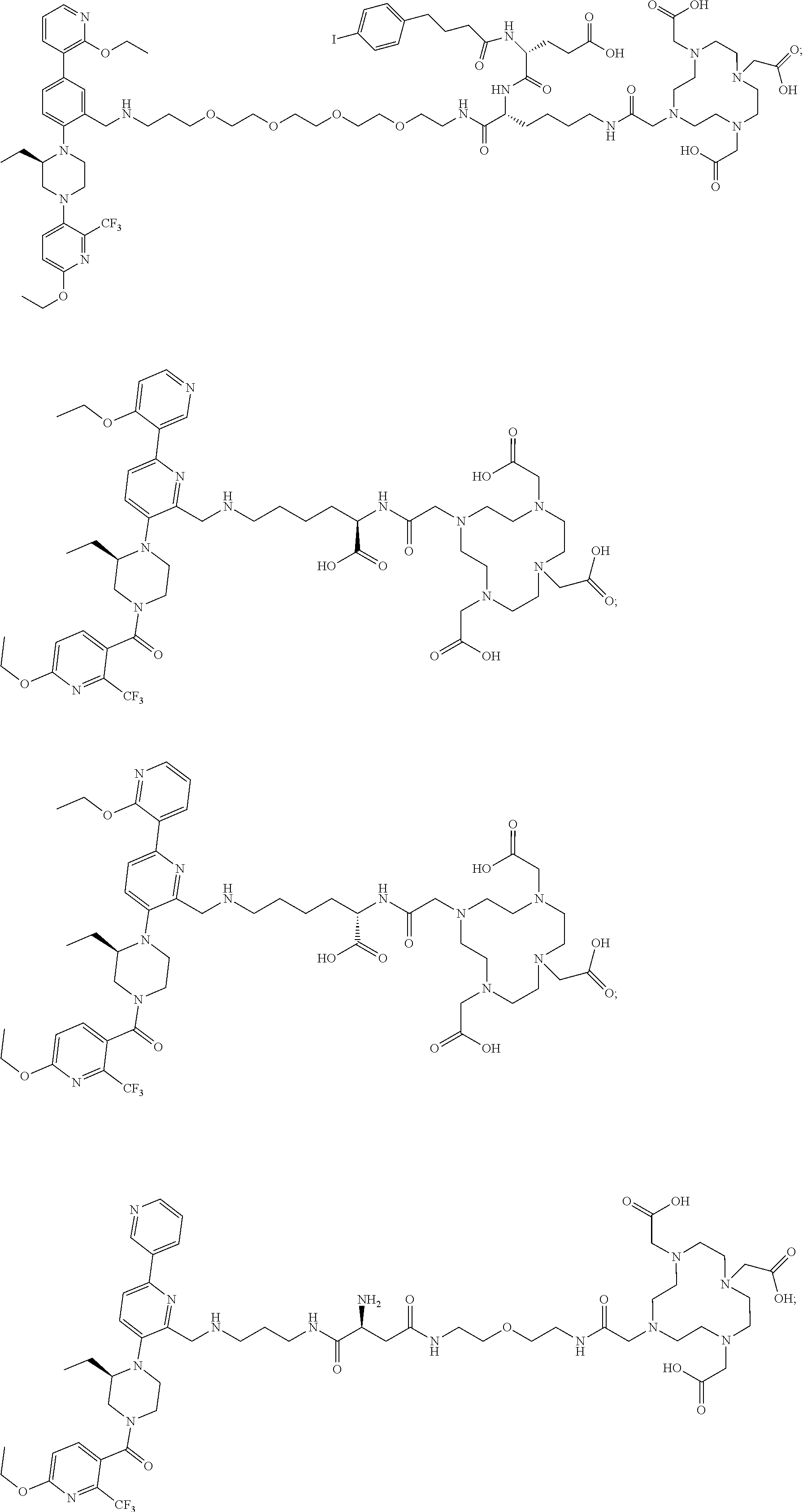

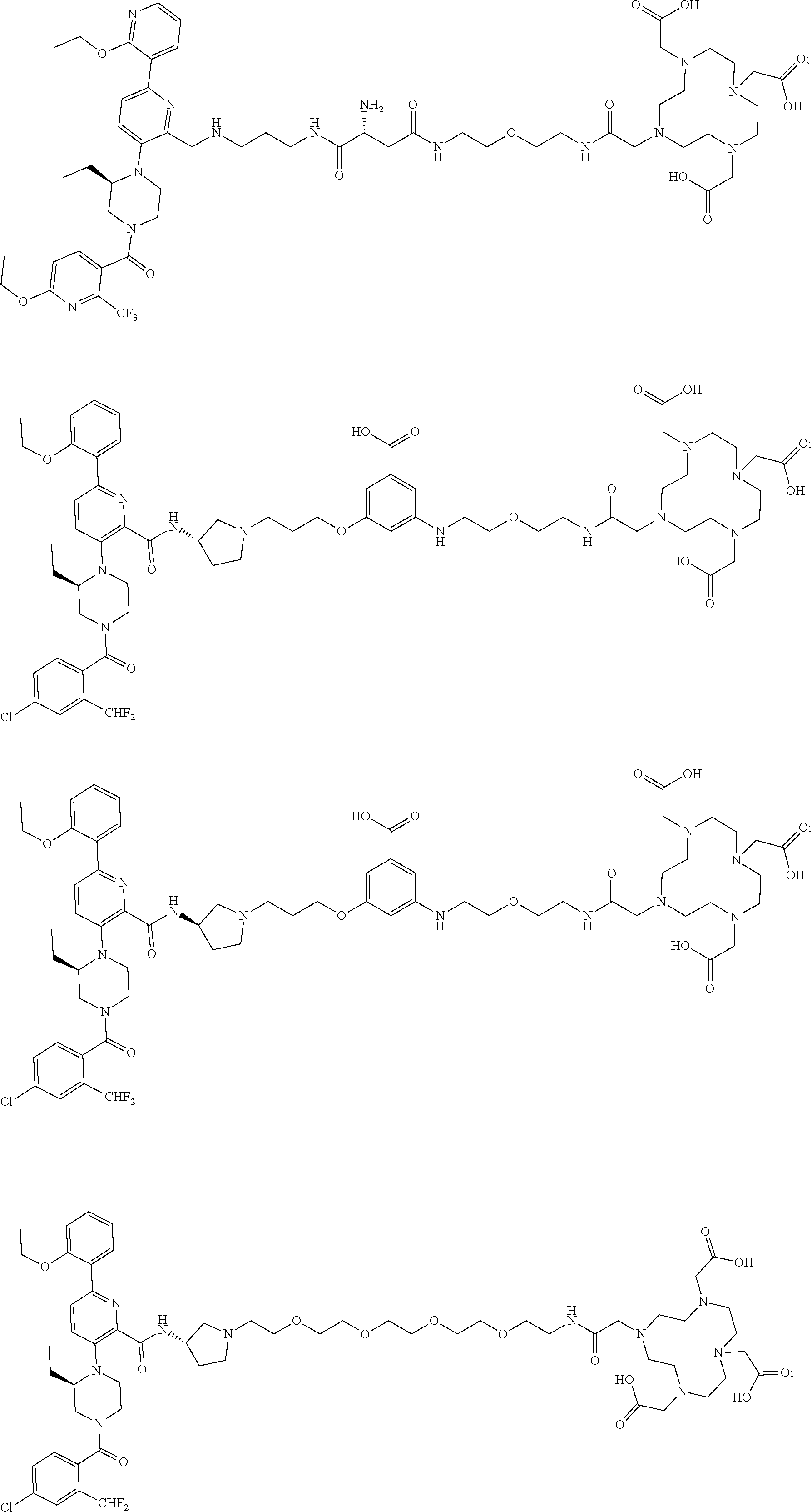

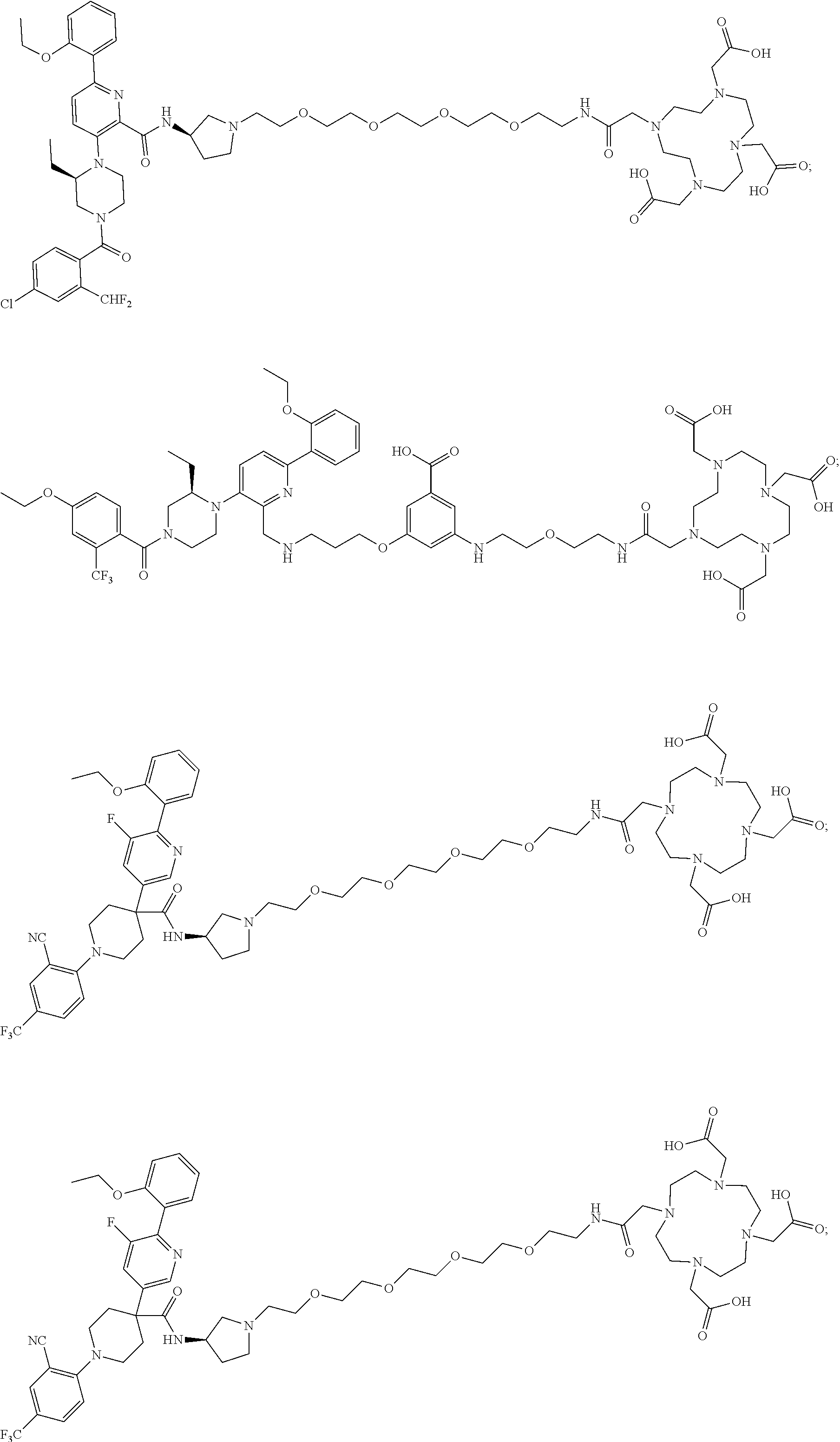

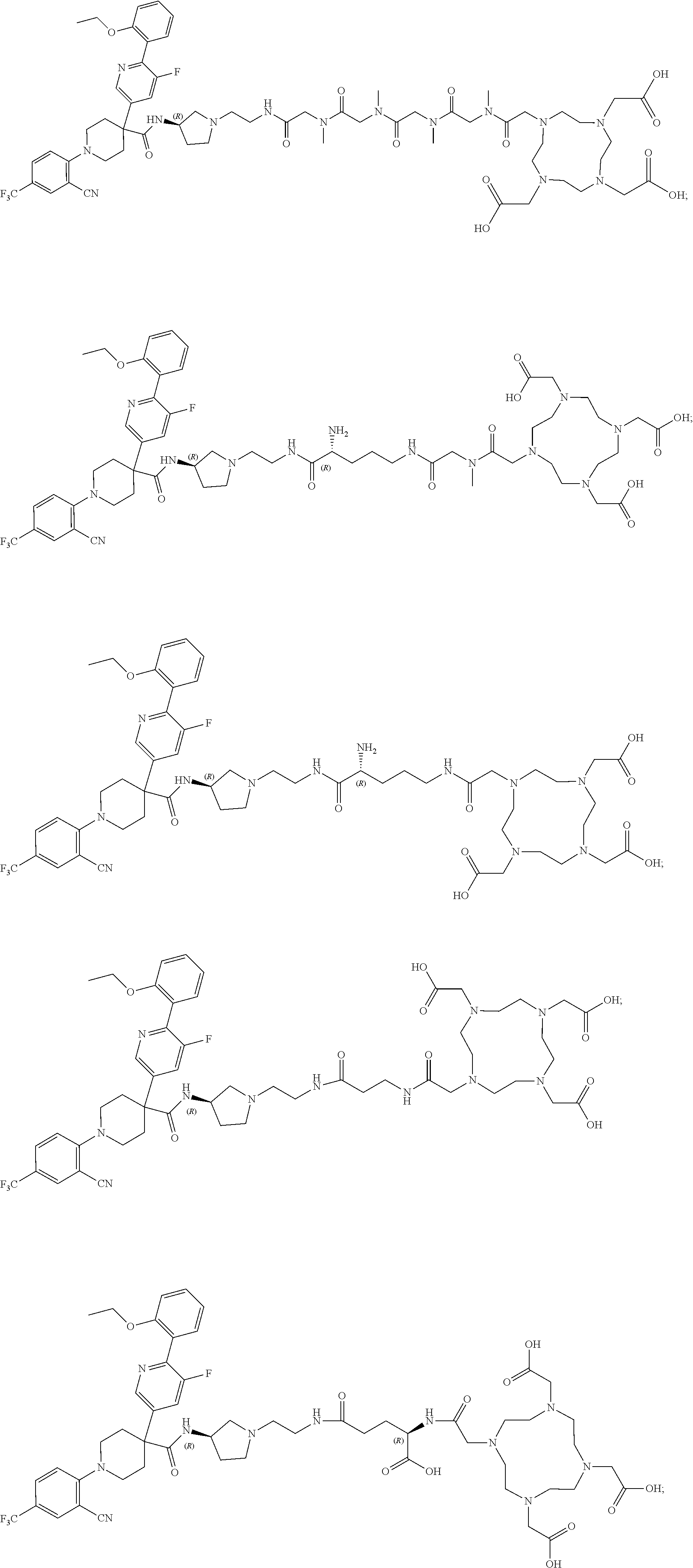

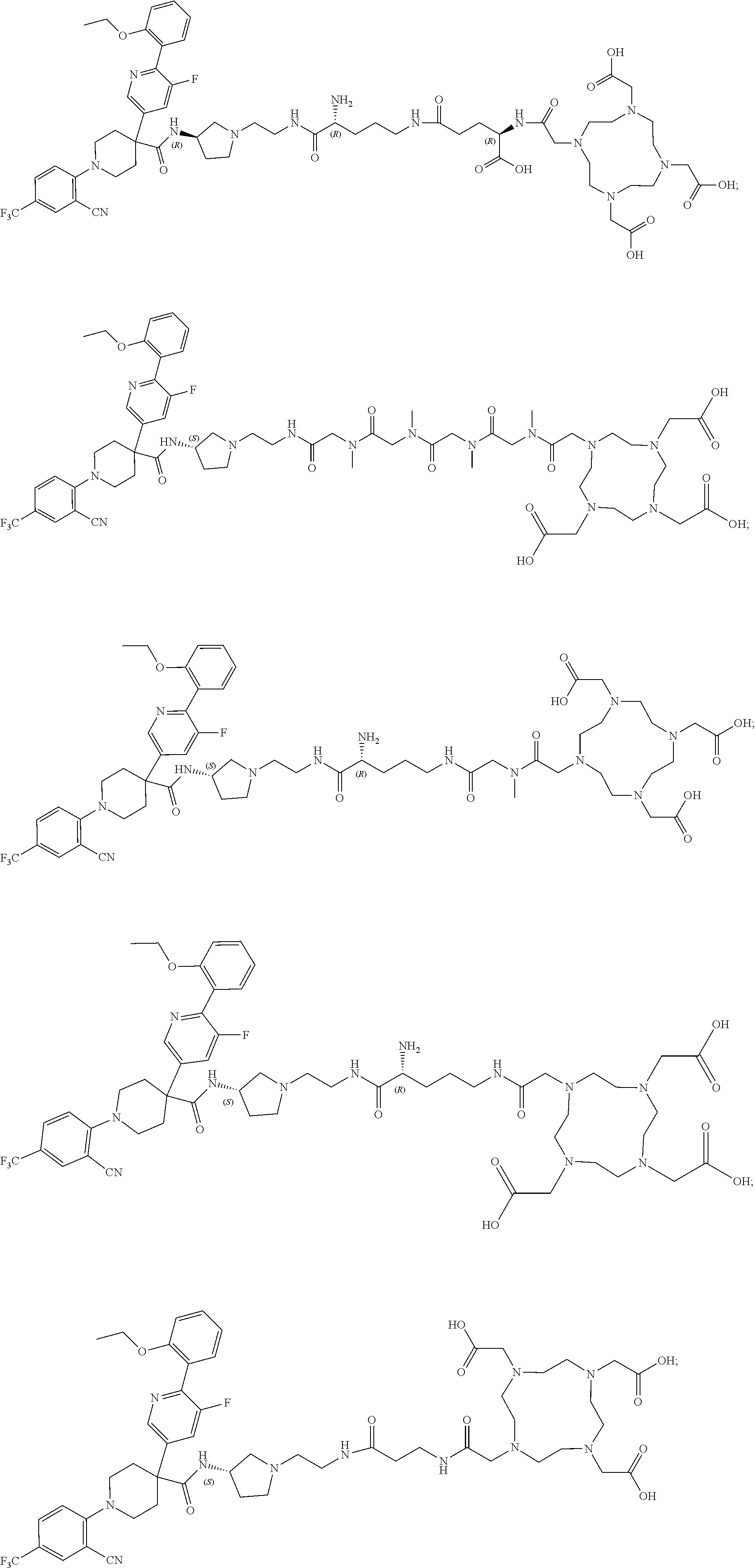

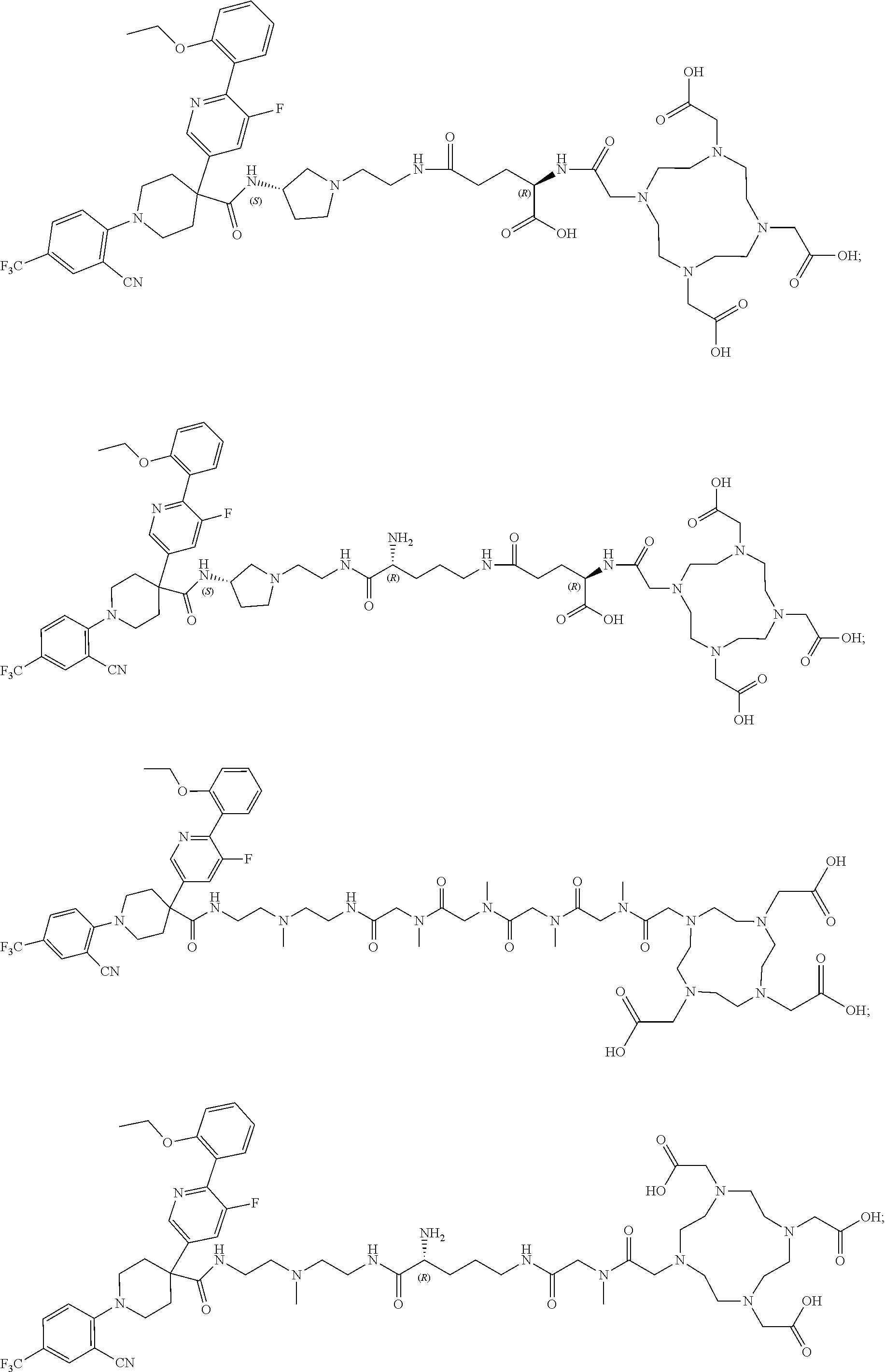

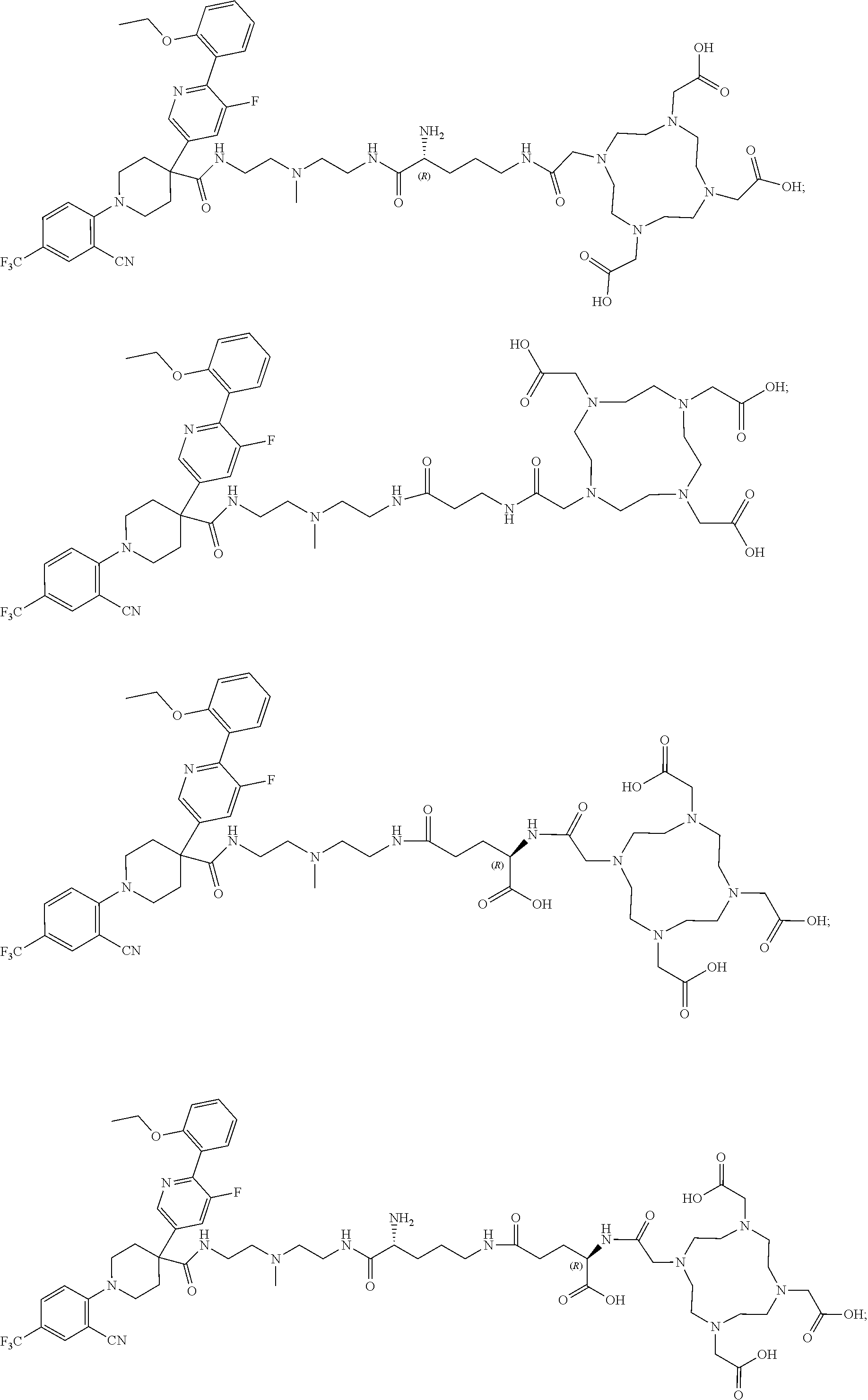

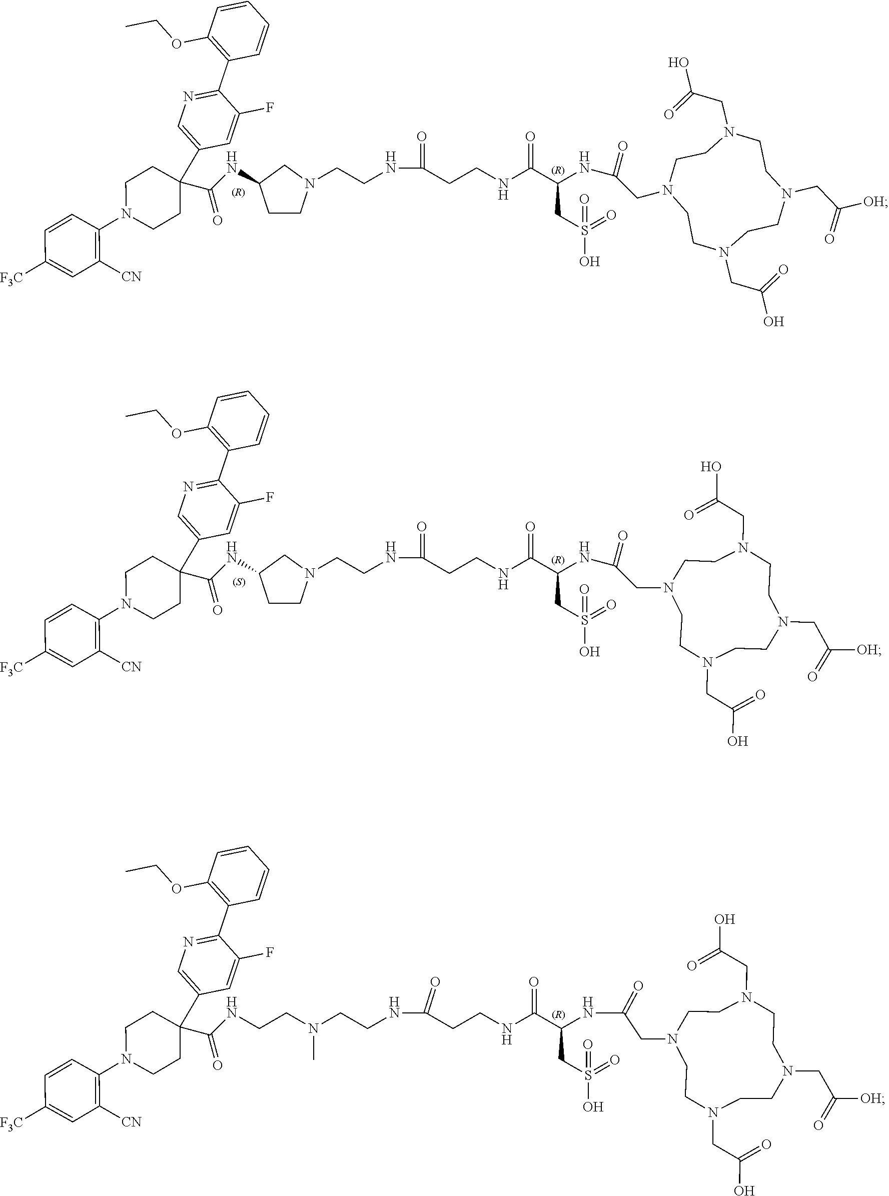

or radionuclide complex thereof.

Any combination of the groups described above for the various variables is contemplated herein. Throughout the specification, groups and substituents thereof are chosen by one skilled in the field to provide stable moieties and compounds.

### Synthesis of Compounds

Compounds described herein are synthesized using standard synthetic techniques or using methods known in the art in combination with methods described herein.

Unless otherwise indicated, conventional methods of mass spectroscopy, NMR, HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In one aspect, compounds described herein are in the form of pharmaceutically acceptable salts. In addition, the compounds described herein can exist in unsolvated as well as solvated forms with pharmaceutically acceptable solvents such as water, ethanol, and the like. The solvated forms of the compounds presented herein are also considered to be disclosed herein.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zirich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I) with an acid. In some embodiments, the compound of Formula (I) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (-L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (-L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (I) is prepared as a chloride salt, sulfate salt, bromide salt, mesylate salt, maleate salt, citrate salt or phosphate salt.

In some embodiments, pharmaceutically acceptable salts are obtained by reacting a compound of Formula (I) with a base. In some embodiments, the compound of Formula (I) is acidic and is reacted with a base. In such situations, an acidic proton of the compound of Formula (I) is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, sites on the organic radicals (e.g. alkyl groups, aromatic rings) of compounds of Formula (I) are deuterated.

In some embodiments, the compounds of Formula (I) possess one or more stereocenters and each stereocenter exists independently in either the R or S configuration. In some embodiments, the compound of Formula (I) exists in the R configuration. In some embodiments, the compound of Formula (I) exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference.

A “metabolite” of a compound disclosed herein is a derivative of that compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral), and parenteral routes (including injection or infusion, and subcutaneous).

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use.

### Methods of Treatment

In some embodiments, the methods comprise administering to a subject a therapeutically effective amount of a compound of Formula (I) or a pharmaceutically acceptable salt or solvate thereof. In some embodiments, the compound of Formula (I) or pharmaceutically acceptable salt or solvate thereof is administered in a pharmaceutical composition. In some embodiments, the subject has cancer. In some embodiments, the cancer is a solid tumor. In some embodiments, the subject has a noncancerous tumor. In some embodiments, the subject has an adenoma.

In embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life, or combinations thereof.

In some embodiments, provided herein are methods for killing a tumor cell comprising contacting the tumor cell with a compound of Formula (I) or a pharmaceutically acceptable salt or solvate thereof. In some embodiments, the compound of Formula (I) or pharmaceutically acceptable salt or solvate thereof releases a number of alpha particles by natural radioactive decay.

In some embodiments, the released alpha particles are sufficient to kill the tumor cell. In some embodiments, the released alpha particles are sufficient to stop cell growth. In some embodiments, the tumor cell is a malignant tumor cell. In some embodiments, the tumor cell is a benign tumor cell. In some embodiments, the method comprises killing a tumor cell with a beta-particle emitting radionuclide. In some embodiments, the method comprises killing a tumor cell with an alpha-particle emitting radionuclide. In some embodiments, the method comprises killing a tumor cell with a gamma-particle emitting radionuclide.

In one aspect, provided herein are methods and compositions for treating cancers.

In one aspect, provided herein are methods and compositions for treating an adenoma.

In one aspect, provided herein are methods and compositions for treating a carcinoma.

In one aspect, provided herein is a method for identifying tissues or organs in a mammal that overexpress MC2R comprising:
(i) administering to the mammal a compound of Formula (I); and
(ii) performing positron emission tomography (PET) analysis on the mammal.

In some embodiments, the mammal was diagnosed with cancer. In some embodiments, the tissues or organs in the mammal that overexpress MC2R are tumors. In some embodiments, the tissues or organs in the mammal that overexpress MC2R are adrenal tumors.

In some embodiments, compounds of Formula (I) disclosed herein are used in a method for in vivo imaging of a subject. In some embodiments, the method includes the steps of.
(i) administering to the mammal a compound of Formula (I);
(ii) waiting a sufficient amount of time to allow the compound of Formula (I) to accumulate at a tissue or cell site to be imaged; and
(iii) imaging the cells or tissues with a non-invasive imaging technique.

In some embodiments, the non-invasive imaging technique is positron emission tomography (PET) analysis. In some embodiments, the non-invasive imaging technique is selected from positron emission tomography imaging, or positron emission tomography with computed tomography imaging, and positron emission tomography with magnetic resonance imaging.

### Methods of Dosing and Treatment Regimens

In one embodiment, compounds of Formula (I), or a pharmaceutically acceptable salt thereof, are used in the preparation of medicaments for the treatment of tumors in a mammal.

Methods for treating any of the diseases or conditions described herein in a mammal in need of such treatment, involves administration of pharmaceutical compositions that include at least one compound of Formula (I) or a pharmaceutically acceptable salt thereof, in therapeutically effective amounts to said mammal.

In certain embodiments, the compositions containing the compound(s) described herein are administered for diagnostic and/or therapeutic treatments.

The amount of a given agent that corresponds to such an amount varies depending upon factors such as the particular conjugate, specific cancer or tumor to be treated (and its severity), the identity (e.g., weight, sex) of the subject or host in need of treatment, but nevertheless is determined according to the particular circumstances surrounding the case, including, e.g., the specific conjugate being administered, the route of administration, the condition being treated, and the subject or host being treated. Optimal doses are generally determined using experimental models and/or clinical trials. The optimal dose depends upon the body mass, weight, or blood volume of the subject.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀.

In certain embodiments, the data obtained from cell culture assays and animal studies are used in formulating the therapeutically effective daily dosage range and/or the therapeutically effective unit dosage amount for use in mammals, including humans.

The amount of a compound of Formula (I) or pharmaceutically acceptable salts thereof and/or pharmaceutical compositions that are administered are sufficient to deliver a therapeutically effective dose to the particular subject. In some embodiments, dosages of a compound of Formula (I) are between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of a compound of Formula (I) or a pharmaceutically acceptable salt thereof described herein for methods of treating a disease as described herein is about 0.001 mg/kg to about 1 mg/kg body weight of the subject per dose. In some embodiments, the dose of a compound of Formula (I) or a pharmaceutically acceptable salt thereof described herein for the described methods is about 0.001 mg to about 1000 mg per dose for the subject being treated. In some embodiments, a compound of Formula (I) or a pharmaceutically acceptable salt thereof described herein is administered to a subject at a dosage of from about 0.01 mg to about 500 mg, from about 0.01 mg to about 100 mg, or from about 0.01 mg to about 50 mg.

In some embodiments, a compound of Formula (I) or a pharmaceutically acceptable salt thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole.

In some embodiments, a compound of Formula (I) or a pharmaceutically acceptable salt thereof described herein is administered to a subject at a dosage of about 0.01 Gbq to about 1000 Gbq, about 0.5 Gbq to about 100 Gbq, or about 1 Gbq to about 50 Gbq.

In some embodiments, the dose is administered once a day, 1 to 3 times a week, 1 to 4 times a month, or 1 to 12 times a year.

In any of the aforementioned aspects are further embodiments in which the effective amount of the compound of Formula (I), or a pharmaceutically acceptable salt thereof, is: (a) systemically administered to the mammal; and/or (b) intravenously administered to the mammal; and/or (c) administered by injection to the mammal.

In certain instances, it is appropriate to administer at least one compound of Formula (I), or a pharmaceutically acceptable salt thereof, in combination with one or more other therapeutic agents.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g, “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a (alkyl)O— group, where alkyl is as defined herein.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a C₃-C₄cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 1 O atoms in its ring system, and with the proviso that any ring does not contain two adjacent O or S atoms. Non-aromatic heterocyclic groups (also known as heterocycloalkyls) include rings having 3 to 1 O atoms in its ring system and aromatic heterocyclic groups include rings having 5 to 1 O atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion). Those of skill in the art are familiar with administration techniques that can be employed with the compounds and methods described herein.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Abbreviations:

Pd(DTBPF)Cl₂: [1,1′-Bis(di-tert-butylphosphino)ferrocene]dichloropalladium(II);
HATU: 1-[Bis(dimethylamino)methylene]-1H-1,2,3-triazolo[4,5-b]pyridinium 3-oxide hexafluorophosphate;
TEA: triethylamine; DIEA or DIPEA: N,N-diisopropylethylamine;
Prep-HPLC: preparative high-performance liquid chromatography;
MS: mass spectrometry; LCMS: Liquid chromatography-mass spectrometry;
TFA: trifluoroacetic acid; AcOH: acetic acid; HCl: hydrochloric acid or hydrochloride;
H₂O: water; MeCN or CH₃CN or ACN: acetonitrile;
DMSO: dimethyl sulfoxide; DMF: dimethylformamide; DCM: dichloromethane;
EtOH: ethanol; IPA: isopropyl alcohol; PE: petroleum ether;
rt: room temperature; atm: atmospheric pressure;
h or hr: hour; hrs: hours; min: minute
mg: milligrams; kg: kilograms; mL: milliliter;
Eq: equivalents; mol: moles; mmol: millimole;
Na₂CO₃: sodium carbonate; K₂CO₃: potassium carbonate; Na₂SO₄: sodium sulfate;
Brine: saturated NaCl solution.

### EXAMPLES

The following examples are provided for illustrative purposes only and not to limit the scope of the claims provided herein.

### Synthesis of Compounds

### Example 1: (R)-(4-(6-(aminomethyl)-2′-ethoxy-[2,3′-bipyridin]-5-yl)-3-ethylpiperazin-1-yl)(6-ethoxy-2-(trifluoromethyl)pyridin-3-yl)methanone

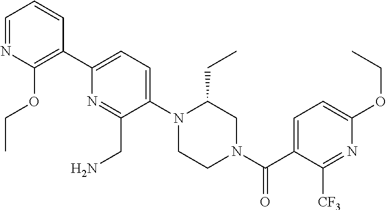 [SMILES: CCOc1ccc(C(=O)N2CCN(c3ccc(-c4cccnc4OCC)nc3CN)[C@H](CC)C2)c(C(F)(F)F)n1]

Step 1: Into a 2000-mL 3-necked round-bottom flask purged and maintained with an inert atmosphere of nitrogen, 6-chloro-3-fluoropicolinonitrile (96 g, 1 Eq, 0.61 mol), tert-butyl (R)-3-ethylpiperazine-1-carboxylate (0.20 kg, 1.5 Eq, 0.92 mol), and DMSO (960 mL) were added. This was followed by the addition of N-ethyl-N-isopropylpropan-2-amine (0.24 kg, 3.0 Eq, 1.8 mol). The resulting solution was stirred for 48 hrs at 90° C. in an oil bath. The reaction mixture was cooled to 30° C. with a water/ice bath. The reaction was then quenched by the addition of 3000 mL of water. The resulting solution was extracted with 3×2500 mL of ethyl acetate, the organic phase was washed with 2×2500 mL of brine, dried over anhydrous sodium sulfate, and concentrated under vacuum. The residue was applied onto a silica gel column eluting with ethyl acetate/petroleum ether (0-15%). This resulted in tert-butyl (R)-4-(6-chloro-2-cyanopyridin-3-yl)-3-ethylpiperazine-1-carboxylate (159.3 g, 454.0 mmol, 74%) as a yellow oil.

Step 2: Into a 5000-mL 3-necked round-bottom flask purged and maintained with an inert atmosphere of nitrogen, was placed tert-butyl (R)-4-(6-chloro-2-cyanopyridin-3-yl)-3-ethylpiperazine-1-carboxylate (159.3 g, 1 Eq, 454.0 mmol), (2-ethoxypyridin-3-yl)boronic acid (113.7 g, 1.5 Eq, 681.1 mmol), 1,4-Dioxane (1593 mL), water (159.3 mL), potassium carbonate (194.5 g, 3.1 Eq, 1.408 mol), and Pd(DTBPF)Cl₂ (14.80 g, 0.05 Eq, 22.70 mmol). The resulting solution was stirred for 2 hrs at 70° C. in an oil bath. The reaction mixture was cooled to 30° C. with a water/ice bath. The reaction was then quenched by the addition of 1500 mL of water. The resulting solution was extracted with 3×2500 ml of ethyl acetate, and the organic phase was washed with 2×1000 mL of brine. The organic phase was dried over anhydrous sodium sulfate and concentrated under vacuum. The remaining residue was applied onto a silica gel column eluting with ethyl acetate/petroleum ether (0-15%). This resulted in tert-butyl (R)-4-(6-cyano-2′-ethoxy-[2,3′-bipyridin]-5-yl)-3-ethylpiperazine-1-carboxylate (180.2 g, 411.8 mmol, 90.71%) as a yellow oil.

Step 3: Into a 2000-mL 1-necked round-bottom flask purged and maintained with an inert atmosphere of nitrogen, was placed tert-butyl (R)-4-(6-cyano-2′-ethoxy-[2,3′-bipyridin]-5-yl)-3-ethylpiperazine-1-carboxylate (125 g, 1 Eq, 286 mmol), DCM (875 mL), and 2,2,2-trifluoroacetic acid (32.6 g, 375 mL, 1 Eq, 286 mmol). The resulting solution was stirred for 4 hrs at 25° C. and then concentrated to remove TFA. The resulting solution was diluted with 1500 mL of DCM. The pH value of the solution was adjusted to 8-9 with Na₂CO₃. The resulting mixture was extracted with 3×1500 mL of dichloromethane, the organic phase was dried over anhydrous sodium sulfate and then concentrated. This resulted in crude (R)-2′-ethoxy-5-(2-ethylpiperazin-1-yl)-[2,3′-bipyridine]-6-carbonitrile (90 g, 0.27 mol, 93%) as a yellow solid.

Step 4: Into a 2000-mL 3-round-bottom flask purged and maintained with an inert atmosphere of nitrogen, was placed 6-ethoxy-2-(trifluoromethyl)nicotinic acid (67 g, 1.2 Eq, 0.28 mol), 2-(3H-[1,2,3]triazolo[4,5-b]pyridin-3-yl)-1,1,3,3-tetramethylisouronium hexafluorophosphate(V) (0.12 kg, 1.3 Eq, 0.31 mol), and N-ethyl-N-isopropylpropan-2-amine (0.11 kg, 3.5 Eq, 0.83 mol). The resulting solution was stirred for 30 min at ambient temperature followed by the addition of crude (R)-2′-ethoxy-5-(2-ethylpiperazin-1-yl)-[2,3′-bipyridine]-6-carbonitrile (80 g, 1 Eq, 0.24 mol). The resulting solution was stirred for 2 hrs at 25° C. The reaction was quenched by the addition of 50 mL of water. The resulting mixture was extracted with 3×1500 mL of ethyl acetate, and the organic phase was washed with 2×500 mL of brine. The organic phase was dried over anhydrous sodium sulfate and concentrated under vacuum. The crude product was purified by Flash-Prep-HPLC using the following conditions (CombiFlash): Column, C18 silica gel; mobile phase, NH₃·H₂O/ACN=50% increasing to NH₃·H₂O/ACN=85% within 50 min; Detector, 210 nm. This resulted in (R)-2′-ethoxy-5-(4-(6-ethoxy-2-(trifluoromethyl)nicotinoyl)-2-ethylpiperazin-1-yl)-[2,3′-bipyridine]-6-carbonitrile (104 g, 188 mmol, 79%) as an off-white solid.

Step 5: Into a 5000 mL hydrogen reactor were added (R)-2′-ethoxy-5-(4-(6-ethoxy-2-(trifluoromethyl)nicotinoyl)-2-ethylpiperazin-1-yl)-[2,3′-bipyridine]-6-carbonitrile (104 g, 1 Eq, 188 mmol), EtOH (2080 mL), ammonium hydroxide (208 mL) and Raney nickel (31.2 g, 30 wt %). The mixture was stirred for 72 hrs at room temperature under 8 atm of hydrogen. The reaction mixture was filtered, and the filtrate was concentrated under vacuum. The remaining material was purified by CombiFlash using silica gel chromatography. This resulted in (R)-(4-(6-(aminomethyl)-2′-ethoxy-[2,3′-bipyridin]-5-yl)-3-ethylpiperazin-1-yl)(6-ethoxy-2-(trifluoromethyl)pyridin-3-yl)methanone (63 g, 0.11 mol, 60%) as an off-white solid.

### Example 2: (R)—N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide

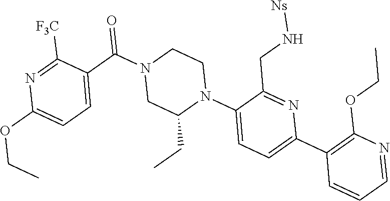

To a DCM (10 mL) solution of (R)-(4-(6-(aminomethyl)-2′-ethoxy-[2,3′-bipyridin]-5-yl)-3-ethylpiperazin-1-yl)(6-ethoxy-2-(trifluoromethyl)pyridin-3-yl)methanone (990 mg, 1 Eq, 1.77 mmol) was added TEA (541 mg, 745 μL, 3.02 Eq, 5.35 mmol) and NₛCl (512 mg, 1.30 Eq, 2.31 mmol) was added at 0° C. The reaction mixture was stirred at 25° C. for 1 hr. The reaction mixture was purified by silica gel chromatography (EtOAc/petroleum ether, 0% to 85% in 15 min) to afford (R)—N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (1.06 g, 1.43 mmol, 80.4%) as a solid. [M+H]=744.3.

### Example 3: (R)-(4-(2-(aminomethyl)-6-(2-ethoxyphenyl)pyridin-3-yl)-3-ethylpiperazin-1-yl)(4-ethoxy-2-(trifluoromethyl)phenyl)methanone

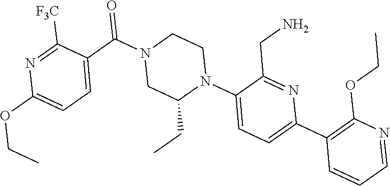 [SMILES: CCOc1ccc(C(=O)N2CCN(c3ccc(-c4cccnc4OCC)nc3CN)[C@H](CC)C2)c(C(F)(F)F)n1]

Step 1: To a 100 mL flask was added 4-bromo-3-(trifluoromethyl)phenol (5 g, 1 Eq, 0.02 mol), K₂CO₃ (0.01 kg, 3 Eq, 0.07 mol), Ethyl iodide (5 g, 3 mL, 2 Eq, 0.03 mol) and MeCN (50 mL). The reaction mixture was stirred at 80° C. for 2 hrs. The resulting mixture was concentrated under reduced pressure to remove 50 mL of EtOH. The remaining residue was diluted with water (70 mL). The pH value was adjusted to 5.0 using saturated NaHSO₄ solution and extracted with DCM (50 mL×3). The organic layer was dried over anhydrous Na₂SO₄ and concentrated under reduced pressure to afford 1-bromo-4-ethoxy-2-(trifluoromethyl)benzene (5.2 g, 19 mmol, 90%) as a red solid.

Step 2: Into a 40-mL vials purged and maintained with an inert atmosphere of nitrogen, was placed tert-butyl (R)-4-(6-bromo-2-cyanopyridin-3-yl)-3-ethylpiperazine-1-carboxylate (3 g, 1 Eq, 8 mmol), (2-ethoxyphenyl)boronic acid (1.39 g, 1 Eq, 8.37 mmol), K₂CO₃ (3.16 g, 3 Eq, 22.9 mmol), 1,1′-Bis(di-t-butylphosphino)ferrocene palladium dichloride (0.2 g, 0.04 Eq, 0.3 mmol), 1,4-Dioxane (30 mL) and Water (3.0 mL). The resulting reaction mixture was stirred at 80° C. for 2 hrs. The reaction mixture was cooled to ambient temperature and quenched with water (40 mL). The resulting solution was extracted with ethyl acetate (3×40 mL). The organic layers were combined, dried over anhydrous sodium sulfate and concentrated under vacuum. The remaining residue was purified by silica gel chromatography eluting with ethyl acetate/petroleum ether (1:1). This resulted in tert-butyl (R)-4-(2-cyano-6-(2-ethoxyphenyl)pyridin-3-yl)-3-ethylpiperazine-1-carboxylate (2.97 g, 6.80 mmol, 90%) as a yellow oil. [M+H]=438.0.

Step 3: Into a 40-mL vial, was placed tert-butyl (R)-4-(2-cyano-6-(2-ethoxyphenyl)pyridin-3-yl)-3-ethylpiperazine-1-carboxylate (1.3 g, 1 Eq, 3.0 mmol), TFA (3 mL) and DCM (9 mL). The resulting reaction mixture was stirred at 20° C. for 1 h. The reaction mixture was concentrated under vacuum. This resulted in crude (R)-6-(2-ethoxyphenyl)-3-(2-ethylpiperazin-1-yl)picolinonitrile (1.4 g, 4.2 mmol, 140%), which was used in next step without further purification. [M+H]=337.2.

Step 4: Into a 40 mL vial, to the mixture of 4-ethoxy-2-(trifluoromethyl)benzoic acid (0.690 g, 1 Eq, 2.95 mmol), HATU (1.4 g, 1.2 Eq, 3.7 mmol), DIEA (1.12 g, 1.51 mL, 2.94 Eq, 8.67 mmol) in DMF (20 mL) was added (R)-6-(2-ethoxyphenyl)-3-(2-ethylpiperazin-1-yl)picolinonitrile (1.3 g, 1.3 Eq, 3.9 mmol). The resulting mixture was stirred at 20° C. for 2 hrs. The reaction mixture was purified by Prep-HPLC using the following conditions: Column, SunFire Prep C18 OBD Column, 19×150 mm, 5 μm; mobile phase: Water (0.05% FA) and ACN (30.0% to 98.0% ACN in 7 min); Total flow rate, 70 mL/min; Detector, UV 220 nm. The collected fractions were combined and concentrated under vacuum to afford (R)-3-(4-(4-ethoxy-2-(trifluoromethyl)benzoyl)-2-ethyl-piperazin-1-yl)-6-(2-ethoxyphenyl)picolinonitrile (1.2 g, 2.2 mmol, 74%) as a solid. [M+H]=553.3.

Step 5: Into a 500-mL round-bottom flask purged and maintained with an inert atmosphere of nitrogen, was placed (R)-3-(4-(4-ethoxy-2-(trifluoromethyl)benzoyl)-2-ethylpiperazin-1-yl)-6-(2-ethoxyphenyl)picolinonitrile (1.2 g, 1 Eq, 2.2 mmol), IPA (200 mL) and aqeuous NH₃ solution (20.0 mL), and nickel (0.64 g, 5.0 Eq, 11 mmol). The reaction flask was evacuated and flushed with nitrogen three times, followed by flushing with hydrogen. The mixture was stirred 20° C. for 16 hrs under hydrogen. The reaction mixture was filtered through a pad of celite. The filtrate was concentrated and purified by Prep-HPLC using the following conditions: Column, C18 120 g; mobile phase, Water (0.1% FA) and ACN (30% ACN up to 98% in 6 min, 98% ACN to 98% in 3 min); Total flow rate, 70 mL/min; Detector, UV 220 nm. The collected fractions were concentrated under reduced pressure and dried under vacuum. This resulted in (R)-(4-(2-(aminomethyl)-6-(2-ethoxyphenyl)pyridin-3-yl)-3-ethylpiperazin-1-yl)(4-ethoxy-2-(trifluoromethyl)phenyl)methanone (1.2 g, 2.2 mmol, 99%) as white solid. [M+H]=557.9

### Example 4: (R)—N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide

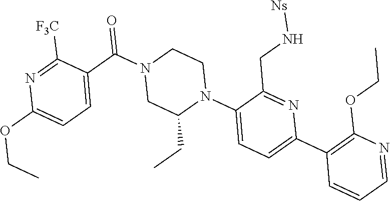

Into a 50-mL round bottom flask, was placed a mixture of (R)-(4-(2-(aminomethyl)-6-(2-ethoxyphenyl)pyridin-3-yl)-3-ethylpiperazin-1-yl)(4-ethoxy-2-(trifluoromethyl)phenyl)methanone (1.08 g, 1 Eq, 1.94 mmol), TEA (600 mg, 826 μL, 3.06 Eq, 5.93 mmol), 2-nitrobenzene sulfonyl chloride (480 mg, 1.12 Eq, 2.17 mmol) and DCM (10 mL). The reaction mixture was stirred at 20° C. for 2 hrs. The reaction mixture was purified by silica gel chromatography (EtOAc/PE, 0% to 90% in 15 min) to afford (R)—N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide (1.15 g, 1.55 mmol, 79.9%) as yellow solid. Lot Number: 0066-0001-7A. [M+H]=742.4

### Example 5: tert-butyl (15-hydroxy-3,6,9,12-tetraoxapentadecyl)carbamate

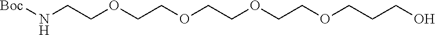 [SMILES: CC(C)(C)OC(=O)NCCOCCOCCOCCOCCCO]

Into a 100-mL round bottom flask, was placed with a mixture of 2,2-dimethyl-4-oxo-3,8,11,14,17-pentaoxa-5-azaicosan-20-oic acid (3.20 g, 1 Eq, 8.76 mmol) and THF (40 mL). BH₃·DMS (10.5 g, 13.1 mL, 2.0 molar, 2.99 Eq, 26.2 mmol) was added dropwise at 0° C. The reaction mixture was stirred at 0° C. for 30 min and then at 20° C. for 1 hour. The reaction mixture was quenched with 30 mL of MeOH, and the resulting mixture was stirred at 20° C. for 30 min. The resulting mixture was concentrated under reduced pressure to afford crude tert-butyl (15-hydroxy-3,6,9,12-tetraoxapentadecyl)carbamate (3.10 g, 8.4 mmol, 96%, 95% Purity) as a solid. [M+Na]=374.2.

### Example 6: Synthesis of 2,2-dimethyl-4-oxo-3,8,11,14,17-pentaoxa-5-azaicosan-20-yl methanesulfonate

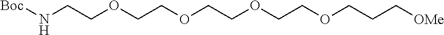 [SMILES: COCCCOCCOCCOCCOCCNC(=O)OC(C)(C)C]

Into a 100-mL round bottom flask, was placed a mixture of tert-butyl (15-hydroxy-3,6,9,12-tetraoxapentadecyl)carbamate (3.10 g, 1 Eq, 8.82 mmol), triethylamine (2.68 g, 3.00 Eq, 26.5 mmol) and DCM (40 mL). Methanesulfonyl chloride (1.52 g, 1.50 Eq, 13.3 mmol) was added dropwise at 0° C. The reaction mixture was stirred at 20° C. for 2 hrs. The resulting mixture was quenched with 60 mL of water and extracted with DCM (2×50 mL). The organic layers were combined, washed with brine (50 mL), dried over anhydrous Na₂SO₄, and concentrated under reduced pressure to afford crude 2,2-dimethyl-4-oxo-3,8,11,14,17-pentaoxa-5-azaicosan-20-yl methanesulfonate (3.25 g, 7.57 mmol, 85.8%) as yellow solid. [M+Na]=452.2.

### Example 7: (R)-2,2′,2″-(10-(1-(2′-ethoxy-5-(4-(6-ethoxy-2-(trifluoromethyl)nicotinoyl)-2-ethylpiperazin-1-yl)-[2,3′-bipyridin]-6-yl)-19-oxo-6,9,12,15-tetraoxa-2,18-diazaicosan-20-yl)-1,4,7,10-tetraazacyclododecane-1,4,7-triyl)triacetic acid (Compound 1)

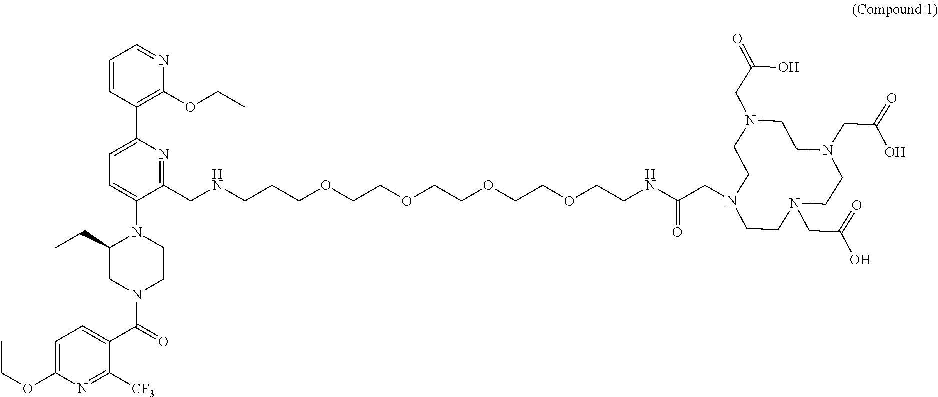 [SMILES: CCOc1ccc(C(=O)N2CCN(c3ccc(-c4cccnc4OCC)nc3CNCCCOCCOCCOCCOCCNC(=O)CN3CCN(CC(=O)O)CCN(CC(=O)O)CCN(CC(=O)O)CC3)[C@H](CC)C2)c(C(F)(F)F)n1]

Step 1: To a DMF (5 mL) solution of (R)-(4-(6-(aminomethyl)-2′-ethoxy-[2,3′-bipyridin]-5-yl)-3-ethylpiperazin-1-yl)(6-ethoxy-2-(trifluoromethyl)pyridin-3-yl)methanone (780.3 mg, 1.8 Eq, 1.397 mmol) and 2,2-dimethyl-4-oxo-3,8,11,14,17-pentaoxa-5-azaicosan-20-yl methanesulfonate (427.9 mg, 77.9% Wt, 1.0 Eq, 776.0 μmol) was added potassium carbonate (321.7 mg, 3.0 Eq, 2.328 mmol). The resulting mixture was heated at 80° C. for 5 hrs. The reaction mixture was diluted with water and extracted with EtOAc (2×). The organic layers were combined and concentrated to dryness. The remaining residue was purified by reverse-phase chromatography. Pure fractions were combined, neutralized with NaHCO₃ (aq), and extracted with EtOAc (2×). The organic layers were combined, washed with brine, dried over anhydrous MgSO₄, and concentrated to give crude tert-butyl (R)-(1-(2′-ethoxy-5-(4-(6-ethoxy-2-(trifluoromethyl)nicotinoyl)-2-ethylpiperazin-1-yl)-[2,3′-bipyridin]-6-yl)-6,9,12,15-tetraoxa-2-azaheptadecan-17-yl)carbamate (361.6 mg, 405.4 μmol, 52.24%) as a light brown gum.

Step 2: To a DCM (7 mL) solution of tert-butyl (R)-(1-(2′-ethoxy-5-(4-(6-ethoxy-2-(trifluoromethyl)nicotinoyl)-2-ethylpiperazin-1-yl)-[2,3′-bipyridin]-6-yl)-6,9,12,15-tetraoxa-2-azaheptadecan-17-yl)carbamate (361.6 mg, 1 Eq, 405.4 mol) and benzyl (2,5-dioxopyrrolidin-1-yl) carbonate (151.5 mg, 1.5 Eq, 608.1 μmol) was added N-ethyl-N-isopropylpropan-2-amine (104.8 mg, 141 μL, 2 Eq, 810.7 mol). The resulting mixture was stirred at 25° C. for 30 min. The reaction mixture was concentrated, and the remaining residue was purified by silica gel chromatography (eluted at 100% EtOAc). Pure fractions were combined and concentrated to give benzyl (R)-(2,2-dimethyl-4-oxo-3,8,11,14,17-pentaoxa-5-azaicosan-20-yl)((2′-ethoxy-5-(4-(6-ethoxy-2-(trifluoromethyl)nicotinoyl)-2-ethylpiperazin-1-yl)-[2,3′-bipyridin]-6-yl)methyl)carbamate (305.6 mg, 297.8 μmol, 73.47%) as a light brown gum.

Step 3: To a DCM (0.5 mL) solution of benzyl (R)-(2,2-dimethyl-4-oxo-3,8,11,14,17-pentaoxa-5-azaicosan-20-yl)((2′-ethoxy-5-(4-(6-ethoxy-2-(trifluoromethyl)nicotinoyl)-2-ethylpiperazin-1-yl)-[2,3′-bipyridin]-6-yl)methyl)carbamate (305.6 mg, 1 Eq, 297.8 μmol) was added 2,2,2-trifluoroacetic acid (679.1 mg, 456 μL, 20 Eq, 5.956 mmol). The resulting mixture was stirred at 25° C. for 1 hr. The reaction mixture was concentrated, and the remaining residue was triturated with ice and basified with saturated NaHCO₃ (aq). The resulting mixture was extracted with EtOAc (2×). Organic layers were combined, dried over MgSO₄ and concentrated to give crude benzyl (R)-(1-amino-3,6,9,12-tetraoxapentadecan-15-yl)((2′-ethoxy-5-(4-(6-ethoxy-2-(trifluoromethyl)-nicotinoyl)-2-ethylpiperazin-1-yl)-[2,3′-bipyridin]-6-yl)methyl)carbamate (264.3 mg, 285.4 μmol, 95.84%) as a light brown gum.

Step 4: To a DMF (0.2 mL) solution of 2-(4,7,10-tris(2-(tert-butoxy)-2-oxoethyl)-1,4,7,10-tetraazacyclododecan-1-yl)acetic acid (245.2 mg, 1.5 Eq, 428.1 μmol) and HATU (151.9 mg, 1.4 Eq, 399.6 μmol) was added N-ethyl-N-isopropylpropan-2-amine (110.7 mg, 149 μL, 3 Eq, 856.2 μmol). The resulting mixture was stirred at 25° C. for 10 min followed by the addition of benzyl (R)-(1-amino-3,6,9,12-tetraoxapentadecan-15-yl)((2′-ethoxy-5-(4-(6-ethoxy-2-(trifluoromethyl)nicotinoyl)-2-ethylpiperazin-1-yl)-[2,3′-bipyridin]-6-yl)methyl)carbamate (264.3 mg, 1 Eq, 285.4 μmol). The reaction mixture was stirred at 25° C. for 10 min. Additional HATU (151.9 mg, 1.4 Eq, 399.6 μmol) was added and the reaction mixture was stirred for 10 min to drive the reaction to completion. The reaction mixture was purified by reversed phase chromatography. Pure fractions were combined, neutralized with saturated NaHCO₃ (aq), and extracted with EtOAc (2×). The organic layers were combined, dried over anhydrous MgSO₄ and concentrated to give tri-tert-butyl 2,2′,2″-(10-(4-((2′-ethoxy-5-(4-(6-ethoxy-2-(trifluoromethyl)nicotinoyl)-2-ethylpiperazin-1-yl)-[2,3′-bipyridin]-6-yl)methyl)-3,21-dioxo-1-phenyl-2,8,11,14,17-pentaoxa-4,20-diazadocosan-22-yl)-1,4,7,10-tetra-azacyclododecane-1,4,7-triyl)(R)-triacetate (147.1 mg, 99.34 μmol, 34.81%) as a light brown gum.

Step 5: To tri-tert-butyl 2,2′,2″-(10-(4-((2′-ethoxy-5-(4-(6-ethoxy-2-(trifluoromethyl)-nicotinoyl)-2-ethylpiperazin-1-yl)-[2,3′-bipyridin]-6-yl)methyl)-3,21-dioxo-1-phenyl-2,8,11,14,17-pentaoxa-4,20-diazadocosan-22-yl)-1,4,7,10-tetraazacyclododecane-1,4,7-triyl)(R)-triacetate (147.1 mg, 1 Eq, 99.34 μmol) was added methyl(phenyl)sulfane (197.4 mg, 187.5 μL, 16 Eq, 1.589 mmol) and 2,2,2-trifluoroacetic acid (1.812 g, 1.22 mL, 160 Eq, 15.89 mmol). The resulting mixture was heated at 50° C. for 30 min. LCMS analysis showed all tert-butyl esters were hydrolyzed but Cbz-group was still retained. The remaining mixture was heated at 60° C. for additional 3 hrs. The resulting mixture was concentrated to remove most TFA. The remaining residue was rinsed with hexanes (2×) and purified by reversed phase chromatography. Pure fractions were freeze dried on lyophilizer to give (R)-2,2′,2″-(10-(1-(2′-ethoxy-5-(4-(6-ethoxy-2-(trifluoromethyl)nicotinoyl)-2-ethylpiperazin-1-yl)-[2,3′-bipyridin]-6-yl)-19-oxo-6,9,12,15-tetraoxa-2,18-diazaicosan-20-yl)-1,4,7,10-tetraazacyclododecane-1,4,7-triyl)triacetic acid-2,2,2-trifluoroacetic acid (1/3) (14.8 mg, 9.73 μmol, 9.80%) as a white solid with 92% purity (215 nm) and 97% purity (254 nm).

### Example 7: ¹⁷⁵Lutetium (III) Complex of Compound 1

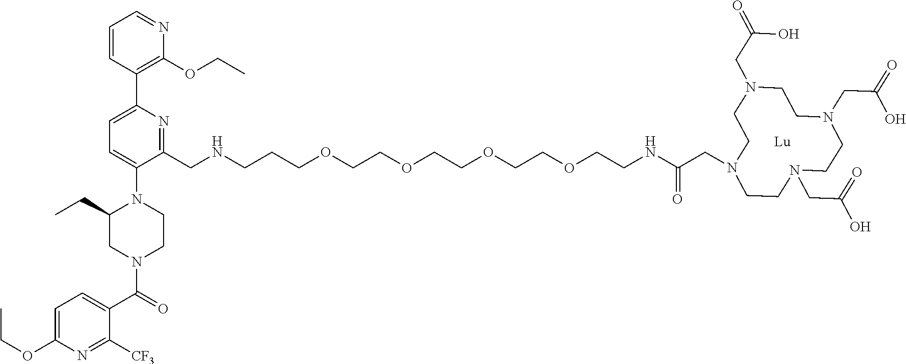

Into an 8 mL flask were added (R)-2,2′,2″-(10-(1-(2′-ethoxy-5-(4-(6-ethoxy-2-(trifluoromethyl)nicotinoyl)-2-ethylpiperazin-1-yl)-[2,3′-bipyridin]-6-yl)-19-oxo-6,9,12,15-tetraoxa-2,18-diazaicosan-20-yl)-1,4,7,10-tetraazacyclododecane-1,4,7-triyl)triacetic acid (120 mg, 1 Eq, 102 μmol), ¹⁷⁵Lutetium (III) chloride (90 mg, 23 μL, 3.1 Eq, 0.32 mmol), sodium bicarbonate (50 mg, 5.8 Eq, 0.60 mmol), Acetonitrile (1 mL) and Water (0.5 mL). The resulting mixture was stirred at 80° C. for 2 hrs. The reaction mixture was diluted with 4 mL of DMSO and filtered. The filtrate was purified by Prep-HPLC using the following conditions: Column, SunFire Prep C18 OBD Column, 19×150 mm, 5 μm, 10 nm; mobile phase, Water (0.05% TFA) and ACN (30% ACN up to 75% in 15 min); Total flow rate, 20 mL/min; Detector, UV 220 nm. This resulted in the Lu Complex of Compound 1 (51.2 mg, 37.9 μmol, 37.2%) as a white solid. [M+H-FA]=1350.8.

### Example 8: ¹¹⁵Indium (III) Complex of Compound 1

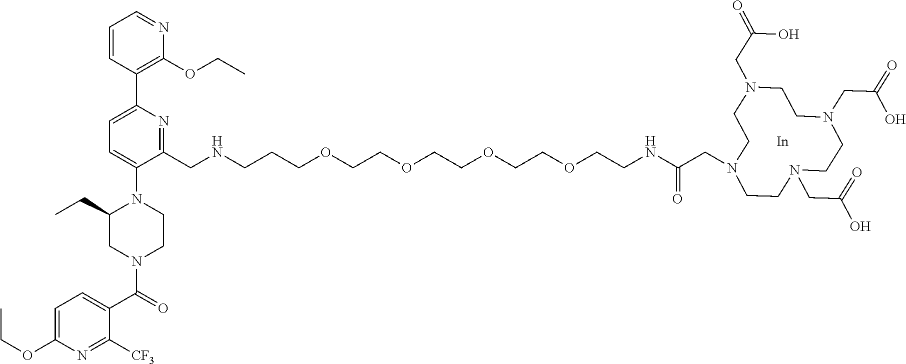

Into an 8 mL flask were added (R)-2,2′,2″-(10-(1-(2′-ethoxy-5-(4-(6-ethoxy-2-(trifluoromethyl)nicotinoyl)-2-ethylpiperazin-1-yl)-[12,3′-bipyridin]-6-yl)-19-oxo-6,9,12,15-tetraoxa-2,18-diazaicosan-20-yl)-1,4,7,10-tetraazacyclododecane-1,4,7-triyl)triacetic acid (112 mg, 1 Eq, 95.1 μmol), ¹¹⁵InCl₃ (65.0 mg, 3.09 Eq, 294 μmol), sodium hydrogen carbonate (258 mg, 32.3 Eq, 3.07 mmol), MeCN (0.5 mL) and water (0.5 mL). The reaction mixture was stirred at 80° C. for 3 hrs. Additional 4 mL of MeCN was added to the mixture and the organic layer was separated. The organic phase was purified by Prep-HPLC using the following conditions: Sunfire Prep C18 OBD Column, 50×250 mm, 5 μm, 10 nm; Mobile Phase A: Water (0.05% TFA), Mobile Phase B: ACN; Flow rate: 90 mL/min; Gradient: 100% B to 45% B in 12 min, 45% B. Pure fractions were combined and concentrated to afford the product (52.0 mg, 37.0 μmol, 39.0%) as a white solid. [M+H-1TFA]=1290.6.

### Example 9: N-((2′-ethoxy-5-(4-(6-ethoxy-2-(trifluoromethyl)nicotinoyl)piperazin-1-yl)-[2,3′-bipyridin]-6-yl)methyl)-2-nitrobenzenesulfonamide

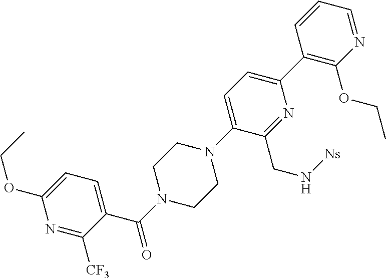

Into a 40-mL vial, was placed a mixture of (4-(6-(aminomethyl)-2′-ethoxy-[2,3′-bipyridin]-5-yl)piperazin-1-yl)(6-ethoxy-2-(trifluoromethyl)pyridin-3-yl)methanone (600 mg, 1 Eq, 1.13 mmol), TEA (343 mg, 472 μL, 3.00 Eq, 3.39 mmol) and DCM (10 mL). Nosyl chloride (276 mg, 1.10 Eq, 1.25 mmol) was added at 0° C. The reaction mixture was stirred at 20° C. for 1 hour. The mixture was concentrated and directly purified by silica gel chromatography (EtOAc/PE, from 0% to 85% in 10 μmin). Pure fractions were combined and concentrated to afford N-((2′-ethoxy-5-(4-(6-ethoxy-2-(trifluoromethyl)nicotinoyl)piperazin-1-yl)-[2,3′-bipyridin]-6-yl)methyl)-2-nitrobenzenesulfonamide (800 mg, 1.12 mmol, 98.8%) as a light yellow solid. [M+H]=716.3.

### Example 10: tert-butyl (1-(2′-ethoxy-5-(4-(6-ethoxy-2-(trifluoromethyl)nicotinoyl)piperazin-1-yl)-[2,3′-bipyridin]-6-yl)-2-((2 nitrophenyl)sulfonyl)-6,9,12,15-tetraoxa-2-azaheptadecan-17-yl)carbamate

Into a 40-mL vial, was placed a mixture of N-((2′-ethoxy-5-(4-(6-ethoxy-2-(trifluoromethyl)nicotinoyl)piperazin-1-yl)-[2,3′-bipyridin]-6-yl)methyl)-2-nitrobenzenesulfonamide (800 mg, 1 Eq, 1.12 mmol), 2,2-dimethyl-4-oxo-3,8,11,14,17-pentaoxa-5-azaicosan-20-yl methanesulfonate (719 mg, 1.50 Eq, 1.67 mmol), K₂CO₃ (463 mg, 3.00 Eq, 3.35 mmol) and MeCN (12 mL). The resulting mixture was stirred at 80° C. for 4 hrs. The reaction mixture was purified by Prep-HPLC using the following conditions: Column, C18 120 g Column; mobile phase, Water (0.05% NH₃) and ACN (30% ACN up to 98% in 7 min, then 98% ACN to 98% in 5 min); Total flow rate, 70 mL/min; Detector, UV 220 nm. Pure fractions were combined and concentrated under vacuum. This resulted in tert-butyl (1-(2′-ethoxy-5-(4-(6-ethoxy-2-(trifluoromethyl)-nicotinoyl)-piperazin-1-yl)-[2,3′-bipyridin]-6-yl)-2-((2-nitrophenyl)sulfonyl)-6,9,12,15-tetraoxa-2-azaheptadecan-17-yl)carbamate (840 mg, 801 μmol, 71.6%) as a light yellow solid. [M+H]=1049.4.

### Example 11: N-(1-amino-3,6,9,12-tetraoxapentadecan-15-yl)-N-((2′-ethoxy-5-(4-(6-ethoxy-2-(trifluoromethyl)nicotinoyl)piperazin-1-yl)-[2,3′-bipyridin]-6-yl)methyl)-2-nitrobenzenesulfonamide

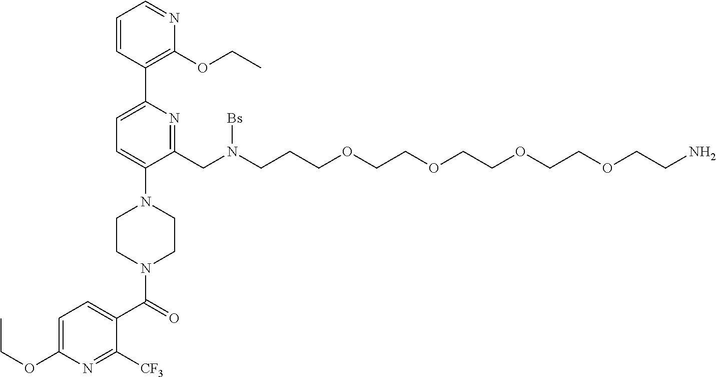

To a DCM (10 mL) solution of tert-butyl (1-(2′-ethoxy-5-(4-(6-ethoxy-2-(trifluoromethyl)-nicotinoyl)piperazin-1-yl)-[2,3′-bipyridin]-6-yl)-2-((2-nitrophenyl)sulfonyl)-6,9,12,15-tetraoxa-2-azaheptadecan-17-yl)carbamate (840 mg, 1 Eq, 801 μmol) was added TFA (3 mL) at 20° C. while stirring. The resulting mixture was stirred at 20° C. for another 1 hr. The reaction mixture was concentrated under vacuum to give crude N-(1-amino-3,6,9,12-tetraoxapentadecan-15-yl)-N-((2′-ethoxy-5-(4-(6-ethoxy-2-(trifluoromethyl)nicotinoyl)piperazin-1-yl)-[2,3′-bipyridin]-6-yl)methyl)-2-nitrobenzenesulfonamide bis(2,2,2-trifluoroacetate) (942 mg, 800 μmol, 100%) as a yellow oil. This material was used in next step without further purification.

### Example 12: 2,2′,2″-(10-(1-(2′-ethoxy-5-(4-(6-ethoxy-2-(trifluoromethyl)nicotinoyl)piperazin-1-yl)-[2,3′-bipyridin]-6-yl)-19-oxo-6,9,12,15-tetraoxa-2,18-diazaicosan-20-yl)-1,4,7,10-tetraazacyclododecane-1,4,7-triyl)triacetic acid

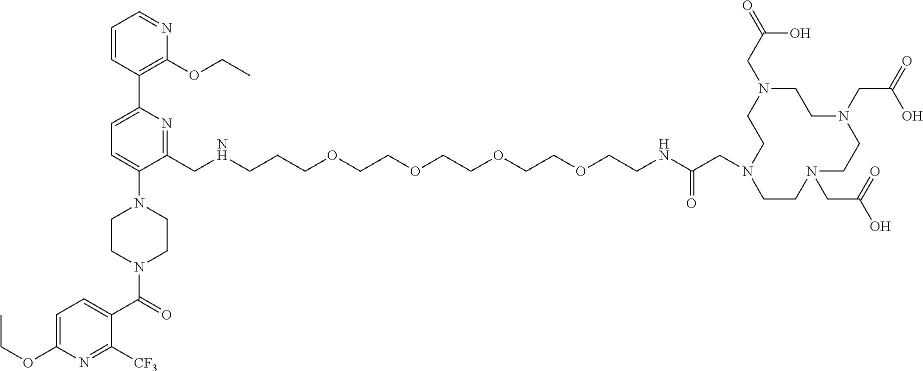 [SMILES: CCOc1ccc(C(=O)N2CCN(c3ccc(-c4cccnc4OCC)nc3CNCCCOCCOCCOCCOCCNC(=O)CN3CCN(CC(=O)O)CCN(CC(=O)O)CCN(CC(=O)O)CC3)CC2)c(C(F)(F)F)n1]

Step 1: Into a 40-mL vial, was placed 2-(4,7,10-tris(2-(tert-butoxy)-2-oxoethyl)-1,4,7,10-tetraazacyclododecan-1-yl)acetic acid (460 mg, 1.00 Eq, 803 μmol), HATU (456 mg, 1.50 Eq, 1.20 mmol), DIEA (517 mg, 697 μL, 5.00 Eq, 4.00 mmol) and DMF (15 mL). The resulting mixture was stirred at 20° C. for 15 min, followed by the addition of N-(1-amino-3,6,9,12-tetraoxapentadecan-15-yl)-N-((2′-ethoxy-5-(4-(6-ethoxy-2-(trifluoromethyl)nicotinoyl)piperazin-1-yl)-[2,3′-bipyridin]-6-yl)methyl)-2-nitrobenzenesulfonamide bis(2,2,2-trifluoroacetate) (942 mg, 1 Eq, 800 μmol). The reaction mixture was stirred at 25° C. for another 1 hr. The crude mixture was purified by Prep-HPLC using the following conditions: Column, C18 120 g Column; mobile phase, Water (0.05% FA) and ACN (30% ACN up to 98% in 7 min, then 98% ACN to 98% in 5 min); Total flow rate, 70 mL/min; Detector, UV 220 nm. Pure fractions were combined and concentrated under vacuum. This resulted in tri-tert-butyl 2,2′,2″-(10-(1-(2′-ethoxy-5-(4-(6-ethoxy-2-(trifluoromethyl)nicotinoyl)piperazin-1-yl)-[2,3′-bipyridin]-6-yl)-2-((2-nitrophenyl)sulfonyl)-19-oxo-6,9,12,15-tetraoxa-2,18-diazaicosan-20-yl)-1,4,7,10-tetraazacyclododecane-1,4,7-triyl)triacetate (550 mg, 366 μmol, 45.7%) as light yellow solid. [M/2+H]=752.7.

Step 2: Into a 40-mL vial, was placed tri-tert-butyl 2,2′,2″-(10-(1-(2′-ethoxy-5-(4-(6-ethoxy-2-(trifluoromethyl)nicotinoyl)piperazin-1-yl)-[2,3′-bipyridin]-6-yl)-2-((2-nitrophenyl)-sulfonyl)-19-oxo-6,9,12,15-tetraoxa-2,18-diazaicosan-20-yl)-1,4,7,10-tetraazacyclododecane-1,4,7-triyl)triacetate (500 mg, 1 Eq, 333 μmol), 2-chlorobenzenethiol (146 mg, 3.04 Eq, 1.01 mmol), K₂CO₃ (185 mg, 4.03 Eq, 1.34 mmol) and MeCN (6 mL). The reaction mixture was stirred at 50° C. for 2 hrs. The reaction mixture was concentrated, mixed with 5 mL of saturated Na₂CO₃, and extracted with DCM (20 mL). The organic layer was washed with water (8 mL) and brine (8 mL), dried over anhydrous sodium sulfate, filtered, and concentrated. This resulted in crude tri-tert-butyl 2,2′,2″-(10-(1-(2′-ethoxy-5-(4-(6-ethoxy-2-(trifluoromethyl)nicotinoyl)piperazin-1-yl)-[2,3′-bipyridin]-6-yl)-19-oxo-6,9,12,15-tetraoxa-2,18-diazaicosan-20-yl)-1,4,7,10-tetraazacyclododecane-1,4,7-triyl)triacetate (740 mg, 143 μmol, 42.9%, 25.4% Purity) as a light yellow oil. This material was used in next step without further purification. [M+H]=1318.7.

Step 3: Into a 50 mL single-necked flask was charged with tri-tert-butyl 2,2′,2″-(10-(1-(2′-ethoxy-5-(4-(6-ethoxy-2-(trifluoromethyl)nicotinoyl)piperazin-1-yl)-[2,3′-bipyridin]-6-yl)-19-oxo-6,9,12,15-tetraoxa-2,18-diazaicosan-20-yl)-1,4,7,10-tetraazacyclododecane-1,4,7-triyl)triacetate (740 mg, 25.4% Wt, 1 Eq, 143 μmol) and TFA (10 mL). The resulting reaction mixture was stirred at 25° C. for 5 hrs. The resulting mixture was concentrated under vacuum. This resulted in crude 2,2′,2″-(10-(1-(2′-ethoxy-5-(4-(6-ethoxy-2-(trifluoromethyl)nicotinoyl)piperazin-1-yl)-[2,3′-bipyridin]-6-yl)-19-oxo-6,9,12,15-tetraoxa-2,18-diazaicosan-20-yl)-1,4,7,10-tetraazacyclododecane-1,4,7-triyl)triacetic acid (590 mg, 161 μmol, 113%, 31.4% Purity) as a light brown oil, which was used in next step without further purification. [M+H]=1150.5.

### Example 13: tert-butyl (R)-2-(((benzyloxy)carbonyl)amino)-6-((methylsulfonyl)oxy)hexanoate

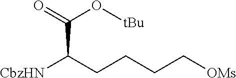 [SMILES: CC(C)(C)OC(=O)[C@@H](CCCCOS(C)(=O)=O)NC(=O)OCc1ccccc1]

Step 1: ((benzyloxy)carbonyl)-D-lysine (1.0 g, 1 Eq, 3.6 mmol) was dissolved in water (14 mL) and 4.0 M NaOH solution was added until pH=9.5. The reaction mixture was heated to 60° C., and sodium nitroprusside (1.6 g, 1.6 Eq, 5.7 mmol) was added portion-wise over 20 min at the same temperature. The pH of reaction mixture was maintained between 9-10 by adding additional 4 M NaOH solution. The reaction mixture was stirred at 60° C. for an additional 5 hrs, cooled down to ambient temperature, and filtered through celite powder. The pH of filtrate was adjusted to 3.5 using 6 N HCl solution and extracted with EtOAc (3×10 mL). The organic layers were combined, washed with brine (10 mL), dried (Na₂SO₄) and concentrated. This resulted in crude (R)-2-(((benzyloxy)-carbonyl)amino)-6-hydroxyhexanoic acid (720 mg, 2.56 mmol, 72%) as a light yellow oil, which was used in next step without further purification. [M+H]=282.2.

Step 2: To a solution of (R)-2-(((benzyloxy)carbonyl)amino)-6-hydroxyhexanoic acid (750 mg, 1 Eq, 2.67 mmol) in DCM (15 mL) was added a solution of tert-butyl (Z)—N,N′-diisopropylcarbamimidate (2.6 g, 4.9 Eq, 13 mmol) in DCM (5 mL) at 0° C. The resulting mixture was stirred at 0° C. for 0.5 hr, warmed up to ambient temperature and stirred for another 14 hrs. The reaction mixture was filtered, and the filtrate was concentrated under vacuum. The remaining residue was purified by silica gel chromatography (PE/EA=1/2) to afford tert-butyl (R)-2-(((benzyloxy)-carbonyl)amino)-6-hydroxyhexanoate (190 mg, 563 μmol, 21.1%) as a colorless oil. M+Na=360.1.

Step 3: Into an 8-mL vial, was placed a mixture of tert-butyl (R)-2-(((benzyloxy)-carbonyl)amino)-6-hydroxyhexanoate (40 mg, 1 Eq, 0.12 mmol) and TEA (36 mg, 50 μL, 3.0 Eq, 0.36 mmol) and DCM (0.5 mL). Methanesulfonyl chloride (21 mg, 1.5 Eq, 0.18 mmol) was added dropwise at 0° C. The reaction mixture was stirred at 0° C. for 1 hr. The resulting mixture was quenched with 3 mL of water and extracted with DCM (8 mL×2). The organic layers were combined, washed with brine (5 mL), dried over anhydrous Na₂SO₄, and concentrated under reduced pressure to afford crude tert-butyl (R)-2-(((benzyloxy)carbonyl)amino)-6-((methylsulfonyl)oxy)-hexanoate (42 mg, 0.10 mmol, 85%) as a light yellow oil. This material was used in next step without further purification. 2M+Na=853.3.

### Example 14: tert-butyl N2-((benzyloxy)carbonyl)-N6-((2′-ethoxy-5-((R)-4-(6-ethoxy-2-(trifluoromethyl)nicotinoyl)-2-ethylpiperazin-1-yl) [2,3′-bipyridin]-6-Amethyl)-N6-((2-nitrophenyl)sulfonyl)-D-lysinate

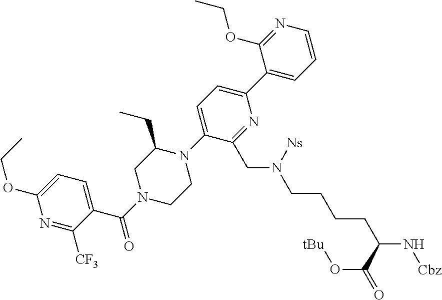

Into an 8 mL vial, was placed a mixture of (R)—N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (40 mg, 1 Eq, 54 μmol), tert-butyl (R)-2-(((benzyloxy)carbonyl)amino)-6-((methylsulfonyl)oxy)hexanoate (42 mg, 1.9 Eq, 0.10 mmol), triethylamine (120 mg, 3.01 Eq, 1.19 mmol) and ACN (1 mL). The reaction mixture was stirred at 80° C. for 15 hrs. The reaction mixture was purified by Prep-HPLC using the following conditions: Column, C18 120 g Column; mobile phase, Water (0.1% TFA) and ACN (30% ACN up to 98% in 7 min); Total flow rate, 70 mL/min; Detector, UV 220 nm. Pure fractions were combined and concentrated under vacuum. This resulted in tert-butyl N2-((benzyloxy)carbonyl)-N6-((2′-ethoxy-5-((R)-4-(6-ethoxy-2-(trifluoromethyl)-nicotinoyl)-2-ethylpiperazin-1-yl) [2,3′-bipyridin]-6-Amethyl)-N6-((2-nitrophenyl)sulfonyl)-D-lysinate (30 mg, 28 μmol, 52%) as a light yellow solid. [M+H]=1063.4.

### Example 15: N6-((2′-ethoxy-5-((R)-4-(6-ethoxy-2-(trifluoromethylnicotinoyl)-2-ethylpiperazin-1-yl)-[2,3′-bipyridin]-6-yl)methyl)-N6-((2-nitrophenyl)sulfonyl)-D-lysine

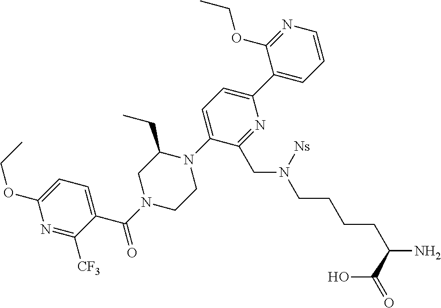

Into an 8-mL vial, was placed tert-butyl N2-((benzyloxy)carbonyl)-N6-((2′-ethoxy-5-((R)-4-(6-ethoxy-2-(trifluoromethyl)nicotinoyl)-2-ethylpiperazin-1-yl)-[2,3′-bipyridin]-6-yl)methyl)-N6-((2-nitrophenyl)sulfonyl)-D-lysinate (30 mg, 1 Eq, 28 μmol) and TFA (0.5 mL). The resulting solution was stirred for 2 hrs at 60° C. The reaction mixture was concentrated under vacuum. This resulted in crude N6-((2′-ethoxy-5-((R)-4-(6-ethoxy-2-(trifluoromethylnicotinoyl)-2-ethylpiperazin-1-yl)-[2,3′-bipyridin]-6-yl)methyl)-N6-((2-nitrophenyl)sulfonyl)-D-lysine (25 mg, 29 μmol, 100%) as a light brown oil, which was used in next step without further purification. M+H=873.3.

### Example 16: N6-((2′-ethoxy-5-((R)-4-(6-ethoxy-2-(trifluoromethylnicotinoyl)-2-ethylpiperazin-1-yl)-[2,3′-bipyridin]-6-yl)methyl)-N6-((2-nitrophenyl)sulfonyl)-N2-(2-(4,7,10-tris(2-(tert-butoxy)-2-oxoethyl)-1,4,7,10-tetraazacyclododecan-1-yl)acetyl)-D-lysine

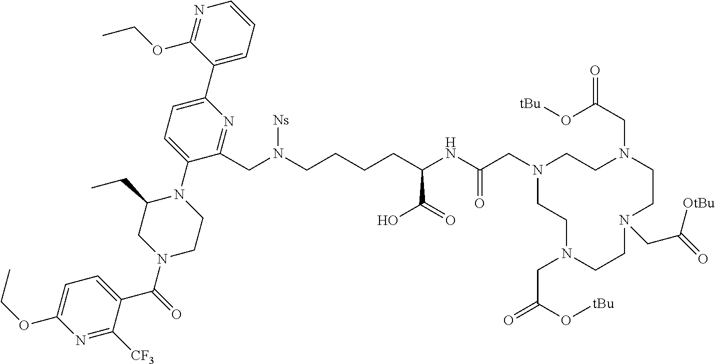

Into an 8-mL vial, was placed a mixture of 2-(4,7,10-tris(2-(tert-butoxy)-2-oxoethyl)-1,4,7,10-tetraazacyclododecan-1-yl)acetic acid (21 mg, 1.3 Eq, 37 μmol), HATU (15 mg, 1.4 Eq, 39 μmol), DIEA (18 mg, 24 μL, 4.9 Eq, 0.14 mmol) and DMF (0.5 ml). The resulting mixture was stirred at ambient temperature for 15 min followed by the addition of N6-((4nitrophenyl)sulfonyl)-D-lysine (25 mg, 1 Eq, 29 μmol). The reaction mixture was stirred at 22° C. for 1 h. The mixture was directly purified by Prep-HPLC with the following conditions: Column, C18 120 g Column; mobile phase, Water (0.05% FA) and ACN (30% ACN up to 98% in 10 min); Total flow rate, 70 mL/min; Detector, UV 220 nm. Pure fractions were collected and concentrated. This resulted in N6-((2′-ethoxy-5-((R)-4-(6-ethoxy-2-(trifluoromethylnicotinoyl)-2-ethylpiperazin-1-yl)-[2,3′-bipyridin]-6-yl)methyl)-N6-((2-nitrophenyl)sulfonyl)-N2-(2-(4,7,10-tris(2-(tert-butoxy)-2-oxoethyl)-1,4,7,10-tetraazacyclododecan-1-yl)acetyl)-D-lysine (10 mg, 7.0 μmol, 24%) as a solid. [M+H]=1428.0.

### Example 17: N6-((2′-ethoxy-5-((R)-4-(6-ethoxy-2-(trifluoromethyl)nicotinoyl)-2-ethylpiperazin-1-yl) [2,3′-bipyridin]-6-yl)methyl)-N2-(2-(4,7,10-tris(2-(tert-butoxy)-2-oxoethyl)-1,4,7,10-tetraazacyclododecan-1-yl)acetyl)-D-lysine

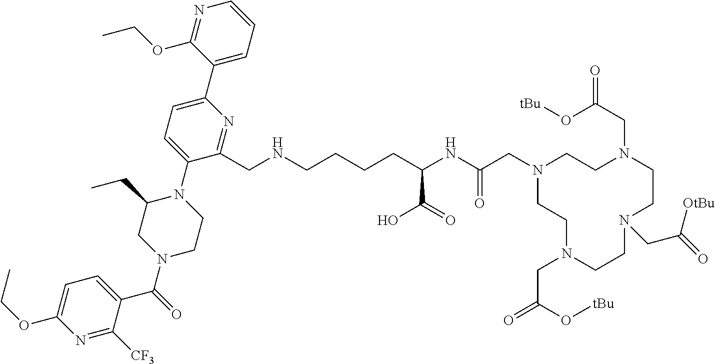 [SMILES: CCOc1ccc(C(=O)N2CCN(c3ccc(-c4cccnc4OCC)nc3CNCCCC[C@@H](NC(=O)CN3CCN(CC(=O)OC(C)(C)C)CCN(CC(=O)OC(C)(C)C)CCN(CC(=O)OC(C)(C)C)CC3)C(=O)O)[C@H](CC)C2)c(C(F)(F)F)n1]

Into an 8-mL vial, was placed a mixture of N6-((2′-ethoxy-5-((R)-4-(6-ethoxy-2-(trifluoromethyl)nicotinoyl)-2-ethylpiperazin-1-yl)-[2,3′-bipyridinn]-6-yl)methyl)-N6-((2-nitrophenyl)sulfonyl)-N2-(2-(4,7,10-tris(2-(tert-butoxy)-2-oxoethyl)-1,4,7,10-tetraazacyclododecan-1-yl)acetyl)-D-lysine (10 mg, 1 Eq, 7.0 μmol), K₂CO₃ (4.3 mg, 4.4 Eq, 31 μmol), 2-chlorobenzenethiol (3.1 mg, 3.1 Eq, 21 μmol) and ACN (0.3 mL). The reaction mixture was stirred at 50° C. for 4 hrs. The reaction crude was purified by Prep-HPLC using the following conditions: Column, C18 120 g Column; mobile phase, Water (0.05% TFA) and ACN (27% ACN up to 47% in 8 min); Total flow rate, 20 mL/min; Detector, UV 220 nm. Pure fractions were collected and concentrated. This resulted in N6-((2′-ethoxy-5-((R)-4-(6-ethoxy-2-(trifluoromethyl)nicotinoyl)-2-ethylpiperazin-1-yl) [2,3′-bipyridin]-6-yl)methyl)-N2-(2-(4,7,10-tris(2-(tert-butoxy)-2-oxoethyl)-1,4,7,10-tetraazacyclododecan-1-yl)acetyl)-D-lysine (7 mg, 6 μmol, 80%) as an oil. [M+H]=1242.6.

### Example 18: 2,2′,2″-(10-(2-(((R)-1-carboxy-5-(((2′-ethoxy-5-((R)-4-(6-ethoxy-2-(trifluoromethyl)nicotinoyl)-2-ethylpiperazin-1-yl)-[2,3′-bipyridin]-6-yl)methyl)amino)penty)-amino)-2-oxoethyl)-1,4,7,10-tetraazacyclododecane-1,4,7-triy)ptriacetic acid (Compound 2)

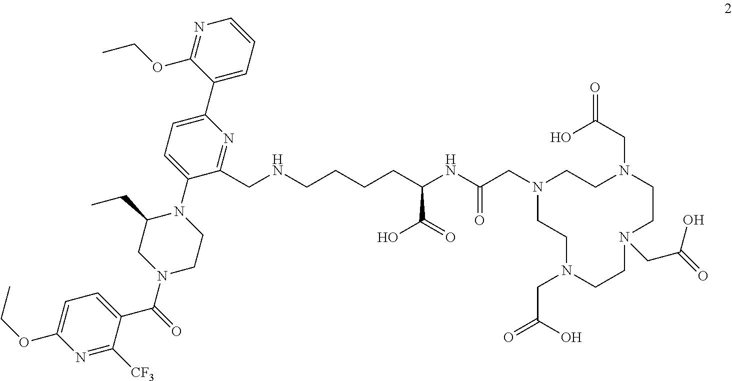 [SMILES: CCOc1ccc(C(=O)N2CCN(c3ccc(-c4cccnc4OCC)nc3CNCCCC[C@@H](NC(=O)CN3CCN(CC(=O)O)CCN(CC(=O)O)CCN(CC(=O)O)CC3)C(=O)O)[C@H](CC)C2)c(C(F)(F)F)n1]

Into an 8-mL vial, was placed N6-((2′-ethoxy-5-((R)-4-(6-ethoxy-2-(trifluoromethylnicotinoyl)-2-ethylpiperazin-1-yl)42,3′-bipyridin]-6-yl)methyl)-N2-(2-(4,7,10-tris(2-(tert-butoxy)-2-oxoethyl)-1,4,7,10-tetraazacyclododecan-1-yl)acetyp-D-lysine (7.0 mg, 1 Eq, 5.6 μmol) and TFA (0.4 mL). The resulting solution was stirred at 25° C. for 5 hrs. The resulting mixture was concentrated under vacuum to afford crude 2,2′,2″-(10-(2-(((R)-1-carboxy-5-(((2′-ethoxy-5-((R)-4-(6-ethoxy-2-(trifluoromethyl)nicotinoyl)-2-ethylpiperazin-1-yl)-[2,3′-bipyridin]-6-yl)methyl)-amino)penty)amino)-2-oxoethyl)-1,4,7,10-tetraazacyclododecane-1,4,7-triy)ptriacetic acid (6.0 mg, 5.6 μmol, 99%) as a light brown oil, which was used in next step without further purification. M+H=1074.4.

### Example 19: ¹¹⁵Indium Complex of Compound 2

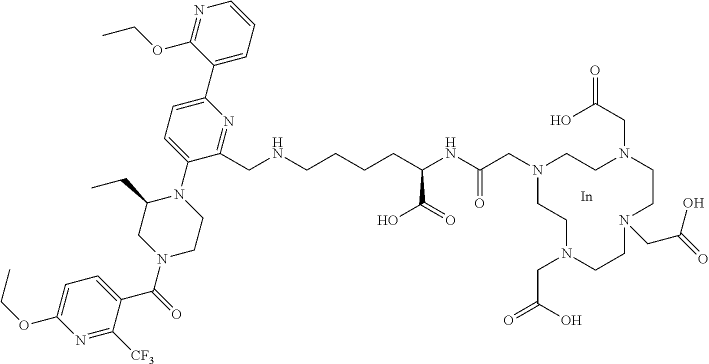

2,2′,2″-(10-(2-(((R)-1-carboxy-5-(((2′-ethoxy-5-((R)-4-(6-ethoxy-2-(trifluoromethyl)-nicotinoyl)-2-ethylpiperazin-1-yl)-[2,3′-bipyridin]-6-yl)methyl)amino)-pentyl)amino)-2-oxoethyl)-1,4,7,10-tetraazacyclododecane-1,4,7-triyl)triacetic acid (6.0 mg, 1 Eq, 5.6 μmol) was combined with sodium bicarbonate (15 mg, 6.9 μL, 32 Eq, 0.18 mmol), ¹¹⁵InCl₃ (6.5 mg, 1.9 μL, 5.3 Eq, 29 μmol), ACN (0.2 mL) and water (0.2 mL). The reaction mixture was stirred at 80° C. for 1.5 hrs. The reaction mixture was purified by Prep-HPLC using the following conditions: Column: Sunfire Prep C18 OBD Column, 50×250 mm, 5 μm 10 nm; Mobile Phase A: Water (0.05% TFA), Mobile Phase B: ACN; Flow rate: 90 mL/min; Gradient: 25% B to 45% B in 8 min. Pure fractions were collected and concentrated to afford the product (1.0 mg, 0.77 μmol, 14%) as a solid. [M+H-1TFA]=1186.6.

### Example 20: tert-butyl (2-(2-oxoethoxy)ethyl)carbamate

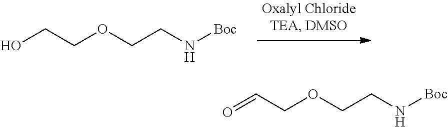 [SMILES: CC(C)(C)OC(=O)NCCOCCO ; CC(C)(C)OC(=O)NCCOCC=O]

Into a 50-mL three-necked round bottom flask purged and maintained with an inert atmosphere of nitrogen, was placed a mixture of DMSO (952 mg, 865 μL, 5.00 Eq, 12.2 mmol) and DCM (5 mL). Oxalyl chloride (618 mg, 429 μL, 2.00 Eq, 4.87 mmol) was added at −78° C. The resulting mixture was stirred at −78° C. for additional 30 min, followed by the addition of tert-butyl (2-(2-hydroxyethoxy)ethyl)carbamate (500 mg, 1 Eq, 2.44 mmol) in DCM (5 mL) at −78° C. dropwise. The reaction mixture was stirred at −78° C. for 45 min. TEA (986 mg, 1.36 mL, 4.00 Eq, 9.74 mmol) was added dropwise and the reaction mixture was stirred at −78° C. for additional 15 min. The resulting mixture was warmed up to 20° C. and stirred at the same temperature for 1 hr. The reaction crude was quenched with 100 mL of saturated NaHCO₃ solution and extracted with EtOAc (50 mL×3). The organic layers were combined and washed with water (100 mL) and brine (100 mL), dried over anhydrous Na₂SO₄ and concentrated under reduced pressure. This resulted in tert-butyl (2-(2-oxoethoxy)ethyl)carbamate (425 mg, 2.09 mmol, 85.8%) as a yellow crude oil, which was used directly for next step without purification.

### Example 21: tert-butyl 3-(3-bromopropoxy)-5-((2-(2-((tert-butoxycarbonypamino)-ethoxy)ethyl)amino)benzoate

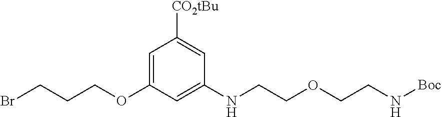 [SMILES: CC(C)(C)OC(=O)NCCOCCNc1cc(OCCCBr)cc(C(=O)OC(C)(C)C)c1]

Step 1: Into a 40-mL vial, was placed a mixture of 3-hydroxy-5-nitrobenzoic acid (500 mg, 1 Eq, 2.73 mmol) and toluene (10 mL). The reaction mixture was heated to 100° C., followed by the addition of 1,1-di-tert-butoxy-N,N-dimethylmethanamine (1.11 g, 2.00 Eq, 5.46 mmol) at the same temperature. The reaction mixture was stirred at 100° C. for 2 hrs. The reaction mixture was concentrated under reduced pressure, and the remaining crude was purified by MPLC (silica gel column, 40 g; Mobile Phase A: PE, Mobile Phase B: EtOAc; Flow rate: 35 mL/min; Gradient: 0% B to 60% B in 8 min; 254 nm). This resulted in tert-butyl 3-hydroxy-5-nitrobenzoate (365 mg, 1.53 mmol, 55.9%) as a yellow oil.

Step 2: Into a 40-mL vial, was placed a mixture of tert-butyl 3-hydroxy-5-nitrobenzoate (340 mg, 1 Eq, 1.42 mmol), 1,3-dibromopropane (861 mg, 3.00 Eq, 4.26 mmol), K₂CO₃ (982 mg, 5.00 Eq, 7.11 mmol) and MeCN (5 mL). The resulting mixture was stirred at 80° C. for 4 hrs. The reaction mixture was concentrated, dissolved in 50 mL of water, and extracted with EtOAc (40 mL×2). The organic layers were combined, washed 40 mL of brine, dried over anhydrous sodium sulfate, filtered, and concentrated. The remaining crude was purified by MPLC (silica gel column, 20 g; Mobile Phase A: PE, Mobile Phase B: EtOAc; Flow rate: 35 mL/min; Gradient: 0% B to 60% B in 8 min; Detector wavelength: 254 nm). This resulted in tert-butyl 3-(3-bromopropoxy)-5-nitrobenzoate (300 mg, 833 μmol, 58.6%) as a yellow oil.

Step 3: Into a 40-mL vial, was placed a mixture of tert-butyl 3-(3-bromopropoxy)-5-nitrobenzoate (300 mg, 1 Eq, 833 μmol), NH₄CI (312 mg, 7.00 Eq, 5.83 mmol), Iron (233 mg, 29.6 μL, 5.01 Eq, 4.17 mmol), EtOH (6.0 mL) and Water (1.0 mL). The reaction mixture was heated at 80° C. for 20 min. The resulting mixture was concentrated, dissolved in 50 mL of water and extracted with EtOAc (40 mL×2). The organic layers were combined, washed 40 mL of brine, dried over anhydrous sodium sulfate, filtered, and concentrated. The remaining crude was purified by MPLC (silica gel column, 20 g; Mobile Phase A: PE, Mobile Phase B: EtOAc; Flow rate: 35 mL/min; Gradient: 0% B to 60% B in 8 min; Detector wavelength: 254 nm). This resulted in tert-butyl 3-amino-5-(3-bromopropoxy)benzoate (251 mg, 760 μmol, 91.3%) as a solid. [M+H+MeCN]=371.1

Step 4: Into a 40-mL vial, was placed a mixture of tert-butyl 3-amino-5-(3-bromopropoxy)-benzoate (190 mg, 1 Eq, 575 μmol), tert-butyl (2-(2-oxoethoxy)ethyl)carbamate (234 mg, 2.00 Eq, 1.15 mmol), Zinc chloride (157 mg, 73.0 μL, 2.00 Eq, 1.15 mmol) and MeOH (4 mL). The reaction mixture was stirred at 60° C. for 2 hrs. NaBH₃CN (145 mg, 4.01 Eq, 2.31 mmol) was added and the reaction mixture was stirred at 60° C. for additional 16 hrs. The reaction mixture was purified by Prep-HPLC using the following conditions: Column, C18 120 g Column; mobile phase, Water (0.05% FA) and ACN (30% ACN up to 98% in 10 min); Total flow rate, 70 mL/min; Detector, UV 220 nm. Pure fractions were collected and concentrated to afford tert-butyl 3-(3-bromopropoxy)-5-((2-(2-((tert-butoxycarbonypamino)ethoxy)ethyl)amino)benzoate (120 mg, 232 μmol, 40.3%) as a yellow solid. [M+H]=517.2, 519.2.

### Example 22: tert-butyl (R)-3-((2-(2-((tert-butoxycarbonypamino)ethoxy)ethylamino)-5-(3-((N-((3-(4-(4-ethoxy-2-(trifluoromethyl)benzoyl)-2-ethylpiperazin-1-yl)-6-(2-ethoxyphenyppyridin-2-yl)methyl)-4-nitrophenypsulfonamido)propoxy)benzoate

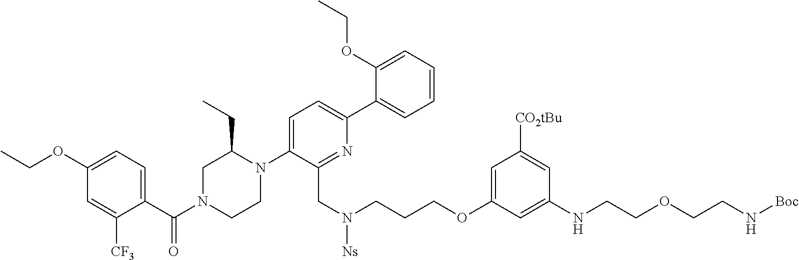

Into an 8-mL vial, was placed a mixture of tert-butyl 3-(3-bromopropoxy)-5-((2-(2-((tert-butoxycarbonypamino)ethoxy)ethyl)amino)benzoate (120 mg, 1 Eq, 232 μmol), (R)—N-((3-(4-(4-ethoxy-2-(trifluoromethylbenzoyl)-2-ethylpiperazin-1-yl)-6-(2-ethoxyphenyl)pyridin-2-yl)methyl)-2-nitrobenzenesulfonamide (172 mg, 1.00 Eq, 232 μmol), K₂CO₃ (96 mg, 3.0 Eq, 0.69 mmol) and MeCN (3 mL). The reaction mixture was stirred at 80° C. for 16 hrs. The reaction crude was purified by Prep-HPLC using the following conditions: Column, C18 120 g Column; mobile phase, Water (0.05% FA) and ACN (30% ACN up to 98% in 10 min); Total flow rate, 70 mL/min; Detector, UV 220 nm. Pure fractions were collected and concentrated to afford tert-butyl (R)-3-((2-(2-((tert-butoxycarbonypamino)ethoxy)ethylamino)-5-(3-((N-((3-(4-(4-ethoxy-2-(trifluoromethyl)benzoyl)-2-ethylpiperazin-1-yl)-6-(2-ethoxyphenyl)pyridin-2-yl)methyl)-4-nitrophenypsulfonamido)-propoxy)benzoate (90 mg, 76 μmol, 33%) as a yellow solid. [M+H]=1178.8.

### Example 23: (R)-3-((2-(2-aminoethoxy)ethyl)amino)-5-(3-((N-((3-(4-(4-ethoxy-2-(trifluoromethyl)benzoyl)-2-ethylpiperazin-1-yl)-6-(2-ethoxyphenyl)pyridin-2-yl)methyl)-2-nitrophenyl)sulfonamido)propoxy)benzoic acid

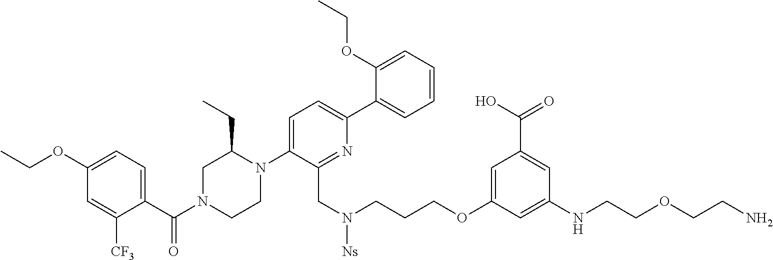

Into an 8-mL vial, was placed a mixture of tert-butyl (R)-3-((2-(2-((tert-butoxycarbonyl-amino)ethoxy)ethyl)amino)-5-(3-((N-((3-(4-(4-ethoxy-2-(trifluoromethylbenzoyl)-2-ethylpiperazin-1-yl)-6-(2-ethoxyphenyl)pyridin-2-yl)methyl)-4-nitrophenypsulfonamido)-propoxy)benzoate (90 mg, 1 Eq, 76 μmol) and DCM (2 mL). TFA (2 mL) was added, and the reaction mixture was stirred at 25° C. for 1 hour. The reaction crude was purified by Prep-HPLC using the following conditions: Column, C18 120 g; mobile phase, Water (0.05% FA) and ACN (30% ACN up to 98% in 10 min); Total flow rate, 70 mL/min; Detector, UV 220 nm. Pure fractions were combined and concentrated. This resulted in (R)-3-((2-(2-aminoethoxy)ethyl)amino)-5-(3-((N-((3-(4-(4-ethoxy-2-(trifluoromethyl)benzoyl)-2-ethylpiperazin-1-yl)-6-(2-ethoxyphenyl)pyridin-2-yl)methyl)-2-nitrophenyl)sulfonamido)propoxy)benzoic acid (70 mg, 68 μmol, 90%) as an oil. [M+H]=1022.5.

### Example 24: (R)-2,2′,2″-(10-(2-((2-(2-((3-carboxy-5-(3-(((3-(4-(4-ethoxy-2-(trifluoromethyl)-benzoyl)-2-ethylpiperazin-1-yl)-6-(2-ethoxyphenyl)pyridin-2-yl)methyl)amino)propoxy)phenyl)-amino)ethoxy)ethyl)amino)-2-oxoethyl)-1,4,7,10-tetraazacyclododecane-1,4,7-triyl)triacetic acid (Compound 3)

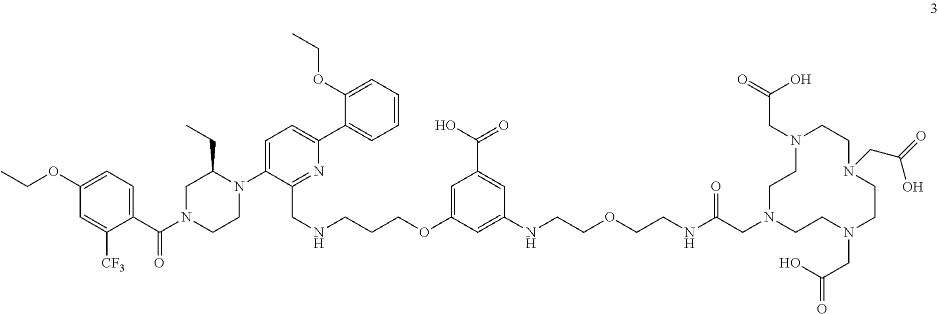 [SMILES: CCOc1ccc(C(=O)N2CCN(c3ccc(-c4ccccc4OCC)nc3CNCCCOc3cc(NCCOCCNC(=O)CN4CCN(CC(=O)O)CCN(CC(=O)O)CCN(CC(=O)O)CC4)cc(C(=O)O)c3)[C@H](CC)C2)c(C(F)(F)F)c1]

Step 1: Into an 8-mL vial, was placed a mixture of 2-(4,7,10-tris(2-(tert-butoxy)-2-oxoethyl)-1,4,7,10-tetraazacyclododecan-1-yl)acetic acid (78 mg, 2.0 Eq, 0.14 mmol), 2-(3H-[1,2,3]triazolo[4,5-b]pyridin-3-yl)-1,1,3,3-tetramethylisouronium hexafluorophosphate(V) (52 mg, 2.0 Eq, 0.14 mmol), DIEA (44 mg, 5.0 Eq, 0.34 mmol) and DMF (2 mL). The resulting mixture was stirred at 25° C. for 10 min, followed by the addition of (R)-3-((2-(2-aminoethoxy)ethyl)amino)-5-(3-((N-((3-(4-(4-ethoxy-2-(trifluoromethyl)benzoyl)-2-ethylpiperazin-1-yl)-6-(2-ethoxyphenyl)pyridin-2-yl)methyl)-2-nitrophenyl)sulfonamido)propoxy)benzoic acid (70 mg, 1 Eq, 68 μmol). The reaction mixture was stirred at 25° C. for 1 hour. The reaction crude was purified by Prep-HPLC using the following conditions: Column, C18 120 g; mobile phase, Water (0.05% FA) and ACN (30% ACN up to 98% in 10 min); Total flow rate, 70 mL/min; Detector, UV 220 nm. This resulted in (R)-3-(3-((N-((3-(4-(4-ethoxy-2-(trifluoromethyl)benzoyl)-2-ethylpiperazin-1-yl)-6-(2-ethoxyphenyl)pyridin-2-yl)methyl)-2-nitrophenyl)sulfonamido)propoxy)-5-((2-(2-(2-(4,7,10-tris(2-(tert-butoxy)-2-oxoethyl)-1,4,7,10-tetraazacyclododecan-1-yl)acetamido)ethoxy)ethyl)amino)benzoic acid (41 mg, 26 μmol, 38%) as a light yellow solid. [M+Na]=1600.2.

Step 2: Into an 8-mL vial, was placed a mixture of (R)-3-(3-((N-((3-(4-(4-ethoxy-2-(trifluoromethyl)benzoyl)-2-ethylpiperazin-1-yl)-6-(2-ethoxyphenyl)pyridin-2-yl)methyl)-2-nitrophenyl)sulfonamido)propoxy)-5-((2-(2-(2-(4,7,10-tris(2-(tert-butoxy)-2-oxoethyl)-1,4,7,10-tetraazacyclododecan-1-yl)acetamido)ethoxy)ethyl)amino)benzoic acid (41 mg, 1 Eq, 26 μmol), 2-chlorobenzenethiol (15 mg, 4.0 Eq, 0.10 mmol), K₂CO₃ (18 mg, 5.0 Eq, 0.13 mmol) and MeCN (0.5 mL). The reaction mixture was stirred at 50° C. for 1 hour. The reaction crude was purified by Prep-HPLC using the following conditions: Column, C18 120 g; mobile phase, Water (0.05% FA) and ACN (27% ACN up to 47% in 8 min); Total flow rate, 20 mL/min; Detector, UV 220 nm. This resulted in (R)-3-(3-(((3-(4-(4-ethoxy-2-(trifluoromethyl)benzoyl)-2-ethylpiperazin-1-yl)-6-(2-ethoxyphenyl)pyridin-2-yl)methyl)amino)propoxy)-5-((2-(2-(2-(4,7,10-tris(2-(tert-butoxy)-2-oxoethyl)-1,4,7,10-tetraazacyclododecan-1-yl)acetamido)ethoxy)ethyl)amino)benzoic acid (22 mg, 16 μmol, 61%) as a light yellow oil. [M+H]=1392.1.

Step 3: Into a 8-mL vial, was placed a mixture of (R)-3-(3-(((3-(4-(4-ethoxy-2-(trifluoromethyl)-benzoyl)-2-ethylpiperazin-1-yl)-6-(2-ethoxyphenyl)pyridin-2-yl)methyl)amino)propoxy)-5-((2-(2-(2-(4,7,10-tris(2-(tert-butoxy)-2-oxoethyl)-1,4,7,10-tetraazacyclododecan-1-yl)acetamido)ethoxy)-ethyl)amino)benzoic acid (11.0 g, 1 Eq, 7.90 mmol) and TFA (0.25 mL). The resulting mixture was stirred at 25° C. for 1.5 hrs. The reaction mixture was concentrated under vacuum to afford crude (R)-2,2′,2″-(10-(2-((2-(2-((3-carboxy-5-(3-(((3-(4-(4-ethoxy-2-(trifluoromethyl)benzoyl)-2-ethyl-piperazin-1-yl)-6-(2-ethoxyphenyl)pyridin-2-yl)methyl)amino)propoxy)phenyl)amino)ethoxy)ethyl)-amino)-2-oxoethyl)-1,4,7,10-tetraazacyclododecane-1,4,7-triyl)triacetic acid (8.0 g, 6.5 mmol, 83%) as a light brown oil, which was used in next step without further purification. [M+H]=1223.9.

### Example 25: ¹¹⁵Indium Complex of Compound 3

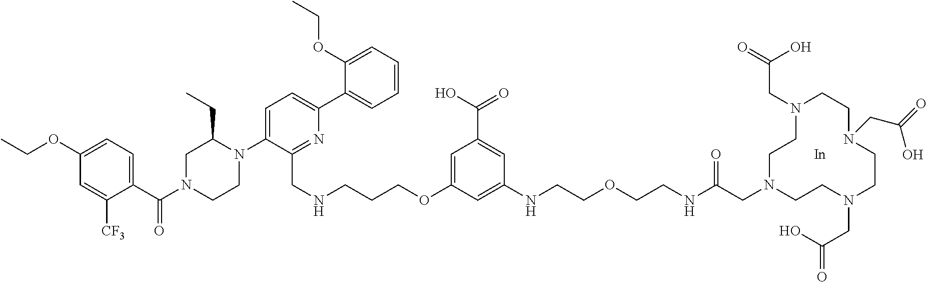

Into a 2-mL vial, was placed with a mixture of (R)-2,2′,2″-(10-(2-((2-(2-((3-carboxy-5-(3-(((3-(4-(4-ethoxy-2-(trifluoromethyl)benzoyl)-2-ethylpiperazin-1-yl)-6-(2-ethoxyphenyl)pyridin-2-yl)methyl)amino)propoxy)phenyl)amino)ethoxy)ethyl)amino)-2-oxoethyl)-1,4,7,10-tetraazacyclododecane-1,4,7-triyl)triacetic acid (8.0 mg, 1 Eq, 6.5 μmol), NaHCO₃ (6.0 mg, 11 Eq, 71 μmol), ¹¹⁵InCl₃ (14 mg, 9.7 Eq, 63 μmol), MeCN (0.15 mL) and Water (0.15 mL). The reaction mixture was stirred at 80° C. for 2 hrs. MeCN (4 mL) was added and the organic layer was separated and purified by Pre-HPLC using the following conditions: Sunfire Prep C18 OBD Column, 50*250 mm, 5 μm 10 nm; Mobile Phase A: Water (0.05% TFA), Mobile Phase B: ACN; Flow rate: 90 mL/min; Gradient: 10% B to 45% B in 12 min, 45% B. Pure fractions were combined and lyophilized to afford the product (3.9 mg, 2.7 μmol, 41%) as a white solid. [M+H-1TFA]=1335.6.

### Example 26: 2,2-dimethyl-4-oxo-3,8,11,14-tetraoxa-5-azahexadecan-16-yl methanesulfonate

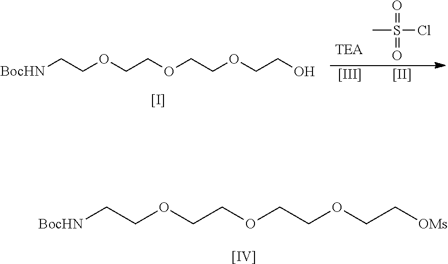

Into a 50-mL flask, was placed a mixture of tert-butyl (2-(2-(2-(2-hydroxyethoxy)-ethoxy)ethoxy)ethyl)carbamate (450 mg, 1 Eq, 1.53 mmol), TEA (465 mg, 640 μL, 3.00 Eq, 4.60 mmol) and DCM (8 mL). Methanesulfonyl chloride (229 mg, 155 μL, 1.30 Eq, 2.00 mmol) was added at 0° C. dropwise. The reaction mixture was stirred at 25° C. for 1 hour. The resulting mixture was quenched with 5 mL of water and extracted with DCM (10 mL×2). Organic layers were combined, washed with brine (5 mL), dried over anhydrous Na₂SO₄, filtered, and concentrated to afford 2,2-dimethyl-4-oxo-3,8,11,14-tetraoxa-5-azahexadecan-16-yl methanesulfonate (529 mg, 1.42 mmol, 92.8%) as an oil, which was used in next step without further purification. M+H=372.1.

### Example 27: tert-butyl (R)-(3-((N-((2′-ethoxy-5-(4-(6-ethoxy-2-(trifluoromethyl)nicotinoyl)-2-ethylpiperazin-1-yl)[2,3′-bipyridin]-6-ylmethyl)-2-nitrophenyl)sulfonamido)propyl)carbamate

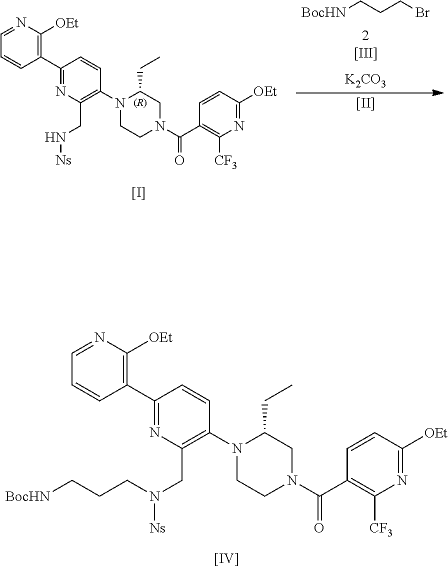

Into an 8 mL vial, was placed a mixture of (R)—N-((2′-ethoxy-5-(4-(6-ethoxy-2-(trifluoromethylnicotinoyl)-2-ethylpiperazin-1-yl)-[2,3′-bipyridin]-6-yl)methyl)-2-nitrobenzenesulfonamide (175 mg, 1 Eq, 235 μmol), K₂CO₃ (101 mg, 3.11 Eq, 731 μmol), tert-butyl (3-bromopropyl)carbamate (139 mg, 2.48 Eq, 584 μmol) and MeCN (3 mL). The reaction mixture was stirred at 80° C. for 5 hrs. The reaction crude was purified by Prep-HPLC using the following conditions: Column, C18 120 g; mobile phase, Water (0.1% FA) and ACN (30% ACN up to 98% in 7 min); Total flow rate, 70 mL/min; Detector, UV 220 nm. This resulted in tert-butyl (R)-(3-((N-((2′-ethoxy-5-(4-(6-ethoxy-2-(trifluoromethyl)nicotinoyl)-2-ethylpiperazin-1-yl) [2,3′-bipyridin]-6-ylmethyl)-2-nitrophenyl)sulfonamido)propyl)carbamate (190 mg, 211 μmol, 89.6%) as a light yellow solid. [M+H]=901.3.

### Example 28: (R)—N-(3-aminopropyl)-N-((2′-ethoxy-5-(4-(6-ethoxy-2-(trifluoromethylnicotinoyl)-2-ethylpiperazin-1-yl)-[2,3′-bipyridin]-6-yl)methyl)-2-nitrobenzenesulfonamide

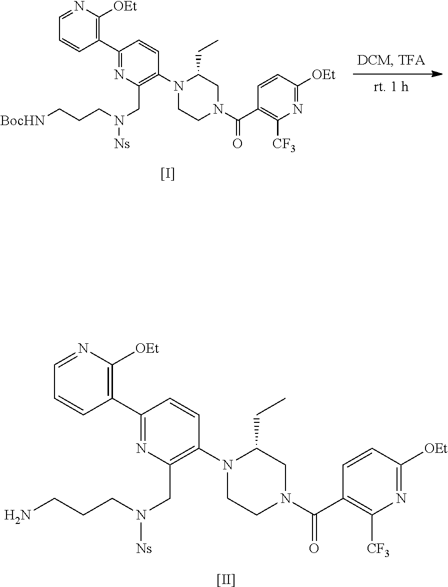

Into a 50 mL flask were added tert-butyl (R)-(3-((N-((2′-ethoxy-5-(4-(6-ethoxy-2-(trifluoromethylnicotinoyl)-2-ethylpiperazin-1-yl)-[2,3′-bipyridin]-6-yhmethyl)-4-nitrophenyl-sulfonamido)propylcarbamate (190 mg, 1 Eq, 211 μmol), DCM (3 mL) and TFA (1 mL). The resulting mixture was stirred at 25° C. for 1 hour. The reaction crude was purified by Prep-HPLC using the following conditions (IntelFlash-1): Column, C18 silica gel; mobile phase, water (0.1 NH₃·H₂O/CH₃CN (CH₃CN:30%-98% in 7 min); Detector, UV 254 & 220 n. This resulted in (R)—N-(3-aminopropyl)-N-((2′-ethoxy-5-(4-(6-ethoxy-2-(trifluoromethyhnicotinoyl)-2-ethylpiperazin-1-yl)-[2,3′-bipyridin]-6-yl)methyl)-2-nitrobenzenesulfonamide (165 mg, 206 μmol, 97.7%) as a yellow solid. M+H=801.3.

### Example 29: di-tert-butyl (12-(3-((N-((2′-ethoxy-5-(4-(6-ethoxy-2-(trifluoromethylnicotinoyl)-2-ethylpiperazin-1-yl)-[2,3′-bipyridin]-6-ylmethyl)-4-nitrophenypsulfonamido)propyl)-3,6,9,15,18,21-hexaoxa-12-azatricosane-1,23-diyl)(R)-dicarbamate

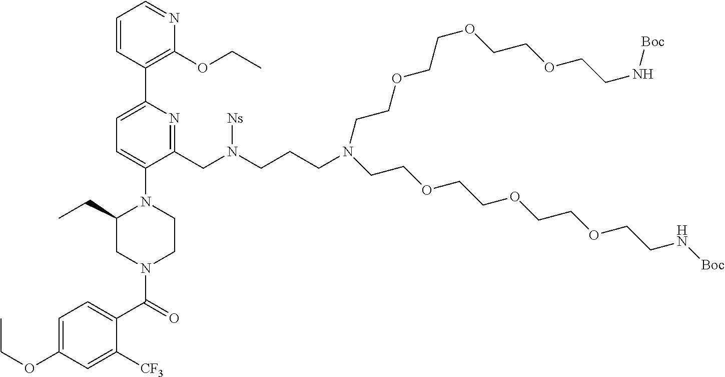

Into a 40 mL vial, was placed a mixture of (R)—N-(3-aminopropyl)-N-((2′-ethoxy-5-(4-(6-ethoxy-2-(trifluoromethyl)nicotinoyl)-2-ethylpiperazin-1-yl)42,3′-bipyridin]-6-yl)methyl)-2-nitrobenzenesulfonamide (165 mg, 1 Eq, 206 μmol), NaI (122 mg, 3.95 Eq, 814 μmol), K₂CO₃ (115 mg, 4.04 Eq, 832 μmol), 2,2-dimethyl-4-oxo-3,8,11,14-tetraoxa-5-azahexadecan-16-yl methanesulfonate (321 mg, 4.19 Eq, 864 μmol) and MeCN (5 mL). The reaction mixture was stirred at 80° C. for 3 hrs. The reaction crude was purified by Prep-HPLC using the following conditions: Column, C18 120 g; mobile phase, Water (0.1% FA) and ACN (30% ACN up to 98% in 7 min); Total flow rate, 70 mL/min; Detector, UV 220 nm. This resulted in di-tert-butyl (12-(3-((N-((2′-ethoxy-5-(4-(6-ethoxy-2-(trifluoromethylnicotinoyl)-2-ethylpiperazin-1-yl)-[2,3′-bipyridin]-6-ylmethyl)-4-nitrophenylsulfonamido)propyl)-3,6,9,15,18,21-hexaoxa-12-azatricosane-1,23-diyl)(R)-dicarbamate (95 mg, 70 μmol, 34%) as a light yellow solid. [M+H]=1352.0.

### Example 30: (R)—N-(1-amino-12-(2-(2-(2-(2-aminoethoxy)ethoxy)ethoxy)ethyl)-3,6,9-trioxa-12-azapentadecan-15-yl)-N-((2′-ethoxy-5-(4-(6-ethoxy-2-(trifluoromethylnicotinoyl)-2-ethylpiperazin-1-yl)[2,3′-bipyridin]-6-yl)methyl)-2-nitrobenzenesulfonamide

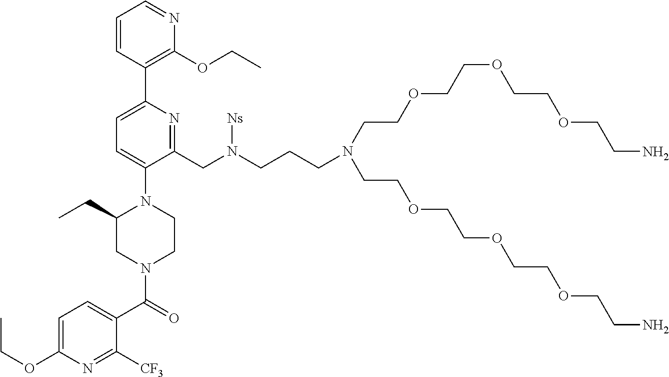

Into a 50 mL flask were added di-tert-butyl (12-(3-((N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)-3,6,9,15,18,21-hexaoxa-12-azatricosane-1,23-diyl)(8)-dicarbamate (95 mg, 1 Eq, 70 μmol), DCM (1 mL) and TFA (0.5 mL). The resulting mixture was stirred at 25° C. for 1 hour. The reaction crude was purified by Prep-HPLC using the following conditions (IntelFlash-1): Column, C18 silica gel; mobile phase, water (0.1% NH₃·H₂O/CH₃CN (CH₃CN:30%-98% in 7 min); Detector, UV 254 &220 nm. This resulted in (R)—N-(1-amino-12-(2-(2-(2-(2-aminoethoxy)ethoxy)ethoxy)ethyl)-3,6,9-trioxa-12-azapentadecan-15-yl)-N-((2′-ethoxy-5-(4-(6-ethoxy-2-(trifluoromethylnicotinoyl)-2-ethylpiperazin-1-yl)[2,3′-bipyridin]-6-yl)methyl)-2-nitrobenzenesulfonamide (65 mg, 56 μmol, 80%) as a yellow oil. M+H=1151.3.

### Example 31: Compound [I-V]

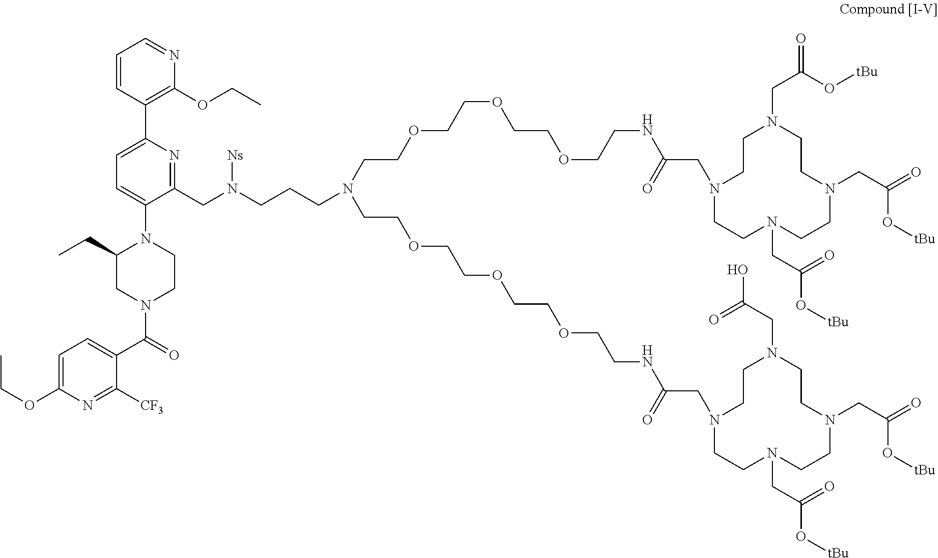

Into a 50-mL flask, was placed a mixture of 2-(4,7,10-tris(2-(tert-butoxy)-2-oxoethyl)-1,4,7,10-tetraazacyclododecan-1-ypacetic acid (49 mg, 1.5 Eq, 86 μmol), 1-Methylimidazole(N—) (33 mg, 32 μL, 7.1 Eq, 0.40 mmol), N,N,N′N-tetramethylchloroformamidinium-hexafluorophosphate (106 mg, 6.7 Eq, 378 μmol) and MeCN (4 mL). The resulting mixture was stirred at 25° C. for 2 hrs, followed by the addition of (R)—N-(1-amino-12-(2-(2-(2-(2-aminoethoxy)ethoxy)ethoxy)ethyl)-3,6,9-trioxa-12-azapentadecan-15-yl)-N-((2′-ethoxy-5-(4-(6-ethoxy-2-(trifluoromethylnicotinoyl)-2-ethylpiperazin-1-yl)-[2,3′-bipyridin]-6-ylmethyl)-2-nitrobenzenesulfonamide (65 mg, 1 Eq, 56 μmol). The reaction mixture was stirred at 25° C. for another 2 hrs. The reaction mixture was concentrated under vacuum to afford crude Compound [I-V] (0.42 g, 0.19 mmol, 330%) as light-yellow oil. This material was used in next step without further purification. [M/2+H]=1131.4.

### Example 32: Compound [I-VI]

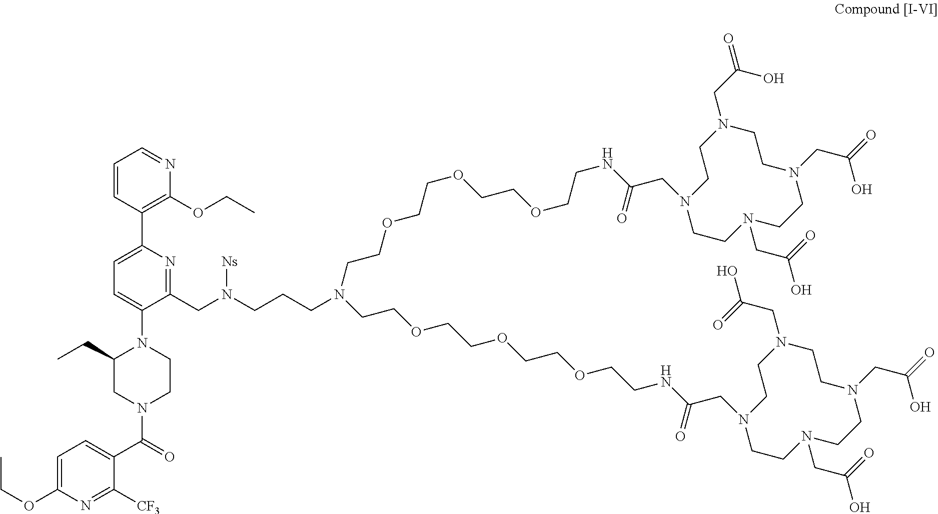

Into a 50 mL flask were added Compound [I-V] from the previous Example (420 mg, 1 Eq, 186 μmol) and TFA (3 mL). The resulting mixture was stirred at 25° C. for 5 hrs. The reaction crude was purified by Prep-HPLC using the following conditions (IntelFlash-1): Column, C18 silica gel; mobile phase, water (0.1% FA/CH₃CN (CH₃CN:30%-98% in 7 min); Detector, UV 254 &220 nm. This resulted in Compound [I-VI] (21 mg, 11 μmol, 5.9%) as a yellow solid. M+H=1924.9.

### Example 33: Compound 4

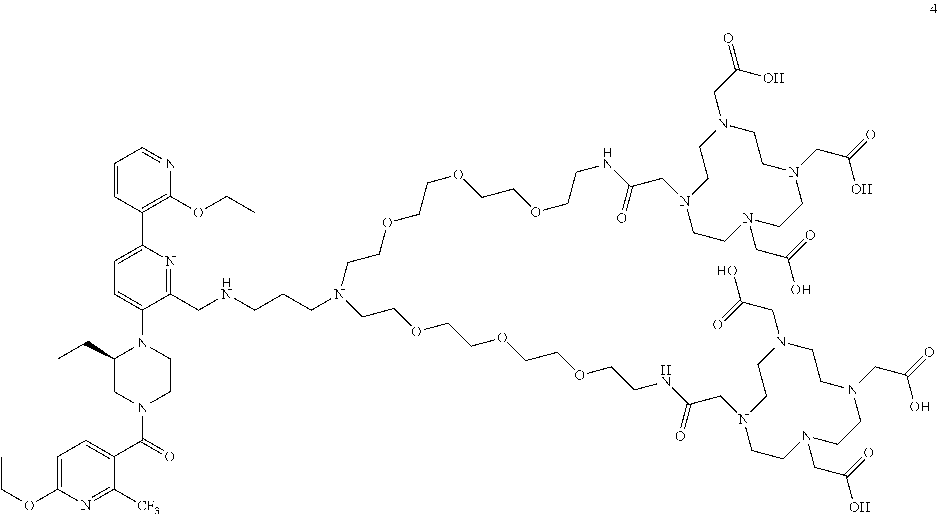 [SMILES: CCOc1ccc(C(=O)N2CCN(c3ccc(-c4cccnc4OCC)nc3CNCCCN(CCOCCOCCOCCNC(=O)CN3CCN(CC(=O)O)CCN(CC(=O)O)CCN(CC(=O)O)CC3)CCOCCOCCOCCNC(=O)CN3CCN(CC(=O)O)CCN(CC(=O)O)CCN(CC(=O)O)CC3)[C@H](CC)C2)c(C(F)(F)F)n1]

Into a 2-mL vial, was placed a mixture of compound [I-VI] from the previous Example (21 mg, 1 Eq, 11 μmol), 2-chlorobenzenethiol (8.1 mg, 5.1 Eq, 56 μmol), K₂CO₃ (4.5 mg, 3.0 Eq, 33 μmol) and MeCN (0.2 mL). The reaction mixture was stirred at 50° C. for 1 hour. The reaction crude was purified by Prep-HPLC using the following conditions: Column, C18 120 g; mobile phase, Water (0.05% TFA) and ACN (35% ACN up to 55% in 8 min); Total flow rate, 20 mL/min; Detector, UV 220 nm. This resulted in Compound 4 (8.1 mg, 4.7 μmol, 43%) as an oil. [M/2+H]=870.4.

### Example 34: ¹¹⁵Indium Complex of Compound 4

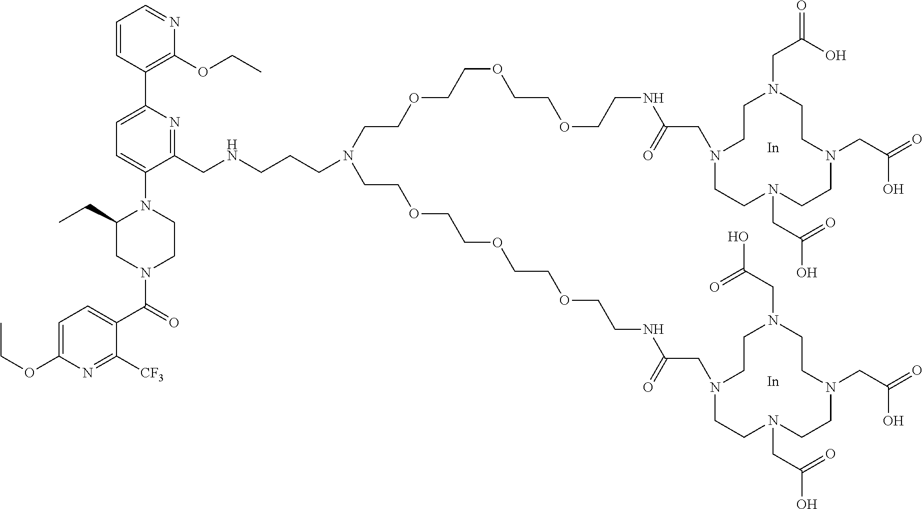

Compound 4 (8.1 mg, 1 Eq, 4.7 μmol, “Example 33”) was combined with ¹¹⁵InCl₃ (5.6 mg, 5.4 Eq, 25 μmol), NaHCO₃ (8.1 mg, 21 Eq, 96 μmol), MeCN (0.3 mL) and water (0.1 mL). The reaction mixture was stirred at 80° C. for 1 hour. The reaction crude was purified by Prep-HPLC using the following conditions: Sunfire Prep C18 OBD Column, 50×250 mm, 5 μm 10 nm; Mobile Phase A: Water (0.05% TFA), Mobile Phase B: ACN; Flow rate: 90 mL/min; Gradient: 25% B to 45% B in 8 min. Pure ructions were combined and concentrated to afford the product (3.5 mg, 1.6 μmol, 34%) as a white solid. [(M-2TFA)/3+1]=655.2.

### Example 35: tert-butyl (R)-(3-((N-((2′-ethoxy-5-(4-(6-ethoxy-2-(trifluoromethyl)nicotinoyl)-2-ethylpiperazin-1-yl[2,3′-bipyridin]-6-ylmethyl)-4-nitrophenypsulfonamido)propyl)carbamate

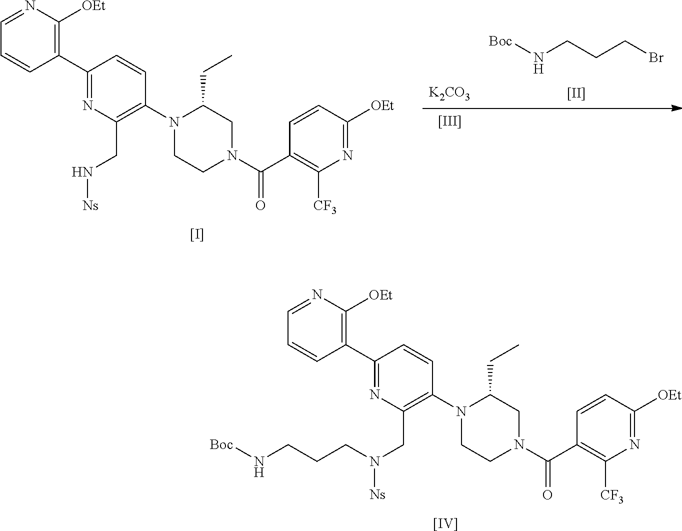

Into a 40 mL vial, was placed a mixture of (R)—N-((2′-ethoxy-5-(4-(6-ethoxy-2-(trifluoromethylnicotinoyl)-2-ethylpiperazin-1-yl)-[2,3′-bipyridin]-6-yOmethyl)-2-nitrobenzenesulfonamide (790 mg, 1 Eq, 1.06 mmol), tert-butyl (3-bromopropyl)carbamate (512 mg, 2.02 Eq, 2.15 mmol), K₂CO₃ (443 mg, 3.02 Eq, 3.21 mmol) and MeCN (10 mL). The reaction mixture was stirred at 80° C. for 5 hrs. The reaction crude was purified by Prep-HPLC using the following conditions: Column, C18 120 g; mobile phase, Water (0.1% FA) and ACN (30% ACN up to 98% in 7 min); Total flow rate, 70 mL/min; Detector, UV 220 nm. This resulted in tert-butyl (R)-(3-((N-((2′-ethoxy-5-(4-(6-ethoxy-2-(trifluoromethyl)nicotinoyl)-2-ethylpiperazin-1-yl[2,3′-bipyridin]-6-ylmethyl)-4-nitrophenypsulfonamido)propyl)carbamate (804 mg, 892 μmol, 84.0%) as a solid. [M+H]=901.3.

### Example 36: (R)—N-(3-aminopropyl)-N-((2′-ethoxy-5-(4-(6-ethoxy-2-(trifluoromethylnicotinoyl)-2-ethylpiperazin-1-yl)[2,3′-bipyridin]-6-yl)methyl)-2-nitrobenzenesulfonamide

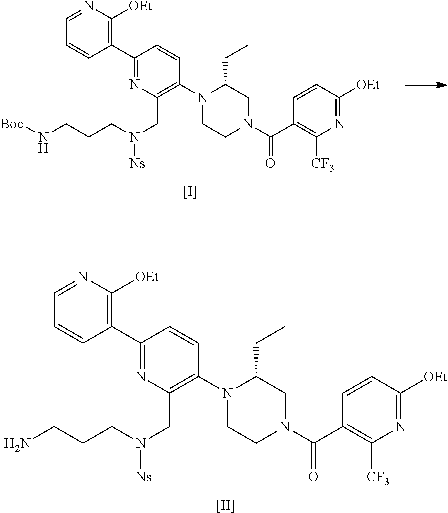

Into an 50 mL flask were added tert-butyl (R)-(3-((N-((2′-ethoxy-5-(4-(6-ethoxy-2-(trifluoromethylnicotinoyl)-2-ethylpiperazin-1-yl)-[2,3′-bipyridin]-6-yl)methyl)-2-nitrophenyl)-sulfonamido)propyl)carbamate (804 mg, 1 Eq, 892 μmol), DCM (6 mL) and TFA (3 mL). The resulting mixture was stirred at 28° C. for 1 hour. The reaction crude was purified by Prep-HPLC using the following conditions (IntelFlash-1): Column, C18 silica gel; mobile phase, water (0.1 NH₃·H₂O/CH₃CN (CH₃CN:30%-98% in 7 min); Detector, UV 254 & 220 nm. This resulted in (R)—N-(3-aminopropyl)-N-((2′-ethoxy-5-(4-(6-ethoxy-2-(trifluoromethylnicotinoyl)-2-ethylpiperazin-1-yl)[2,3′-bipyridin]-6-yl)methyl)-2-nitrobenzenesulfonamide (621 mg, 775 μmol, 86.9%) as a yellow solid. M+H=801.4.

### Example 37: (R)-(3-((N-((2′-ethoxy-5-(4-(6-ethoxy-2-(trifluoromethylnicotinoyl)-2-ethylpiperazin-1-yl)42,3′-bipyridin]-6-yl)methyl)-2-nitrophenyl)sulfonamido)propyl)glycinate

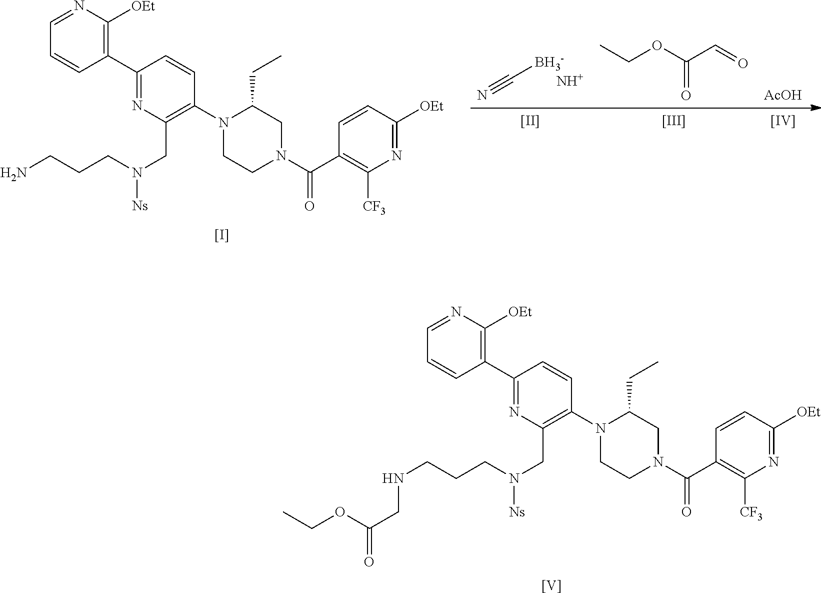

(R)—N-(3-aminopropyl)-N-((2′-ethoxy-5-(4-(6-ethoxy-2-(trifluoromethyl)nicotinoyl)-2-ethylpiperazin-1-yl)42,3′-bipyridin]-6-yl)methyl)-2-nitrobenzenesulfonamide (123 mg, 1 Eq, 154 μmol) was combined with AcOH (11 mg, 10 μL, 1.2 Eq, 0.18 mmol), ethyl 2-oxoacetate (32 mg, 50% Wt, 1.0 Eq, 0.16 mmol) and EtOH (2 mL). The resulting mixture was stirred at 28° C. for 10 min, followed by the addition of NaCNBH₃ (21 mg, 2.2 Eq, 0.33 mmol). The resulting mixture was stirred at 28° C. for 1 hour. The reaction crude was purified by Prep-HPLC using the following conditions: Column, C18 silica gel; mobile phase, water (0.1% FA)/CH₃CN (CH₃CN: 30%-98% in 5 min); Detector, UV 254 & 220 nm. This resulted in ethyl (R)-(3-((N-((2′-ethoxy-5-(4-(6-ethoxy-2-(trifluoromethylnicotinoyl)-2-ethylpiperazin-1-yl)42,3′-bipyridin]-6-yl)methyl)-2-nitrophenyl)-sulfonamido)propyl)glycinate (63 mg, 71 μmol, 46%) as a light yellow solid. M+H=887.2.

### Example 38: ethyl (R)-17-(3-((N-((2′-ethoxy-5-(4-(6-ethoxy-2-(trifluoromethylnicotinoyl)-2-ethylpiperazin-1-yl)-[2,3′-bipyridin]-6-ylmethyl)-4-nitrophenypsulfonamido)propyl)-2,2-dimethyl-4-oxo-3,8,11,14-tetraoxa-5,17-diazanonadecan-19-oate

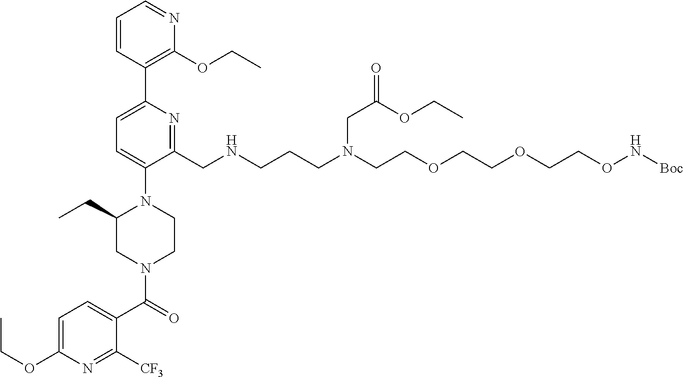 [SMILES: CCOC(=O)CN(CCCNCc1nc(-c2cccnc2OCC)ccc1N1CCN(C(=O)c2ccc(OCC)nc2C(F)(F)F)C[C@H]1CC)CCOCCOCCONC(=O)OC(C)(C)C]

Into an 8 mL vial, was placed a mixture of ethyl (R)-(3-((N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrophenyl)-sulfonamido)propyl)glycinate (63 mg, 1 Eq, 71 μmol), K₂CO₃ (29 mg, 3.0 Eq, 0.21 mmol), NaI (31 mg, 2.9 Eq, 0.21 mmol), 2,2-dimethyl-4-oxo-3,8,11,14-tetraoxa-5-azahexadecan-16-yl methanesulfonate (55 mg, 2.1 Eq, 0.15 mmol) and MeCN (3 mL). The resulting mixture was stirred at 80° C. for 3 hrs. The reaction crude was purified by Prep-HPLC using the following conditions: Column, C18 120 g; mobile phase, Water (0.1% FA) and ACN (30% ACN up to 98% in 7 min); Total flow rate, 70 mL/min; Detector, UV 220 nm. This resulted in ethyl (R)-17-(3-((N-((2′-ethoxy-5-(4-(6-ethoxy-2-(trifluoromethylnicotinoyl)-2-ethylpiperazin-1-yl)-[2,3′-bipyridin]-6-ylmethyl)-4-nitrophenylsulfonamido)propyl)-2,2-dimethyl-4-oxo-3,8,11,14-tetraoxa-5,17-diazanonadecan-19-oate (39 mg, 34 μmol, 47%) as a light yellow solid. [M+H]=1162.7.

### Example 39: Ethyl (R)-14-amino-3-(3-((N-((2′-ethoxy-5-(4-(6-ethoxy-2-(trifluoromethyl)-nicotinoyl)-2-ethylpiperazin-1-yl)42,3′-bipyridin]-6-yl)methyl)-2-nitrophenyl)sulfonamido)-propyl)-6,9,12-trioxa-3-azatetradecanoate

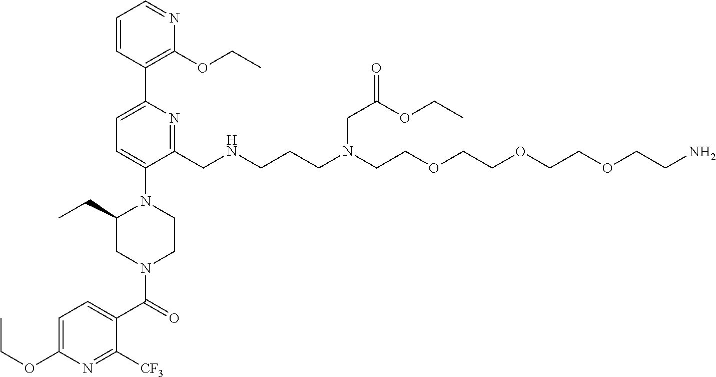 [SMILES: CCOC(=O)CN(CCCNCc1nc(-c2cccnc2OCC)ccc1N1CCN(C(=O)c2ccc(OCC)nc2C(F)(F)F)C[C@H]1CC)CCOCCOCCOCCN]

Into an 8 mL vial were added ethyl (R)-17-(3-((N-((2′-ethoxy-5-(4-(6-ethoxy-2-(trifluoromethyl)nicotinoyl)-2-ethylpiperazin-1-yl)42,3′-bipyridin]-6-yl)methyl)-2-nitrophenyl)-sulfonamido)propyl)-2,2-dimethyl-4-oxo-3,8,11,14-tetraoxa-5,17-diazanonadecan-19-oate (39 mg, 1 Eq, 34 μmol), DCM (1 mL) and TFA (0.5 mL). The resulting mixture was stirred at 28° C. for 1 hour. The reaction crude was purified by Prep-HPLC using the following conditions: Column, C18 silica gel; mobile phase, water (0.1% NH₃·H₂O/CH₃CN (CH₃CN:30%-98% in 2 min); Detector, UV 254 & 220 nm. This resulted in ethyl (R)-14-amino-3-(3-((N-((2′-ethoxy-5-(4-(6-ethoxy-2-(trifluoromethyl) nicotinoyl)-2-ethylpiperazin-1-yl)42,3′-bipyridin]-6-yl)methyl)-2-nitrophenyl)-sulfonamido)propyl)-6,9,12-trioxa-3-azatetradecanoate (27 mg, 25 μmol, 76%) as a solid. M+H=1062.3.

### Example 40: (R)-2,2′,2″-(10-(6-(carboxymethyl)-1-(2′-ethoxy-5-(4-(6-ethoxy-2-(trifluoromethyl-nicotinoyl)-2-ethylpiperazin-1-yl)-[2,3′-bipyridin]-6-yl)-19-oxo-9,12,15-trioxa-2,6,18-triazaicosan-20-yl)-1,4,7,10-tetraazacyclododecane-1,4,7-triyl)triacetic acid (Compound 19)

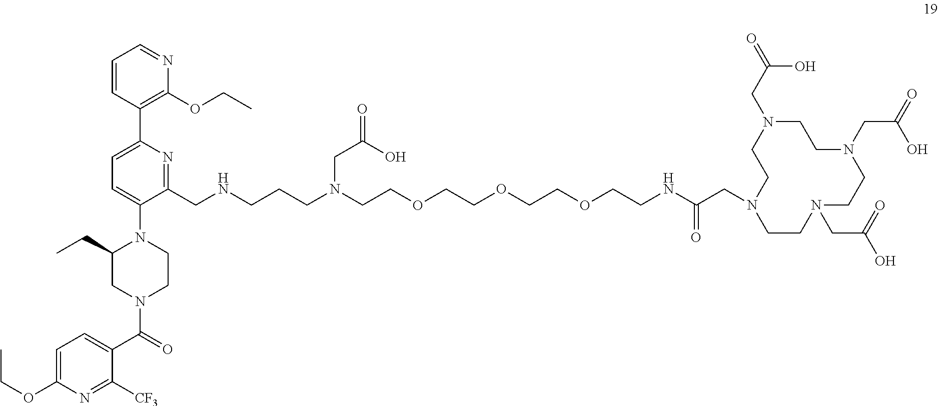 [SMILES: CCOc1ccc(C(=O)N2CCN(c3ccc(-c4cccnc4OCC)nc3CNCCCN(CCOCCOCCOCCNC(=O)CN3CCN(CC(=O)O)CCN(CC(=O)O)CCN(CC(=O)O)CC3)CC(=O)O)[C@H](CC)C2)c(C(F)(F)F)n1]

Step 1: Into an 8-mL vial, was placed a mixture of 2-(4,7,10-tris(2-(tert-butoxy)-2-oxoethyl)-1,4,7,10-tetraazacyclododecan-1-ypacetic acid (19 mg, 1.3 Eq, 33 μmol), HATU (15 mg, 1.6 Eq, 39 μmol), DIEA (11 mg, 15 μL, 3.3 Eq, 85 μmol) and DMF (0.5 mL). The resulting mixture was stirred at 28° C. for 5 min, followed by the addition of ethyl (R)-14-amino-3-(3-((N-((2′-ethoxy-5-(4-(6-ethoxy-2-(trifluoromethylnicotinoyl)-2-ethylpiperazin-1-yl)42,3′-bipyridin]-6-yl)methyl)-2-nitrophenyl)sulfonamido)propyl)-6,9,12-trioxa-3-azatetradecanoate (27 mg, 1 Eq, 25 μmol). The reaction mixture was stirred at 28° C. for 1 hour. The reaction crude was purified by Prep-HPLC using the following conditions: Column, C18 120 g; mobile phase, Water (0.05% FA) and ACN (30% ACN up to 98% in 7 min); Total flow rate, 70 mL/min; Detector, UV 220 nm. This resulted in tri-tert-butyl 2,2′,2″-(10-(6-(2-ethoxy-2-oxoethyl)-1-(2′-ethoxy-5-(4-(6-ethoxy-2-(trifluoromethyl-nicotinoyl)-2-ethylpiperazin-1-yl)-[2,3′-bipyridin]-6-yl)-2-((4-nitrophenyl)sulfonyl)-19-oxo-9,12,15-trioxa-2,6,18-triazaicosan-20-yl)-1,4,7,10-tetraazacyclododecane-1,4,7-triyl)(R)-triacetate (17 mg, 11 μmol, 41%) as a light yellow solid. [M+H]=1617.7.

Step 2: Into an 8 mL vial were added tri-tert-butyl 2,2′,2″-(10-(6-(2-ethoxy-2-oxoethyl)-1-(2′-ethoxy-5-(4-(6-ethoxy-2-(trifluoromethylnicotinoyl)-2-ethylpiperazin-1-yl)-[2,3′-bipyridin]-6-yl)-2-((4-nitrophenyl)sulfonyl)-19-oxo-9,12,15-trioxa-2,6,18-triazaicosan-20-yl)-1,4,7,10-tetraazacyclododecane-1,4,7-triyl)(R)-triacetate (17 mg, 1 Eq, 11 μmol), lithium hydroxide (3.1 mg, 12 Eq, 0.13 mmol), MeOH (0.3 mL) water (0.1 mL). The resulting mixture was stirred at 80° C. for 2 hrs. The reaction crude was purified by Prep-HPLC using the following conditions: Column, C18 silica gel; mobile phase, water (0.1% FA/CH₃CN (CH₃CN:30%-98% in 2 min); Detector, UV 254 & 220 nm. This resulted in (R)-2,2′,2″-(10-(6-(carboxymethyl)-1-(2′-ethoxy-5-(4-(6-ethoxy-2-(trifluoromethyl)nicotinoyl)-2-ethylpiperazin-1-yl)-(2,3′-bipyridin]-6-yl)-2-((2-nitrophenyl)-sulfonyl)-19-oxo-9,12,15-trioxa-2,6,18-triazaicosan-20-yl)-1,4,7,10-tetraazacyclododecane-1,4,7-triyi)triacetic acid (12 mg, 8.4 μmol, 80%) as a light yellow solid. M+H=1420.9.

Step 3: Into a 2-mL vial, was placed a mixture of (R)-2,2′,2″-(10-(6-(carboxymethyl)-1-(2′-ethoxy-5-(4-(6-ethoxy-2-(trifluoromethylnicotinoyl)-2-ethylpiperazin-1-yl)-[2,3′-bipyridin]-6-yl)-2-((4-nitrophenyl)sulfonyl)-19-oxo-9,12,15-trioxa-2,6,18-triazaicosan-20-yl)-1,4,7,10-tetraazacyclo-dodecane-1,4,7-triyptriacetic acid (12 mg, 1 Eq, 8.4 μmol), 2-chlorobenzenethiol (12 mg, 9.8 Eq, 83 μmol), K₂CO₃ (4.1 mg, 3.5 Eq, 30 μmol) and MeCN (0.3 mL). The reaction mixture was stirred at 80° C. for 1 hour. The reaction crude was purified by Prep-HPLC using the following conditions: Column, C18 120 g; mobile phase, Water (0.05% FA) and ACN (30% ACN up to 98% in 8 min); Total flow rate, 20 mL/min; Detector, UV 220 nm. This resulted in (R)-2,2′,2″-(10-(6-(carboxymethyl)-1-(2′-ethoxy-5-(4-(6-ethoxy-2-(trifluoromethylnicotinoyl)-2-ethylpiperazin-1-yl)-[2,3′-bipyridin]-6-yl)-19-oxo-9,12,15-trioxa-2,6,18-triazaicosan-20-yl)-1,4,7,10-tetraazacyclo-dodecane-1,4,7-triyl)triacetic acid (8.0 mg, 6.5 μmol, 77%) as a light yellow oil. [M+H]=1236.0.

### Example 41: ¹¹⁵Indium Complex of Compound 19

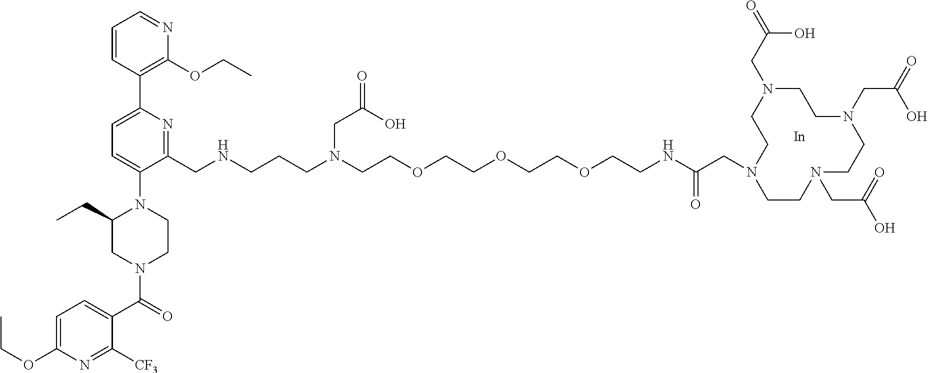

Into an 8 mL vial were placed (R)-2,2′,2″-(10-(6-(carboxymethyl)-1-(2′-ethoxy-5-(4-(6-ethoxy-2-(trifluoromethyl)nicotinoyl)-2-ethylpiperazin-1-yl)-[2,3′-bipyridin]-6-yl)-19-oxo-9,12,15-trioxa-2,6,18-triazaicosan-20-yl)-1,4,7,10-tetraazacyclododecane-1,4,7-triyl)triacetic acid (8 mg, 1 Eq, 6 μmol), ¹¹⁵InCl₃ (7.2 mg, 5 Eq, 33 μmol), NaHCO₃ (11 mg, 2e+1 Eq, 0.13 mmol), MeCN (0.3 mL) and Water (0.1 mL). The reaction mixture was stirred at 80° C. for 0.5 hour. The reaction crude was purified by Prep-HPLC using the following conditions: Sunfire Prep C18 OBD Column, 50×250 mm, 5 μm 10 nm; Mobile Phase A: Water (0.05% TFA), Mobile Phase B: ACN; Flow rate: 90 mL/min; Gradient: 28% B to 48% B in 8 min. Pure fractions were combined and concentrated to afford the product (4.9 mg, 3.1 μmol, 50%) as a colorless oil. [M+H-2TFA]=1347.8.

### Example 42: tert-butyl (S)-3-((((9H-fluoren-9-yl)methoxy)carbonyl)amino)-4-((3-((N-((2′-ethoxy-5-((R)-4-(6-ethoxy-2-(trifluoromethylnicotinoyl)-2-ethylpiperazin-1-yl)42,3′-bipyridin]-6-yl)methyl)-2-nitrophenyl)sulfonamido)propyl)amino)-4-oxobutanoate

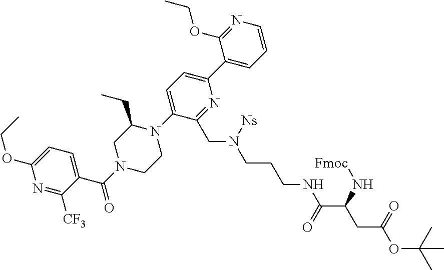

Into an 8-mL vial, was placed a mixture of (S)-2-((((9H-fluoren-9-yl)methoxy)carbonyl)-amino)-4-(tert-butoxy)-4-oxobutanoic acid (34 mg, 1.3 Eq, 83 μmol), HATU (36 mg, 1.5 Eq, 95 μmol), DIEA (27 mg, 36 μL, 3.3 Eq, 0.21 mmol) and DMF (1.0 mL). The reaction mixture was stirred at 28° C. for 5 min, followed by the addition of (R)—N-(3-aminopropyl)-N-((2′-ethoxy-5-(4-(6-ethoxy-2-(trifluoromethylnicotinoyl)-2-ethylpiperazin-1-yl)-[2,3′-bipyridin]-6-ylmethyl)-2-nitrobenzenesulfonamide (50 mg, 1 Eq, 62 μmol). The reaction mixture was stirred at the same temperature for 1 hour. The reaction crude was purified by Prep-HPLC using the following conditions: Column, C18 120 g; mobile phase, Water (0.05% FA) and ACN (30% ACN up to 98% in 5 min); Total flow rate, 70 mL/min; Detector, UV 220 nm. This resulted in tert-butyl (S)-3-((((9H-fluoren-9-yl)methoxy)carbonyl)amino)-4-((3-((N-((2′-ethoxy-5-((R)-4-(6-ethoxy-2-(trifluoromethyl-nicotinoyl)-2-ethylpiperazin-1-yl)42,3′-bipyridin]-6-yl)methyl)-2-nitrophenyl)sulfonamido)-propyl)amino)-4-oxobutanoate (60 mg, 50 μmol, 80%) as a light yellow solid. [M+H]=1194.6.

### Example 43: (S)-3-((((9H-fluoren-9-yl)methoxy)carbonypamino)-4-((3-((N-((2′-ethoxy-5-((R)-4-(6-ethoxy-2-(trifluoromethylnicotinoyl)-2-ethylpiperazin-1-yl)-[2,3′-bipyridin]-6-yl)methyl)-4-nitrophenypsulfonamido)propyl)amino)-4-oxobutanoic acid

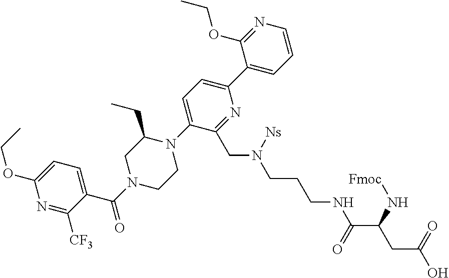

Into an 8-mL vial, was placed tert-butyl (S)-3-((((9H-fluoren-9-ylmethoxy)carbonylamino)-4-((3-((N-((2′-ethoxy-5-((R)-4-(6-ethoxy-2-(trifluoromethylnicotinoyl)-2-ethylpiperazin-1-yl)-[2,3′-bipyridin]-6-yl)methyl)-2-nitrophenyl)sulfonamido)propyl)amino)-4-oxobutanoate (60 mg, 1 Eq, 50 μmol) and TFA (1 mL). The resulting mixture was stirred at 28° C. for 1 hour. The reaction mixture was concentrated under vacuum. The remaining residue was purified by Prep-HPLC using the following conditions: Column, C18 120 g; mobile phase, Water (0.05% FA) and ACN (30% ACN up to 98% in 2 min); Total flow rate, 70 mL/min; Detector, UV 220 nm. This resulted in (S)-3-((((9H-fluoren-9-yl)methoxy)carbonypamino)-4-((3-((N-((2′-ethoxy-5-((R)-4-(6-ethoxy-2-(trifluoromethyl-nicotinoyl)-2-ethylpiperazin-1-yl)-[2,3′-bipyridin]-6-yl)methyl)-4-nitrophenypsulfonamido)propyl)-amino)-4-oxobutanoic acid (49 mg, 43 μmol, 86%) as a light brown oil. M+H=1138.6.

### Example 44: (9H-fluoren-9-yl)methyl tert-butyl ((S)-1-(2′-ethoxy-5-((R)-4-(6-ethoxy-2-(trifluoromethylnicotinoyl)-2-ethylpiperazin-1-yl)42,3′-bipyridin]-6-yl)-2-((2-nitrophenyl)sulfonyl)-7,10-dioxo-14-oxa-2,6,11-triazahexadecane-8,16-diyl)dicarbamate

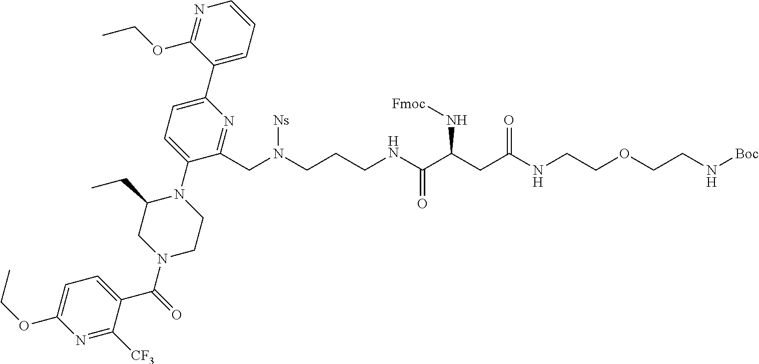

Into an 8-mL vial, was placed a mixture of (S)-3-((((9H-fluoren-9-yl)methoxy)carbonyl)-amino)-4-((3-((N-((2′-ethoxy-5-((R)-4-(6-ethoxy-2-(trifluoromethylnicotinoyl)-2-ethylpiperazin-1-yl)42,3′-bipyridin]-6-yl)methyl)-2-nitrophenyl)sulfonamido)propyl)amino)-4-oxobutanoic acid (49 mg, 1 Eq, 43 μmol), HATU (22 mg, 1.3 Eq, 58 μmol), DIEA (31 mg, 42 μL, 5.6 Eq, 0.24 mmol) and DMF (0.5 mL). The resulting mixture was stirred at 28° C. for 5 min, followed by the addition of tert-butyl (2-(2-aminoethoxy)ethyl)carbamate (11 mg, 1.3 Eq, 54 μmol). The reaction mixture was stirred at 28° C. for 1 hour. The reaction crude was purified by Prep-HPLC using the following conditions: Column, C18 120 g; mobile phase, Water (0.05% FA) and ACN (30% ACN up to 98% in 5 min); Total flow rate, 70 mL/min; Detector, UV 220 nm. This resulted in (9H-fluoren-9-yl)methyl tert-butyl ((S)-1-(2′-ethoxy-5-((R)-4-(6-ethoxy-2-(trifluoromethylnicotinoyl)-2-ethylpiperazin-1-yl)42,3′-bipyridin]-6-yl)-2-((2-nitrophenyl)sulfonyl)-7,10-dioxo-14-oxa-2,6,11-triazahexadecane-8,16-diyl)dicarbamate (45 mg, 34 μmol, 79%) as light yellow solid. [M+1-1]=1324.7.

### Example 45: (9H-fluoren-9-yl)methyl ((S)-16-amino-1-(2′-ethoxy-5-((R)-4-(6-ethoxy-2-(trifluoromethyl)nicotinoyl)-2-ethylpiperazin-1-yl)42,3′-bipyridin]-6-yl)-2-((2-nitrophenyl)sulfonyl)-7,10-dioxo-14-oxa-2,6,11-triazahexadecan-8-yl)carbamate

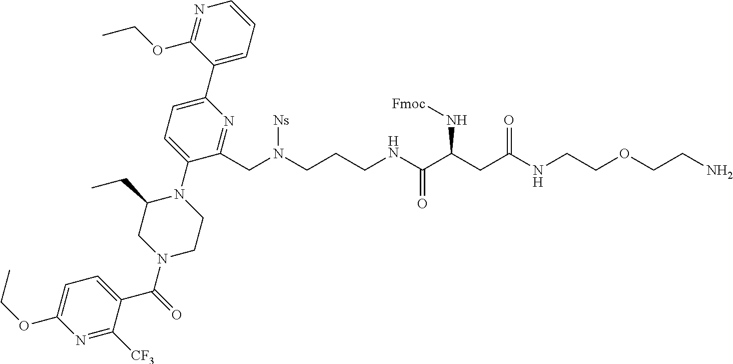

Into an 8 mL vial were added (9H-fluoren-9-yl)methyl tert-butyl ((S)-1-(2′-ethoxy-5-((R)-4-(6-ethoxy-2-(trifluoromethylnicotinoyl)-2-ethylpiperazin-1-yl)42,3′-bipyridin]-6-yl)-2-((2-nitrophenyl)sulfonyl)-7,10-dioxo-14-oxa-2,6,11-triazahexadecane-8,16-diyl)dicarbamate (45 mg, 1 Eq, 34 μmol), DCM (1 mL), and TFA (0.5 mL). The resulting mixture was stirred at 28° C. for 1 hour. The mixture was purified by Prep-HPLC using the following conditions: Column, C18 silica gel; mobile phase, water (0.1% FA)/CH₃CN (CH₃CN:30%-98% in 2 min); Detector, UV 254&220 nm. This resulted in (9H-fluoren-9-yl)methyl ((S)-16-amino-1-(2′-ethoxy-5-((R)-4-(6-ethoxy-2-(trifluoromethyl)nicotinoyl)-2-ethylpiperazin-1-yl)42,3′-bipyridin]-6-yl)-2-((2-nitrophenyl)-sulfonyl)-7,10-dioxo-14-oxa-2,6,11-triazahexadecan-8-yl)carbamate (35 mg, 29 μmol, 84%) as a light yellow oil. M+H=1224.7.

### Example 46: 2,2′,2″-(10-((S)-8-amino-1-(2′-ethoxy-5-((R)-4-(6-ethoxy-2-(trifluoromethyl-nicotinoyl)-2-ethylpiperazin-1-yl)42,3′-bipyridin]]-6-yl)-7,10,18-trioxo-14-oxa-2,6,11,17-tetra-azanonadecan-19-VI)-1,4,7,10-tetraazacyelododecane-1,4,7-triyl)triacetic acid (Compound 5)

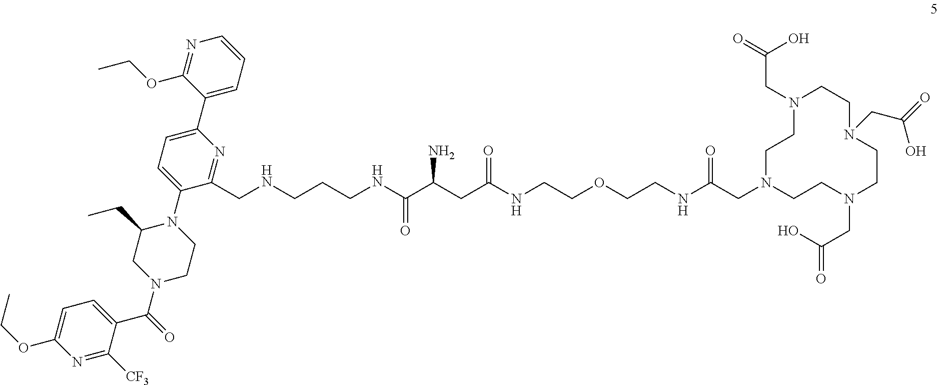 [SMILES: CCOc1ccc(C(=O)N2CCN(c3ccc(-c4cccnc4OCC)nc3CNCCCNC(=O)[C@@H](N)CC(=O)NCCOCCNC(=O)CN3CCN(CC(=O)O)CCN(CC(=O)O)CCN(CC(=O)O)CC3)[C@H](CC)C2)c(C(F)(F)F)n1]

Step 1: Into an 8-mL vial, was placed a mixture of 2-(4,7,10-tris(2-(tert-butoxy)-2-oxoethyl)-1,4,7,10-tetraazacyclododecan-1-ylacetic acid (22 mg, 1.3 Eq, 38 μmol), HATU (16 mg, 1.5 Eq, 42 μmol), DIEA (19 mg, 26 μL, 5.1 Eq, 0.15 mmol) and DMF (0.5 mL). The resulting mixture was stirred at 28° C. for 5 min, followed by the addition of (9H-fluoren-9-yl)methyl ((S)-16-amino-1-(2′-ethoxy-5-((R)-4-(6-ethoxy-2-(trifluoromethylnicotinoyl)-2-ethylpiperazin-1-yl)42,3′-bipyridin]-6-yl)-2-((2-nitrophenyl)sulfonyl)-7,10-dioxo-14-oxa-2,6,11-triazahexadecan-8-carbamate (35 mg, 1 Eq, 29 pmol). The reaction mixture was stirred at 28° C. for 1 hour. The reaction crude was purified by Prep-HPLC using the following conditions: Column, C18 120 g; mobile phase, Water (0.05% FA) and ACN (30% ACN up to 98% in 7 min); Total flow rate, 70 mL/min; Detector, UV 220 nm. This resulted in tri-tert-butyl 2,2′,2″-(10-((S)-8-((((9H-fluoren-9-yl)methoxy)carbonyl)-amino)-1-(2′-ethoxy-5-((R)-4-(6-ethoxy-2-(trifluoromethylnicotinoyl)-2-ethylpiperazin-1-yl)-[2,3′-bipyridin]-6-yl)-2-((2-nitrophenyl)sulfonyl)-7,10,18-trioxo-14-oxa-2,6,11,17-tetraazanonadecan-19-yl)-1,4,7,10-tetraazacyclododecane-1,4,7-triyl)triacetate (29 mg, 16 μmol, 57%) as a light yellow solid. [M+H]=1779.7.

Step 2: Into an 8 mL vial were added tri-tert-butyl 2,2′,2″-(10-((S)-8-((((9H-fluoren-9-yl)methoxy)carbonyl)amino)-1-(2′-ethoxy-5-((R)-4-(6-ethoxy-2-(trifluoromethylnicotinoy0-2-ethylpiperazin-1-yl)42,3′-bipyridin]-6-yl)-2-((2-nitrophenyl)sulfonyl)-7,10,18-trioxo-14-oxa-2,6,11,17-tetraazanonadecan-19-14)-1,4,7,10-tetraazacyclododecane-1,4,7-triyl)triacetate (29 mg, 1 Eq, 16 μmol), piperidine (12 mg, 8.6 Eq, 0.14 mmol) and MeCN (0.5 mL). The resulting mixture was stirred at 28° C. for 1 hour. The mixture was concentrated to give crude tri-tert-butyl 2,2′,2″-(10-((S)-8-amino-1-(2′-ethoxy-5-((R)-4-(6-ethoxy-2-(trifluoromethylnicotinoyl)-2-ethylpiperazin-1-yl)42,3′-bipyridin]-6-yl)-2-((2-nitrophenyl)sulfonyl)-7,10,18-trioxo-14-oxa-2,6,11,17-tetraaza-nonadecan-19-yl)-1,4,7,10-tetraazacyclododecane-1,4,7-triyltriacetate (31 mg, 20 μmol, 120%) as a light yellow oil. This material was used in next step without further purification. [M+H]=1557.0.

Step 3: Into an 8 mL vial were added tri-tert-butyl 2,2′,2″-(10-((S)-8-amino-1-(2′-ethoxy-5-((8)-4-(6-ethoxy-2-(trifluoromethyl)nicotinoyl)-2-ethylpiperazin-1-yl)42,3′-bipyridin]-6-yl)-2-((2-nitrophenyl)sulfonyl)-7,10,18-trioxo-14-oxa-2,6,11,17-tetraazanonadecan-19-yl)-1,4,7,10-tetraazacyclododecane-1,4,7-triyl)triacetate (31 mg, 1 Eq, 20 μmol) and TFA (0.5 mL). The resulting mixture was stirred at 28° C. for 5 hrs. The reaction crude was purified by prep-HPLC with using the following conditions (IntelFlash-1): Column, C18 silica gel; mobile phase, water (0.1% FA)/CH₃CN (CH₃CN:30%-98% in 3 min, Flow rate: 70 mL/min); Detector, UV 254&220 nm. This resulted in 2,2′,2″-(10-((S)-8-amino-1-(2′-ethoxy-5-((8)-4-(6-ethoxy-2-(trifluoromethyl)nicotinoyl)-2-ethylpiperazin-1-yl)42,3′-bipyridin]-6-yl)-2-((2-nitrophenyl)sulfonyl)-7,10,18-trioxo-14-oxa-2,6,11,17-tetraazanonadecan-19-yl)-1,4,7,10-tetraazacyclododecane-1,4,7-triyl)triacetic acid (19 mg, 14 μmol, 69%) as a light yellow solid. M+H=1388.4.

Step 4: Into an 8-mL vial, was placed a mixture of 2,2′,2″-(10-((S)-8-amino-1-(2′-ethoxy-5-((8)-4-(6-ethoxy-2-(trifluoromethylnicotinoyl)-2-ethylpiperazin-1-yl)42,3′-bipyridin]-6-yl)-2-((2-nitrophenyl)sulfonyl)-7,10,18-trioxo-14-oxa-2,6,11,17-tetraazanonadecan-19-yl)-1,4,7,10-tetraazacyclododecane-1,4,7-triyl)triacetic acid (19 mg, 1 Eq, 14 μmol), 2-chlorobenzenethiol (21 mg, 11 Eq, 0.15 mmol), K₂CO₃ (6.1 mg, 3.2 Eq, 44 μmol) and MeCN (0.3 mL). The reaction mixture was stirred at 50° C. for 1 hour. The reaction crude was purified by Prep-HPLC using the following conditions: Sunfire Prep C18 OBD Column, 50×250 mm, 5 μm 10 nm; Mobile Phase A: Water (0.05% TFA), Mobile Phase B: ACN; Flow rate: 90 mL/min; Gradient: 35% B to 55% B in 8 min. This resulted in 2,2′,2″-(10-((S)-8-amino-1-(2′-ethoxy-5-((R)-4-(6-ethoxy-2-(trifluoromethylnicotinoyl)-2-ethylpiperazin-1-yl)42,3′-bipyridin]-6-yl)-7,10,18-trioxo-14-oxa-2,6,11,17-tetraazanonadecan-19-yl)-1,4,7,10-tetraazacyclododecane-1,4,7-triyl)triacetic acid (3.1 mg, 2.6 μmol, 19%) as a light yellow oil. [M+H]=1203.4.

### Example 47: ¹¹⁵Indium Complex of Compound 5

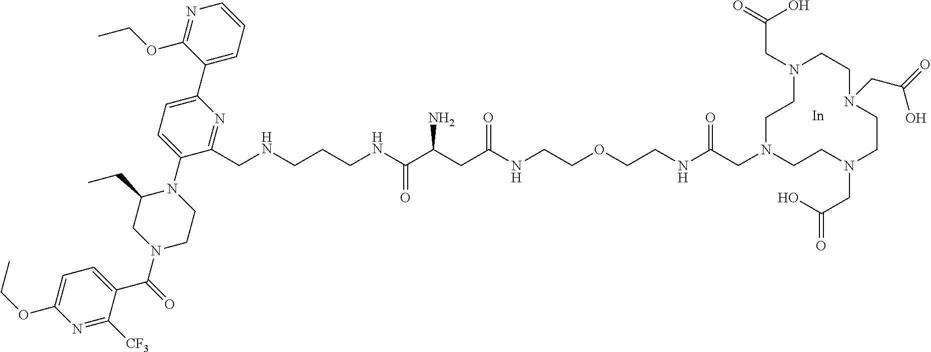

Into an 8 mL vial were placed 2,2′,2″-(10-((S)-8-amino-1-(2′-ethoxy-5-((8)-4-(6-ethoxy-2-(trifluoromethylnicotinoyl)-2-ethylpiperazin-1-yl)-(2,3′-bipyridin]-6-yl)-7,10,18-trioxo-14-oxa-2,6,11,17-tetraazanonadecan-19-yl)-1,4,7,10-tetraazacyclododecane-1,4,7-triyl)triacetic acid (3.1 mg, 1 Eq, 2.6 μmol), ¹¹⁵InCl₃ (2.9 mg, 5.1 Eq, 13 μmol), NaHCO₃ (4.7 mg, 22 Eq, 56 μmol), MeCN (0.3 mL) and water (0.1 mL). The reaction mixture was stirred at 80° C. for 1 hour. The reaction crude was purified by Prep-HPLC using the following conditions: Sunfire Prep C18 OBD Column, 50×250 mm, 5 μm 10 nm; Mobile Phase A: Water (0.05% TFA), Mobile Phase B: ACN; Flow rate: 90 mL/min; Gradient: 25% B to 48% B in 8 min. Pure fractions were combined and concentrated to afford the product (0.9 mg, 0.6 μmol, 20%) as a colorless oil. [M+H-2TFA]=1315.7.

### Example 48: 2-((4R,6R)-6-(2-((tert-butoxycarbonyl)amino)ethyl)-2,2-dimethyl-1,3-dioxan-4-yl)acetic acid

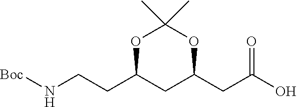 [SMILES: CC(C)(C)OC(=O)NCC[C@@H]1C[C@H](CC(=O)O)OC(C)(C)O1]

Step 1: Into a 40-mL vial, was placed a mixture of tert-butyl 2-((4R,6R)-6-(2-aminoethyl)-2,2-dimethyl-1,3-dioxan-4-yl)acetate (500 mg, 1 Eq, 1.83 mmol), TEA (407 mg, 561 μL, 2.20 Eq, 4.02 mmol) and DCM (15 mL). Boc₂O (439 mg, 462 μL, 1.10 Eq, 2.01 mmol) was added. The reaction mixture was stirred at 25° C. for 1 hour. The reaction mixture was diluted with water (50 mL) and extracted with DCM (30 mL×3). Organic layers were combined, washed with brine (50 mL), dried over anhydrous Na₂SO₄ and concentrated under reduced pressure to afford tert-butyl 2-((48,68)-6-(2-((tert-butoxycarbonyl)amino)ethyl)-2,2-dimethyl-1,3-dioxan-4-ylacetate (685 mg, 1.83 mmol, 100%) as a yellow oil. [M+Na]=396.3.

Step 2: Into a 40-mL vial, was placed a mixture of tert-butyl 2-((48,68)-6-(2-((tert-butoxycarbonyl)amino)ethyl)-2,2-dimethyl-1,3-dioxan-4-yl)acetate (660 mg, 1 Eq, 1.77 mmol), LiOH (423 mg, 9.99 Eq, 17.7 mmol), MeOH (10 mL) and Water (3 mL). The reaction mixture was stirred at 25° C. for 16 hrs. The reaction mixture was concentrated under reduced pressure to remove most MeOH, and the remaining residue was diluted with water (50 mL). The resulting solution was acidified with saturated NaHSO₄ solution until pH=6.0 and extracted with DCM (50 mL×3). Organic layers were combined, dried over anhydrous Na₂SO₄, filtered, and concentrated under reduced pressure to afford 2-((4R,6R)-6-(2-((tert-butoxycarbonyl)amino)ethyl)-2,2-dimethyl-1,3-dioxan-4-yl)acetic acid (450 mg, 1.42 mmol, 80.2%) as light yellow solid. [M+Na]=340.2.

### Example 49: tert-butyl (2-((4R,6R)-6-(2-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)amino)-2-oxoethyl)-2,2-dimethyl-1,3-dioxan-4-yl)ethyl)carbamate

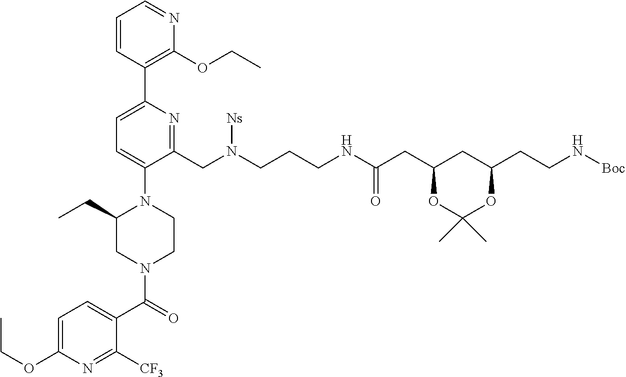

Into an 8-mL vial, was placed with a mixture of 2-((4R,6R)-6-(2-((tert-butoxycarbonyl)-amino)ethyl)-2,2-dimethyl-1,3-dioxan-4-ylacetic acid (100 mg, 2.52 Eq, 315 μmol), chloro-N,N,N′,N′-tetramethylformamidinium hexafluorophosphate (TCFH) (175 mg, 5.00 Eq, 624 μmol), 1-Methylimidazole (NMI) (100 mg, 96.8 μL, 9.75 Eq, 1.22 mmol) and DMF (2 mL). The reaction mixture was stirred at 30° C. for 10 minutes, followed by the addition of (R)—N-(3-aminopropyl)-N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (100 mg, 1 Eq, 125 μmol). The reaction mixture was stirred at 30° C. for 1 hour. The reaction crude was purified by Prep-HPLC using the following conditions: Column, C18 120 g; mobile phase, Water (0.1% FA) and ACN (30% ACN up to 98% in 6 min, 98% ACN to 98% in 3 min); Total flow rate, 70 mL/min; Detector, UV 220 nm. This resulted in tert-butyl (2-((4R,6R)-6-(2-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)amino)-2-oxoethyl)-2,2-dimethyl-1,3-dioxan-4-yl)ethyl)carbamate (112 mg, 102 μmol, 81.5%) as yellow solid. [M+H]=1100.4.

### Example 50: (3R,5R)-7-amino-N-(3-((N-((2′-ethoxy-5-((R)-4-(6-ethoxy-2-(trifluoromethyl)-nicotinoyl)-2-ethylpiperazin-1-yl)-[2,3′-bipyridin]-6-yl)methyl)-2-nitrophenyl)sulfonamido)-propyl)-3,5-dihydroxyheptanamide

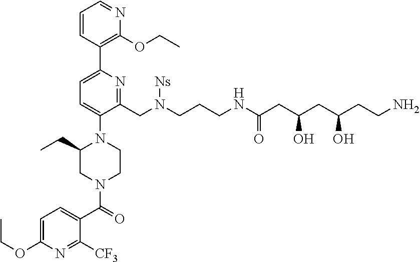

To a DCM (1 mL) solution of tert-butyl (2-((4R,6R)-6-(2-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)amino)-2-oxoethyl)-2,2-dimethyl-1,3-dioxan-4-yl)ethyl)carbamate (112 mg, 1 Eq, 102 μmol) was added TFA (1 mL). The resulting mixture was stirred at 30° C. for 1 hour. The reaction mixture was concentrated and purified by Prep-HPLC using the following conditions: Column, C18 120 g; mobile phase, Water (0.05% FA) and ACN (30% ACN up to 98% in 6 min, 98% ACN to 98% in 3 min); Total flow rate, 70 mL/min; Detector, UV 220 nm. This resulted in (3R,5R)-7-amino-N-(3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)-3,5-dihydroxyheptanamide (85 mg, 89 μmol, 87%) as a light yellow oil. [M+H]=960.7.

### Example 51: 2,2′,2″-(10-(2-(((3R,5R)-7-((3-(((2′-ethoxy-5-((R)-4-(6-ethoxy-2-(trifluoromethyl)-nicotinoyl)-2-ethylpiperazin-1-yl)-[2,3′-bipyridin]-6-yl)methyl)amino)propyl)-amino)-3,5-dihydroxy-7-oxoheptyl)amino)-2-oxoethyl)-1,4,7,10-tetraazacyclododecane-1,4,7-triyl)triacetic acid (Compound 6)

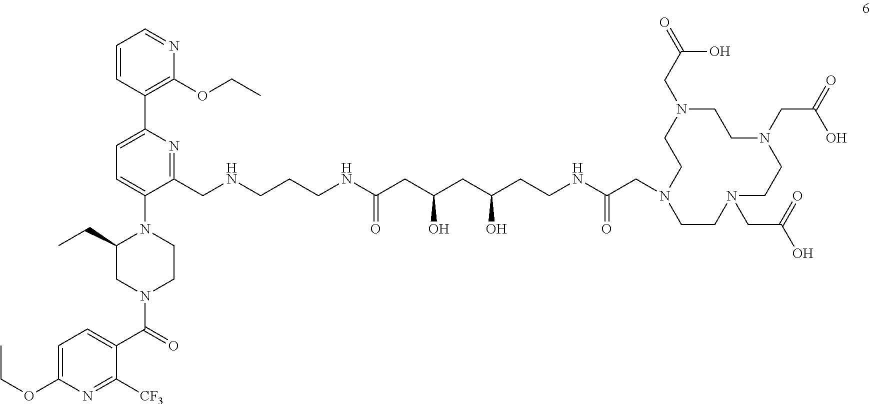 [SMILES: CCOc1ccc(C(=O)N2CCN(c3ccc(-c4cccnc4OCC)nc3CNCCCNC(=O)C[C@H](O)C[C@H](O)CCNC(=O)CN3CCN(CC(=O)O)CCN(CC(=O)O)CCN(CC(=O)O)CC3)[C@H](CC)C2)c(C(F)(F)F)n1]

Step 1: Into an 8-mL vial, was placed a mixture of 2-(4,7,10-tris(2-(tert-butoxy)-2-oxoethyl)-1,4,7,10-tetraazacyclododecan-1-yl)acetic acid (150 mg, 3.0 Eq, 262 μmol), chloro-N,N,N′,N′-tetramethylformamidinium hexafluorophosphate (TCFH) (120 mg, 4.8 Eq, 428 μmol), N-methylimidazole (NMI) (80 mg, 11 Eq, 0.97 mmol) and DMF (1 mL). The resulting mixture was stirred at 30° C. for 30 min, followed by the addition of (3R,5R)-7-amino-N-(3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)-3,5-dihydroxyheptanamide (85 mg, 1 Eq, 89 μmol). The reaction mixture was stirred at 30° C. for 1 hour. The reaction residue was purified by MPLC using the following conditions: Column, C18, 120 g; mobile phase, Water (0.1% FA) and ACN (30% ACN up to 98% in 10 min); Total flow rate, 70 mL/min: Detector, UV 220 nm. The collected fractions were concentrated. This resulted in tri-tert-butyl 2,2′,2″-(10-(2-(((3R,5R)-7-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)amino)-3,5-dihydroxy-7-oxoheptyl)amino)-2-oxoethyl)-1,4,7,10-tetraazacyclododecane-1,4,7-triyl)triacetate (102 mg, 67.3 μmol, 76%) as light yellow solid. [M+Na]=1537.0, [M/2+H]=758.4.

Step 2: Into an 8-mL vial, was placed a mixture of tri-tert-butyl 2,2′,2″-(10-(2-(((3R,5R)-7-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)amino)-3,5-dihydroxy-7-oxoheptyl)amino)-2-oxoethyl)-1,4,7,10-tetraazacyclododecane-1,4,7-triyl)triacetate (102 mg, 1 Eq, 67.3 μmol), 2-chlorobenzenethiol (50 mg, 5.1 Eq, 0.35 mmol), K₂CO₃ (75 mg, 8.1 Eq, 0.54 mmol) and MeCN (2 mL). The reaction mixture was stirred at 50° C. for 1 hour. The reaction crude mixture was purified by MPLC using the following conditions: Column, C18, 120 g; mobile phase, Water (0.1% FA) and ACN (30% ACN up to 98% in 4 min, 98% ACN to 98% in 3 min); Total flow rate, 70 mL/min; Detector, UV 220 nm. Fractions collected were concentrated under reduced pressure and dried to afford tri-tert-butyl 2,2′,2″-(10-(2-(((3R,5R)-7-((3-(((2′-ethoxy-5-((R)-4-(6-ethoxy-2-(trifluoromethyl)nicotinoyl)-2-ethylpiperazin-1-yl)-[2,3′-bipyridin]-6-yl)methyl)amino)propyl)-amino)-3,5-dihydroxy-7-oxoheptyl)amino)-2-oxoethyl)-1,4,7,10-tetraazacyclododecane-1,4,7-triyl)triacetate (70 mg, 53 μmol, 78%) as a yellow solid. [M+Na]=1359.1, [M/2+H]=665.7.

Step 3: Into an 8-mL vial, was placed a mixture of tri-tert-butyl 2,2′,2″-(10-(2-(((3R,5R)-7-((3-(((2′-ethoxy-5-((R)-4-(6-ethoxy-2-(trifluoromethyl)nicotinoyl)-2-ethylpiperazin-1-yl)-[2,3′-bipyridin]-6-yl)methyl)amino)propyl)amino)-3,5-dihydroxy-7-oxoheptyl)amino)-2-oxoethyl)-1,4,7,10-tetraazacyclododecane-1,4,7-triyl)triacetate (30 mg, 1 Eq, 23 μmol) and TFA (0.5 mL). The reaction mixture was stirred at 30° C. for 2 hrs. The reaction mixture was concentrated under reduced pressure to afford crude 2,2′,2″-(10-(2-(((3R,5R)-7-((3-(((2′-ethoxy-5-((R)-4-(6-ethoxy-2-(trifluoromethyl)-nicotinoyl)-2-ethylpiperazin-1-yl)-[2,3′-bipyridin]-6-yl)methyl)amino)propyl)-amino)-3,5-dihydroxy-7-oxoheptyl)amino)-2-oxoethyl)-1,4,7,10-tetraazacyclododecane-1,4,7-triyl)triacetic acid (26 mg, 22 μmol, 99%) as a yellow crude oil. This material was used for next step without any purification. [M+Na]=1183.8, [M/2+H]=581.6.

### Example 52: ¹¹⁵Indium Complex of Compound 6

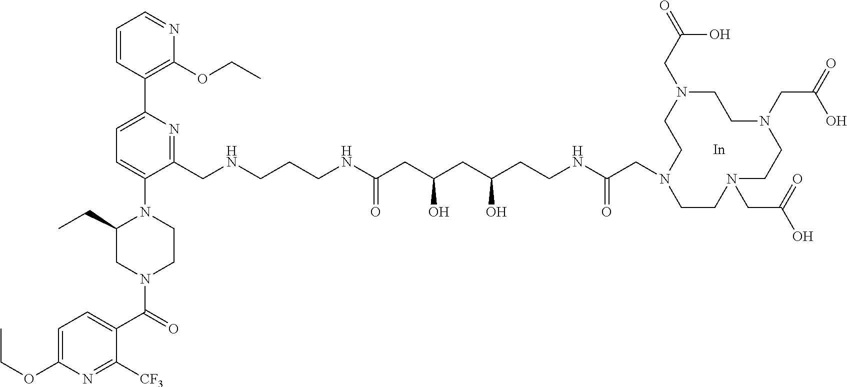

Into an 8-mL vial, was placed a mixture of 2,2′,2″-(10-(2-(((3R,5R)-7-((3-(((2′-ethoxy-5-((R)-4-(6-ethoxy-2-(trifluoromethyl)nicotinoyl)-2-ethylpiperazin-1-yl)-[2,3′-bipyridin]-6-yl)methyl)amino)propyl)amino)-3,5-dihydroxy-7-oxoheptyl)amino)-2-oxoethyl)-1,4,7,10-tetraazacyclododecane-1,4,7-triyl)triacetic acid (24 mg, 1 Eq, 21 μmol), ¹¹⁵InCl₃ (25 mg, 5.5 Eq, 0.11 mmol), NaHCO₃ (20 mg, 12 Eq, 0.24 mmol), MeCN (1 mL) and Water (0.3 mL). The reaction mixture was stirred at 80° C. for 1 hour and cool down to ambient temperature. The reaction crude was diluted with MeCN (4 mL) and the organic layer was separated and purified by prep-HPLC using the following conditions: Column, Sunfire Prep C18 OBD Column, 50×250 mm, 5 m 10 nm; Mobile Phase A: Water (0.05% TFA), Mobile Phase B: ACN; Flow rate: 90 mL/min; Gradient: 20% B to 65% B in 12 min. Pure fractions were collected and lyophilized. This resulted indium(III) 2,2′,2″-(10-(2-(((3R,5R)-7-((3-(((2′-ethoxy-5-((R)-4-(6-ethoxy-2-(trifluoromethyl)-nicotinoyl)-2-ethylpiperazin-1-yl)-[2,3′-bipyridin]-6-yl)methyl)amino)propyl)amino)-3,5-dihydroxy-7-oxoheptyl)amino)-2-oxoethyl)-1,4,7,10-tetraazacyclododecane-1,4,7-triyl)triacetate TFA salt (5.6 mg, 4.0 μmol, 20%) as a white solid. [M+H-1TFA]=1273.7.

### Example 53: 3-((R)-4-(4-chloro-2-(difluoromethyl)benzoyl)-2-ethylpiperazin-1-yl)-6-(2-ethoxyphenyl)-N—((R)-pyrrolidin-3-yl)picolinamide

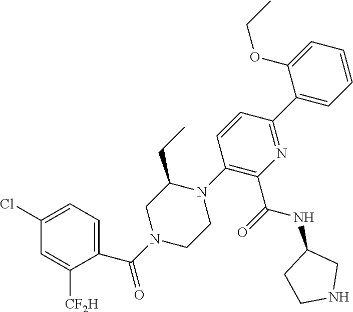 [SMILES: CCOc1ccccc1-c1ccc(N2CCN(C(=O)c3ccc(Cl)cc3C(F)F)C[C@H]2CC)c(C(=O)N[C@@H]2CCNC2)n1]

Step 1: Into a 250-mL round bottom flask, was placed a mixture of 6-bromo-3-fluoropicolinonitrile (10.0 g, 1 Eq, 49.8 mmol), tert-butyl (R)-3-ethylpiperazine-1-carboxylate (11.7 g, 1.10 Eq, 54.6 mmol), DIEA (16.1 g, 21.7 mL, 2.50 Eq, 125 mmol) and DMSO (100 mL). The reaction mixture was stirred at 90° C. for 24 hrs. The reaction mixture was dilute with water (500 mL) and extracted with EtOAc (350 mL×3). Organic layers were combined, washed with water (300 mL×2) and brine (300 mL), dried over anhydrous Na₂SO₄, filtered and concentrated under reduced pressure. The remaining residue was purified by silica gel chromatography (EtOAc/PE from 0% to 85% in 15 min) to afford tert-butyl (R)-4-(6-bromo-2-cyanopyridin-3-yl)-3-ethylpiperazine-1-carboxylate (11.1 g, 28.1 mmol, 56.4%) as yellow oil. [M+H-56]=338.9, 340.9.

Step 2: Into a 50 mL round bottom flask, purged and maintained with an atmosphere of nitrogen, was placed a mixture of tert-butyl (R)-4-(6-bromo-2-cyanopyridin-3-yl)-3-ethylpiperazine-1-carboxylate (2.00 g, 1 Eq, 5.06 mmol), (2-ethoxyphenyl)boronic acid (1.68 g, 2.00 Eq, 10.1 mmol), Pd(DTBPF)C12 (150 mg, 0.0455 Eq, 230 μmol), K₂CO₃ (2.10 g, 3.00 Eq, 15.2 mmol), 1,4-Dioxane (25 mL) and Water (5 mL). The reaction mixture was stirred at 70° C. for 2 hrs. The reaction mixture was concentrated, and the remaining residue was purified by silica gel chromatography (EtOAc/PE from 0% to 85% in 15 min). This resulted in tert-butyl (R)-4-(2-cyano-6-(2-ethoxyphenyl)pyridin-3-yl)-3-ethylpiperazine-1-carboxylate (1.85 g, 4.24 mmol, 83.8%) as yellow oil. [M+H]=437.2.

Step 3: Into a 40-mL vial, was placed a mixture of tert-butyl (R)-4-(2-cyano-6-(2-ethoxyphenyl)pyridin-3-yl)-3-ethylpiperazine-1-carboxylate (1.00 g, 1 Eq, 2.29 mmol), KOH (1.29 g, 10.0 Eq, 23.0 mmol), EtOH (5 mL) and Water (5 mL). The reaction mixture was stirred at 100° C. for 48 hrs. The reaction mixture was and concentrated and the remaining residue was purified by silica gel chromatography (EtOAc/PE from 0% to 85% in 15 min). This resulted in (R)-3-(4-(tert-butoxycarbonyl)-2-ethylpiperazin-1-yl)-6-(2-ethoxyphenyl)picolinic acid (820 mg, 1.80 mmol, 78.6%) as yellow oil. [M+H]=456.3.

Step 4: Into a 40-mL vial, was placed a mixture of (R)-3-(4-(tert-butoxycarbonyl)-2-ethylpiperazin-1-yl)-6-(2-ethoxyphenyl)picolinicacid (540 mg, 1 Eq, 1.19 mmol), HATU (676 mg, 1.50 Eq, 1.78 mmol), DIEA (613 mg, 826 μL, 4.00 Eq, 4.74 mmol) and DMF (10 mL). The reaction mixture was stirred at 20° C. for 10 minutes, followed by the addition of benzyl (R)-3-aminopyrrolidine-1-carboxylate (392 mg, 1.50 Eq, 1.78 mmol) was added and the reaction mixture was stirred at 30° C. for 1 hour. The reaction mixture was dilute with 250 mL of water and extracted with EtOAc (150 mL×3). Organic layers were combined, washed with water (150 mL×2) and brine (150 mL), dried over anhydrous Na₂SO₄, filtered and concentrated under reduced pressure. The residue was purified by silica gel chromatography (EtOAc/PE from 0% to 85% in 15 min), resulting in tert-butyl (R)-4-(2-(((R)-1-((benzyloxy)carbonyl)pyrrolidin-3-yl)carbamoyl)-6-(2-ethoxyphenyl) pyridin-3-yl)-3-ethylpiperazine-1-carboxylate (650 mg, 988 μmol, 83.4%) as an oil. [M+H]=658.4.

Step 5: To a DCM (10 mL) solution of tert-butyl (R)-4-(2-(((R)-1-((benzyloxy)carbonyl)-pyrrolidin-3-yl)carbamoyl)-6-(2-ethoxyphenyl)pyridin-3-yl)-3-ethylpiperazine-1-carboxylate (650 mg, 1 Eq, 988 μmol) was added TFA (3 mL) under an atmosphere of nitrogen. The reaction mixture was stirred at 30° C. for 1 hour. The reaction mixture was concentrated under reduced pressure to give crude benzyl(R)-3-(6-(2-ethoxyphenyl)-3-((R)-2-ethylpiperazin-1-yl)picolinamido)pyrrolidine-1-carboxylate (550 mg, 986 μmol, 99.8%) as a yellow crude oil. [M+H]=558.4.

Step 6: Into a 40-mL vial, was placed a mixture of 4-chloro-2-(difluoromethyl)benzoic acid (204 mg, 1.00 Eq, 988 μmol), HATU (562 mg, 1.50 Eq, 1.48 mmol), DIEA (382 mg, 515 μL, 3.00 Eq, 2.96 mmol) and DMF (10 mL). The reaction mixture was stirred at 30° C. for 10 minutes, followed by the addition of benzyl (R)-3-(6-(2-ethoxyphenyl)-3-((R)-2-ethylpiperazin-1-yl) picolinamido)pyrrolidine-1-carboxylate (550 mg, 1 Eq, 986 μmol). The resulting mixture was stirred at 30° C. for 1 hour. The reaction mixture was dilute with 250 mL of water and extracted with EtOAc (150 mL×3). Organic layers were the combined, washed with water (150 mL×2) and brine (150 mL), dried over anhydrous Na₂SO₄, filtered and concentrated under reduced pressure. The residue was purified by silica gel chromatography (EtOAc/PE from 0% to 85% in 15 min) providing benzyl (R)-3-(3-((R)-4-(4-chloro-2-(difluoromethyl)benzoyl)-2-ethylpiperazin-1-yl)-6-(2-ethoxy-phenyl) picolinamido)pyrrolidine-1-carboxylate (480 mg, 643 μmol, 65.2%) as an oil. [M+H]=746.2, 748.2.

Step 7: Into an 8-mL vial, was placed benzyl (R)-3-(3-((R)-4-(4-chloro-2-(difluoromethyl) benzoyl)-2-ethylpiperazin-1-yl)-6-(2-ethoxyphenyl)picolinamido)pyrrolidine-1-carboxylate (470 mg, 1 Eq, 630 μmol) and TFA (4 mL). The resulting solution was stirred at 60° C. for 16 hrs. The reaction mixture was concentrated, and the remaining residue was purified by Prep-HPLC using the following conditions: Column, C18 120 g; mobile phase, Water (0.1% NH₃·H₂O) and ACN (30% ACN up to 98% in 3 min); Total flow rate, 70 mL/min; Detector, UV 220 nm. This resulted in 3-((R)-4-(4-chloro-2-(difluoromethyl)benzoyl)-2-ethylpiperazin-1-yl)-6-(2-ethoxyphenyl)-N—((R)-pyrrolidin-3-yl)picolinamide (358 mg, 585 μmol, 92.9%) as a light brown oil. [M+H]=612.2.

### Example 54: tert-butyl (14-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)-3,6,9,12-tetraoxatetradecyl)carbamate

Into an 8 mL vial, was placed a mixture of 3-((R)-4-(4-chloro-2-(difluoromethyl)benzoyl)-2-ethylpiperazin-1-yl)-6-(2-ethoxyphenyl)-N—((R)-pyrrolidin-3-yl)picolinamide (100 mg, 1 Eq, 163 μmol), tert-butyl (14-bromo-3,6,9,12-tetraoxatetradecyl)carbamate (135 mg, 2.06 Eq, 337 μmol), NaI (27 mg, 1.1 Eq, 0.18 mmol) K₂CO₃ (71 mg, 3.1 Eq, 0.51 mmol) and MeCN (3 mL). The reaction mixture was stirred at 80° C. for 3 hrs. The reaction crude was purified by Prep-HPLC using the following conditions: Column, C18 120 g; mobile phase, Water (0.1% TFA) and ACN (30% ACN up to 98% in 7 min); Total flow rate, 70 mL/min; Detector, UV 220 nm. This resulted in tert-butyl (14-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)-3,6,9,12-tetraoxatetradecyl)carbamate (89 mg, 96 μmol, 58%) as a light yellow solid. [M+H]=931.5.

### Example 55: N—((R)-1-(14-amino-3,6,9,12-tetraoxatetradecyl)pyrrolidin-3-yl)-3-((R)-4-(4-chloro-2-(difluoromethyl)benzoyl)-2-ethylpiperazin-1-yl)-6-(2-ethoxyphenyl)picolinamide

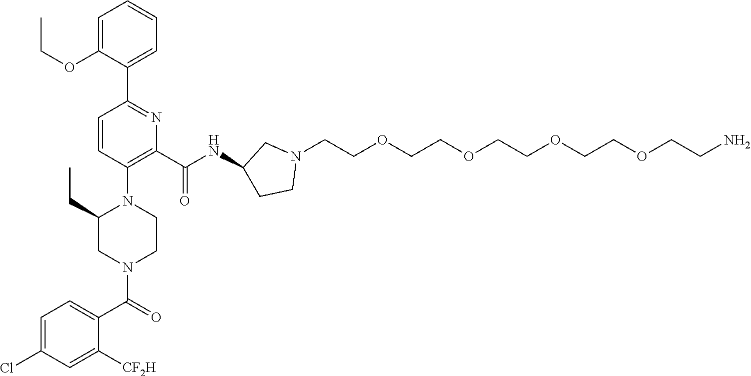 [SMILES: CCOc1ccccc1-c1ccc(N2CCN(C(=O)c3ccc(Cl)cc3C(F)F)C[C@H]2CC)c(C(=O)N[C@@H]2CCN(CCOCCOCCOCCOCCN)C2)n1]

To a DCM (1 mL) solution of tert-butyl (14-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)-3,6,9,12-tetraoxatetradecyl)carbamate (89 mg, 1 Eq, 96 μmol) was added TFA (0.5 mL). The resulting mixture was stirred at 28° C. for 1 hour. The reaction crude was purified by Prep-HPLC using the following conditions (IntelFlash-1): Column, C18 silica gel; mobile phase, water (0.1 aq. NH₃/CH₃CN (CH₃CN: 30%-98% in 3 min); Detector, UV 254&220 nm. This resulted in N—((R)-1-(14-amino-3,6,9,12-tetraoxatetradecyl)pyrrolidin-3-yl)-3-((R)-4-(4-chloro-2-(difluoromethyl) benzoyl)-2-ethylpiperazin-1-yl)-6-(2-ethoxyphenyl)picolinamide (73 mg, 88 μmol, 92%) as a light brown oil. M+H=831.4.

### Example 56: 2,2′,2″-(10-(17-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)-2-oxo-6,9,12,15-tetraoxa-3-azaheptadecyl)-1,4,7,10-tetraazacyclododecane-1,4,7-triyl)triacetic acid (Compound 7)

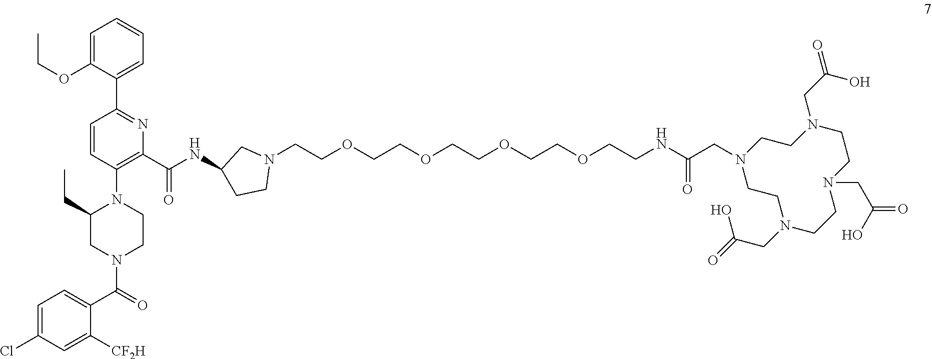 [SMILES: CCOc1ccccc1-c1ccc(N2CCN(C(=O)c3ccc(Cl)cc3C(F)F)C[C@H]2CC)c(C(=O)N[C@@H]2CCN(CCOCCOCCOCCOCCNC(=O)CN3CCN(CC(=O)O)CCN(CC(=O)O)CCN(CC(=O)O)CC3)C2)n1]

Step 1: Into an 8-mL vial, was placed a mixture of 2-(4,7,10-tris(2-(tert-butoxy)-2-oxoethyl)-1,4,7,10-tetraazacyclododecan-1-yl)acetic acid (31 mg, 1.4 Eq, 54 μmol), HATU (23 mg, 1.5 Eq, 60 μmol), DIEA (26 mg, 35 μL, 5.1 Eq, 0.20 mmol) and DMF (1 mL). The resulting mixture was stirred at 28° C. for 5 min, followed by the addition of N—((R)-1-(14-amino-3,6,9,12-tetraoxatetradecyl)pyrrolidin-3-yl)-3-((R)-4-(4-chloro-2-(difluoromethyl)benzoyl)-2-ethylpiperazin-1-yl)-6-(2-ethoxyphenyl)picolinamide (33 mg, 1 Eq, 40 μmol). The reaction mixture was stirred at the same temperature for 1 hour. The reaction crude was purified by Prep-HPLC using the following conditions: Column, C18 120 g; mobile phase, Water (0.05% FA) and ACN (30% ACN up to 98% in 7 min); Total flow rate, 70 mL/min; Detector, UV 220 nm. This resulted in tri-tert-butyl 2,2′,2″-(10-(17-((R)-3-(3-((R)-4-(4-chloro-2-(difluoromethyl)benzoyl)-2-ethylpiperazin-1-yl)-6-(2-ethoxy-phenyl)picolinamido)pyrrolidin-1-yl)-2-oxo-6,9,12,15-tetraoxa-3-azaheptadecyl)-1,4,7,10-tetraazacyclododecane-1,4,7-triyl)triacetate (28 mg, 20 μmol, 51%) as an oil. [M+H]=1385.7.

Step 2: Into an 8-mL vial, was placed tri-tert-butyl 2,2′,2″-(10-(17-((R)-3-(3-((R)-4-(4-chloro-2-(difluoromethyl)benzoyl)-2-ethylpiperazin-1-yl)-6-(2-ethoxyphenyl)picolinamido)-pyrrolidin-1-yl)-2-oxo-6,9,12,15-tetraoxa-3-azaheptadecyl)-1,4,7,10-tetraazacyclododecane-1,4,7-triyl)triacetate (28 mg, 1 Eq, 20 μmol) and TFA (0.5 mL). The resulting solution was stirred at 28° C. for 5 hours. The resulting mixture was concentrated under vacuum. The remaining residue was purified by Prep-HPLC using the following conditions: Column, C18 120 g Column; mobile phase, Water (0.1% FA) and ACN (30% ACN up to 98% in 2 min); Total flow rate, 70 mL/min; Detector, UV 220 nm. This resulted in 2,2′,2″-(10-(17-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)-2-oxo-6,9,12,15-tetraoxa-3-azaheptadecyl)-1,4,7,10-tetraazacyclododecane-1,4,7-triyl)triacetic acid (15 mg, 12 μmol, 61%) as a light yellow oil. M+H=1217.7.

### Example 57: ¹¹⁵Indium Complex of Compound 7

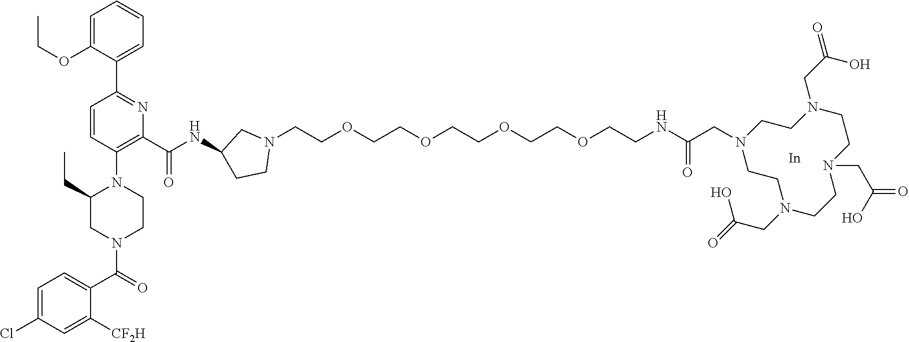

Into an 8 mL vial, was placed 2,2′,2″-(10-(17-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)-2-oxo-6,9,12,15-tetraoxa-3-azaheptadecyl)-1,4,7,10-tetraazacyclododecane-1,4,7-triyl)triacetic acid (15 mg, 1 Eq, 12 μmol), ¹¹⁵InCl₃ (14 mg, 5.1 Eq, 63 μmol), sodium hydrogen carbonate (22 mg, 21 Eq, 0.26 mmol), MeCN (0.15 mL) and water (0.15 mL). The reaction mixture was stirred at 80° C. for 1 hour. The resulting mixture was purified by Prep-HPLC: Sunfire Prep C18 OBD Column, 50*250 mm, 5 m 10 nm; Mobile Phase A: Water (0.05% TFA), Mobile Phase B: ACN; Flow rate: 90 mL/min; Gradient: 35% B to 55% B in 8 min. This resulted in the product (5.1 mg, 3.3 μmol, 27%) as a yellow solid. [M+H-2TFA]=1329.7.

### Example 58: tert-butyl (15-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)-3,6,9,12-tetraoxapentadecyl)carbamate

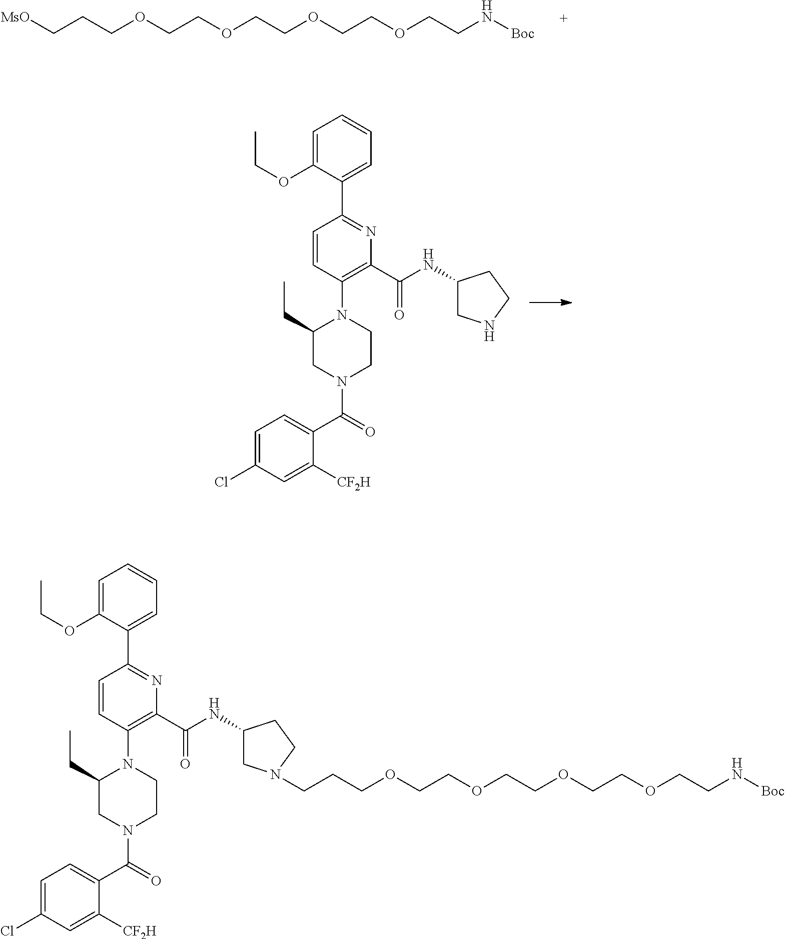 [SMILES: CC(C)(C)OC(=O)NCCOCCOCCOCCOCCCOS(C)(=O)=O ; CCOc1ccccc1-c1ccc(N2CCN(C(=O)c3ccc(Cl)cc3C(F)F)C[C@H]2CC)c(C(=O)N[C@@H]2CCNC2)n1 ; CCOc1ccccc1-c1ccc(N2CCN(C(=O)c3ccc(Cl)cc3C(F)F)C[C@H]2CC)c(C(=O)N[C@@H]2CCN(CCCOCCOCCOCCOCCNC(=O)OC(C)(C)C)C2)n1]

Into an 80 mL vial, was placed with a mixture of 2,2-dimethyl-4-oxo-3,8,11,14,17-pentaoxa-5-azaicosan-20-yl methanesulfonate (143 mg, 2.04 Eq, 333 μmol), 3-((R)-4-(4-chloro-2-(difluoromethyl)benzoyl)-2-ethylpiperazin-1-yl)-6-(2-ethoxyphenyl)-N—((R)-pyrrolidin-3-yl)-picolinamide (100 mg, 1 Eq, 163 μmol), K₂CO₃ (68 mg, 3.0 Eq, 0.49 mmol), NaI (71 mg, 2.9 Eq, 0.47 mmol) and MeCN (3 mL). The reaction mixture was stirred at 80° C. for 5 hours. The reaction crude was purified by Prep-HPLC using the following conditions: Column, C18 120 g Column; mobile phase, Water (0.1% FA) and ACN (30% ACN up to 98% in 7 min); Total flow rate, 70 mL/min; Detector, UV 220 nm. This resulted in the title compound (95 mg, 0.10 mmol, 62%) as a solid. [M+H]=945.5, 947.5.

### Example 59: N—((R)-1-(1-amino-3,6,9,12-tetraoxapentadecan-15-yl)pyrrolidin-3-yl)-3-((R)-4-(4-chloro-2-(difluoromethyl)benzoyl)-2-ethylpiperazin-1-yl)-6-(2-ethoxyphenyl)picolinamide

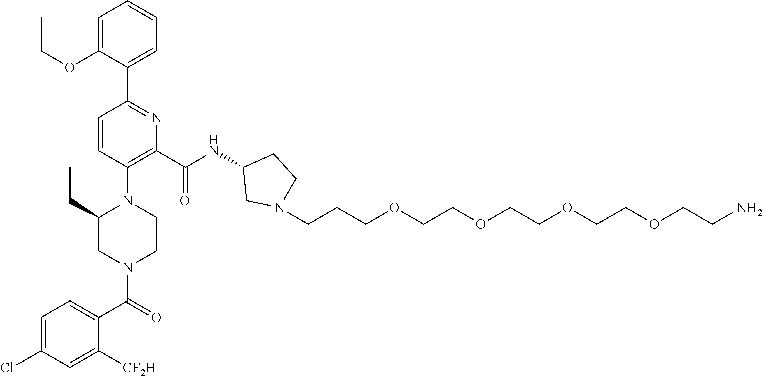 [SMILES: CCOc1ccccc1-c1ccc(N2CCN(C(=O)c3ccc(Cl)cc3C(F)F)C[C@H]2CC)c(C(=O)N[C@@H]2CCN(CCCOCCOCCOCCOCCN)C2)n1]

Into an 8 mL vial, were added tert-butyl (15-((R)-3-(3-((R)-4-(4-chloro-2-difluoromethyl)-benzoyl)-2-ethylpiperazin-1-yl)-6-(2-ethoxyphenyl)picolinamido)pyrrolidin-1-yl)-3,6,9,12-tetraoxapentadecyl)carbamate (95 mg, 1 Eq, 0.10 mmol), DCM (1.5 mL) and TFA (0.5 mL). The resulting mixture was stirred at 28° C. for 1 hr. The reaction crude was purified by Prep-HPLC using the following conditions (IntelFlash-1): Column, C18 silica gel; mobile phase, water (0.1 FA)/CH₃CN (CH₃CN:30%-98% in 3 min, 98-98% in 6 min); Detector, UV 254&220 nm. This resulted in the title compound (81 mg, 96 μmol, 95%) as a light yellow solid. M+H=845.4, 847.4.

### Example 60: 2,2′,2″-(10-(18-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)-2-oxo-6,9,12,15-tetraoxa-3-azaoctadecyl)-1,4,7,10-tetraazacyclododecane-1,4,7-triyl)triacetic acid (Compound 8)

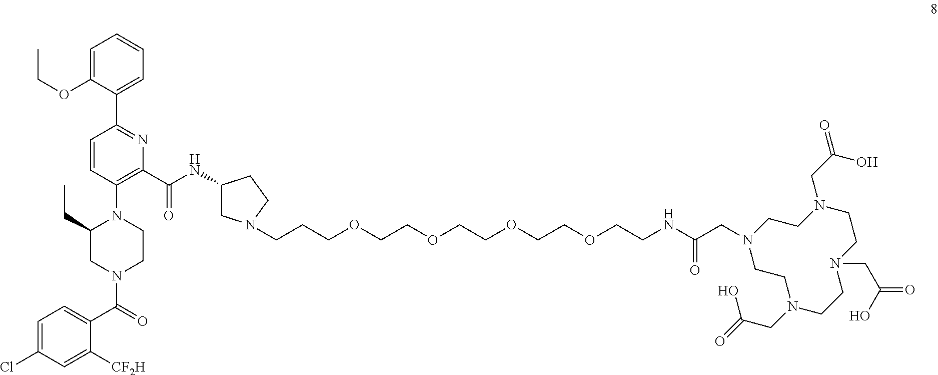 [SMILES: CCOc1ccccc1-c1ccc(N2CCN(C(=O)c3ccc(Cl)cc3C(F)F)C[C@H]2CC)c(C(=O)N[C@@H]2CCN(CCCOCCOCCOCCOCCNC(=O)CN3CCN(CC(=O)O)CCN(CC(=O)O)CCN(CC(=O)O)CC3)C2)n1]

Step 1: Into an 8-mL vial, was placed a mixture of 2-(4,7,10-tris(2-(tert-butoxy)-2-oxoethyl)-1,4,7,10-tetraazacyclododecan-1-yl)acetic acid (35 mg, 1.3 Eq, 61 μmol), HATU (46 mg, 2.6 Eq, 0.12 mmol), DIEA (26 mg, 35 μL, 4.3 Eq, 0.20 mmol) and DMF (1 mL). The resulting mixture was stirred at 28° C. for 5 min, followed by the addition of N—((R)-1-(1-amino-3,6,9,12-tetraoxapentadecan-15-yl)pyrrolidin-3-yl)-3-((R)-4-(4-chloro-2-(difluoromethyl)benzoyl)-2-ethylpiperazin-1-yl)-6-(2-ethoxyphenyl)picolinamide (40 mg, 1 Eq, 47 μmol). The reaction mixture was stirred at 28° C. for 1 hr. The reaction crude was purified by Prep-HPLC using the following conditions: Column, C18 120 g Column; mobile phase, Water (0.05% FA) and ACN (30% ACN up to 98% in 7 min); Total flow rate, 70 mL/min; Detector, UV 220 nm. Pure fractions were collected and concentrated. This resulted in tri-tert-butyl 2,2′,2″-(10-(18-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)-2-oxo-6,9,12,15-tetraoxa-3-azaoctadecyl)-1,4,7,10-tetraazacyclododecane-1,4,7-triyl)triacetate (39 mg, 28 μmol, 59%) as light yellow solid. [M+H]=1399.7, 1401.7.

Step 2: Into an 8 mL vial, were added tri-tert-butyl 2,2′,2″-(10-(18-((R)-3-(3-((R)-4-(4-chloro-2-(difluoromethyl)benzoyl)-2-ethylpiperazin-1-yl)-6-(2-ethoxyphenyl)picolinamido)-pyrrolidin-1-yl)-2-oxo-6,9,12,15-tetraoxa-3-azaoctadecyl)-1,4,7,10-tetraazacyclododecane-1,4,7-triyl)triacetate (39 mg, 1 Eq, 28 μmol) and TFA (0.5 mL). The resulting mixture was stirred at 28° C. for 5 hrs. The reaction crude was purified by Prep-HPLC using the following conditions (IntelFlash-1): Column, C18 silica gel; mobile phase, water (0.1% FA/CH₃CN (CH₃CN:30%-98% in 3 min, Flow rate: 70 mL/min); Detector, UV 254&220 nm. This resulted in 2,2′,2″-(10-(18-((R)-3-(3-((R)-4-(4-chloro-2-(difluoromethyl)benzoyl)-2-ethylpiperazin-1-yl)-6-(2-ethoxyphenyl)picolinamido)-pyrrolidin-1-yl)-2-oxo-6,9,12,15-tetraoxa-3-azaoctadecyl)-1,4,7,10-tetraazacyclododecane-1,4,7-triyl)triacetic acid (24 mg, 19 μmol, 70%) as a light yellow solid. M+H=1231.5, 1233.5.

### Example 61: ¹¹⁵Indium Complex of Compound 8

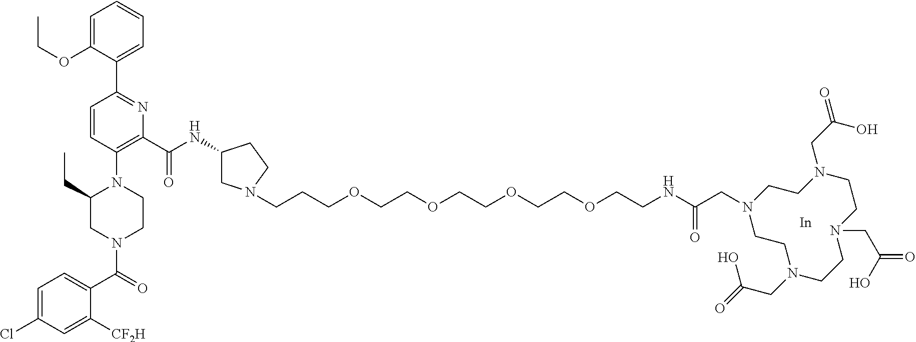

Into an 8 mL vial, was placed 2,2′,2″-(10-(18-((R)-3-(3-((R)-4-(4-chloro-2-difluoromethyl)benzoyl)-2-ethylpiperazin-1-yl)-6-(2-ethoxyphenyl)picolinamido)pyrrolidin-1-yl)-2-oxo-6,9,12,15-tetraoxa-3-azaoctadecyl)-1,4,7,10-tetraazacyclododecane-1,4,7-triyl)triacetic acid (24 mg, 1 Eq, 19 μmol), ¹¹⁵InCl₃ (22 mg, 5.1 Eq, 99 μmol), NaHCO₃ (47 mg, 29 Eq, 0.56 mmol), MeCN (0.3 mL) and water (0.1 mL). The resulting mixture was stirred at 80° C. for 1 hour. The reaction crude was purified by Prep-HPLC: SunfirePrep C18 OBD Column, 50×250 mm, 5 m, 10 nm; Mobile Phase A: Water (0.05% TFA), Mobile Phase B: ACN; Flowrate: 90 mL/min; Gradient: 25% B to 45% B in 8 min. Pure fractions were collected concentrated to afford the product (6.4 mg, 4.1 μmol, 21%) as a light-yellow solid. [M+H-2TFA]=1343.7, 1345.7.

### Example 62: (R)-1-(2-cyano-4-(trifluoromethyl)phenyl)-4-(6-(2-ethoxyphenyl)-5-fluoropyridin-3-yl)-N-(pyrrolidin-3-yl)piperidine-4-carboxamide

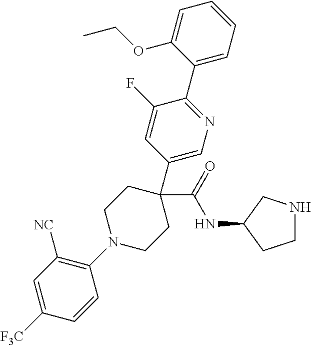 [SMILES: CCOc1ccccc1-c1ncc(C2(C(=O)N[C@@H]3CCNC3)CCN(c3ccc(C(F)(F)F)cc3C#N)CC2)cc1F]

Step 1: Into a 1000-mL round-bottom flask, purged and maintained with an inert atmosphere of nitrogen, was placed 5-bromo-2-chloro-3-fluoropyridine (15 g, 1 Eq, 71 mmol), 1,4-Dioxane (150 mL) and H₂O (15 mL). 4-(4,4,5,5-Tetramethyl-1,3,2-dioxaborolan-2-yl)isoxazole (17 g, 1.2 Eq, 87 mmol), Pd(DtBPF)Cl₂ (1.4 g, 0.030 Eq, 2.1 mmol), and potassium fluoride (12 g, 2.9 Eq, 0.21 mol) were added. The resulting mixture was stirred at 100° C. for 1 hr. The reaction mixture was filtered, and the filtrate was concentrated under vacuum. This resulted in crude 4-(6-chloro-5-fluoropyridin-3-yl)isoxazole (14.2 g, 71.5 mmol, 100%) as yellow oil. This material was used in next step without further purification. LCMS[M+H]⁺: 199.0, 201.0.

Step 2: Into a 500-mL round-bottom flask, was placed 4-(6-chloro-5-fluoropyridin-3-yl)isoxazole (14.2 g, 1 Eq, 71.5 mmol), MeOH (100 mL), H₂O (10 mL), and potassium fluoride (8.3 g, 2.0 Eq, 0.14 mol). The resulting solution was stirred at 90° C. for 16 hrs. The reaction mixture was filtered, and the filtrate was concentrated under vacuum. The remaining residue was purified by silica gel chromatography eluting with ethyl acetate/petroleum ether (1:3). This resulted in 2-(6-chloro-5-fluoropyridin-3-yl)acetonitrile (10.1 g, 59.2 mmol, 82.8%) as a solid. MS [M+H]⁺: 117.0, 119.0.

Step 3: Into a 250-mL 3-necked round-bottom flask, purged and maintained with an inert atmosphere of nitrogen, was placed 2-(6-chloro-5-fluoropyridin-3-yl)acetonitrile (10.1 g, 1 Eq, 59.2 mmol) and DMSO (100 mL), followed by the addition of KOH (9.5 g, 2.9 Eq, 0.17 mol) and N-benzyl-2-bromo-N-(2-bromoethyl)ethan-1-amine (20.9 g, 1.10 Eq, 65.1 mmol). The resulting mixture was stirred at 20° C. for 1 hr. This resulted in 1-benzyl-4-(6-chloro-5-fluoropyridin-3-yl)piperidine-4-carbonitrile (13.8 g, 41.8 mmol, 70.7%) as a solid. LCMS[M+H]⁺: 330.0, 332.0.

Step 4: 1-Benzyl-4-(6-chloro-5-fluoropyridin-3-yl)piperidine-4-carbonitrile (500 mg, 1 Eq, 1.52 mmol), (2-ethoxyphenyl)boronic acid (377 mg, 1.5 Eq, 2.27 mmol), potassium carbonate (629 mg, 3 Eq, 4.55 mmol), and Pd(dtbpf)Cl₂ (98.8 mg, 0.1 Eq, 152 μmol) were placed in a sealed tube. 1,4-Dioxane (4 mL) and Water (0.4 mL) were added, and the reaction mixture reaction was bubbled with N2 for 1 minute. The resulting solution was stirred at 100° C. for 4 hrs. The reaction crude was extracted with EtOAc, washed with brine, concentrated and purified by silica gel chromatography to give 1-benzyl-4-(6-(2-ethoxyphenyl)-5-fluoropyridin-3-yl)piperidine-4-carbonitrile (507 mg, 1.22 mmol, 80.5%) as a light yellow solid. MS (M+H)+=416.6.

Step 5: 1-benzyl-4-(6-(2-ethoxyphenyl)-5-fluoropyridin-3-yl)piperidine-4-carbonitrile (500 mg, 1 Eq, 1.20 mmol) was dissolved in EtOH (3 mL) and Water (3 mL) in a sealed tube. KOH (675 mg, 10 Eq, 12.0 mmol) was added, and the reaction mixture was stirred at 100° C. for 5 hours. The reaction crude was diluted with water and neutralized to pH=7 with 1 N HCl solution. The resulting suspension was filtered, and the light brown solid was collected and dried under vacuum. MS (M+H)+=434.7

Step 6: To a DMF (3 mL) solution of 1-benzyl-4-(6-(2-ethoxyphenyl)-5-fluoropyridin-3-yl)piperidine-4-carboxylic acid (196 mg, 1 Eq, 451 μmol) was added HATU (257 mg, 1.5 Eq, 677 μmol) and DIPEA (233 mg, 314 μL, 4 Eq, 1.80 mmol). The resulting mixture was stirred at ambient temperature for 2 minutes, followed by the addition of tert-butyl (R)-3-aminopyrrolidine-1-carboxylate (126 mg, 1.5 Eq, 677 μmol). The resulting mixture was stirred at 20° C. for 5 hours. The reaction mixture was diluted with ethyl acetate, washed with water and brine, concentrated and purified by silica gel chromatography (0-100% EtOAc/Hexanes) to give tert-butyl (R)-3-(1-benzyl-4-(6-(2-ethoxyphenyl)-5-fluoropyridin-3-yl)piperidine-4-carboxamido)pyrrolidine-1-carboxylate (237 mg, 393 μmol, 87.2%) as a off-white solid. MS (M+H)+=603.9

Step 7: To a solution of tert-butyl (R)-3-(1-benzyl-4-(6-(2-ethoxyphenyl)-5-fluoropyridin-3-yl)piperidine-4-carboxamido)pyrrolidine-1-carboxylate (237.0 mg, 1 Eq, 393.2 μmol) in MeOH (5 mL) was added Pd/C (89.03 mg, 4.7% Wt, 0.1 Eq, 39.32 μmol) and ammonium formate (248.0 mg, 10 Eq, 3.932 mmol). The resulting mixture was heated at 80° C. for 2.5 hrs. The reaction mixture was filtered through a Celite pad, and the filtrate was concentrated in vacuo to dryness. The remaining residue was dissolved in EtOAc and washed with water. The organic layer was dried over anhydrous MgSO₄, filtered and concentrated to give crude product tert-butyl (R)-3-(4-(6-(2-ethoxyphenyl)-5-fluoropyridin-3-yl)piperidine-4-carboxamido)pyrrolidine-1-carboxylate (201.1 mg, 392.3 μmol, 99.77%) as an white solid. MS (M+H)+=513.7

Step 8: To a mixture of tert-butyl (R)-3-(4-(6-(2-ethoxyphenyl)-5-fluoropyridin-3-yl)piperidine-4-carboxamido)pyrrolidine-1-carboxylate (200 mg, 1 Eq, 390 μmol), 2-fluoro-5-(trifluoromethyl)benzonitrile (111 mg, 1.5 Eq, 585 μmol) and DIPEA (151 mg, 204 μL, 3 Eq, 1.17 mmol) was added DMSO (1 mL). The resulting mixture was heated at 90° C. for 2 hrs. The reaction mixture was diluted with water, extracted with EtOAc, washed with brine, concentrated and purified by silica gel chromatography to give tert-butyl (R)-3-(1-(2-cyano-4-(trifluoromethyl)phenyl)-4-(6-(2-ethoxyphenyl)-5-fluoropyridin-3-yl)piperidine-4-carboxamido)pyrrolidine-1-carboxylate as a light yellow solid. MS (M+H)+=682.6.

Step 9: To a DCM (0.6 mL) solution of tert-butyl (R)-3-(1-(2-cyano-4-(trifluoromethyl)-phenyl)-4-(6-(2-ethoxyphenyl)-5-fluoropyridin-3-yl)piperidine-4-carboxamido)pyrrolidine-1-carboxylate (266 mg, 1 Eq, 390 μmol) was added TFA (0.7 g, 0.5 mL, 6 mmol). The resulting mixture was stirred at ambient temperature for 0.5 h. The reaction crude was concentrated and purified by silica gel chromatography to give (R)-1-(2-cyano-4-(trifluoromethyl)phenyl)-4-(6-(2-ethoxyphenyl)-5-fluoropyridin-3-yl)-N-(pyrrolidin-3-yl)piperidine-4-carboxamide (217.4 mg, 373.8 μmol, 95.8%) as a white solid. MS (M+H)+=582.6.

### Example 63: tert-butyl (R)-(14-(3-(1-(2-cyano-4-(trifluoromethyl)phenyl)-4-(6-(2-ethoxyphenyl)-5-fluoropyridin-3-yl)piperidine-4-carboxamido)pyrrolidin-1-yl)-3,6,9,12-tetraoxatetradecyl)carbamate

To an acetonitrile (0.5 mL) solution of (R)-1-(2-cyano-4-(trifluoromethyl)phenyl)-4-(6-(2-ethoxyphenyl)-5-fluoropyridin-3-yl)-N-(pyrrolidin-3-yl)piperidine-4-carboxamide (49.2 mg, 1 Eq, 84.6 μmol) was added potassium carbonate (35.1 mg, 3 Eq, 254 μmol), sodium iodide (12.7 mg, 1 Eq, 84.6 μmol), and tert-butyl (14-bromo-3,6,9,12-tetraoxatetradecyl)carbamate (50.8 mg, 1.5 Eq, 127 μmol). The resulting mixture was stirred at 80° C. for 3 hrs. The reaction mixture was cooled to ambient temperature and extracted with EtOAc. The organic layer was washed with brine, concentrated and purified by silica gel chromatography to give tert-butyl (R)-(14-(3-(1-(2-cyano-4-(trifluoromethyl)phenyl)-4-(6-(2-ethoxyphenyl)-5-fluoropyridin-3-yl)piperidine-4-carboxamido)-pyrrolidin-1-yl)-3,6,9,12-tetraoxatetradecyl)carbamate as an off-white solid. MS (M+H)⁺=901.6.

### Example 64: (R)—N-(1-(14-amino-3,6,9,12-tetraoxatetradecyl)pyrrolidin-3-yl)-1-(2-cyano-4-(trifluoromethyl)phenyl)-4-(6-(2-ethoxyphenyl)-5-fluoropyridin-3-yl)piperidine-4-carboxamide

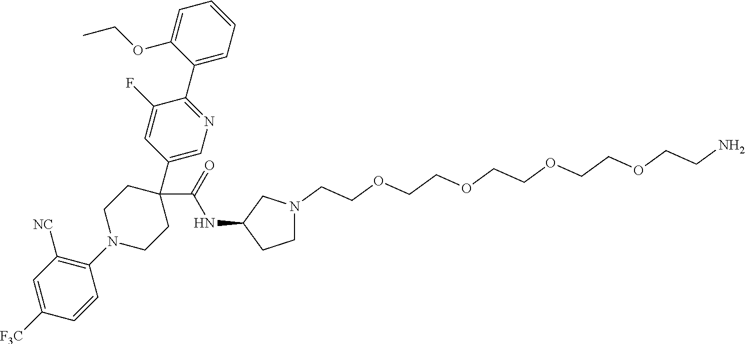 [SMILES: CCOc1ccccc1-c1ncc(C2(C(=O)N[C@@H]3CCN(CCOCCOCCOCCOCCN)C3)CCN(c3ccc(C(F)(F)F)cc3C#N)CC2)cc1F]

To a DCM (0.6 mL) solution of tert-butyl (R)-(14-(3-(1-(2-cyano-4-(trifluoromethyl)-phenyl)-4-(6-(2-ethoxyphenyl)-5-fluoropyridin-3-yl)piperidine-4-carboxamido)pyrrolidin-1-yl)-3,6,9,12-tetraoxatetradecyl)carbamate (76.2 mg, 1 Eq, 84.6 μmol) was added 2,2,2-trifluoroacetic acid (9.64 mg, 0.5 mL, 1 Eq, 84.6 μmol). The resulting mixture was stirred at ambient temperature for 0.5 h. The reaction mixture was concentrated, and the remaining residue was purified by C18 reverse phase chromatography eluting with MeCN (0.1% TFA)/water (0.1% TFA). Pure fractions were combined and concentrated to give product (R)—N-(1-(14-amino-3,6,9,12-tetraoxatetradecyl)-pyrrolidin-3-yl)-1-(2-cyano-4-(trifluoromethyl)phenyl)-4-(6-(2-ethoxyphenyl)-5-fluoropyridin-3-yl)piperidine-4-carboxamide as a TFA salt. MS (M+H)⁺=801.8.

### Example 65: (R)-2,2′,2″-(10-(17-(3-(1-(2-cyano-4-(trifluoromethyl)phenyl)-4-(6-(2-ethoxy-phenyl)-5-fluoropyridin-3-yl)piperidine-4-carboxamido)pyrrolidin-1-yl)-2-oxo-6,9,12,15-tetra-oxa-3-azaheptadecyl)-1,4,7,10-tetraazacyclododecane-1,4,7-triyl)triacetic acid (Compound 9)

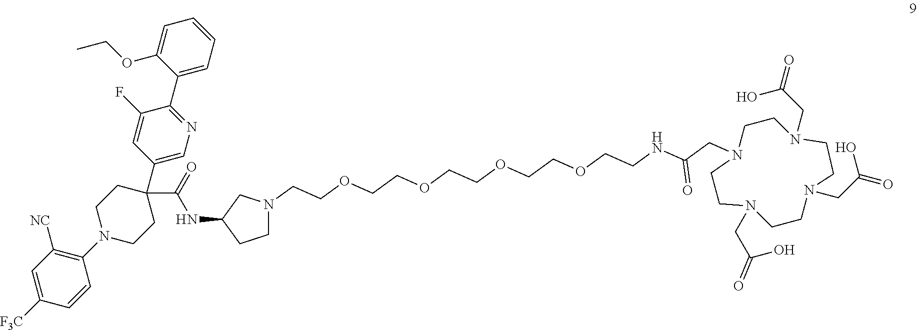 [SMILES: CCOc1ccccc1-c1ncc(C2(C(=O)N[C@@H]3CCN(CCOCCOCCOCCOCCNC(=O)CN4CCN(CC(=O)O)CCN(CC(=O)O)CCN(CC(=O)O)CC4)C3)CCN(c3ccc(C(F)(F)F)cc3C#N)CC2)cc1F]

Step 1: To a DMF (0.5 mL) solution of 2-(4,7,10-tris(2-(tert-butoxy)-2-oxoethyl)-1,4,7,10-tetraazacyclododecan-1-yl)acetic acid (43.8 mg, 1 Eq, 76.4 μmol) was added 2-(3H-[1,2,3]triazolo[4,5-b]pyridin-3-yl)-1,1,3,3-tetramethylisouronium hexafluorophosphate(V)(43.6 mg, 1.5 Eq, 115 μmol), (R)—N-(1-(14-amino-3,6,9,12-tetraoxatetradecyl)pyrrolidin-3-yl)-1-(2-cyano-4-(trifluoromethyl)phenyl)-4-(6-(2-ethoxyphenyl)-5-fluoropyridin-3-yl)piperidine-4-carboxamide (61.2 mg, 1 Eq, 76.4 μmol) and N-ethyl-N-isopropylpropan-2-amine (29.6 mg, 39.9 μL, 3 Eq, 229 μmol). The reaction mixture was stirred at room temperature for 2 hours. The reaction crude was diluted with ethyl acetate, washed with water and brine, concentrated and purified by silica gel chromatography (EtOAc-Hex) to give the target compound tri-tert-butyl 2,2′,2″-(10-(17-(3-(1-(2-cyano-4-(trifluoromethyl)phenyl)-4-(6-(2-ethoxyphenyl)-5-fluoropyridin-3-yl)piperidine-4-carboxamido)pyrrolidin-1-yl)-2-oxo-6,9,12,15-tetraoxa-3-azaheptadecyl)-1,4,7,10-tetraazacyclododecane-1,4,7-triyl)(R)-triacetate as a white solid. MS (M+H)⁺=1356.1.

Step 2: Tri-tert-butyl 2,2′,2″-(10-(17-(3-(1-(2-cyano-4-(trifluoromethyl)phenyl)-4-(6-(2-ethoxyphenyl)-5-fluoropyridin-3-yl)piperidine-4-carboxamido)pyrrolidin-1-yl)-2-oxo-6,9,12,15-tetraoxa-3-azaheptadecyl)-1,4,7,10-tetraazacyclododecane-1,4,7-triyl)(R)-triacetate (104 mg, 1 Eq, 76.7 μmol) was combined with 2,2,2-trifluoroacetic acid (8.75 mg, 1 mL, 1 Eq, 76.7 μmol). The resulting mixture was stirred at ambient temperature for 1 h. The reaction crude was concentrated and purified by C18 reverse phase chromatography eluting with MeCN (0.1% TFA)/water (0.1% TFA). Pure fractions were combined and dried to give the product as a TFA salt. MS (M+H)+=1188.0.

### Example 66: ¹¹⁵Indium Complex of Compound 9

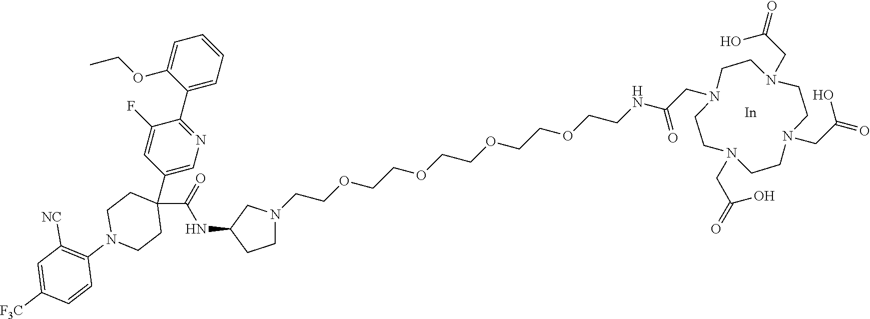

To an acetonitrile (0.3 mL) solution of (R)-2,2′,2″-(10-(17-(3-(1-(2-cyano-4-(trifluoromethyl)phenyl)-4-(6-(2-ethoxyphenyl)-5-fluoropyridin-3-yl)piperidine-4-carboxamido)-pyrrolidin-1-yl)-2-oxo-6,9,12,15-tetraoxa-3-azaheptadecyl)-1,4,7,10-tetraazacyclododecane-1,4,7-triyl)triacetic acid (39.4 mg, 1 Eq, 33.2 μmol) was added sodium hydrogen carbonate (27.9 mg, 10 Eq, 332 μmol), ¹¹⁵InCl₃ (22.0 mg, 3 Eq, 99.6 μmol) and Water (0.3 mL). The resulting mixture was stirred at 20° C. for 1 hour. The reaction crude was purified by C18 reverse phase chromatography eluting with MeCN (0.1% TFA)/water (0.1% TFA). Pure fractions were dried and combined to give indium(III) (R)-2,2′,2″-(10-(17-(3-(1-(2-cyano-4-(trifluoromethyl)phenyl)-4-(6-(2-ethoxyphenyl)-5-fluoropyridin-3-yl)piperidine-4-carboxamido)-pyrrolidin-1-yl)-2-oxo-6,9,12,15-tetraoxa-3-azaheptadecyl)-1,4,7,10-tetraazacyclododecane-1,4,7-triyl)triacetate (28.1 mg, 21.6 μmol, 65.2%) as a TFA salt. MS (M+H)+=1300.2.

### Example 67: ¹⁷⁵Lutetium (III) Complex of Compound 9

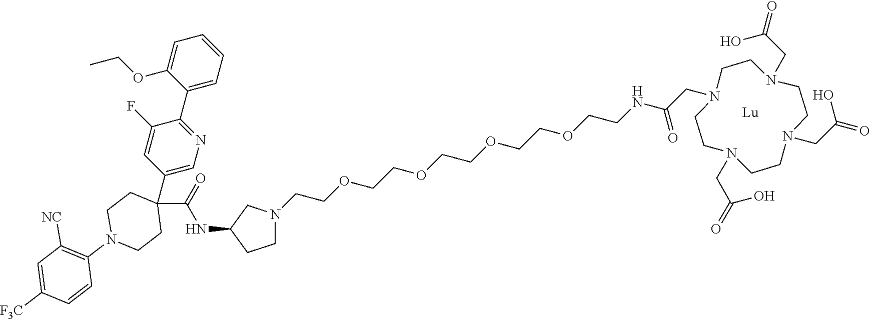

Into a 40 mL flask were added a mixture of (R)-2,2′,2″-(10-(17-(3-(1-(2-cyano-4-(trifluoromethyl)phenyl)-4-(6-(2-ethoxyphenyl)-5-fluoropyridin-3-yl)piperidine-4-carboxamido)-pyrrolidin-1-yl)-2-oxo-6,9,12,15-tetraoxa-3-azaheptadecyl)-1,4,7,10-tetraazacyclododecane-1,4,7-triyl)triacetic acid (250 mg, 1 Eq, 211 μmol), ¹⁷⁵Lutetium (III) chloride (300 mg, 75.4 μL, 5.06 Eq, 1.07 mmol), Sodium bicarbonate (180 mg, 83.3 μL, 10.2 Eq, 2.14 mmol), ACN (2.6 mL) and Water (1.3 mL). The resulting mixture was stirred for 2 hours at 80° C. The reaction mixture was diluted with 4 mL of DMSO, filtered and the filtrate was purified by Prep-HPLC using the following conditions (Prep-HPLC-007): Column, SunFire Prep C18 OBD Column, 19×150 mm 5 μm 10 nm; mobile phase, Water (0.05% TFA) and ACN (30% ACN up to 75% in 15 min); Total flow rate, 20 mL/min; Detector, UV 220 nm. This resulted in the product (216.5 mg, 136.4 μmol, 64.8%) as a yellow solid. [M+H-2TFA]=1359.7

### Example 68: tert-butyl (R)-(3-((N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)carbamate

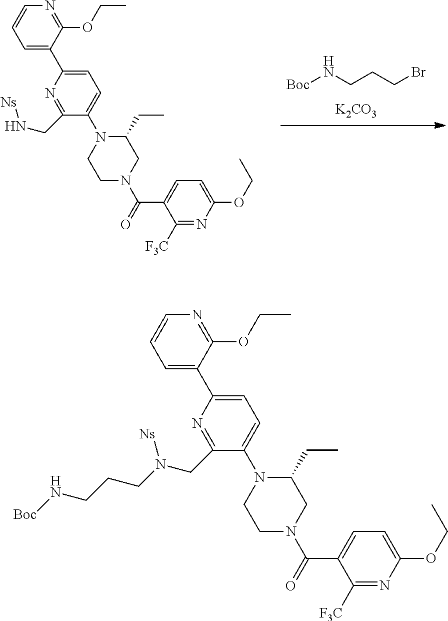

Into a 40 mL vial, was placed a mixture of (R)—N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (1.06 g, 1 Eq, 1.43 mmol), K₂CO₃ (991 mg, 5.03 Eq, 7.17 mmol), tert-butyl (3-bromopropyl)carbamate (1.75 g, 5.16 Eq, 7.35 mmol) and MeCN (5 mL). The reaction mixture was stirred at 80° C. for 5 hours. The reaction crude was purified by Prep-HPLC using the following conditions: Column, C18 120 g Column; mobile phase, Water (0.1% FA) and ACN (30% ACN up to 98% in 4 min, 98-98% in 7 min); Total flow rate, 90 mL/min; Detector, UV 220 nm. This resulted in the title compound (1.03 g, 1.14 mmol, 80.2%) as a light-yellow solid. [M+H]=901.3.

### Example 69: (R)—N-(3-aminopropyl)-N-((2′-ethoxy-5-(4-(6-ethoxy-2-(trifluoromethyl)-nicotinoyl)-2-ethylpiperazin-1-yl)-[2,3′-bipyridin]-6-yl)methyl)-2-nitrobenzenesulfonamide

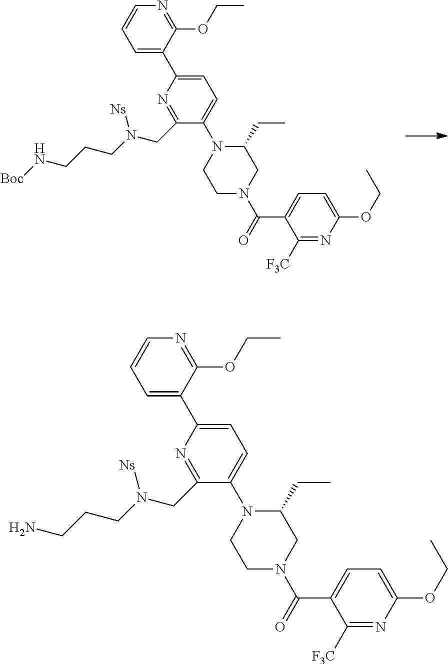

Into a 50 mL flask were added tert-butyl (R)-(3-((N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrophenyl)-sulfonamido)propyl)carbamate (1.03 g, 1 Eq, 1.14 mmol), DCM (10 mL) and TFA (4 mL). The resulting mixture was stirred at 25° C. for 1 hour. The reaction mixture was concentrated under vacuum. This resulted in crude (R)—N-(3-aminopropyl)-N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (1.1 g, 1.4 mmol, 120%) as a light brown oil, which was used in next step without further purification. M+H=801.3.

### Example 70: tert-butyl (R)-3-((((9H-fluoren-9-yl)methoxy)carbonyl)amino)-4-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-4-nitrophenyl)sulfonamido)propyl)amino)-4-oxobutanoate

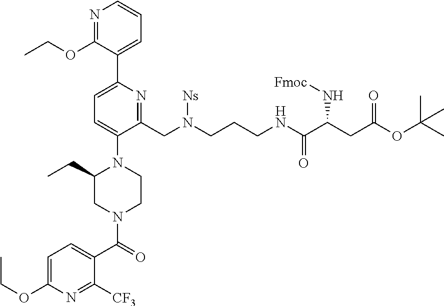

Into a 40-mL vial, was placed a mixture of (R)-2-((((9H-fluoren-9-yl)methoxy)carbonyl)-amino)-4-(tert-butoxy)-4-oxobutanoic acid (621 mg, 1.51 Eq, 1.51 mmol), DIEA (395 mg, 532 μL, 3.06 Eq, 3.06 mmol), HATU (492 mg, 1.30 Eq, 1.29 mmol) and DMF (10 mL). The resulting mixture was stirred at 25° C. for 5 min, followed by the addition of (R)—N-(3-aminopropyl)-N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (800 mg, 1 Eq, 999 μmol). The reaction mixture was stirred at 25° C. for 1 hour. The reaction crude was purified by Prep-HPLC using the following conditions: Column, C18 120 g Column; mobile phase, Water (0.05% FA) and ACN (30% ACN up to 98% in 10 min); Total flow rate, 90 mL/min; Detector, UV 220 nm. Pure fractions were collected and concentrated. This resulted in tert-butyl (R)-3-((((9H-fluoren-9-yl)methoxy)carbonyl)amino)-4-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-4-nitrophenyl)sulfonamido)propyl)amino)-4-oxobutanoate (711 mg, 595 μmol, 59.6%) as a light-yellow solid. [M+H]=1195.9.

### Example 71: (R)-3-((((9H-fluoren-9-yl)methoxy)carbonyl)amino)-4-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)amino)-4-oxobutanoic acid

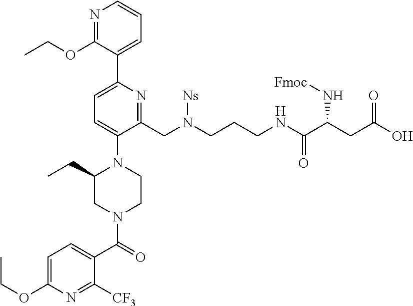

Into a 50 mL flask, were added tert-butyl (R)-3-((((9H-fluoren-9-yl)methoxy)carbonyl)amino)-4-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-2-nitrophenyl)sulfonamido)propyl)amino)-4-oxobutanoate (711 mg, 1 Eq, 595 μmol) and TFA (8 mL). The resulting mixture was stirred at 25° C. for 1 hour. The reaction mixture was concentrated to give crude product (624 mg, 548 μmol, 92.1%) as a light brown oil. This material was used in next step without further purification. M+H=801.3.

### Example 72: (9H-fluoren-9-yl)methyl tert-butyl ((R)-1-(2′-ethoxy-5-((R)-4-(6-ethoxy-2-(trifluoromethyl)nicotinoyl)-2-ethylpiperazin-1-yl)-[2,3′-bipyridin]-6-yl)-2-((2-nitrophenyl)sulfonyl)-7,10-dioxo-14-oxa-2,6,11-triazahexadecane-8,16-diyl)dicarbamate

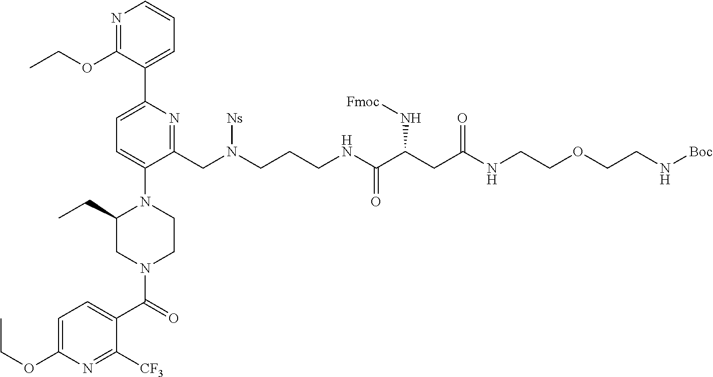

Into a 50-mL flask, was placed a mixture of (R)-3-((((9H-fluoren-9-yl)methoxy)carbonyl)-amino)-4-((3-((N-((2′-ethoxy-5-((R)-4-(6-ethoxy-2-(trifluoromethyl)nicotinoyl)-2-ethylpiperazin-1-yl)-[2,3′-bipyridin]-6-yl)methyl)-4-nitrophenyl)sulfonamido)propyl)amino)-4-oxobutanoic acid (624 mg, 1 Eq, 548 μmol), HATU (308 mg, 1.48 Eq, 810 μmol), DIEA (219 mg, 295 μL, 3.09 Eq, 1.69 mmol) and DMF (7 mL). The resulting mixture was stirred at 25° C. for 5 min, followed by the addition of tert-butyl (2-(2-aminoethoxy)ethyl)carbamate (221 mg, 1.97 Eq, 1.08 mmol). The reaction mixture was stirred at 25° C. for 1 hour. The reaction crude was purified by Prep-HPLC using the following conditions. Column, C18 120 g; mobile phase, Water (0.05% FA) and ACN (30% ACN up to 98% in 15 min); Total flow rate, 90 mL/min; Detector, UV 220 nm. This resulted in the title compound (410 mg, 0.23 mmol, 42%, 75% Purity) as a light-yellow solid. [M+H]=1326.1.

### Example 73: (9H-fluoren-9-yl)methyl ((R)-16-amino-1-(2′-ethoxy-5-((R)-4-(6-ethoxy-2-(trifluoromethyl)nicotinoyl)-2-ethylpiperazin-1-yl)-[2,3′-bipyridin]-6-yl)-2-((2-nitrophenyl)sulfonyl)-7,10-dioxo-14-oxa-2,6,11-triazahexadecan-8-yl)carbamate

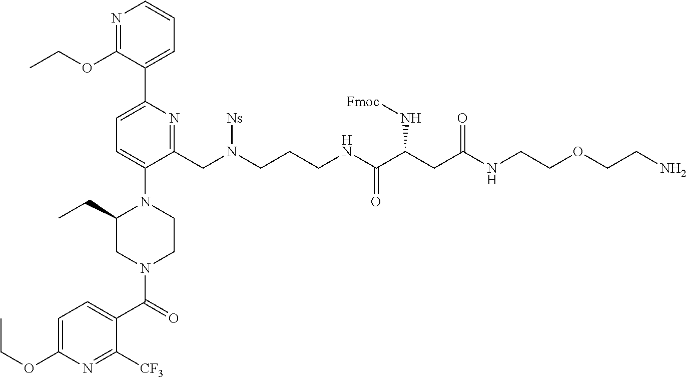

Into a 50 mL flask, were added (9H-fluoren-9-yl)methyl tert-butyl ((R)-1-(2′-ethoxy-5-((R)-4-(6-ethoxy-2-(trifluoromethyl)nicotinoyl)-2-ethylpiperazin-1-yl)-2-((2-nitrophenyl)sulfonyl)-7,10-dioxo-14-oxa-2,6,11-triazahexadecane-8,16-diyl)dicarbamate (410 mg, 1 Eq, 310 μmol), DCM (3 mL) and TFA (1 mL). The resulting mixture was stirred at 25° C. for 1 hour. The reaction mixture was concentrated under vacuum to afford crude product (433 mg, 354 μmol, 114%) as an oil. This material was used in next step without further purification. M+H=801.3.

### Example 74: 2,2′,2″-(10-((R)-8-amino-1-(2′-ethoxy-5-((R)-4-(6-ethoxy-2-(trifluoromethyl)-nicotinoyl)-2-ethylpiperazin-1-yl)-[2,3′-bipyridin]-6-yl)-7,10,18-trioxo-14-oxa-2,6,11,17-tetra-azanonadecan-19-yl)-1,4,7,10-tetraazacyclododecane-1,4,7-triyl)triacetic acid (Compound 10)

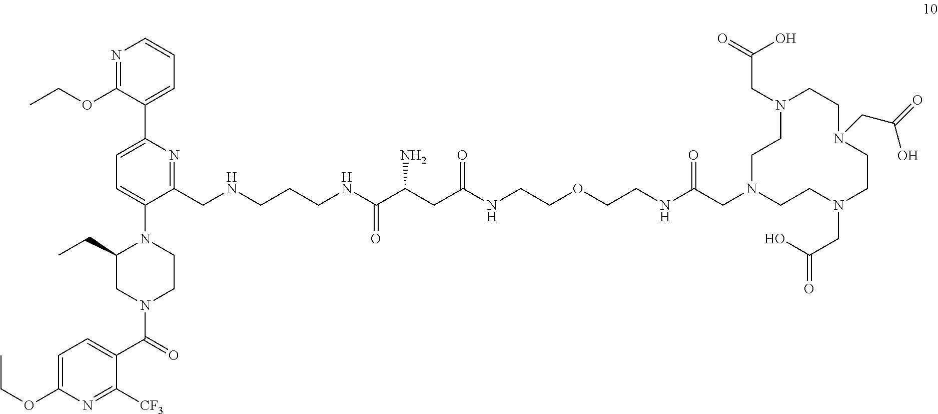 [SMILES: CCOc1ccc(C(=O)N2CCN(c3ccc(-c4cccnc4OCC)nc3CNCCCNC(=O)[C@H](N)CC(=O)NCCOCCNC(=O)CN3CCN(CC(=O)O)CCN(CC(=O)O)CCN(CC(=O)O)CC3)[C@H](CC)C2)c(C(F)(F)F)n1]

Step 1: Into a 40-mL vial, was placed a mixture of 2-(4,7,10-tris(2-(tert-butoxy)-2-oxoethyl)-1,4,7,10-tetraazacyclododecan-1-yl)acetic acid (267 mg, 1.32 Eq, 466 μmol), DIEA (141 mg, 190 μL, 3.08 Eq, 1.09 mmol), HATU (279 mg, 2.07 Eq, 734 μmol) and DMF (5 mL). The resulting mixture was stirred at 25° C. for 5 min, followed by the addition of (9H-fluoren-9-yl)methyl ((R)-16-amino-1-(2′-ethoxy-5-((R)-4-(6-ethoxy-2-(trifluoromethyl)nicotinoyl)-2-ethylpiperazin-1-yl)-[2,3′-bipyridin]-6-yl)-2-((2-nitrophenyl)sulfonyl)-7,10-dioxo-14-oxa-2,6,11-triazahexadecan-8-yl)carbamate (433 mg, 1 Eq, 354 μmol). The resulting mixture was stirred at 25° C. for 1 hour. The reaction crude was purified by Prep-HPLC using the following conditions: Column, C18 120 g; mobile phase, Water (0.05% FA) and ACN (30% ACN up to 98% in 15 min); Total flow rate, 90 mL/min; Detector, UV 220 nm. This resulted in tri-tert-butyl 2,2′,2″-(10-((R)-8-((((9H-fluoren-9-yl)methoxy)carbonyl)amino)-1-(2′-ethoxy-5-((R)-4-(6-ethoxy-2-(trifluoromethyl)nicotinoyl)-2-ethylpiperazin-1-yl)-[2,3′-bipyridin]-6-yl)-2-((4-nitrophenyl)sulfonyl)-7,10,18-trioxo-14-oxa-2,6,11,17-tetraazanonadecan-19-yl)-1,4,7,10-tetraazacyclododecane-1,4,7-triyl)triacetate (330 mg, 185 μmol, 52.4%) as a light-yellow solid. [M+H]=1780.2.

Step 2: Into a 40-mL vial, was placed a mixture of tri-tert-butyl 2,2′,2″-(10-((R)-8-((((9H-fluoren-9-yl)methoxy)carbonyl)amino)-1-(2′-ethoxy-5-((R)-4-(6-ethoxy-2-(trifluoromethyl)-nicotinoyl)-2-ethylpiperazin-1-yl)-[2,3′-bipyridin]-6-yl)-2-((2-nitrophenyl)sulfonyl)-7,10,18-trioxo-14-oxa-2,6,11,17-tetraazanonadecan-19-yl)-1,4,7,10-tetraazacyclododecane-1,4,7-triyl)triacetate (330 mg, 1 Eq, 185 μmol), K₂CO₃ (258 mg, 10.1 Eq, 1.87 mmol), 2-chlorobenzenethiol (137 mg, 5.11 Eq, 947 μmol) and MeCN (5 mL). The reaction mixture was stirred at 80° C. for 1 hour. The reaction crude was purified by Prep-HPLC using the following conditions: Column, C18 120 g; mobile phase, Water (0.05% FA) and ACN (30% ACN up to 98% in 7 min); Total flow rate, 70 mL/min; Detector, UV 220 nm. Pure fractions were collected, concentrated, and lyophilized to afford tri-tert-butyl 2,2′,2″-(10-((R)-8-amino-1-(2′-ethoxy-5-((R)-4-(6-ethoxy-2-(trifluoromethyl)nicotinoyl)-2-ethylpiperazin-1-yl)-[2,3′-bipyridin]-6-yl)-7,10,18-trioxo-14-oxa-2,6,11,17-tetraazanonadecan-19-yl)-1,4,7,10-tetraazacyclododecane-1,4,7-triyl)triacetate (161 mg, 117 μmol, 63.3%) as an off-white solid. [M+H]=1395.2.

Step 3: Tri-tert-butyl 2,2′,2″-(10-((R)-8-amino-1-(2′-ethoxy-5-((R)-4-(6-ethoxy-2-(trifluoromethyl)nicotinoyl)-2-ethylpiperazin-1-yl)-[2,3′-bipyridin]-6-yl)-7,10,18-trioxo-14-oxa-2,6,11,17-tetraazanonadecan-19-yl)-1,4,7,10-tetraazacyclododecane-1,4,7-triyl)triacetate (161 mg, 1 Eq, 117 μmol) was combined with TFA (2 mL) and the resulting mixture was stirred at 25° C. for 3 hrs. The reaction crude was concentrated and purified by Prep-HPLC: Sunfire Prep C18 OBD Column, 50×250 mm, 5 m 10 nm; Mobile Phase A: Water (0.05% TFA), Mobile Phase B: ACN; Flow rate: 90 mL/min; Gradient: 25% B to 55% B in 8 min. This resulted in 2,2′,2″-(10-((R)-8-amino-1-(2′-ethoxy-5-((R)-4-(6-ethoxy-2-(trifluoromethyl)nicotinoyl)-2-ethylpiperazin-1-yl)-[2,3′-bipyridin]-6-yl)-7,10,18-trioxo-14-oxa-2,6,11,17-tetraazanonadecan-19-yl)-1,4,7,10-tetraazacyclododecane-1,4,7-triyl)triacetic acid-2,2,2-trifluoroacetic acid (1/1) (126.2 mg, 95.80 μmol, 81.6%) as a white solid. [M+H-1TFA]=1315.7.

### Example 75: tert-butyl (R)-(1-(3-(4-(4-ethoxy-2-(trifluoromethyl)benzoyl)-2-ethylpiperazin-1-yl)-6-(2-ethoxyphenyl)pyridin-2-yl)-2-((2-nitrophenyl)sulfonyl)-6,9,12,15-tetraoxa-2-azaheptadecan-17-yl)carbamate

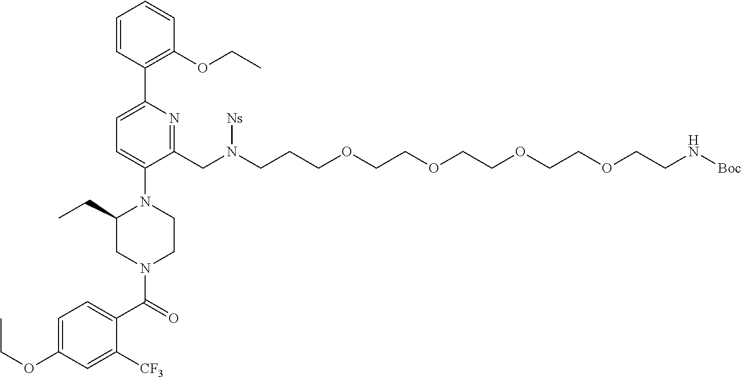

Into a 40 mL vial, was placed a mixture of (R)—N-((3-(4-(4-ethoxy-2-(trifluoromethyl)-benzoyl)-2-ethylpiperazin-1-yl)-6-(2-ethoxyphenyl)pyridin-2-yl)methyl)-2-nitrobenzenesulfonamide (800 mg, 1 Eq, 1.08 mmol), 2,2-dimethyl-4-oxo-3,8,11,14,17-pentaoxa-5-azaicosan-20-yl methanesulfonate (700 mg, 1.51 Eq, 1.63 mmol), K₂CO₃ (450 mg, 3.02 Eq, 3.26 mmol), potassium iodide (360 mg, 2.01 Eq, 2.17 mmol) and MeCN (10 mL). The reaction mixture was stirred at 80° C. for 3 hours. The reaction crude was purified by Prep-HPLC using the following conditions: Column, C18 120 g; mobile phase, Water (0.1% FA) and ACN (30% ACN up to 98% in 7 min); Total flow rate, 70 mL/min; Detector, UV 220 nm. This resulted in the title compound (931 mg, 866 μmol, 80.3%) as a light-yellow solid. [M+H]=1076.4.

### Example 76: (R)—N-(1-amino-3,6,9,12-tetraoxapentadecan-15-yl)-N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide

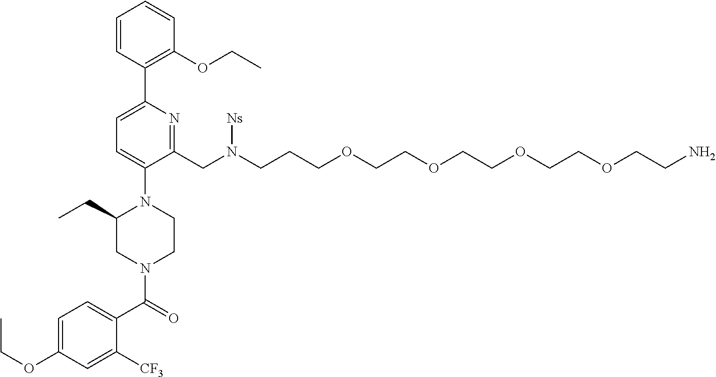

Into an 8-mL vial, was placed tert-butyl (R)-(1-(3-(4-(4-ethoxy-2-(trifluoromethyl)benzoyl)-2-ethylpiperazin-1-yl)-6-(2-ethoxyphenyl)pyridin-2-yl)-2-((2-nitrophenyl)sulfonyl)-6,9,12,15-tetraoxa-2-azaheptadecan-17-yl)carbamate (400 mg, 1 Eq, 372 μmol) and HCl/EA (2 mL). The resulting mixture was stirred at ambient temperature for 30 min. The reaction crude was purified by Prep-HPLC using the following conditions: Column, C18 120 g Column; mobile phase, Water (0.1% FA) and ACN (30% ACN up to 98% in 7 min); Total flow rate, 70 mL/min; Detector, UV 220 nm. The collected fractions were concentrated. The resulting mixture was concentrated under vacuum. This resulted in the title compound (350 mg, 359 μmol, 96.5%) as a light yellow solid. [M+H]=976.7.

### Example 77: (R)-2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-19-oxo-6,9,12,15-tetraoxa-2,18-diazaicosan-20-yl)-1,4,7,10-tetraazacyclododecane-1,4,7-triyl)triacetic acid (Compound 11)

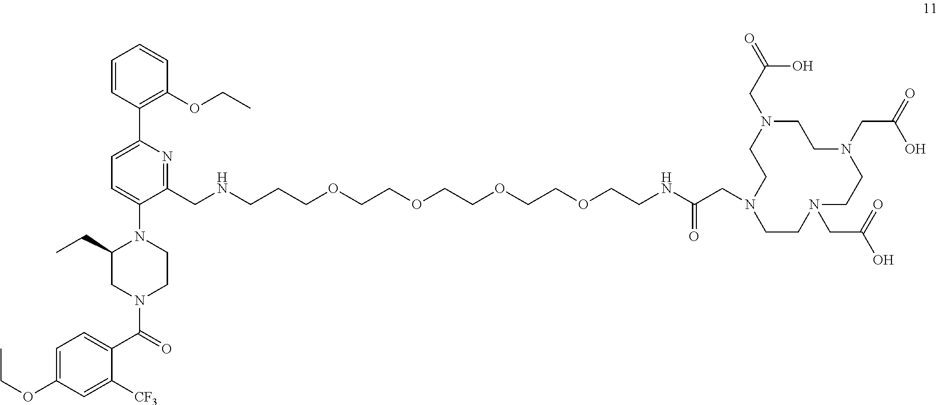 [SMILES: CCOc1ccc(C(=O)N2CCN(c3ccc(-c4ccccc4OCC)nc3CNCCCOCCOCCOCCOCCNC(=O)CN3CCN(CC(=O)O)CCN(CC(=O)O)CCN(CC(=O)O)CC3)[C@H](CC)C2)c(C(F)(F)F)c1]

Step 1: Into a 40-mL vial, was placed a mixture of 2-(4,7,10-tris(2-(tert-butoxy)-2-oxoethyl)-1,4,7,10-tetraazacyclododecan-1-yl)acetic acid (250 mg, 1.22 Eq, 436 μmol), DIEA (140 mg, 189 μL, 3.02 Eq, 1.08 mmol), HATU (180 mg, 1.32 Eq, 473 μmol) and DMF (4 mL). The resulting mixture was stirred at 25° C. for 5 min, followed by the addition of (R)—N-(1-amino-3,6,9,12-tetraoxapentadecan-15-yl)-N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide (350 mg, 1 Eq, 359 μmol). The reaction mixture was stirred at 25° C. for 1 hour. The reaction crude was purified by Prep-HPLC using the following conditions: Column, C18 120 g; mobile phase, Water (0.05% FA) and ACN (30% ACN up to 98% in 15 min); Total flow rate, 90 mL/min; Detector, UV 220 nm. This resulted in tri-tert-butyl 2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-2-((2-nitrophenyl)sulfonyl)-19-oxo-6,9,12,15-tetraoxa-2,18-diazaicosan-20-yl)-1,4,7,10-tetraazacyclododecane-1,4,7-triyl)(R)-triacetate (412 mg, 269 μmol, 75.0%) as a light yellow solid. [M+H]=1529.9.

Step 2: Into a 40-mL vial, was placed a mixture of tri-tert-butyl 2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-2-((2-nitrophenyl)sulfonyl)-19-oxo-6,9,12,15-tetraoxa-2,18-diazaicosan-20-yl)-1,4,7,10-tetraazacyclododecane-1,4,7-triyl)(R)-triacetate (400 mg, 1 Eq, 261 μmol), 2-(4,7,10-tris(2-(tert-butoxy)-2-oxoethyl)-1,4,7,10-tetraazacyclododecan-1-yl)acetic acid (250 mg, 1.22 Eq, 436 μmol), K₂CO₃ (360 mg, 9.96 Eq, 2.60 mmol) and MeCN (5 mL). The reaction mixture was stirred at 80° C. for 1 hour. The reaction crude was purified by Prep-HPLC using the following conditions: Column, C18 120 g; mobile phase, Water (0.05% FA) and ACN (30% ACN up to 98% in 7 min); Total flow rate, 70 mL/min; Detector, UV 220 nm. Pure fractions were collected, concentrated, and lyophilized to afford tri-tert-butyl 2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-19-oxo-6,9,12,15-tetraoxa-2,18-diazaicosan-20-yl)-1,4,7,10-tetraazacyclododecane-1,4,7-triyl)(R)-triacetate (280 mg, 208 μmol, 79.6%) as an off-white solid. [M+Na]=1367.

Step 3: Into an 8-mL vial, was placed a mixture of tri-tert-butyl 2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-19-oxo-6,9,12,15-tetraoxa-2,18-diazaicosan-20-yl)-1,4,7,10-tetraazacyclododecane-1,4,7-triyl)(R)-triacetate (280 mg, 1 Eq, 208 μmol) and TFA (3 mL). The resulting mixture was stirred at 25° C. for 3 hours. The reaction crude was concentrated, and the remaining residue was purified by Prep-HPLC using the following conditions: Sunfire Prep C18 OBD Column, 50×250 mm, 5 m 10 nm; Mobile Phase A: Water (0.05% FA), Mobile Phase B: ACN; Flow rate: 90 mL/min; Gradient: 25% B to 55% B in 8 min. This resulted in (R)-2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-19-oxo-6,9,12,15-tetraoxa-2,18-diazaicosan-20-yl)-1,4,7,10-tetraazacyclododecane-1,4,7-triyl)triacetic acid (154.5 mg, 131.3 μmol, 63.1%) as a white solid. [M+H]=1176.7.

### Example 78: ¹¹⁵Indium Complex of Compound 11

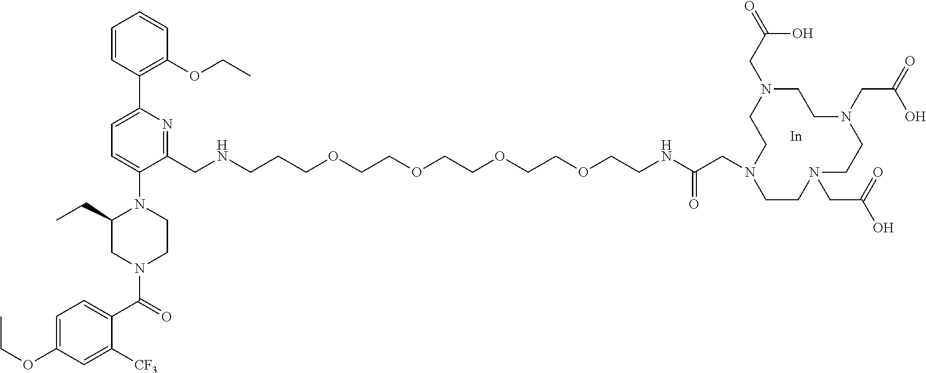

Into an 8 mL vial, was placed (R)-2,2′,2″-(10-(1-(3-(4-(4-ethoxy-2-(trifluoromethyl)-benzoyl)-2-ethylpiperazin-1-yl)-6-(2-ethoxyphenyl)pyridin-2-yl)-19-oxo-6,9,12,15-tetraoxa-2,18-diazaicosan-20-yl)-1,4,7,10-tetraazacyclododecane-1,4,7-triyl)triacetic acid (180 mg, 1 Eq, 153 μmol), ¹¹⁵InCl₃ (169 mg, 5 Eq, 765 μmol), sodium hydrogen carbonate (270 mg, 21 Eq, 3.21 mmol), MeCN (2 mL) and Water (2 mL). The resulting mixture was stirred at 80° C. for 1 hour. The reaction crude was purified by Prep-HPLC using the following conditions: Sunfire Prep C18 OBD Column, 50*250 mm, 5 m 10 nm; Mobile Phase A: Water (0.05% TFA), Mobile Phase B: ACN; Flow rate: 90 mL/min; Gradient: 35% B to 55% B in 8 min. This resulted in indium(III) (R)-2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-19-oxo-6,9,12,15-tetraoxa-2,18-diazaicosan-20-yl)-1,4,7,10-tetraazacyclododecane-1,4,7-triyl)triacetate TFA salt (63.3 mg, 45.1 μmol, 29.5%) as an off-white solid. [M+H-TFA]=1288.7.

### Example 79: methyl N2-((R)-2-((((9H-fluoren-9-yl)methoxy)carbonyl)amino)-5-(tert-butoxy)-5-oxopentanoyl)-N6-(tert-butoxycarbonyl)-D-lysinate

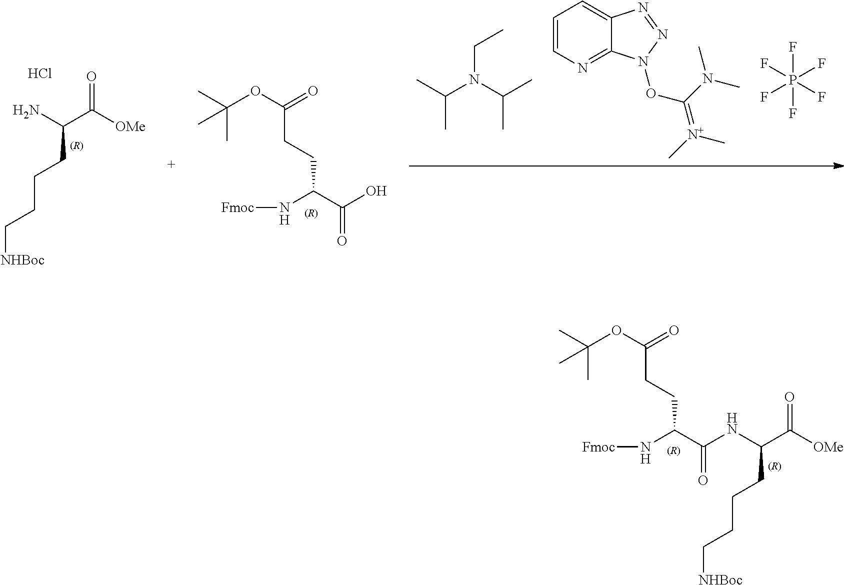

Into a 100-mL round bottom flask, was placed a mixture of (R)-2-((((9H-fluoren-9-yl)methoxy)carbonyl)amino)-5-(tert-butoxy)-5-oxopentanoic acid (2.50 g, 1.0 Eq, 5.88 mmol), HATU (2.68 g, 1.20 Eq, 7.05 mmol), DIEA (2.28 g, 3.00 Eq, 17.6 mmol) and DMF (30 mL). The resulting mixture was stirred at 25° C. for 30 min, followed by the addition of methyl N6-(tert-butoxycarbonyl)-D-lysinate hydrochloride (2.00 g, 1.15 Eq, 6.74 mmol). The resulting mixture was stirred at 25° C. for 1 hour. The reaction crude was quenched with 150 mL of water and extracted with EtOAc (80 mL×3). Organic layers were combined, washed with water (50 mL×3) and brine (100 mL), dried over anhydrous Na₂SO₄ and concentrated under reduced pressure. The remaining residue was purified by silica gel chromatography eluting with EtOAc/PE (EtOAc from 0% to 65% in 10 min). This resulted in methyl N2-((R)-2-((((9H-fluoren-9-yl)methoxy)carbonyl)amino)-5-(tert-butoxy)-5-oxopentanoyl)-N6-(tert-butoxycarbonyl)-D-lysinate (4.30 g, 5.8 mmol, 99%, 90% Purity) as a light yellow solid. [M+Na]=690.3.

### Example 80: methyl N2-((R)-2-amino-5-(tert-butoxy)-5-oxopentanoyl)-N6-(tert-butoxycarbonyl)-D-lysinate

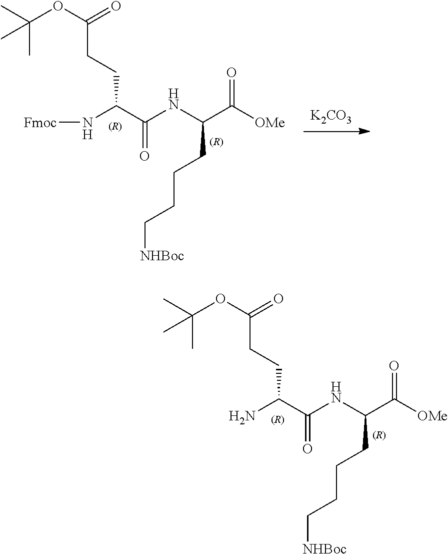

Into a 40 mL vial was added a mixture of methyl N2-((R)-2-((((9H-fluoren-9-yl)methoxy)carbonyl)amino)-5-(tert-butoxy)-5-oxopentanoyl)-N6-(tert-butoxycarbonyl)-D-lysinate (1.5 g, 1 Eq, 2.2 mmol), K₂CO₃ (6.2 g, 20 Eq, 45 mmol) and MeCN (20 mL). The resulting mixture was stirred at 50° C. for 3 hours. The reaction crude was quenched with 50 mL of water and extracted with DCM (50 mL×3). Organic layers were combined, washed with water (50 mL×3) and brine (50 mL), dried over anhydrous Na₂SO₄ and concentrated under reduced pressure. This resulted in crude product (1.2 g, 2.7 mmol, 120%) as a light-yellow oil. This material was used in next step without further purification. [M+H]=446.3.

### Example 81: methyl ((R)-5-(tert-butoxy)-2-(4-(4-iodophenyl)butanamido)-5-oxopentanoyl)-D-lysinate

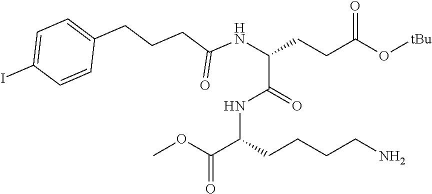 [SMILES: COC(=O)[C@@H](CCCCN)NC(=O)[C@@H](CCC(=O)OC(C)(C)C)NC(=O)CCCc1ccc(I)cc1]

Step 1: To a DMF (10 mL) solution of 4-(4-iodophenyl)butanoic acid (0.94 g, 1.2 Eq, 3.2 mmol) was added HATU (1.3 g, 1.3 Eq, 3.4 mmol) and DIEA (1.0 g, 2.9 Eq, 7.7 mmol). Methyl N2-((R)-2-amino-5-(tert-butoxy)-5-oxopentanoyl)-N6-(tert-butoxycarbonyl)-D-lysinate (1.2 g, 1 Eq, 2.7 mmol) was added subsequently. The resulting mixture was stirred at 25° C. for 1 hour. The reaction crude was purified by Prep-HPLC using the following conditions: Column, C18 19*150 mm 5 μm; mobile phase, Water (0.05% FA) and CAN (30.0% ACN up to 98.0% in 7 min); Total flow rate, 70 mL/min; Detector, UV 220 nm. This resulted in methyl N2-((R)-5-(tert-butoxy)-2-(4-(4-iodophenyl)butanamido)-5-oxopentanoyl)-N6-(tert-butoxycarbonyl)-D-lysinate (1.1 g, 1.5 mmol, 57%) was obtained as a light yellow solid. [M+H]=718.4.

Step 2: Into an 8-mL vial, was placed a mixture of methyl N2-((R)-5-(tert-butoxy)-2-(4-(4-iodophenyl)butanamido)-5-oxopentanoyl)-N6-(tert-butoxycarbonyl)-D-lysinate (450 mg, 1 Eq, 627 μmol) and MeCN (3 mL). TMS-I (140 mg, 95.2 μL, 1.12 Eq, 700 μmol) was added and the resulting mixture was stirred at 25 C for 20 min. The reaction crude was purified by Prep-HPLC using the following conditions: Column, C18 120 g; mobile phase, Water (0.1% FA) and ACN (30% ACN up to 98% in 7 min); Total flow rate, 70 mL/min; Detector, UV 220 nm. This resulted in methyl ((R)-5-(tert-butoxy)-2-(4-(4-iodophenyl)butanamido)-5-oxopentanoyl)-D-lysinate (410 mg, 664 μmol, 106%) as a colorless solid. [M+H]=618.3.

### Example 82: N2-((R)-5-(tert-butoxy)-2-(4-(4-iodophenyl)butanamido)-5-oxopentanoyl)-N6-(2-(4,7,10-tris(2-(tert-butoxy)-2-oxoethyl)-1,4,7,10-tetraazacyclododecan-1-yl)acetyl)-D-lysine

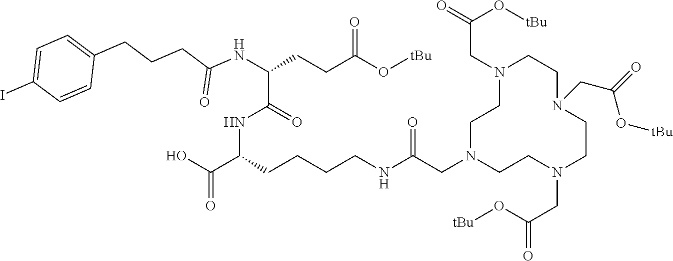 [SMILES: CC(C)(C)OC(=O)CC[C@@H](NC(=O)CCCc1ccc(I)cc1)C(=O)N[C@H](CCCCNC(=O)CN1CCN(CC(=O)OC(C)(C)C)CCN(CC(=O)OC(C)(C)C)CCN(CC(=O)OC(C)(C)C)CC1)C(=O)O]

Step 1: Into a 40-mL vial, was placed a mixture of 2-(4,7,10-tris(2-(tert-butoxy)-2-oxoethyl)-1,4,7,10-tetraazacyclododecan-1-yl)acetic acid (400 mg, 1.20 Eq, 698 μmol), DIEA (230 mg, 3.05 Eq, 1.78 mmol), HATU (290 mg, 1.31 Eq, 763 μmol) and DMF (5 mL). The resulting mixture was stirred at 20° C. for 5 min, followed by the addition of tri-tert-butyl 2,2′,2″-(10-(2-(((R)-5-((R)-5-(tert-butoxy)-2-(4-(4-iodophenyl)butanamido)-5-oxopentanamido)-6-methoxy-6-oxohexyl)amino)-2-oxoethyl)-1,4,7,10-tetraazacyclododecane-1,4,7-triyl)triacetate (250 mg, 1.0 Eq, 213 μmol). The reaction mixture was stirred at 20° C. for 1 hour. The reaction crude was purified by Prep-HPLC using the following conditions: Column, C18 120 g; mobile phase, Water (0.05% FA) and ACN (30% ACN up to 98% in 15 min); Total flow rate, 90 mL/min; Detector, UV 220 nm. This resulted in tri-tert-butyl 2,2′,2″-(10-(2-(((R)-5-((R)-5-(tert-butoxy)-2-(4-(4-iodophenyl)butanamido)-5-oxopentanamido)-6-methoxy-6-oxohexyl)amino)-2-oxoethyl)-1,4,7,10-tetraazacyclododecane-1,4,7-triyl)triacetate (250 mg, 213 μmol, 36.6%) as colorless oil. [M+H]=1173.3.

Step 2: Into a 8-mL vial, was placed a mixture of tri-tert-butyl 2,2′,2″-(10-(2-(((R)-5-((R)-5-(tert-butoxy)-2-(4-(4-iodophenyl)butanamido)-5-oxopentanamido)-6-methoxy-6-oxohexyl)amino)-2-oxoethyl)-1,4,7,10-tetraazacyclododecane-1,4,7-triyl)triacetate (240 mg, 1 Eq, 205 μmol), LiOH (15 mg, 3.1 Eq, 0.63 mmol), MeOH (3 mL) and water (1 mL). The resulting mixture was stirred at 20° C. for 2 hrs. The reaction crude was purified by Prep-HPLC using the following conditions: Column, C18 120 g, 19×150 mm 5 μm; mobile phase, Water (0.05% FA) and ACN (30.0% ACN up to 98.0% in 3 min); Total flow rate, 70 mL/min; Detector, UV 220 nm. This resulted in N2-((R)-5-(tert-butoxy)-2-(4-(4-iodophenyl)butanamido)-5-oxopentanoyl)-N6-(2-(4,7,10-tris(2-(tert-butoxy)-2-oxoethyl)-1,4,7,10-tetraazacyclododecan-1-yl)acetyl)-D-lysine (213 mg, 184 μmol, 89.8%) as a colorless oil. [M+H]=1158.6.

### Example 83: 2,2′,2″-(10-((R)-20-((R)-4-carboxy-2-(4-(4-iodophenyl)butanamido)butanamido)-1-(2′-ethoxy-5-((R)-4-(6-ethoxy-2-(trifluoromethyl)nicotinoyl)-2-ethylpiperazin-1-yl)-[2,3′-bipyridin]-6-yl)-19,26-dioxo-6,9,12,15-tetraoxa-2,18,25-triazaheptacosan-27-yl)-1,4,7,10-tetraazacyclododecane-1,4,7-triyl)triacetic acid (Compound 12)

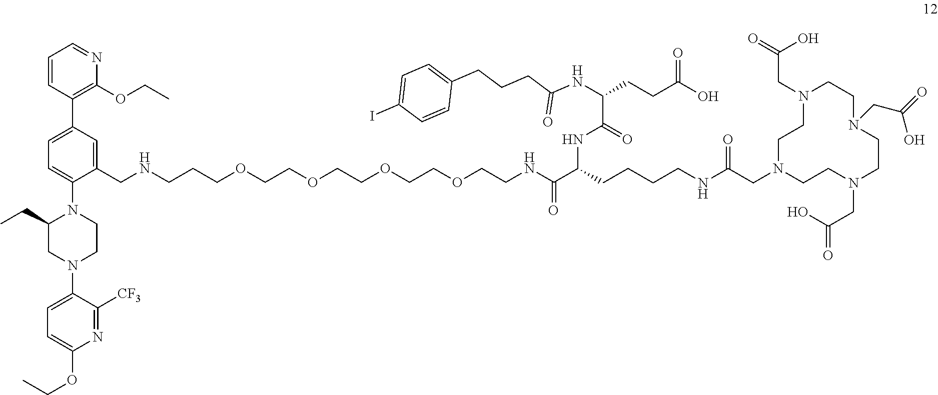 [SMILES: CCOc1ccc(N2CCN(c3ccc(-c4cccnc4OCC)cc3CNCCCOCCOCCOCCOCCNC(=O)[C@@H](CCCCNC(=O)CN3CCN(CC(=O)O)CCN(CC(=O)O)CCN(CC(=O)O)CC3)NC(=O)[C@@H](CCC(=O)O)NC(=O)CCCc3ccc(I)cc3)[C@H](CC)C2)c(C(F)(F)F)n1]

Step 1: Into an 8-mL vial, was placed a mixture of N2-((R)-5-(tert-butoxy)-2-(4-(4-iodophenyl)butanamido)-5-oxopentanoyl)-N6-(2-(4,7,10-tris(2-(tert-butoxy)-2-oxoethyl)-1,4,7,10-tetraazacyclododecan-1-yl)acetyl)-D-lysine (40 mg, 1 Eq, 35 μmol), HATU (18 mg, 1.4 Eq, 47 μmol), DIEA (15 mg, 3.4 Eq, 0.12 mmol) and DMF (1 mL). The resulting mixture was stirred at 20° C. for 5 min, followed by the addition of (R)—N-(1-amino-3,6,9,12-tetraoxapentadecan-15-yl)-N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (34 mg, 1.0 Eq, 35 μmol). The reaction mixture was stirred at 20° C. for 1 hour. The reaction crude was purified by Prep-HPLC using the following conditions: Column, Sunfire Prep C18 OBD Column; mobile phase, Water (0.05% TFA) and ACN (30% ACN up to 98% in 15 min); Total flow rate, 90 mL/min; Detector, UV 220 nm. This resulted in tri-tert-butyl 2,2′,2″-(10-((R)-20-((R)-5-(tert-butoxy)-2-(4-(4-iodophenyl)butanamido)-5-oxopentanamido)-1-(2′-ethoxy-5-((R)-4-(6-ethoxy-2-(trifluoromethyl)nicotinoyl)-2-ethylpiperazin-1-yl)-[2,3′-bipyridin]-6-yl)-2-((2-nitrophenyl)-sulfonyl)-19,26-dioxo-6,9,12,15-tetraoxa-2,18,25-triazaheptacosan-27-yl)-1,4,7,10-tetraazacyclododecane-1,4,7-triyl)triacetate (19 mg, 9.0 μmol, 26%) as a light yellow oil. [M+H]/2=1059.6.

Step 2: Into a 2-mL vial, was placed a mixture of tri-tert-butyl 2,2′,2″-(10-((R)-20-((R)-5-(tert-butoxy)-2-(4-(4-iodophenyl)butanamido)-5-oxopentanamido)-1-(2′-ethoxy-5-((R)-4-(6-ethoxy-2-(trifluoromethyl)nicotinoyl)-2-ethylpiperazin-1-yl)-[2,3′-bipyridin]-6-yl)-2-((4-nitrophenyl)sulfonyl)-19,26-dioxo-6,9,12,15-tetraoxa-2,18,25-triazaheptacosan-27-yl)-1,4,7,10-tetraazacyclododecane-1,4,7-triyl)triacetate (18 mg, 1 Eq, 8.5 μmol), K₂CO₃ (12 mg, 10 Eq, 87 μmol), 2-chlorobenzenethiol (6 mg, 5 Eq, 0.04 mmol) and MeCN (0.2 mL). The resulting mixture was stirred at 80° C. for 1 hour. The reaction crude was purified by Prep-HPLC using the following conditions: Column, Prep C18 OBD; mobile phase, Water (0.05% TFA) and ACN (30% ACN up to 98% in 7 min); Total flow rate, 70 mL/min; Detector, UV 220 nm. Pure fractions were collected, concentrated, and lyophilized to afford tri-tert-butyl 2,2′,2″-(10-((R)-20-((R)-5-(tert-butoxy)-2-(4-(4-iodophenyl)butanamido)-5-oxopentanamido)-1-(2′-ethoxy-5-((R)-4-(6-ethoxy-2-(trifluoromethyl)nicotinoyl)-2-ethylpiperazin-1-yl)-[2,3′-bipyridin]-6-yl)-19,26-dioxo-6,9,12,15-tetraoxa-2,18,25-triazaheptacosan-27-yl)-1,4,7,10-tetraazacyclododecane-1,4,7-triyl)triacetate (12 mg, 6.2 μmol, 73%) as an off-white oil. [M+Na]=1956.1.

Step 3: Into a 8-mL vial, was placed a mixture of tri-tert-butyl 2,2′,2″-(10-((R)-20-((R)-5-(tert-butoxy)-2-(4-(4-iodophenyl)butanamido)-5-oxopentanamido)-1-(2′-ethoxy-5-((R)-4-(6-ethoxy-2-(trifluoromethyl)nicotinoyl)-2-ethylpiperazin-1-yl)-[2,3′-bipyridin]-6-yl)-19,26-dioxo-6,9,12,15-tetraoxa-2,18,25-triazaheptacosan-27-yl)-1,4,7,10-tetraazacyclododecane-1,4,7-triyl)triacetate (12 mg, 1 Eq, 6.2 μmol) and TFA (0.2 mL). The resulting mixture was stirred at 20° C. for 1 hour. The reaction mixture was concentrated, and the remaining residue was purified by Prep-HPLC: Sunfire Prep C18 OBD Column, 50×250 mm, 5 m 10 nm; Mobile Phase A: Water (0.05% TFA), Mobile Phase B: ACN; Flow rate: 90 mL/min; Gradient: 25% B to 55% B in 8 min. This resulted in 2,2′,2″-(10-((R)-20-((R)-4-carboxy-2-(4-(4-iodophenyl)butanamido)-butanamido)-1-(2′-ethoxy-5-((R)-4-(6-ethoxy-2-(trifluoromethyl)nicotinoyl)-2-ethylpiperazin-1-yl)-[2,3′-bipyridin]-6-yl)-19,26-dioxo-6,9,12,15-tetraoxa-2,18,25-triazaheptacosan-27-yl)-1,4,7,10-tetraazacyclododecane-1,4,7-triyl)triacetic acid-2,2,2-trifluoroacetic acid (1/1) (1.7 mg, 0.93 μmol, 15%) as colorless oil. [M+H]/2=854.8.

### Example 84: tert-butyl 3-((2-(2-((tert-butoxycarbonyl)amino)ethoxy)ethyl)amino)-5-(3-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)propoxy)benzoate

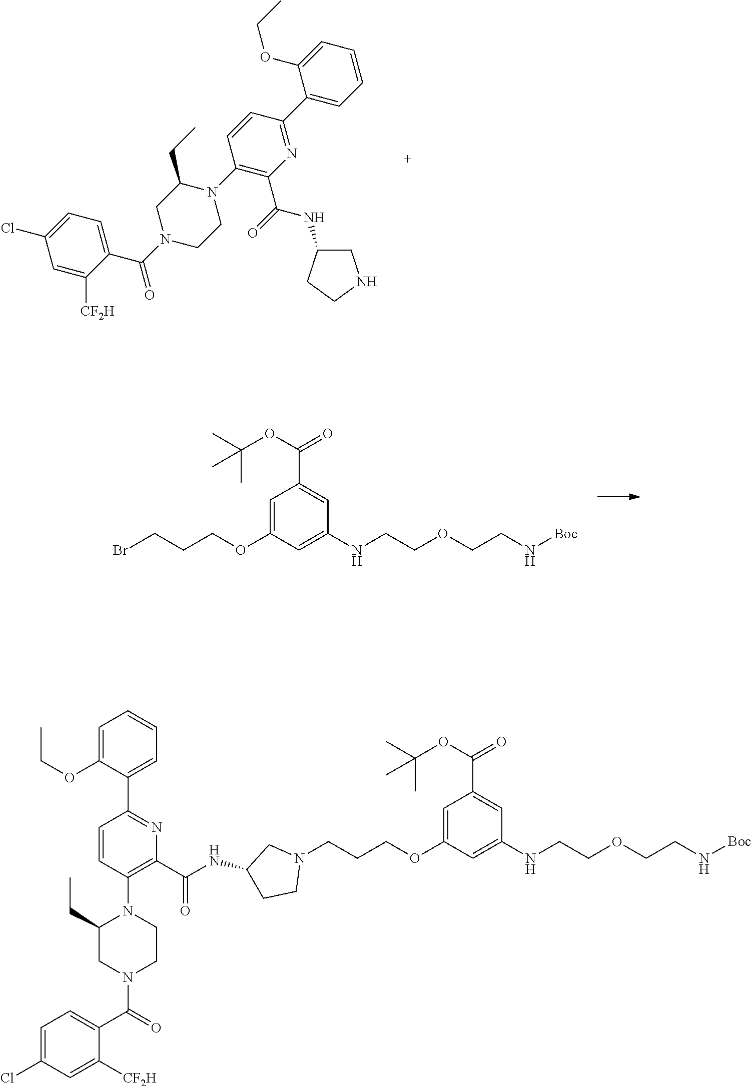 [SMILES: CCOc1ccccc1-c1ccc(N2CCN(C(=O)c3ccc(Cl)cc3C(F)F)C[C@H]2CC)c(C(=O)N[C@H]2CCNC2)n1 ; CC(C)(C)OC(=O)NCCOCCNc1cc(OCCCBr)cc(C(=O)OC(C)(C)C)c1 ; CCOc1ccccc1-c1ccc(N2CCN(C(=O)c3ccc(Cl)cc3C(F)F)C[C@H]2CC)c(C(=O)N[C@H]2CCN(CCCOc3cc(NCCOCCNC(=O)OC(C)(C)C)cc(C(=O)OC(C)(C)C)c3)C2)n1]

To an ACN (6 mL) solution of 3-((R)-4-(4-chloro-2-(difluoromethyl)benzoyl)-2-ethylpiperazin-1-yl)-6-(2-ethoxyphenyl)-N—((R)-pyrrolidin-3-yl)picolinamide (560 mg, 1 Eq, 915 μmol) was added K₂CO₃ (370 mg, 2.93 Eq, 2.68 mmol) and tert-butyl 3-(3-bromopropoxy)-5-((2-(2-((tert-butoxycarbonyl)amino)ethoxy)ethyl)amino)benzoate (570 mg, 1.20 Eq, 1.10 mmol). The resulting mixture was stirred at 80° C. for 16 hrs. The reaction crude was purified by Flash-HPLC using the following conditions (IntelFlash-1): Column, C18 silica gel; mobile phase, water (0.05% TFA)/CH₃CN (CH₃CN: 30%-98% in 6 min, 98%˜98% in 4 min); Detector, UV 254&220 nm. This resulted in the title compound (380 mg, 362 μmol, 39.6%) as a brown solid. [M+H]=1048.7, 1050.7.

### Example 85: 3-((2-(2-aminoethoxy)ethyl)amino)-5-(3-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)propoxy)benzoic acid

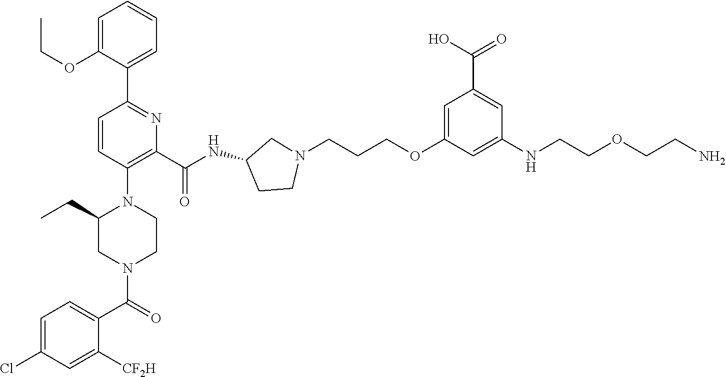 [SMILES: CCOc1ccccc1-c1ccc(N2CCN(C(=O)c3ccc(Cl)cc3C(F)F)C[C@H]2CC)c(C(=O)N[C@H]2CCN(CCCOc3cc(NCCOCCN)cc(C(=O)O)c3)C2)n1]

Into a 50 mL single-necked flask were added tert-butyl 3-((2-(2-((tert-butoxycarbonyl)amino)-ethoxy)ethyl)amino)-5-(3-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)propoxy)benzoate (320 mg, 1 Eq, 305 μmol), DCM (3 mL) and TFA (1 mL). The resulting mixture was stirred at 25 C for 1 hr. The reaction mixture was concentrated and the remaining residue was purified by Prep-HPLC using the following conditions (Flash-HPLC-013): Column, SunFire Prep C18 OBD Column, 19×150 mm 5 μm; mobile phase, Water (0.05% TFA) and ACN (10.0% ACN up to 98.0% in 4 min); Total flow rate, 70 mL/min; Detector, UV 220 nm. Pure fractions were collected, combined, and concentrated under vacuum. This resulted in the title compound (300 mg, 0.24 mmol, 77%, 70% Purity) as a light brown solid. [M+H]=892.5, 894.5.

### Example 86: 2,2′,2″-(10-(2-((2-(2-((3-carboxy-5-(3-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)propoxy)phenyl)amino)ethoxy)ethyl)amino)-2-oxoethyl)-1,4,7,10-tetraazacyclododecane-1,4,7-triyl)triacetic acid (Compound 13)

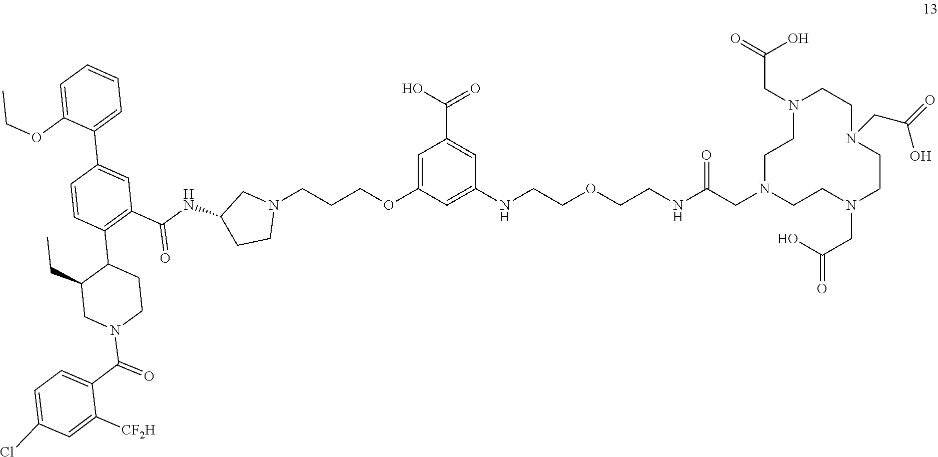 [SMILES: CCOc1ccccc1-c1ccc(C2CCN(C(=O)c3ccc(Cl)cc3C(F)F)C[C@H]2CC)c(C(=O)N[C@H]2CCN(CCCOc3cc(NCCOCCNC(=O)CN4CCN(CC(=O)O)CCN(CC(=O)O)CCN(CC(=O)O)CC4)cc(C(=O)O)c3)C2)c1]

Step 1: To a DMF (3 mL) solution of 2-(4,7,10-tris(2-(tert-butoxy)-2-oxoethyl)-1,4,7,10-tetraazacyclododecan-1-yl)acetic acid (300 mg, 1.56 Eq, 524 μmol) was added HATU (250 mg, 1.96 Eq, 657 μmol) and DIEA (150 mg, 202 μL, 3.45 Eq, 1.16 mmol). The resulting mixture was stirred at 25° C. for 10 min followed by the addition of 3-((2-(2-aminoethoxy)ethyl)-amino)-5-(3-((R)-3-(3-((R)-4-(4-chloro-2-(difluoromethyl)benzoyl)-2-ethylpiperazin-1-yl)-6-(2-ethoxyphenyl)-picolinamido)pyrrolidin-1-yl)propoxy)benzoic acid (300 mg, 1 Eq, 336 μmol). The reaction mixture was stirred at 25° C. for 1 hour. The reaction crude was purified by Flash-HPLC using the following conditions (IntelFlash-1): Column, C18 silica gel; mobile phase, water (0.05% TFA)/CH₃CN (CH₃CN:27%-50% in 6 min); Detector, UV 254&220 nm. This resulted in 3-(3-((R)-3-(3-((R)-4-(4-chloro-2-(difluoromethyl)benzoyl)-2-ethylpiperazin-1-yl)-6-(2-ethoxyphenyl)picolinamido)-pyrrolidin-1-yl)propoxy)-5-((2-(2-(2-(4,7,10-tris(2-(tert-butoxy)-2-oxoethyl)-1,4,7,10-tetraazacyclododecan-1-yl)acetamido)ethoxy)ethyl)amino)benzoic acid (295 mg, 204 μmol, 60.6%) as a white solid. [M+H]=1447.1, 1449.1.

Step 2: Into a 50 mL single-necked flask, were added 3-(3-((R)-3-(3-((R)-4-(4-chloro-2-(difluoromethyl)benzoyl)-2-ethylpiperazin-1-yl)-6-(2-ethoxyphenyl)picolinamido)pyrrolidin-1-yl)propoxy)-5-((2-(2-(2-(4,7,10-tris(2-(tert-butoxy)-2-oxoethyl)-1,4,7,10-tetraazacyclododecan-1-yl)acetamido)ethoxy)ethyl)amino)benzoic acid (375 mg, 1 Eq, 259 μmol) and TFA (3.7 mL). The resulting reaction mixture was stirred at 25° C. for 1.5 hrs. The reaction mixture was concentrated under vacuum. The remaining residue was purified by Prep-HPLC using the following conditions (Flash-HPLC-013): Column, SunFire Prep C18 OBD Column, 19×150 mm 5 μm; mobile phase, Water (0.05% TFA) and ACN (10.0% ACN up to 98.0% in 4 min); Total flow rate, 20 mL/min; Detector, UV 220 nm. Fractions were collected, combined, and lyophilized to afford 62.5 mg of desired product as a TFA salt with 90% purity. Part of this material (35 mg) was further purified by pre-HPLC using NH₃ as an additive. This resulted in 2,2′,2″-(10-(2-((2-(2-((3-carboxy-5-(3-((R)-3-(3-((R)-4-(4-chloro-2-(difluoromethyl)-benzoyl)-2-ethylpiperazin-1-yl)-6-(2-ethoxyphenyl)-picolinamido)pyrrolidin-1-yl)propoxy)-phenyl)amino)ethoxy)ethyl)amino)-2-oxoethyl)-1,4,7,10-tetraazacyclododecane-1,4,7-triyl)triacetic acid (11.2 mg, 8.54 μmol, 3.30%, 97.5% Purity) as a white solid. [M/2+H]=640.4

### Example 87: ¹¹⁵Indium Complex of Compound 13

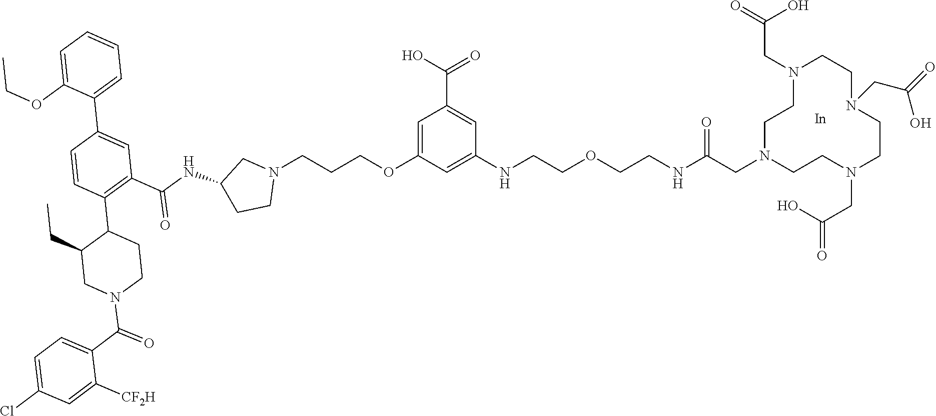

Into an 8 mL flask, were added 2,2′,2″-(10-(2-((2-(2-((3-carboxy-5-(3-((R)-3-(3-((R)-4-(4-chloro-2-(difluoromethyl)benzoyl)-2-ethylpiperazin-1-yl)-6-(2-ethoxyphenyl)picolinamido)-pyrrolidin-1-yl)propoxy)phenyl)amino)ethoxy)ethyl)amino)-2-oxoethyl)-1,4,7,10-tetraazacyclododecane-1,4,7-triyl)triacetic acid (20 mg, 1 Eq, 16 μmol), sodium bicarbonate (10 mg, 7.6 Eq, 0.12 mmol), ¹¹⁵InCl₃ (10 mg, 2.9 Eq, 45 μmol), water (0.25 mL) and Acetonitrile (0.5 mL). The resulting mixture was stirred at 80° C. for 1 hour. The reaction mixture was concentrated under vacuum. The remaining residue was purified by Prep-HPLC using the following conditions (Prep-HPLC-007): Column, SunFire Prep C18 OBD Column, 19×150 mm, 5 μm; mobile phase, Water (0.05% TFA) and ACN (30% ACN up to 75% in 15 min); Total flow rate, 20 mL/min; Detector, UV 220 nm. This resulted in the product (5.9 mg, 3.6 μmol, 23%) as a white solid. [M+H-2TFA]=1390.7, 1392.7

### Example 88: (R)-2,2′,2″-(10-(2-((15-(((2′-ethoxy-5-(4-(6-ethoxy-2-(trifluoromethyl)nicotinoyl)-2-ethylpiperazin-1-yl)-[2,3′-bipyridin]-6-yl)methyl)amino)pentadecyl)amino)-2-oxoethyl)-1,4,7,10-tetraazacyclododecane-1,4,7-triyl)triacetic acid (Compound 14)

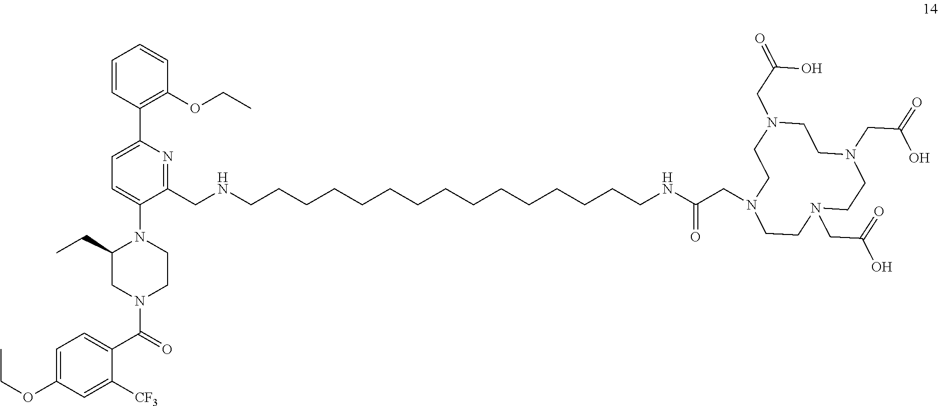 [SMILES: CCOc1ccc(C(=O)N2CCN(c3ccc(-c4ccccc4OCC)nc3CNCCCCCCCCCCCCCCCNC(=O)CN3CCN(CC(=O)O)CCN(CC(=O)O)CCN(CC(=O)O)CC3)[C@H](CC)C2)c(C(F)(F)F)c1]

Step 1: Into an 8 mL vial were added (R)—N-((2′-ethoxy-5-(4-(6-ethoxy-2-(trifluoromethyl)-nicotinoyl)-2-ethylpiperazin-1-yl)-[2,3′-bipyridin]-6-yl)methyl)-2-nitrobenzenesulfonamide (100 mg, 1 Eq, 134 μmol), ACN (1.5 mL), cesium carbonate (130 mg, 2.97 Eq, 399 μmol), and 15-((tert-butoxycarbonyl)amino)pentadecyl methanesulfonate (120 mg, 2.12 Eq, 285 μmol). The resulting reaction mixture was stirred at 80° C. for 2 hours. The reaction crude was purified by Prep-HPLC using the following conditions (Flash-HPLC-013): Column, SunFire Prep C18 OBD Column, 19×150 mm, 5 μm; mobile phase, Water (0.1% FA) and ACN (30.0% ACN up to 98.0% in 18 min); Total flow rate, 70 mL/min; Detector, UV 220 nm. This resulted in tert-butyl (R)-(15-((N-((2′-ethoxy-5-(4-(6-ethoxy-2-(trifluoromethyl)-nicotinoyl)-2-ethylpiperazin-1-yl)-[2,3′-bipyridin]-6-yl)methyl)-2-nitrophenyl)sulfonamido)-pentadecyl)carbamate (100 mg, 93.5 μmol, 69.6%) as a yellow solid. [M+H]=1069.4.

Step 2: Into a 100 mL round bottom flask, was added tert-butyl (R)-(15-((N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrophenyl)sulfonamido)pentadecyl)carbamate (100 mg, 1 Eq, 93.5 μmol), DCM (4 mL) and TFA (1 mL). The resulting reaction mixture was stirred at 20° C. for 1 hour. The reaction mixture was concentrated under vacuum. The remaining residue was diluted with water (20 mL), neutralized with saturated aq NaHCO₃ until pH=7, and extracted with ethyl acetate (3×20 mL). Organic layers were combined, dried over anhydrous sodium sulfate and concentrated under vacuum. This resulted in (R)—N-(15-aminopentadecyl)-N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (90 mg, 93 μmol, 99%) as a light yellow solid. [M+H]=969.4.

Step 3: To a DMF (0.2 mL) solution of 2-(4,7,10-tris(2-(tert-butoxy)-2-oxoethyl)-1,4,7,10-tetraazacyclododecan-1-yl)acetic acid (70 mg, 1.4 Eq, 0.12 mmol) was added 2-(3H-[1,2,3]triazolo[4,5-b]pyridin-3-yl)-1,1,3,3-tetramethylisouronium hexafluorophosphate(V) (50 mg, 1.5 Eq, 0.13 mmol) and DIEA (3 mg, 4 μL, 4 Eq, 0.02 mmol). The resulting mixture was stirred at 20° C. for 10 min, followed by the addition of (R)—N-(15-aminopentadecyl)-N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (85 mg, 1 Eq, 88 μmol). The reaction mixture was stirred at 20° C. for 1 hour. The reaction crude was purified by Flash-HPLC using the following conditions (IntelFlash-1): Column, C18 silica gel; mobile phase, water (0.1% FA)/CH₃CN (CH₃CN:30%-98% in 6 min, 98%˜98% in 3 min); Detector, UV 254&220 nm. This resulted in tri-tert-butyl 2,2′,2″-(10-(2-((15-((N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrophenyl)sulfonamido)pentadecyl)amino)-2-oxoethyl)-1,4,7,10-tetraaza-cyclododecane-1,4,7-triyl)(R)-triacetate (80 mg, 52 μmol, 60%) as a yellow solid. [M+H]=1523.7.

Step 4: To an ACN (1 mL) solution of tri-tert-butyl 2,2′,2″-(10-(2-((15-((N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrophenyl)sulfonamido)pentadecyl)amino)-2-oxoethyl)-1,4,7,10-tetraazacyclododecane-1,4,7-triyl)(R)-triacetate (80 mg, 1 Eq, 52 μmol) was added K₂CO₃ (25 mg, 3.4 Eq, 0.18 mmol) and 2-chlorobenzenethiol (35 mg, 4.6 Eq, 0.24 mmol). The resulting mixture was stirred at 50° C. for 1 hour. The reaction crude was purified by Flash-HPLC using the following conditions (IntelFlash-1): Column, C18 silica gel; mobile phase, water (0.1% FA)/CH₃CN (CH₃CN:30%-70% in 10 min); Detector, UV 254&220 nm. This resulted in tri-tert-butyl 2,2′,2″-(10-(2-((15-(((2′-ethoxy-5-(4-(6-ethoxy-2-(trifluoromethyl)nicotinoyl)-2-ethylpiperazin-1-yl)-[2,3′-bipyridin]-6-yl)methyl)amino)-pentadecyl)amino)-2-oxoethyl)-1,4,7,10-tetraazacyclododecane-1,4,7-triyl)(R)-triacetate (60 mg, 45 μmol, 85%) as a yellow solid. [M+H]=1389.9.

Step 5: Into a 50 mL single-necked flask, were added tri-tert-butyl 2,2′,2″-(10-(2-((15-(((2′-ethoxy-5-(4-(6-ethoxy-2-(trifluoromethyl)nicotinoyl)-2-ethylpiperazin-1-yl)-[2,3′-bipyridin]-6-yl)methyl)amino)pentadecyl)amino)-2-oxoethyl)-1,4,7,10-tetraazacyclododecane-1,4,7-triyl)(R)-triacetate (60 mg, 1 Eq, 45 μmol) and TFA (1.5 mL). The resulting mixture was stirred at 30° C. for 2 hours. The reaction mixture was concentrated and the remaining residue was purified by Prep-HPLC using the following conditions (Flash-HPLC-013): Column, SunFire Prep C18 OBD Column, 19×150 mm, 5 μm; mobile phase, Water (0.05% TFA) and ACN (20.0% ACN up to 68.0% in 10 min); Total flow rate, 20 mL/min; Detector, UV 220 nm. This resulted in (R)-2,2′,2″-(10-(2-((15-(((2′-ethoxy-5-(4-(6-ethoxy-2-(trifluoromethyl)nicotinoyl)-2-ethylpiperazin-1-yl)-[2,3′-bipyridin]-6-yl)methyl)amino)pentadecyl)amino)-2-oxoethyl)-1,4,7,10-tetraazacyclododecane-1,4,7-triyl)triacetic acid-2,2,2-trifluoroacetic acid (1/2) (35 mg, 25 μmol, 56%) as a white solid. [M+H-2TFA]=1170.8.

### Example 89: ¹¹⁵Indium Complex of Compound 14

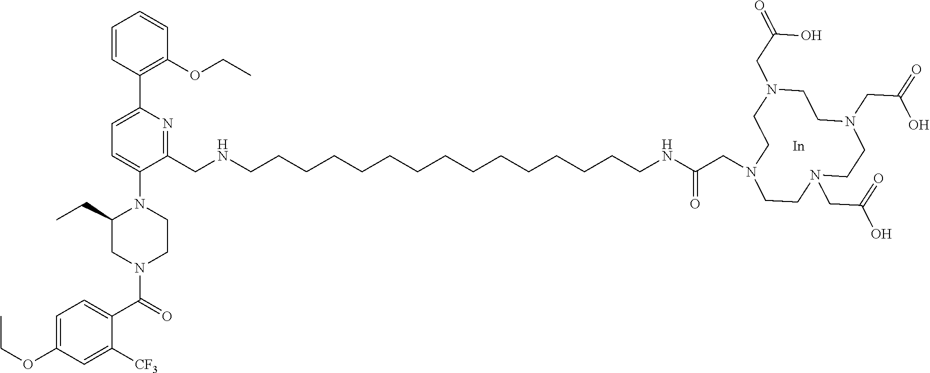

Into an 8 mL flask were added (R)-2,2′,2″-(10-(2-((15-(((2′-ethoxy-5-(4-(6-ethoxy-2-(trifluoromethyl)nicotinoyl)-2-ethylpiperazin-1-yl)-[2,3′-bipyridin]-6-yl)methyl)amino)-pentadecyl)amino)-2-oxoethyl)-1,4,7,10-tetraazacyclododecane-1,4,7-triyl)triacetic acid (20 mg, 1 Eq, 17 μmol), ¹¹⁵InCl₃ (10 mg, 2.6 Eq, 45 μmol), sodium bicarbonate (10 mg, 7.0 Eq, 0.12 mmol), Water (0.25 mL) and Acetonitrile (0.5 mL). The resulting mixture was stirred at 80° C. for 1 hour. The reaction solution was concentrated and the remaining residue was purified by Prep-HPLC using the following conditions (Prep-HPLC-007): Column, SunFire Prep C18 OBD Column, 19×150 mm, 5 μm; mobile phase, Water (0.1% FA) and ACN (30% ACN up to 70% in 16 min); Total flow rate, 20 mL/min; Detector, UV 220 nm. This resulted in the product (8 mg, 6 μmol, 40%) as a white solid. [M+H-FA]=1282.8

### Example 90: tert-butyl (R)-4-(4-bromo-2-cyanophenyl)-3-ethylpiperazine-1-carboxylate

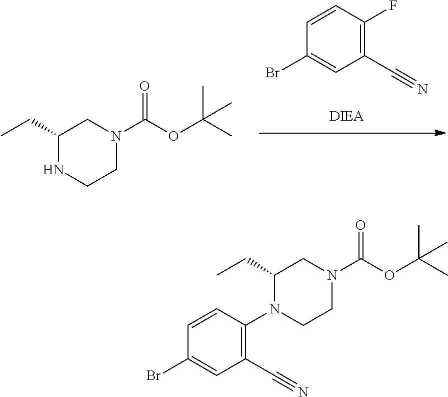 [SMILES: CC[C@@H]1CN(C(=O)OC(C)(C)C)CCN1 ; N#Cc1cc(Br)ccc1F ; CC[C@@H]1CN(C(=O)OC(C)(C)C)CCN1c1ccc(Br)cc1C#N]

Into a 40-mL vial, was placed a mixture of tert-butyl (R)-3-ethylpiperazine-1-carboxylate (4.05 g, 1 Eq, 18.9 mmol), DIEA (6.1 g, 8.2 mL, 2.5 Eq, 47 mmol), 5-bromo-2-fluorobenzonitrile (4.2 g, 1.1 Eq, 21 mmol) and DMSO (50 mL). The resulting mixture was stirred at 130° C. for 24 hours. The reaction mixture was dilute with 500 mL of water and extracted with EtOAc (350 mL×3). Organic layers were combined, washed with water (300 mL×2) and brine (300 mL), dried over anhydrous Na₂SO₄, and concentrated under reduced pressure. The remaining residue was purified by silica gel chromatography (EtOAc/PE, EtOAc from 0% to 85% in 15 min) to afford tert-butyl (R)-4-(4-bromo-2-cyanophenyl)-3-ethylpiperazine-1-carboxylate (3.01 g, 7.63 mmol, 40.4%) as yellow oil.

### Example 91: tert-butyl (R)-4-(2-cyano-4-(2-ethoxypyridin-3-yl)phenyl)-3-ethylpiperazine-1-carboxylate

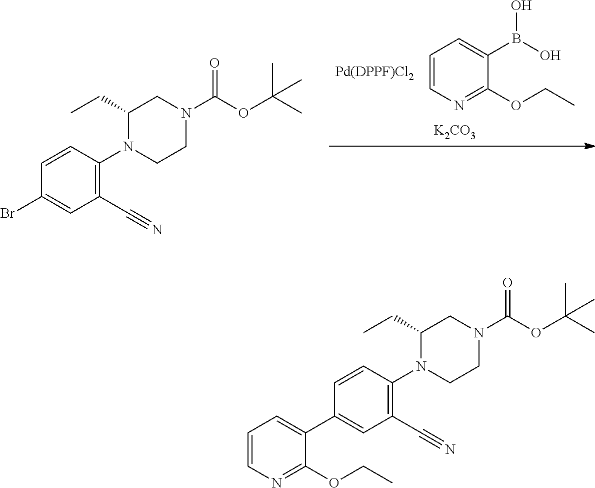

Into a 40-mL vial, purged and maintained with an inert atmosphere of nitrogen, was placed a mixture of tert-butyl (R)-4-(4-bromo-2-cyanophenyl)-3-ethylpiperazine-1-carboxylate (3 g, 1 Eq, 8 mmol), 1,1′-Bis(di-t-butylphosphino)ferrocene palladium dichloride (10 g, 2 Eq, 15 mmol), (2-ethoxypyridin-3-yl)boronic acid (0.06 g, 0.05 Eq, 0.4 mmol), K₂CO₃ (3 g, 3 Eq, 0.02 mol), 1,4-Dioxane (20 mL) and H₂O (4.0 mL). The resulting mixture was stirred at 100° C. for 2 hours. The reaction mixture was and concentrated and the remaining residue was purified by silica gel chromatography (EtOAc/PE, EtOAc from 0% to 85% in 15 min). This resulted in the title compound (3.2 g, 7.3 mmol, 100%) as a yellow oil. [M+Na]=459.3.

### Example 92: tert-butyl (R)-4-(2-(aminomethyl)-4-(2-ethoxypyridin-3-yl)phenyl)-3-ethylpiperazine-1-carboxylate

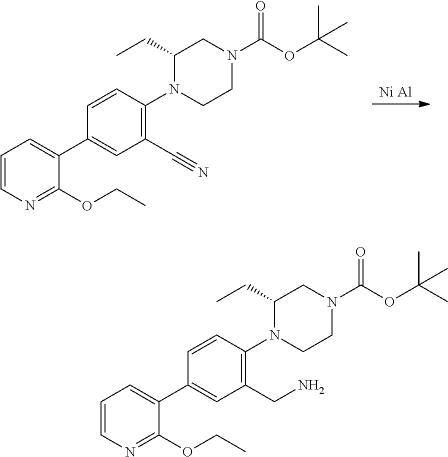 [SMILES: CCOc1ncccc1-c1ccc(N2CCN(C(=O)OC(C)(C)C)C[C@H]2CC)c(C#N)c1 ; "Ni Al" ; CCOc1ncccc1-c1ccc(N2CCN(C(=O)OC(C)(C)C)C[C@H]2CC)c(CN)c1]

Into a 500-mL round-bottom flask purged and maintained with an inert atmosphere of nitrogen, were placed tert-butyl (R)-4-(2-cyano-4-(2-ethoxypyridin-3-yl)phenyl)-3-ethylpiperazine-1-carboxylate (3.2 g, 1 Eq, 7.3 mmol), IPA (200 mL) and H₂O (4.0 mL). Raney Nickel (0.63 g, 0.18 mL, 1.0 Eq, 7.4 mmol) was added and the reaction flask was evacuated and flushed with hydrogen three times. The resulting mixture was stirred at 20° C. for 16 hours under 8 atm of hydrogen. The reaction mixture was filtered through a celite pad and the filtrate was concentrated under reduced pressure. This resulted in crude tert-butyl (R)-4-(2-(aminomethyl)-4-(2-ethoxypyridin-3-yl)phenyl)-3-ethylpiperazine-1-carboxylate (2.442 g, 5.543 mmol, 76%) as a yellow solid. This material was used for next step without further frication. [M+H]=441.7.

### Example 93: tert-butyl (R)-4-(4-(2-ethoxypyridin-3-yl)-2-(((2-nitrophenyl)sulfonamido)-methyl)phenyl)-3-ethylpiperazine-1-carboxylate

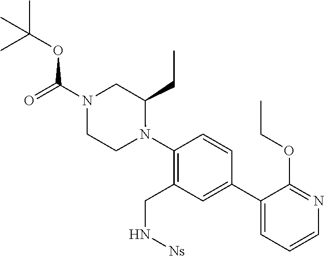

Into a 50-mL round bottom flask, was placed a mixture of tert-butyl (R)-4-(2-(aminomethyl)-4-(2-ethoxypyridin-3-yl)phenyl)-3-ethylpiperazine-1-carboxylate (2.4 g, 1 Eq, 5.4 mmol), TEA (1.7 g, 2.3 mL, 3.1 Eq, 17 mmol), 2-nitrobenzenesulfonyl chloride (1.4 g, 1.2 Eq, 6.3 mmol) and DCM (20 mL). The reaction mixture was stirred at 20° C. for 1 hour. The reaction crude was purified by silica gel chromatography (EtOAc/PE, EtOAc from 0%-90% in 15 min) to afford the title compound (2.7 g, 4.3 mmol, 79%) as a yellow solid. [M+H]=626.4.

### Example 94: (R)—N-(5-(2-ethoxypyridin-3-yl)-2-(2-ethylpiperazin-1-yl)benzyl)-2-nitrobenzenesulfonamide

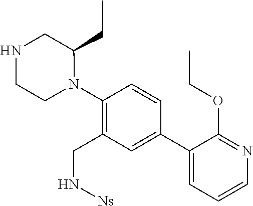

Into 50-mL round bottom flask, were placed tert-butyl (R)-4-(4-(2-ethoxypyridin-3-yl)-2-(((2-nitrophenyl)sulfonamido)methyl)phenyl)-3-ethylpiperazine-1-carboxylate (1.2 g, 1 Eq, 1.9 mmol) and TFA (3 mL). The resulting mixture was stirred at 20° C. for 1 hr. The reaction mixture was concentrated under vacuum to afford crude (R)—N-(5-(2-ethoxypyridin-3-yl)-2-(2-ethylpiperazin-1-yl)benzyl)-2-nitrobenzenesulfonamide (966 mg, 1.84 mmol, 96%) as a yellow oil, which was used in next step without further purification. [M+H]=526.8.

### Example 95: (R)—N-(2-(4-(6-ethoxy-2-(trifluoromethyl)nicotinoyl)-2-ethylpiperazin-1-yl)-5-(2-ethoxypyridin-3-yl)benzyl)-2-nitrobenzenesulfonamide

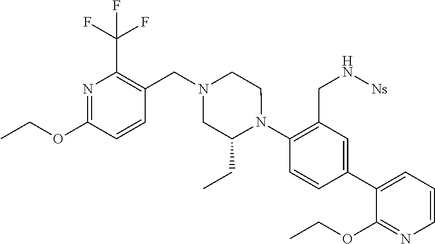

To a DMF (10 mL) solution of 6-ethoxy-2-(trifluoromethyl)nicotinic acid (400 mg, 1 Eq, 1.70 mmol) was added HATU (711 mg, 1.10 Eq, 1.87 mmol), DIEA (660 mg, 889 μL, 3.00 Eq, 5.11 mmol) and (R)—N-(5-(2-ethoxypyridin-3-yl)-2-(2-ethylpiperazin-1-yl)benzyl)-2-nitrobenzenesulfonamide (966 mg, 1.08 Eq, 1.84 mmol). The resulting mixture was stirred at 20° C. for 2 hours. The reaction crude was purified by Prep-HPLC using the following conditions: Column, C18 120 g, 19×150 mm 5 μm; mobile phase, Water (0.05% FA) and ACN (30.0% ACN up to 98.0% in 7 min); Total flow rate, 70 mL/min; Detector, UV 220 nm. This resulted in the title compound (834 mg, 1.12 mmol, 66.0%) as a light yellow solid. [M+H]=743.5.

### Example 96: tert-butyl (R)-(2-((N-(2-(4-(6-ethoxy-2-(trifluoromethyl)nicotinoyl)-2-ethylpiperazin-1-yl)-5-(2-ethoxypyridin-3-yl)benzyl)-2-nitrophenyl)sulfonamido)-ethyl)carbamate

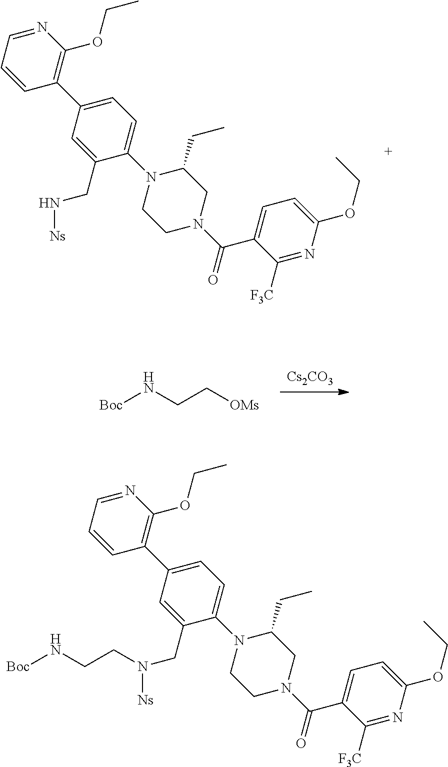

In a 40 mL vial was added (R)—N-(2-(4-(6-ethoxy-2-(trifluoromethyl)nicotinoyl)-2-ethylpiperazin-1-yl)-5-(2-ethoxypyridin-3-yl)benzyl)-2-nitrobenzenesulfonamide (1.8 g, 1 Eq, 2.4 mmol), 2-((tert-butoxycarbonyl)amino)ethyl methanesulfonate (2.9 g, 5.0 Eq, 12 mmol), Cs₂CO₃ (2.4 g, 3.0 Eq, 7.4 mmol) and MeCN (20 mL). The resulting mixture was stirred at 80° C. for 1 hour. The reaction crude was purified by Flash-Prep-HPLC using the following conditions (IntelFlash-1): Column, C18 silica gel; mobile phase, water (0.1% TFA)/CH₃CN (CH₃CN:30%-90% in 12 min); Detector, UV 254&220 nm. The resulted in the title compound (680 mg, 768 μmol, 32%) as a yellow solid. [M+H]=866.2.

### Example 97: (R)—N-(2-aminoethyl)-N-(2-(4-(6-ethoxy-2-(trifluoromethyl)nicotinoyl)-2-ethylpiperazin-1-yl)-5-(2-ethoxypyridin-3-yl)benzyl)-2-nitrobenzenesulfonamide

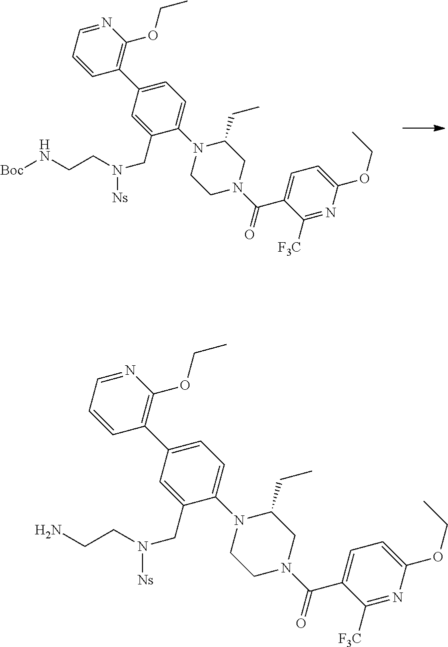

To a DCM (9 mL) solution of tert-butyl (R)-(2-((N-(2-(4-(6-ethoxy-2-(trifluoromethyl)-nicotinoyl)-2-ethylpiperazin-1-yl)-5-(2-ethoxypyridin-3-yl)benzyl)-2-nitrophenyl)sulfonamido)-ethyl)carbamate (680 mg, 1 Eq, 768 μmol) was added TFA (3 mL). The resulting mixture was stirred at 25° C. for 1 hour. Then reaction mixture was concentrated under vacuum. The remaining residue was neutralized with sat. NaHCO₃ till no bubbles produced and the resulting mixture was extracted with EtOAc (2×20 mL). Organic layers were combined, dried over anhydrous sodium sulfate, filtered and concentrated to afford crude product (600 mg, 764 μmol, 99.5%) as a yellow oil, which was used in next step without further purification. [M+H]=786.3.

### Example 98: (R)—N-2-(4-(6-ethoxy-2-(trifluoromethyl)nicotinoyl)-2-ethylpiperazin-1-yl)-5-(2-ethoxypyridin-3-yl)benzyl)-2-nitro-N-(2-((2-nitrophenyl)sulfonamido)ethyl)-benzenesulfonamide

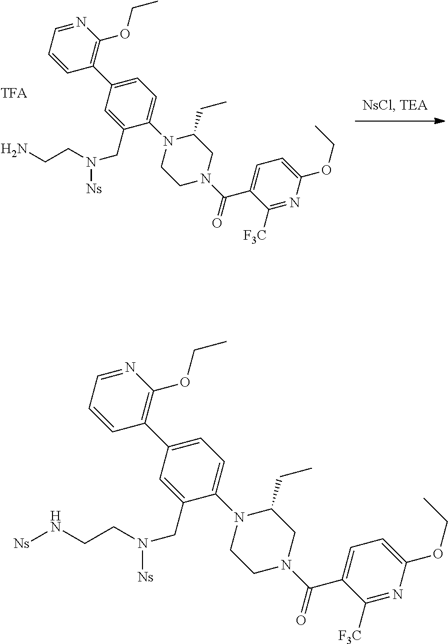

To a DCM (7 mL) solution of (R)—N-(2-aminoethyl)-N-(2-(4-(6-ethoxy-2-(trifluoromethyl)nicotinoyl)-2-ethylpiperazin-1-yl)-5-(2-ethoxypyridin-3-yl)benzyl)-2-nitrobenzenesulfonamide-2,2,2-trifluoroacetaldehyde (1/1) (600 mg, 1 Eq, 679 μmol) was added TEA (343 mg, 472 μL, 4.99 Eq, 3.39 mmol). NsCl (226 mg, 1.50 Eq, 1.02 mmol) was added into the reaction mixture at 0° C. in an ice/water bath. The resulting mixture was stirred at 25° C. for 1 hour.

The reaction mixture was concentrated and the remaining residue was purified by silica gel chromatography eluting with ethyl acetate/petroleum ether (EA:0˜90% in 15 mins). The collected fractions were combined and concentrated to afford the title compound (500 mg, 515 μmol, 75.9%) as a yellow solid. [M+H]=971.1.

### Example 99: (R)-2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-22-oxo-9,12,15,18-tetraoxa-2,5,21-triazatricosan-23-yl)-1,4,7,10-tetraazacyclododecane-1,4,7-triyl)triacetic acid (Compound 15)

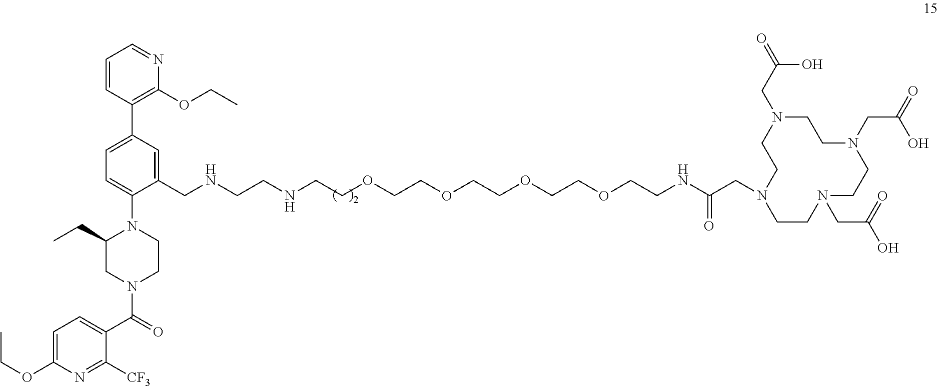

Step 1: Into a 40 mL vial were added (R)—N-(2-(4-(6-ethoxy-2-(trifluoromethyl)-nicotinoyl)-2-ethylpiperazin-1-yl)-5-(2-ethoxypyridin-3-yl)benzyl)-4-nitro-N-(2-((2-nitrophenyl)sulfonamido)ethyl)benzenesulfonamide (500 mg, 1 Eq, 515 μmol), K₂CO₃ (123 mg, 1.73 Eq, 890 μmol) and DCM (15 mL). Subsequently, 2,2-dimethyl-4-oxo-3,8,11,14,17-pentaoxa-5-azaicosan-20-yl methanesulfonate (442 mg, 2.00 Eq, 1.03 mmol) was added. The resulting mixture was stirred at 80° C. for 16 hours. The reaction crude was purified by Prep-HPLC with the following conditions: Column, SunFire Prep C18 OBD Column, 19×150 mm, 5 μm; mobile phase, Water (0.1% TFA) and ACN (30.0% ACN up to 90.0% in 7 min); Total flow rate, 20 mL/min; Detector, UV 220 nm. The collected fractions were combined, concentrated under vacuum, and lyophilized to afford tert-butyl (R)-(1-(2-(4-(6-ethoxy-2-(trifluoromethyl)-nicotinoyl)-2-ethylpiperazin-1-yl)-5-(2-ethoxypyridin-3-yl)phenyl)-2,5-bis((2-nitrophenyl)-sulfonyl)-9,12,15,18-tetraoxa-2,5-diazaicosan-20-yl)carbamate (600 mg, 460 μmol, 89.3%) as a light yellow solid. [M+H]=1304.5.

Step 2: To a stirred DCM (15 mL) solution of tert-butyl (R)-(1-(2-(4-(6-ethoxy-2-(trifluoromethyl)nicotinoyl)-2-ethylpiperazin-1-yl)-5-(2-ethoxypyridin-3-yl)phenyl)-2,5-bis((2-nitrophenyl)sulfonyl)-9,12,15,18-tetraoxa-2,5-diazaicosan-20-yl)carbamate (600 mg, 1 Eq, 460 μmol) was added TFA (5 mL). The resulting mixture was stirred at 25° C. for 1 hour. Then reaction mixture was concentrated under vacuum. The remaining residue was neutralized with sat. NaHCO₃ was added till no bubbles produced, and then extracted with EtOAc. Organic layers were combined, dried over anhydrous sodium sulfate, filtered, and concentrated. This resulted in crude (R)—N-(1-amino-16-((2-nitrophenyl)sulfonyl)-3,6,9,12-tetraoxa-16-azaoctadecan-18-yl)-N-(2-(4-(6-ethoxy-2-(trifluoromethyl)nicotinoyl)-2-ethylpiperazin-1-yl)-5-(2-ethoxypyridin-3-yl)benzyl)-2-nitrobenzenesulfonamide (500 mg, 415 μmol, 90.3%) as a yellow oil, which was used in next step without further purification. [M+H]=1204.4.

Step 3: In a 40 ml vial were added 2-(4,7,10-tris(2-(tert-butoxy)-2-oxoethyl)-1,4,7,10-tetraazacyclododecan-1-yl)acetic acid (238 mg, 1.00 Eq, 416 μmol), HATU (189 mg, 1.20 Eq, 497 μmol), DIEA (161 mg, 217 μL, 3.00 Eq, 1.25 mmol) and DMF (5 mL). The resulting mixture was stirred at 25° C. for 15 min, followed by the addition of (R)—N-(1-amino-16-((2-nitrophenyl)sulfonyl)-3,6,9,12-tetraoxa-16-azaoctadecan-18-yl)-N-(2-(4-(6-ethoxy-2-(trifluoromethyl)nicotinoyl)-2-ethylpiperazin-1-yl)-5-(2-ethoxypyridin-3-yl)benzyl)-2-nitrobenzenesulfonamide (500 mg, 1 Eq, 415 μmol). The reaction mixture was stirred at 25° C. for 1 hour. The reaction crude was purified by Prep-HPLC using the following conditions: Column, SunFire Prep C18 OBD Column, 19*150 mm 5 μm; mobile phase, Water (0.1% TFA) and ACN (30.0% ACN up to 90.0% in 7 min); Total flow rate, 20 mL/min; Detector, UV 220 nm. The resulted in tri-tert-butyl 2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-2,5-bis((2-nitrophenyl)sulfonyl)-22-oxo-9,12,15,18-tetraoxa-2,5,21-triazatricosan-23-yl)-1,4,7,10-tetraazacyclododecane-1,4,7-triyl)(R)-triacetate (350 mg, 199 μmol, 47.9%) as a yellow solid. [M/2+H]=880.4.

Step 4: In a 40 mL vial were added tri-tert-butyl 2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-2,5-bis((2-nitrophenyl)sulfonyl)-22-oxo-9,12,15,18-tetraoxa-2,5,21-triazatricosan-23-yl)-1,4,7,10-tetraazacyclododecane-1,4,7-triyl)(R)-triacetate (350 mg, 1 Eq, 199 μmol), K₂CO₃ (83 mg, 3.0 Eq, 0.60 mmol) and MeCN (5 mL). The resulting mixture was stirred at 50° C. for 1 hour. The reaction crude was purified by Prep-HPLC using the following conditions: Column, SunFire Prep C18 OBD Column, 19*150 mm 5 μm; mobile phase, Water (0.1% TFA) and ACN (30.0% ACN up to 90.0% in 7 min); Total flow rate, 20 mL/min; Detector, UV 220 nm. The fractions collected were combined, concentrated under vacuum, and lyophilized to give tri-tert-butyl 2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-22-oxo-9,12,15,18-tetraoxa-2,5,21-triazatricosan-23-yl)-1,4,7,10-tetraazacyclododecane-1,4,7-triyl)(R)-triacetate (165 mg, 119 μmol, 59.7%) as a light yellow solid. [M/2+H]=695.1.

Step 5: In a 40 mL vial were added tri-tert-butyl 2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-22-oxo-9,12,15,18-tetraoxa-2,5,21-triazatricosan-23-yl)-1,4,7,10-tetraazacyclododecane-1,4,7-triyl)(R)-triacetate (165 mg, 1 Eq, 119 μmol) and TFA (3 mL). The resulting mixture was stirred at 25° C. for 5 hours. The reaction crude was purified by Prep-HPLC using the following conditions: Column, SunFire Prep C18 OBD Column, 19*150 mm 5 μm; mobile phase, Water (0.1% TFA) and ACN (30.0% ACN up to 90.0% in 7 min); Total flow rate, 20 mL/min; Detector, UV 220 nm. The collected fractions were combined, concentrated under vacuum, and lyophilized to give (R)-2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-22-oxo-9,12,15,18-tetraoxa-2,5,21-triazatricosan-23-yl)-1,4,7,10-tetraazacyclododecane-1,4,7-triyl)triacetic acid-2,2,2-trifluoroacetic acid (1/1) (100 mg, 72.8 μmol, 61.3%, 97.2% Purity) as a light yellow solid. [M/2+H-TFA]=611.2.

### Example 100: ¹¹⁵Indium Complex of Compound 15

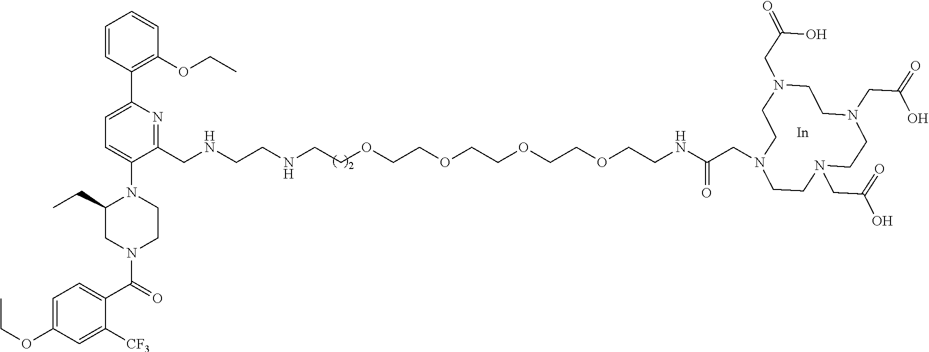

Into an 8 mL flask was added a mixture of (R)-2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-22-oxo-9,12,15,18-tetraoxa-2,5,21-triazatricosan-23-yl)-1,4,7,10-tetraazacyclododecane-1,4,7-triyl)triacetic acid (40 mg, 1 Eq, 33 μmol), ¹¹⁵InCl₃ (25 mg, 3.4 Eq, 0.11 mmol), sodium bicarbonate (15 mg, 5.4 Eq, 0.18 mmol), Water (0.25 mL) and Acetonitrile (0.5 mL). The resulting mixture was stirred at 80° C. for 2 hours. The reaction mixture was diluted with 4 mL of DMSO, filtered and the filtrate was purified by Prep-HPLC using the following conditions: Column, SunFire Prep C18 OBD Column, 19×150 mm, 5 μm; mobile phase, Water and ACN (30% ACN up to 80% in 15 min); Total flow rate, 20 mL/min; Detector, UV 220 nm. This resulted in indium(III) (R)-2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-22-oxo-9,12,15,18-tetraoxa-2,5,21-triazatricosan-23-yl)-1,4,7,10-tetraazacyclododecane-1,4,7-triyl)triacetate (23.4 mg, 17.6 μmol, 54%) as a white solid. [M+H]=1332.6.

### Example 100: ^{69/71}Gallium Complex of Compound 15

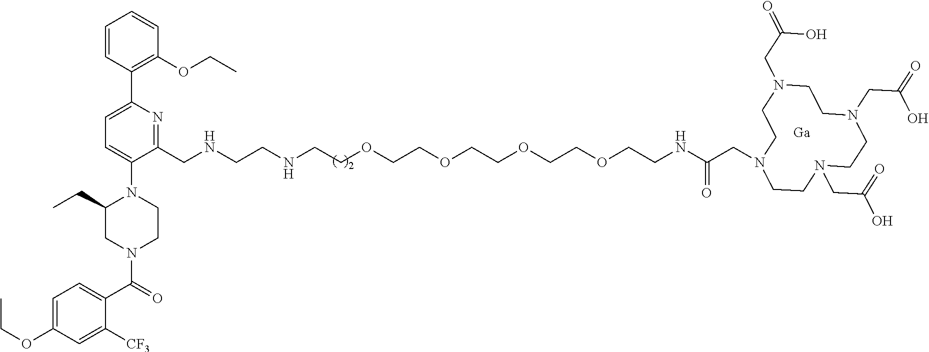

Into an 8 mL flask was added a mixture of (R)-2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-22-oxo-9,12,15,18-tetraoxa-2,5,21-triazatricosan-23-yl)-1,4,7,10-tetraazacyclododecane-1,4,7-triyl)triacetic acid (10 mg, 1 Eq, 8.2 μmol), ^{69/71}gallium(III) chloride (5 mg, 3 Eq, 0.03 mmol), sodium bicarbonate (5 mg, 7 Eq, 0.06 mmol), Acetonitrile (0.3 mL) and Water (0.15 mL). The resulting mixture was stirred at 80° C. for 2 hours. The reaction mixture was diluted with 4 mL of DMSO, filtered and the filtrate was purified by Prep-HPLC using the following conditions: Column, SunFire Prep C18 OBD Column, 19×150 mm, 5 μm; mobile phase, Water and ACN (30% ACN up to 80% in 15 min); Total flow rate, 20 mL/min; Detector, UV 220 nm. This resulted in gallium (R)-2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-22-oxo-9,12,15,18-tetraoxa-2,5,21-triazatricosan-23-yl)-1,4,7,10-tetraazacyclododecane-1,4,7-triyl)triacetate (1.8 mg, 1.4 μmol, 17%) as a white solid. [M+H]=1286.7.

### Example 102: tert-butyl (R)-4-(3-(aminomethyl)-2′-ethoxy-[1,1′-biphenyl]-4-yl)-3-ethylpiperazine-1-carboxylate

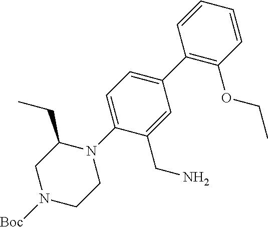 [SMILES: CCOc1ccccc1-c1ccc(N2CCN(C(=O)OC(C)(C)C)C[C@H]2CC)c(CN)c1]

Step 1: Into a 250 mL round bottom flask, were placed tert-butyl (R)-4-(4-bromo-2-cyanophenyl)-3-ethylpiperazine-1-carboxylate (4.3 g, 1 Eq, 11 mmol), 1,1′-Bis(di-t-butylphosphino)ferrocene palladium dichloride (360 mg, 0.051 Eq, 552 μmol), (2-ethoxyphenyl)boronic acid (2.7 g, 1.5 Eq, 16 mmol), K₂CO₃ (4.5 g, 3 Eq, 33 mmol), 1,4-Dioxane (45 mL), and Water (5 mL). The resulting mixture was stirred at 80° C. for 2 hrs. The reaction mixture was cooled to room temperature and concentrated under vacuum. The remaining residue was purified by silica gel chromatography eluting with ethyl acetate/petroleum ether (1:3). This resulted in tert-butyl (R)-4-(3-cyano-2′-ethoxy-[1,1′-biphenyl]-4-yl)-3-ethylpiperazine-1-carboxylate (3.1 g, 7.1 mmol, 65%) as a yellow solid. [M+H]=436.2.

Step 2: Into a 250 mL round bottom flask, were placed tert-butyl (R)-4-(3-cyano-2′-ethoxy-[1,1′-biphenyl]-4-yl)-3-ethylpiperazine-1-carboxylate (2.8 g, 1 Eq, 6.4 mmol), IPA (60 mL), NH₃ in H₂O (12 mL), and nickel (300 mg, 0.80 Eq, 5.11 mmol). The reaction flask was evacuated and flushed with nitrogen three times, followed by flushing with hydrogen. The resulting mixture was stirred at 21° C. for 16 hrs under an atmospheric of hydrogen (balloon). The reaction crude was filtered and the filtrate was concentrated under vacuum. This resulted in crude tert-butyl (R)-4-(3-(aminomethyl)-2′-ethoxy-[1,1′-biphenyl]-4-yl)-3-ethylpiperazine-1-carboxylate (2 g, 5 mmol, 70%) as a yellow solid. [M+H]=440.5

### Example 103: tert-butyl (R)-4-(2′-ethoxy-3-(((2-nitrophenyl)sulfonamido)methyl)-[1,1′-biphenyl]-4-yl)-3-ethylpiperazine-1-carboxylate

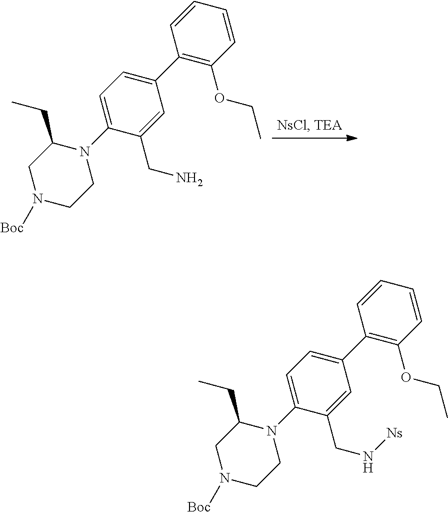

Into a 250 mL round bottom flask, were placed tert-butyl (R)-4-(3-(aminomethyl)-2′-ethoxy-[1,1′-biphenyl]-4-yl)-3-ethylpiperazine-1-carboxylate (2.0 g, 1 Eq, 4.5 mmol), DCM (25 mL) and TEA (1.5 g, 2.1 mL, 3.3 Eq, 15 mmol), followed by the addition of 2-nitrobenzene sulfonyl chloride (1.4 g, 1.4 Eq, 6.3 mmol) in DCM (6 mL) at 0° C. The resulting mixture was stirred at 20° C. for 1 hour. The reaction mixture was diluted with water (40 mL) and extracted with ethyl acetate (3×50 mL). Organic layers combined, washed with brine (2×20 mL), dried over anhydrous sodium sulfate and concentrated under vacuum. The remaining residue was purified by silica gel chromatography eluting with ethyl acetate/petroleum ether (1:3). This resulted in the title compound (1.02 g, 1.63 mmol, 36%) as a yellow solid. [M+H]=625.8.

### Example 104: (R)—N-((2′-ethoxy-4-(2-ethylpiperazin-1-yl)-[1,1′-biphenyl]-3-yl)methyl)-2-nitrobenzenesulfonamide

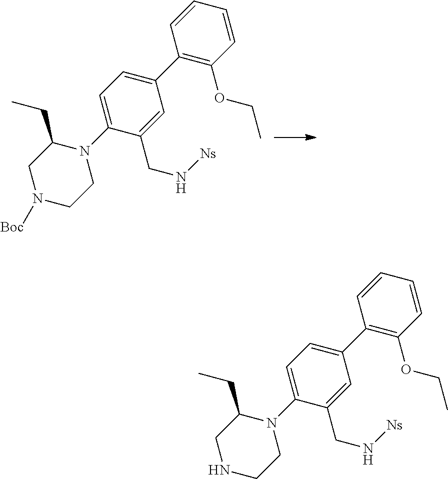

Into a 100 mL round bottom flask, were added tert-butyl (R)-4-(2′-ethoxy-3-(((2-nitrophenyl)sulfonamido)methyl)-[1,1′-biphenyl]-4-yl)-3-ethylpiperazine-1-carboxylate (1.02 g, 1 Eq, 1.63 mmol), TFA (10 mL) and DCM (2 mL). The resulting reaction mixture was stirred at 20° C. for 1 hour. The reaction mixture was concentrated under vacuum and diluted with water (30 mL). Saturated aq NaHCO₃ was added until pH ˜7 and the resulting solution was extracted with ethyl acetate (3×30 mL). Organic layers were combined, dried over anhydrous sodium sulfate and concentrated under vacuum. This resulted in the title compound (790 mg, 1.51 mmol, 92.2%) as a light yellow solid. [M+H]=525.2.

### Example 105: (R)—N-((2′-ethoxy-4-(4-(4-ethoxy-2-(trifluoromethyl)benzoyl)-2-ethylpiperazin-1-yl)-[1,1′-biphenyl]-3-yl)methyl)-2-nitrobenzenesulfonamide

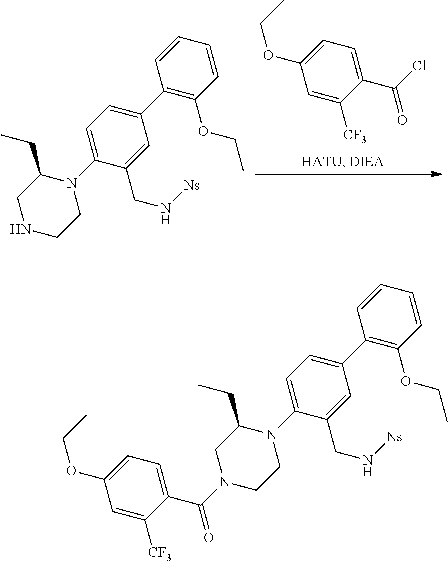

Into a 40 mL vial, were placed (R)—N-((2′-ethoxy-4-(2-ethylpiperazin-1-yl)-[1,1′-biphenyl]-3-yl)methyl)-2-nitrobenzenesulfonamide (790 mg, 1 Eq, 1.51 mmol), HATU (800 mg, 1.40 Eq, 2.10 mmol), DMF (8 mL) and DIEA (590 mg, 795 μL, 3.03 Eq, 4.56 mmol). The resulting mixture was stirred at 21° C. for 10 min., followed by the addition of 4-ethoxy-2-(trifluoromethyl)benzoic acid (450 mg, 1.28 Eq, 1.92 mmol). The reaction mixture was stirred at 21° C. for 1 hour. The reaction crude was purified by Flash-HPLC using the following conditions (IntelFlash-1): Column, C18 OBD Column; mobile phase, water (0.1% FA)/CH₃CN (CH₃CN:3 0%-98% in 7 min, 98%˜98% in 4 min); Detector, UV 254&220 nm. This resulted in the title compound (700 mg, 945 μmol, 62.8%) as a yellow solid. [M+H]=741.3.

### Example 106: tert-butyl (R)-(2-((N-((2′-ethoxy-4-(4-(4-ethoxy-2-(trifluoromethyl)benzoyl)-2-ethylpiperazin-1-yl)-[1,1′-biphenyl]-3-yl)methyl)-2-nitrophenyl)sulfonamido)ethyl)carbamate

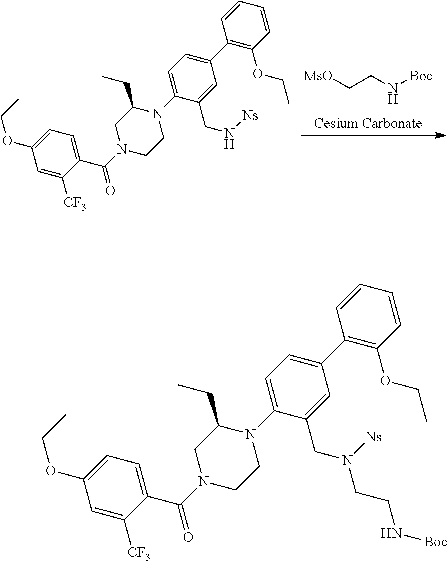

Into a 8 mL vial, were added (R)—N-((2′-ethoxy-4-(4-(4-ethoxy-2-(trifluoromethyl)benzoyl)-2-ethylpiperazin-1-yl)-[1,1′-biphenyl]-3-yl)methyl)-2-nitrobenzenesulfonamide (650 mg, 1 Eq, 877 μmol), ACN (7 mL), cesium carbonate (850 mg, 2.97 Eq, 2.61 mmol), and 2-((tert-butoxycarbonyl)amino)ethyl methanesulfonate (420 mg, 2.00 Eq, 1.76 mmol). The resulting mixture was stirred for 1 hour at 80° C. The reaction crude was purified by Prep-HPLC using the following conditions (Flash-HPLC-013): Column, SunFire Prep C18 OBD Column, 19×150 mm 5 μm; mobile phase, Water (0.1% FA) and ACN (30.0% ACN up to 98.0% in 16 min); Total flow rate, 70 mL/min; Detector, UV 220 nm. Fractions collected were combined and concentrated under vacuum. This resulted in tert-butyl (R)-(2-((N-((2′-ethoxy-4-(4-(4-ethoxy-2-(trifluoromethyl)benzoyl)-2-ethylpiperazin-1-yl)-[1,1′-biphenyl]-3-yl)methyl)-2-nitrophenyl)sulfonamido)ethyl)carbamate (300 mg, 339 μmol, 38.7%) as a yellow solid. [M+H]=884.3.

### Example 107: (R)—N-(2-aminoethyl)-N-((2′-ethoxy-4-(4-(4-ethoxy-2-(trifluoromethyl)benzoyl)-2-ethylpiperazin-1-yl)-[1,1′-biphenyl]-3-yl)methyl)-2-nitrobenzenesulfonamide

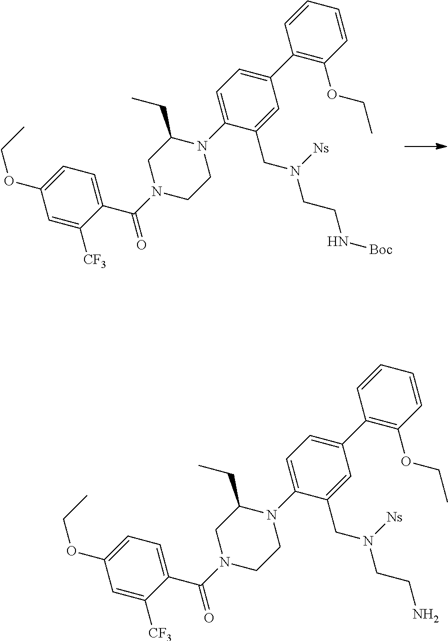

Into a 100 mL round bottom flask, were added tert-butyl (R)-(2-((N-((2′-ethoxy-4-(4-(4-ethoxy-2-(trifluoromethyl)benzoyl)-2-ethylpiperazin-1-yl)-[1,1′-biphenyl]-3-yl)methyl)-2-nitrophenyl)sulfonamido)ethyl)carbamate (295 mg, 1 Eq, 334 μmol), DCM (5 mL) and TFA (1 mL). The resulting reaction mixture was stirred at 20° C. for 1 hour. The reaction mixture was concentrated under vacuum and diluted with water (20 mL). Saturated aq NaHCO₃ was added until pH ˜7 and the resulting solution was extracted with ethyl acetate (3×20 mL). Organic layers were combined, dried over anhydrous sodium sulfate and concentrated under vacuum. This resulted in crude product (270 mg, 0.31 mmol, 93%, 90% Purity) as a light yellow solid. [M+H]=784.2.

### Example 108: (R)—N-((2′-ethoxy-4-(4-(4-ethoxy-2-(trifluoromethyl)benzoyl)-2-ethylpiperazin-1-yl)-[1,1′-biphenyl]-3-yl)methyl)-4-nitro-N-(2-((2-nitrophenyl)sulfonamido)ethyl)benzenesulfonamide

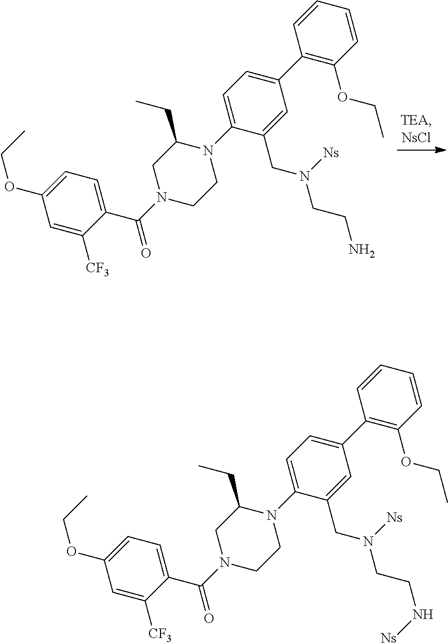

Into a 250 mL round bottom flask, were placed (R)—N-(2-aminoethyl)-N-((2′-ethoxy-4-(4-(4-ethoxy-2-(trifluoromethyl)benzoyl)-2-ethylpiperazin-1-yl)-[1,1′-biphenyl]-3-yl)methyl)-2-nitrobenzenesulfonamide (270 mg, 1 Eq, 344 μmol), DCM (3 mL) and TEA (120 mg, 165 μL, 3.44 Eq, 1.19 mmol), followed by the addition of 2-nitrobenzenesulfonyl chloride (100 mg, 1.31 Eq, 451 μmol) in DCM (1 mL) at 0° C. The resulting solution was stirred at 20° C. for 1 hour. The reaction mixture was diluted with water (40 mL) and extracted with ethyl acetate (3×50 mL). Organic layers were combined, washed with brine (2×20 mL), dried over anhydrous sodium sulfate and concentrated under vacuum. The remaining residue was purified by silica gel chromatography eluting with ethyl acetate/petroleum ether (1:3). This resulted in the title compound (310 mg, 320 μmol, 92.9%) as a yellow solid. [M+H]=969.0.

### Example 109: tert-butyl (R)-(1-(2′-ethoxy-4-(4-(4-ethoxy-2-(trifluoromethyl)benzoyl)-2-ethylpiperazin-1-yl)-[1,1′-biphenyl]-3-yl)-2,5-bis((2-nitrophenyl)sulfonyl)-9,12,15,18-tetraoxa-2,5-diazaicosan-20-yl)carbamate

Into an 8 mL vial, were added (R)—N-((2′-ethoxy-4-(4-(4-ethoxy-2-(trifluoromethyl)benzoyl)-2-ethylpiperazin-1-yl)-[1,1′-biphenyl]-3-yl)methyl)-2-nitro-N-(2-((2-nitrophenyl)sulfonamido)ethyl)benzenesulfonamide (290 mg, 1 Eq, 299 μmol), ACN (3 mL), cesium carbonate (300 mg, 3.08 Eq, 921 μmol), and 2,2-dimethyl-4-oxo-3,8,11,14,17-pentaoxa-5-azaicosan-20-yl methanesulfonate (270 mg, 2.10 Eq, 629 μmol). The resulting mixture was stirred at 80° C. for 4 hours. The reaction crude was purified by Prep-HPLC using the following conditions (Flash-HPLC-013): Column, C18 OBD Column, 19×150 mm, 5 μm; mobile phase, Water (0.1% FA) and ACN (30.0% ACN up to 98.0% in 18 min); Total flow rate, 70 mL/min; Detector, UV 220 nm. Fractions collected were combined and concentrated under vacuum. This resulted in crude product (305 mg, 234 μmol, 78.2%) as a yellow solid. [M+H]=1302.7.

### Example 110: (R)—N-(1-amino-16-((2-nitrophenyl)sulfonyl)-3,6,9,12-tetraoxa-16-azaoctadecan-18-yl)-N-((2′-ethoxy-4-(4-(4-ethoxy-2-(trifluoromethyl)benzoyl)-2-ethylpiperazin-1-yl)-[111′-biphenyl]-3-yl)methyl)-2-nitrobenzenesulfonamide

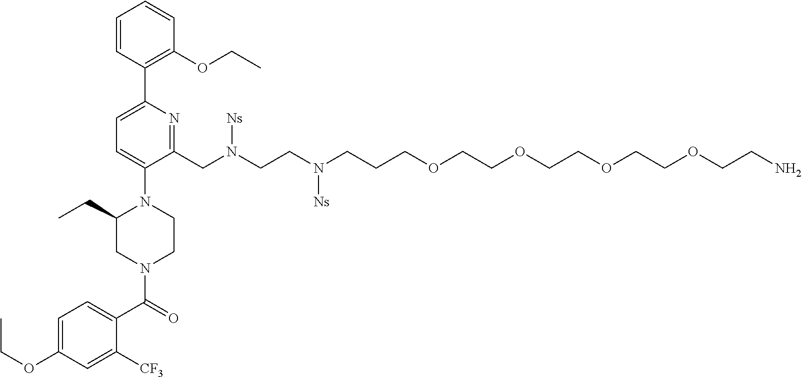

Into a 100 mL round bottom flask, were added tert-butyl (R)-(1-(2′-ethoxy-4-(4-(4-ethoxy-2-(trifluoromethyl)benzoyl)-2-ethylpiperazin-1-yl)-[1,1′-biphenyl]-3-yl)-2,5-bis((2-nitrophenyl) sulfonyl)-9,12,15,18-tetraoxa-2,5-diazaicosan-20-yl)carbamate (300 mg, 1 Eq, 230 μmol), DCM (4 mL) and TFA (1 mL). The resulting mixture was stirred at 20° C. for 1 hour. The reaction mixture was concentrated under vacuum and diluted with water (20 mL). Saturated aq. NaHCO₃ was added until pH ˜7 and the resulting solution was extracted with ethyl acetate (3×20 mL). Organic layers were combined, washed with brine (2×20 mL), dried over anhydrous sodium sulfate and concentrated under vacuum. This resulted in crude product (280 mg, 90% purity). [M+H]=1202.4.

### Example 111: (R)-2,2′,2″-(10-(1-(2′-ethoxy-4-(4-(4-ethoxy-2-(trifluoromethyl)benzoyl)-2-ethylpiperazin-1-yl)-[1,1′-biphenyl]-3-yl)-22-oxo-9,12,15,18-tetraoxa-2,5,21-triazatricosan-23-yl)-1,4,7,10-tetraazacyclododecane-1,4,7-triyl)triacetic acid (Compound 16)

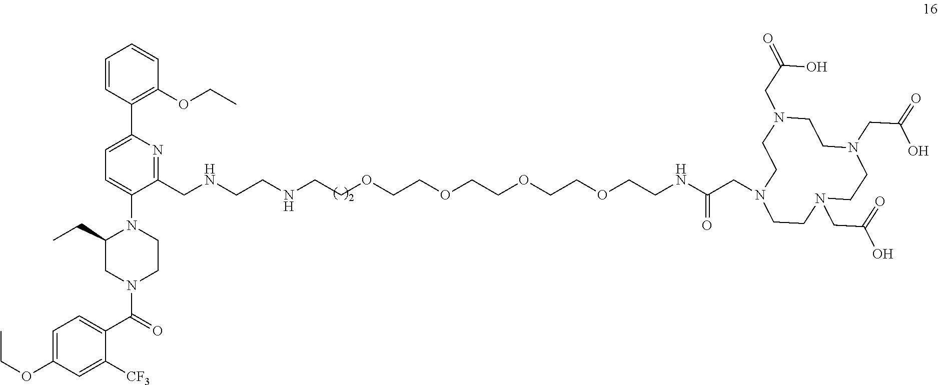 [SMILES: CCOc1ccc(C(=O)N2CCN(c3ccc(-c4ccccc4OCC)nc3CNCCNCCOCCOCCOCCOCCNC(=O)CN3CCN(CC(=O)O)CCN(CC(=O)O)CCN(CC(=O)O)CC3)[C@H](CC)C2)c(C(F)(F)F)c1]

Step 1: To a DMF (3 mL) solution of 2-(4,7,10-tris(2-(tert-butoxy)-2-oxoethyl)-1,4,7,10-tetraazacyclododecan-1-yl)acetic acid (160 mg, 1.24 Eq, 279 μmol) was added HATU (120 mg, 1.41 Eq, 316 μmol) and DIEA (120 mg, 162 μL, 4.13 Eq, 928 μmol). The resulting mixture was stirred at 20° C. for 10 min, followed by the addition of (R)—N-(1-amino-16-((2-nitrophenyl)sulfonyl)-3,6,9,12-tetraoxa-16-azaoctadecan-18-yl)-N-((2′-ethoxy-4-(4-(4-ethoxy-2-(trifluoromethyl)benzoyl)-2-ethylpiperazin-1-yl)-[1,1′-biphenyl]-3-yl)methyl)-2-nitrobenzenesulfonamide (270 mg, 1 Eq, 225 μmol). The reaction mixture was stirred at 20° C. for 1 hour. The reaction crude was purified by Flash-HPLC using the following conditions: (IntelFlash-1): Column, C18 OBD Column; mobile phase, water (0.1% FA)/CH₃CN (CH₃CN: 30-98% in 8 min, and remained at 98% for 3 min); Detector, UV 254&220 nm. This resulted in tri-tert-butyl 2,2′,2″-(10-(1-(2′-ethoxy-4-(4-(4-ethoxy-2-(trifluoromethyl)benzoyl)-2-ethylpiperazin-1-yl)-[1,1′-biphenyl]-3-yl)-2,5-bis((2-nitrophenyl) sulfonyl)-22-oxo-9,12,15,18-tetraoxa-2,5,21-triazatricosan-23-yl)-1,4,7,10-tetraazacyclododecane-1,4,7-triyl)(R)-triacetate (260 mg, 148 μmol, 65.9%) as a yellow solid. [M+H]=1757.1.

Step 2: To an ACN (3 mL) solution of tri-tert-butyl 2,2′,2″-(10-(1-(2′-ethoxy-4-(4-(4-ethoxy-2-(trifluoromethyl)benzoyl)-2-ethylpiperazin-1-yl)-[1,1′-biphenyl]-3-yl)-2,5-bis((2-nitrophenyl)sulfonyl)-22-oxo-9,12,15,18-tetraoxa-2,5,21-triazatricosan-23-yl)-1,4,7,10-tetraazacyclododecane-1,4,7-triyl)(R)-triacetate (250 mg, 1 Eq, 142 μmol) was added potassium carbonate (100 mg, 5.09 Eq, 724 μmol), and 2-chlorobenzenethiol (100 mg, 4.86 Eq, 691 μmol). The resulting mixture was stirred at 50° C. for 1 hour. The reaction crude was purified by Flash-HPLC using the following conditions: Column, C18 silica gel; mobile phase, water (0.1% FA)/CH₃CN (CH₃CN: 30-70% in 13 min); Detector, UV 254&220 nm. This resulted in tri-tert-butyl 2,2′,2″-(10-(1-(2′-ethoxy-4-(4-(4-ethoxy-2-(trifluoromethyl)benzoyl)-2-ethylpiperazin-1-yl)-[1,1′-biphenyl]-3-yl)-22-oxo-9,12,15,18-tetraoxa-2,5,21-triazatricosan-23-yl)-1,4,7,10-tetraazacyclododecane-1,4,7-triyl)(R)-triacetate (180 mg, 130 μmol, 91.2%) as a yellow solid. [M+H]=1386.8.

Step 3: Into an 8 mL vial, were added tri-tert-butyl 2,2′,2″-(10-(1-(2′-ethoxy-4-(4-(4-ethoxy-2-(trifluoromethyl)benzoyl)-2-ethylpiperazin-1-yl)-[1,1′-biphenyl]-3-yl)-22-oxo-9,12,15,18-tetraoxa-2,5,21-triazatricosan-23-yl)-1,4,7,10-tetraazacyclododecane-1,4,7-triyl)(R)-triacetate (170 mg, 1 Eq, 123 μmol) and TFA (2 mL). The resulting reaction mixture was stirred at 30° C. for 3 hrs. The reaction mixture was concentrated under vacuum. The remaining residue was purified by Prep-HPLC using the following conditions: Column, SunFire Prep C18 OBD Column, 19×150 mm 5 μm; mobile phase, Water (0.05% TFA) and ACN (20.0% ACN up to 73.0% in 10 min); Total flow rate, 20 mL/min; Detector, UV 220 nm. This resulted in (R)-2,2′,2″-(10-(1-(2′-ethoxy-4-(4-(4-ethoxy-2-(trifluoromethyl)benzoyl)-2-ethylpiperazin-1-yl)-[1,1′-biphenyl]-3-yl)-22-oxo-9,12,15,18-tetraoxa-2,5,21-triazatricosan-23-yl)-1,4,7,10-tetraazacyclododecane-1,4,7-triyl)triacetic acid-2,2,2-trifluoroacetic acid (1/2) (110 mg, 76.0 μmol, 62.0%) as a white solid. [M+H-2TFA]=1218.8.

### Example 112: tert-butyl (R)-4-(2-(aminomethyl)-6-(2-ethoxyphenyl)pyridin-3-yl)-3-ethylpiperazine-1-carboxylate

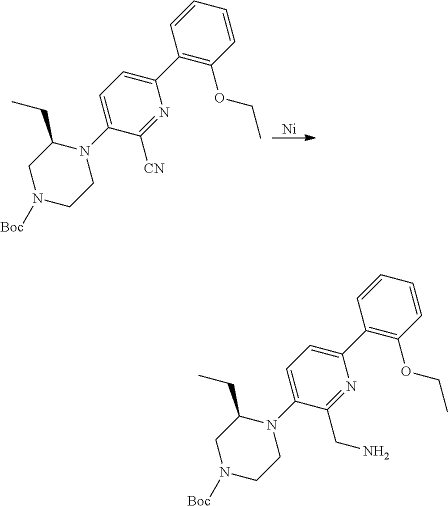

Into a 250 mL round bottom flask, were placed tert-butyl (R)-4-(2-cyano-6-(2-ethoxyphenyl)pyridin-3-yl)-3-ethylpiperazine-1-carboxylate (2.8 g, 1 Eq, 6.4 mmol), IPA (60 mL), NH₃·H₂O (12 mL), and nickel (300 mg, 0.80 Eq, 5.11 mmol). The reaction flask was evacuated and flushed with nitrogen three times, followed by flushing with hydrogen. The reaction mixture was stirred at 21° C. for 16 hrs under an atmospheric of hydrogen (balloon). The reaction crude was filtered and the filtrate was concentrated under vacuum. This resulted in crude product (2.5 g, 5.7 mmol, 88%) as a yellow solid. [M+H]=441.3.

### Example 113: tert-butyl (R)-4-(6-(2-ethoxyphenyl)-2-(((2-nitrophenyl)sulfonamido)methyl)pyridin-3-yl)-3-ethylpiperazine-1-carboxylate

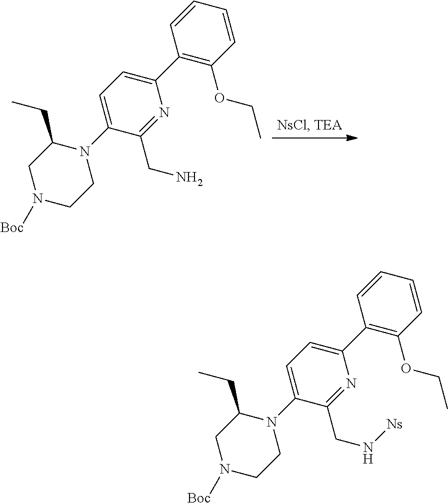

Into a 250 mL round bottom flask, were placed tert-butyl (R)-4-(2-(aminomethyl)-6-(2-ethoxyphenyl)pyridin-3-yl)-3-ethylpiperazine-1-carboxylate (2.8 g, 1 Eq, 6.4 mmol), DCM (0.4 mL) and triethylamine (2 g, 3 Eq, 0.02 mol), followed by the addition of 2-nitrobenzenesulfonyl chloride (2.1 g, 1.5 Eq, 9.5 mmol) in DCM (6 mL) at 0° C. The resulting solution was stirred at 20° C. for 1 hour. The reaction mixture was diluted with water (40 mL) and extracted with ethyl acetate (3×50 mL). Organic layers were combined, washed with brine (2×20 mL), dried over anhydrous sodium sulfate and concentrated under vacuum. The remaining residue was purified by silica gel chromatography eluting with ethyl acetate/petroleum ether (1:3). This resulted in the title compound (1.8 g, 2.9 mmol, 45%) as a yellow solid. [M+H]=626.2.

### Example 114: (R)—N-((6-(2-ethoxyphenyl)-3-(2-ethylpiperazin-1-yl)pyridin-2-yl)methyl)-2-nitrobenzenesulfonamide

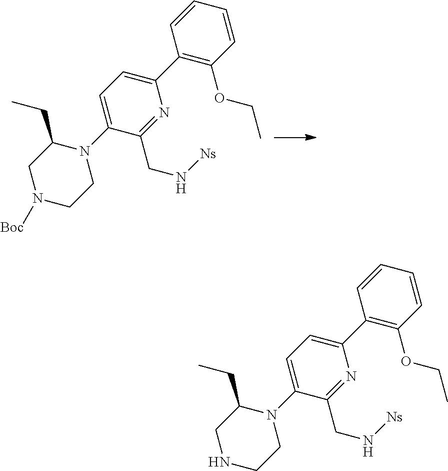

Into a 100 mL round bottom flask, were added tert-butyl (R)-4-(6-(2-ethoxyphenyl)-2-(((2-nitrophenyl)sulfonamido)methyl)pyridin-3-yl)-3-ethylpiperazine-1-carboxylate (1.0 g, 1 Eq, 1.6 mmol), DCM (10 mL) and TFA (2 mL). The resulting mixture was stirred at 20° C. for 1 hour. The reaction mixture was concentrated under vacuum and diluted with water. Saturated aq NaHCO₃ was added until pH -7 and the resulting solution was extracted with ethyl acetate (3×30 mL). Organic layers were combined, dried over anhydrous sodium sulfate, filtered and concentrated under vacuum. This resulted in the title compound (900 mg, 1.5 mmol, 96%, 90% Purity) as a solid. [M+H]=526.4.

### Example 115: (R)—N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide

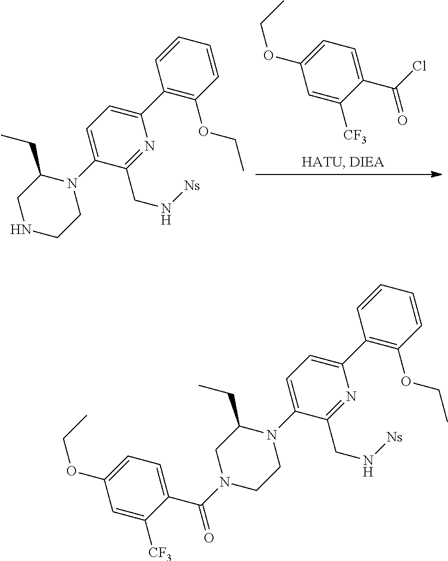

To a DMF (10 mL) solution of 4-ethoxy-2-(trifluoromethyl)benzoic acid (450 mg, 1.19 Eq, 1.92 mmol) was added 2-(3H-[1,2,3]triazolo[4,5-b]pyridin-3-yl)-1,1,3,3-tetramethylisouronium hexafluorophosphate(V) (750 mg, 1.22 Eq, 1.97 mmol) and N-ethyl-N-isopropylpropan-2-amine (650 mg, 3.11 Eq, 5.03 mmol). The resulting mixture was stirred at 20° C. for 10 min, followed by the addition of (R)—N-((6-(2-ethoxyphenyl)-3-(2-ethylpiperazin-1-yl)pyridin-2-yl)methyl)-2-nitrobenzenesulfonamide (850 mg, 1 Eq, 1.62 mmol). The reaction mixture was stirred at 20° C. for 1 hour. The reaction crude was purified by Flash-HPLC using the following conditions: Column, C18 silica gel; mobile phase, water (0.1% FA)/CH₃CN (CH₃CN:30-98% in 6 min, 98% for 3 min); Detector, UV 254&220 nm. This resulted in the title compound (830 mg, 1.12 mmol, 69.2%) as a yellow solid. [M+H]=742.3.

### Example 116: tert-butyl (R)-(2-((N-((3-(4-(4-ethoxy-2-(trifluoromethyl)benzoyl)-2-ethylpiperazin-1-yl)-6-(2-ethoxyphenyl)pyridin-2-yl)methyl)-2-nitrophenyl)sulfonamido)ethyl)-carbamate

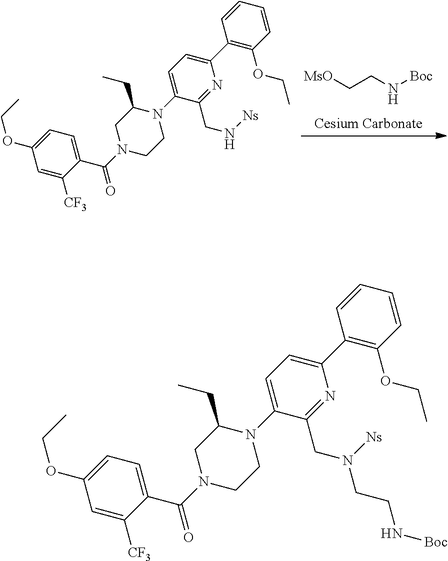

Into a 40 mL vial, were added (R)—N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide (740 mg, 1 Eq, 998 μmol), ACN (8 mL), cesium carbonate (1.1 g, 3.4 Eq, 3.4 mmol), and 2-((tert-butoxycarbonyl)amino)ethyl methanesulfonate (500 mg, 2.09 Eq, 2.09 mmol). The resulting mixture was stirred at 80° C. for 3 hours. The reaction crude was purified by Prep-HPLC using the following conditions: Column, SunFire Prep C18 OBD Column, 19×150 mm, 5 μm; mobile phase, Water (0.1% FA) and ACN (30.0% ACN up to 98.0% in 8 min); Total flow rate, 70 mL/min; Detector, UV 220 nm. Fractions collected were combined and concentrated under vacuum. This resulted in the title compound (650 mg, 734 μmol, 73.6%) as a yellow solid. [M+H]=886.1.

### Example 117: (R)—N-(2-aminoethyl)-N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide

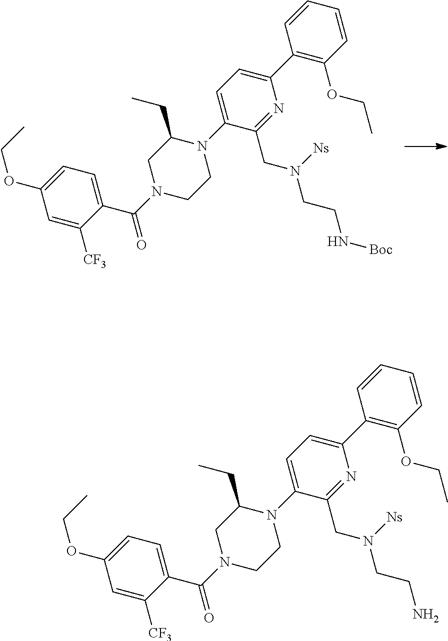

Into a 100 mL round bottom flask, were added tert-butyl (R)-(2-((N-((3-(4-(4-ethoxy-2-(trifluoromethyl)benzoyl)-2-ethylpiperazin-1-yl)-6-(2-ethoxyphenyl)pyridin-2-yl)methyl)-2-nitrophenyl)sulfonamido)ethyl)carbamate (630 mg, 1 Eq, 712 μmol), DCM (10 mL) and TFA (2 mL). The resulting mixture was stirred at 20° C. for 1 hour. The reaction mixture was concentrated under vacuum and diluted with water. Saturated aq NaHCO₃ was added until pH ˜7 and the resulting solution was extracted with ethyl acetate (3×30 mL). Organic layers were combined, washed with brine (2×20 mL), dried over anhydrous sodium sulfate and concentrated under vacuum. This resulted in the title compound (600 mg, 0.69 mmol, 97%, 90% Purity) as a light yellow solid. [M+H]=785.3.

### Example 118: (R)—N-((3-(4-(4-ethoxy-2-(trifluoromethyl)benzoyl)-2-ethylpiperazin-1-yl)-6-(2-ethoxyphenyl)pyridin-2-yl)methyl)-4-nitro-N-(2-((2-nitrophenyl)sulfonamido)ethyl)-benzenesulfonamide

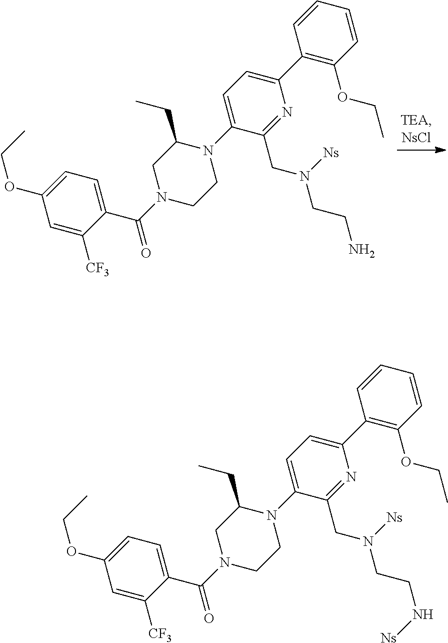

Into a 250 mL round bottom flask, were placed (R)—N-(2-aminoethyl)-N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide (580 mg, 1 Eq, 739 μmol), DCM (10 mL), and TEA (250 mg, 344 μL, 3.34 Eq, 2.47 mmol), followed by the addition of 2-nitrobenzenesulfonyl chloride (200 mg, 1.22 Eq, 902 μmol) in DCM (2 mL) at 0° C. The resulting solution was stirred at 20° C. for 1 hour. The resulting solution was diluted with water (30 mL) and extracted with ethyl acetate (3×30 mL). Organic layers were combined, washed with brine (2×20 mL), dried over anhydrous sodium sulfate and concentrated under vacuum. The remaining residue was purified by silica gel chromatography eluting with ethyl acetate/petroleum ether (1:1). This resulted in the title compound (600 mg, 619 μmol, 83.7%) as a yellow solid. [M+H]=970.2.

### Example 119: tert-butyl (R)-(1-(3-(4-(4-ethoxy-2-(trifluoromethyl)benzoyl)-2-ethylpiperazin-1-yl)-6-(2-ethoxyphenyl)pyridin-2-yl)-2,5-bis((2-nitrophenyl)sulfonyl)-9,12,15,18-tetraoxa-2,5-diazaicosan-20-yl)carbamate

Into a 40 mL vial, were added (R)—N-((3-(4-(4-ethoxy-2-(trifluoromethyl)benzoyl)-2-ethylpiperazin-1-yl)-6-(2-ethoxyphenyl)pyridin-2-yl)methyl)-4-nitro-N-(2-((2-nitrophenyl)sulfonamido)ethyl)benzenesulfonamide (520 mg, 1 Eq, 536 μmol), ACN (5 mL), cesium carbonate (580 mg, 3.32 Eq, 1.78 mmol), and 2,2-dimethyl-4-oxo-3,8,11,14,17-pentaoxa-5-azaicosan-20-yl methanesulfonate (400 mg, 1.74 Eq, 931 μmol). The resulting mixture was stirred at 80° C. for 16 hours. The reaction crude was purified by Prep-HPLC using the following conditions: Column, SunFire Prep C18 OBD Column, 19×150 mm, 5 μm; mobile phase, Water (0.1% FA) and ACN (30.0% ACN up to 98.0% in 18 min); Total flow rate, 70 mL/min; Detector, UV 220 nm. Fractions collected were combined and concentrated under vacuum. This resulted in the title compound (500 mg, 384 μmol, 71.6%) as a yellow solid. [M+H]=1303.2.

### Example 120: (R)—N-(1-amino-16-((2-nitrophenyl)sulfonyl)-3,6,9,12-tetraoxa-16-azaoctadecan-18-yl)-N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide

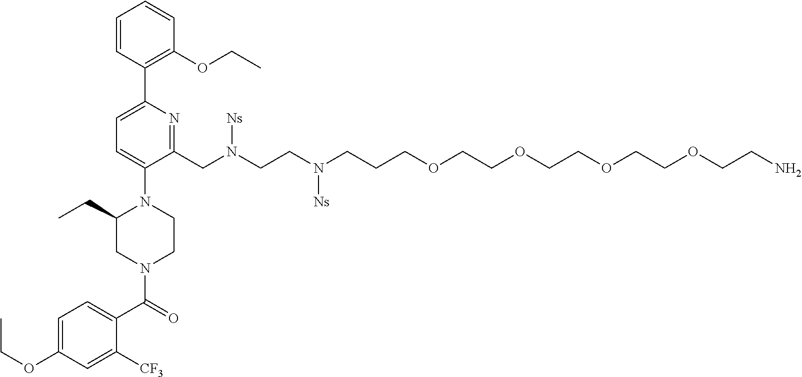

Into a 100 mL round bottom flask, were added tert-butyl (R)-(1-(3-(4-(4-ethoxy-2-(trifluoromethyl)benzoyl)-2-ethylpiperazin-1-yl)-6-(2-ethoxyphenyl)pyridin-2-yl)-2,5-bis((2-nitrophenyl)sulfonyl)-9,12,15,18-tetraoxa-2,5-diazaicosan-20-yl)carbamate (480 mg, 1 Eq, 368 μmol), DCM (6 mL) and TFA (2 mL). The resulting mixture was stirred at 20° C. for 1 hour. The reaction mixture was concentrated under vacuum and diluted with water. Saturated aq. NaHCO₃ was added until pH ˜7 and the resulting solution was extracted with ethyl acetate (3×30 mL). Organic layers were combined, washed with brine (2×20 mL), dried over anhydrous sodium sulfate, and concentrated under vacuum. This resulted in the title compound (450 mg, 0.34 mmol, 91%, 90% Purity) as light yellow solid. [M+H]=1203.7.

### Example 121: (R)-2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-22-oxo-9,12,15,18-tetraoxa-2,5,21-triazatricosan-23-yl)-1,4,7,10-tetraazacyclododecane-1,4,7-triyl)triacetic acid (Compound 17)

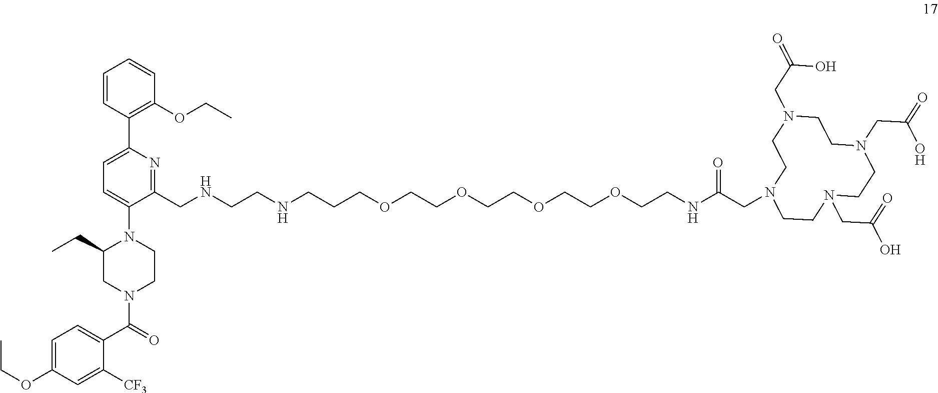 [SMILES: CCOc1ccc(C(=O)N2CCN(c3ccc(-c4ccccc4OCC)nc3CNCCNCCCOCCOCCOCCOCCNC(=O)CN3CCN(CC(=O)O)CCN(CC(=O)O)CCN(CC(=O)O)CC3)[C@H](CC)C2)c(C(F)(F)F)c1]

Step 1: To a DMF (5 mL) solution of 2-(4,7,10-tris(2-(tert-butoxy)-2-oxoethyl)-1,4,7,10-tetraazacyclododecan-1-yl)acetic acid (250 mg, 1.19 Eq, 436. mmol) was added HATU (180 mg, 1.29 Eq, 473. mmol) and DIEA (180 mg, 243. mL, 3.81 Eq, 1.39 mmol). The resulting mixture was stirred at 20° C. for 10 min, followed by the addition of (R)—N-(1-amino-16-((2-nitrophenyl)sulfonyl)-3,6,9,12-tetraoxa-16-azaoctadecan-18-yl)-N-((3-(4-(4-ethoxy-2-(trifluoromethyl)benzoyl)-2-ethylpiperazin-1-yl)-6-(2-ethoxyphenyl)pyridin-2-yl)methyl)-2-nitrobenzenesulfonamide (440 mg, 1 Eq, 366. mmol). The reaction mixture was stirred at 20° C. for 1 hour. The reaction crude was purified by Flash-HPLC using the following conditions: Column, C18 silica gel; mobile phase, water (0.1% FA)/CH₃CN (CH₃CN: 30-98% in 8 min, 98% for 3 min); Detector, UV 254&220 nm. This resulted in tri-tert-butyl 2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-2,5-bis((2-nitrophenyl)sulfonyl)-22-oxo-9,12,15,18-tetraoxa-2,5,21-triazatricosan-23-yl)-1,4,7,10-tetraazacyclododecane-1,4,7-triyl)(R)-triacetate (440 mg, 250 μmol, 68.4%) as a yellow solid. [M+H]=1758.1.

Step 2: To an ACN (5 mL) solution of tri-tert-butyl 2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-2,5-bis((2-nitrophenyl)sulfonyl)-22-oxo-9,12,15,18-tetraoxa-2,5,21-triazatricosan-23-yl)-1,4,7,10-tetraazacyclododecane-1,4,7-triyl)(R)-triacetate (430 mg, 1 Eq, 245 μmol) was added K₂CO₃ (100 mg, 2.96 Eq, 724 μmol) and 2-chlorobenzenethiol (150 mg, 4.24 Eq, 1.04 mmol). The resulting mixture was stirred at 50° C. for 1 hour. The reaction crude was purified by Flash-HPLC using the following conditions: Column, C18 silica gel; mobile phase, water (0.1% FA)/CH₃CN (CH₃CN: 30-70% in 13 min); Detector, UV 254&220 nm. This resulted in tri-tert-butyl 2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-22-oxo-9,12,15,18-tetraoxa-2,5,21-triazatricosan-23-yl)-1,4,7,10-tetraazacyclododecane-1,4,7-triyl)(R)-triacetate (300 mg, 216 μmol, 88.4%) as a yellow solid. [M+H]=1387.7.

Step 3: Into an 8 mL vial were added tri-tert-butyl 2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-22-oxo-9,12,15,18-tetraoxa-2,5,21-triazatricosan-23-yl)-1,4,7,10-tetraazacyclododecane-1,4,7-triyl)(R)-triacetate (290 mg, 1 Eq, 209 μmol) and TFA (3 mL). The resulting mixture was stirred at 30° C. for 3 hrs. The resulting mixture was concentrated under vacuum. The remaining residue was purified by Prep-HPLC using the following conditions: Column, SunFire Prep C18 OBD Column, 19×150 mm 5 μm; mobile phase, Water (0.05% TFA) and ACN (20.0% ACN up to 70.0% in 10 min); Total flow rate, 20 mL/min; Detector, UV 220 nm. This resulted in (R)-2,2′,2″-(10-(1-(3-(4-(4-ethoxy-2-(trifluoromethyl)benzoyl)-2-ethylpiperazin-1-yl)-6-(2-ethoxyphenyl)pyridin-2-yl)-22-oxo-9,12,15,18-tetraoxa-2,5,21-triazatricosan-23-yl)-1,4,7,10-tetraazacyclododecane-1,4,7-triyl)triacetic acid-2,2,2-trifluoroacetic acid (1/2) (170 mg, 117 μmol, 56.2%) as a white solid. [M+H-2TFA]=1219.7.

### Example 122: ¹⁷⁵Lutetium Complex of Compound 17

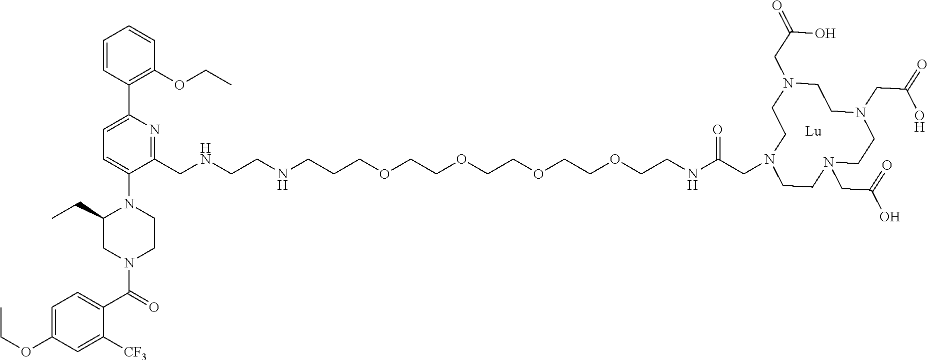

Into an 8 mL flask was added a mixture of (R)-2,2′,2″-(10-(1-(2-(4-(6-ethoxy-2-(trifluoromethyl)nicotinoyl)-2-ethylpiperazin-1-yl)-5-(2-ethoxypyridin-3-yl)phenyl)-22-oxo-9,12,15,18-tetraoxa-2,5,21-triazatricosan-23-yl)-1,4,7,10-tetraazacyclododecane-1,4,7-triyl)triacetic acid (10 mg, 1 Eq, 8.2 μmol), ¹⁷⁵Lutetium (III) chloride (7 mg, 2 μL, 3 Eq, 0.02 mmol), sodium bicarbonate (4 mg, 6 Eq, 0.05 mmol), ACN (0.5 mL) and Water (0.25 mL). The resulting mixture was stirred at 80° C. for 2 hours. The reaction mixture was diluted with 4 mL of DMSO and filtered. The filtrate was purified by Prep-HPLC using the following conditions: Column, SunFire Prep C18 OBD Column, 19×150 mm, 5 μm; mobile phase, Water and ACN (30% ACN up to 80% in 15 min); Total flow rate, 20 mL/min; Detector, UV 220 nm. This resulted in the product (1.7 mg, 1.2 μmol, 15%) as a white solid. [M+H]=1392.7.

### Example 123: ¹¹⁵Indium Complex of Compound 17

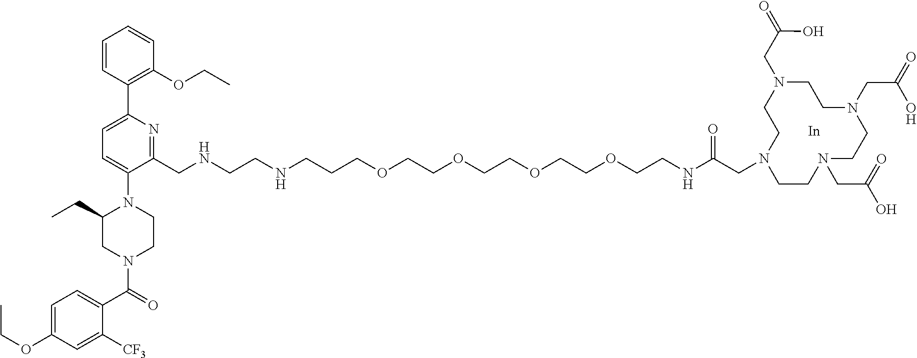

Into an 8 mL flask were added a mixture of (R)-2,2′,2″-(10-(1-(2′-ethoxy-4-(4-(4-ethoxy-2-(trifluoromethyl)benzoyl)-2-ethylpiperazin-1-yl)-(1,1′-biphenyl]-3-yl)-22-oxo-9,12,15,18-tetraoxa-2,5,21-triazatricosan-23-yl)-1,4,7,10-tetraazacyclododecane-1,4,7-triyl)triacetic acid (40 mg, 1 Eq, 33 μmol), ¹¹⁵InCl₃ (25 mg, 7.2 μL, 3.4 Eq, 0.11 mmol), sodium bicarbonate (15 mg, 5.4 Eq, 0.18 mmol), ACN (0.5 mL) and Water (0.25 mL). The resulting mixture was stirred at 80° C. for 2 hours. The reaction mixture was diluted with 4 mL of DMSO and filtered. The filtrate was purified by Prep-HPLC using the following conditions: Column, SunFire Prep C18 OBD Column, 19×150 mm, 5 μm; mobile phase, Water (0.05% TFA) and ACN (30% ACN up to 75% in 15 min); Total flow rate, 20 mL/min; Detector, UV 220 nm. This resulted in indium(III) (R)-2,2′,2″-(10-(1-(2′-ethoxy-4-(4-(4-ethoxy-2-(trifluoromethyl)benzoyl)-2-ethylpiperazin-1-yl)-[1,1′-biphenyl]-3-yl)-22-oxo-9,12,15,18-tetraoxa-2,5,21-triazatricosan-23-yl)-1,4,7,10-tetraazacyclododecane-1,4,7-triyl)triacetate (41.9 mg, 26.9 μmol, 82%) as a white solid. [M+H-2TFA]=1330.7.

### Example 124: tert-butyl (R)-(1-(2′-ethoxy-5-(4-(6-ethoxy-2-(trifluoromethyl)nicotinoyl)-2-ethylpiperazin-1-yl)-[2,3′-bipyridin]-6-yl)-2-((2-nitrophenyl)sulfonyl)-6,9,12,15-tetraoxa-2-azaheptadecan-17-yl)carbamate

Into a 40 mL vial, were added (R)—N-((2′-ethoxy-5-(4-(6-ethoxy-2-(trifluoromethyl)-nicotinoyl)-2-ethylpiperazin-1-yl)-[2,3′-bipyridin]-6-yl)methyl)-2-nitrobenzenesulfonamide (300 mg, 1 Eq, 403 μmol), ACN (3 mL), Cs₂CO₃ (400 mg, 3.04 Eq, 1.23 mmol), and 2,2-dimethyl-4-oxo-3,8,11,14,17-pentaoxa-5-azaicosan-20-yl methanesulfonate (350 mg, 2.02 Eq, 815 μmol). The resulting mixture was stirred for 4 hours at 80° C. The reaction crude was purified by Prep-HPLC using the following conditions (Flash-HPLC-013): Column, C18 OBD Column, 19×150 mm, 5 μm; mobile phase, Water (0.1% FA) and ACN (30.0% ACN up to 98.0% in 8 min); Total flow rate, 70 mL/min; Detector, UV 220 nm. Fractions collected were combined and concentrated under vacuum. This resulted in the title compound (300 mg, 279 μmol, 69.0%) as a yellow solid. [M+H]=1077.4.

### Example 125: (R)—N-(1-amino-3,6,9,12-tetraoxapentadecan-15-yl)-N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide

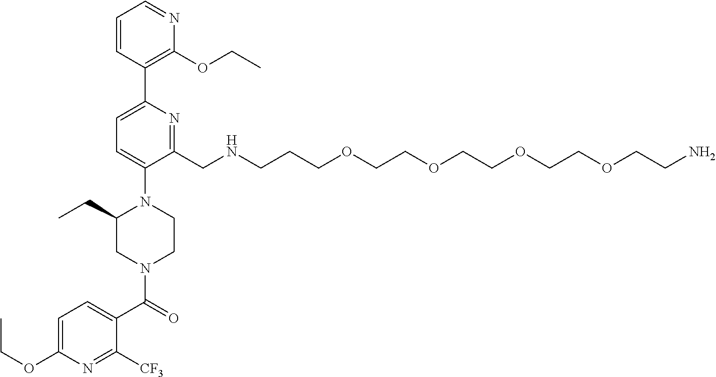 [SMILES: CCOc1ccc(C(=O)N2CCN(c3ccc(-c4cccnc4OCC)nc3CNCCCOCCOCCOCCOCCN)[C@H](CC)C2)c(C(F)(F)F)n1]

Into a 100 mL round bottom flask, were added tert-butyl (R)-(1-(2′-ethoxy-5-(4-(6-ethoxy-2-(trifluoromethyl)nicotinoyl)-2-ethylpiperazin-1-yl)-[2,3′-bipyridin]-6-yl)-2-((2-nitrophenyl)sulfonyl)-6,9,12,15-tetraoxa-2-azaheptadecan-17-yl)carbamate (290 mg, 1 Eq, 269 μmol), DCM (3 mL) and TFA (1 mL). The resulting mixture was stirred at 20° C. for 1 hour. The reaction mixture was concentrated under vacuum and diluted with water (20 mL). Saturated aq NaHCO₃ was added until pH ˜7 and the resulting solution was extracted with ethyl acetate (3×30 mL). Organic layers were combined, washed with brine (2×20 mL), dried over anhydrous sodium sulfate and concentrated under vacuum. This resulted in (R)—N-(1-amino-3,6,9,12-tetraoxapentadecan-15-yl)-N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (250 mg, 256 μmol, 95.0%) as light yellow solid. [M+H]=977.9.

### Example 126: 2,2′,2″-(10-(22-carboxy-1-(2′-ethoxy-5-((R)-4-(6-ethoxy-2-(trifluoromethyl)-nicotinoyl)-2-ethylpiperazin-1-yl)-(2,3′-bipyridin]-6-yl)-19-oxo-6,9,12,15-tetraoxa-2,18-diazadocosan-22-yl)-1,4,7,10-tetraazacyclododecane-1,4,7-triyl)triacetic acid (Compound 18)

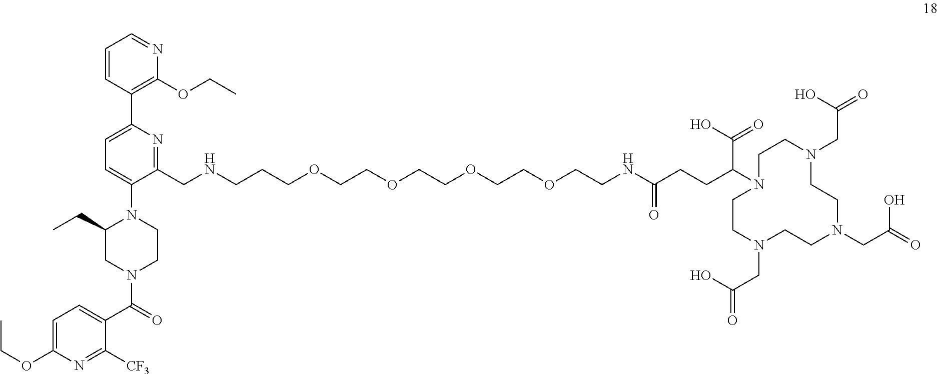 [SMILES: CCOc1ccc(C(=O)N2CCN(c3ccc(-c4cccnc4OCC)nc3CNCCCOCCOCCOCCOCCNC(=O)CCC(C(=O)O)N3CCN(CC(=O)O)CCN(CC(=O)O)CCN(CC(=O)O)CC3)[C@H](CC)C2)c(C(F)(F)F)n1]

Step 1: To a DMF (2 mL) solution of 5-(tert-butoxy)-5-oxo-4-(4,7,10-tris(2-(tert-butoxy)-2-oxoethyl)-1,4,7,10-tetraazacyclododecan-1-yl)pentanoic acid (160 mg, 1.12 Eq, 228 μmol) was added 2-(3H-[1,2,3]triazolo[4,5-b]pyridin-3-yl)-1,1,3,3-tetramethylisouronium hexafluorophosphate(V) (100 mg, 1.28 Eq, 263 μmol) and N-ethyl-N-isopropylpropan-2-amine (100 mg, 3.78 Eq, 774 μmol). The resulting mixture was stirred at 20° C. for 10 min, followed by the addition of (R)—N-(1-amino-3,6,9,12-tetraoxapentadecan-15-yl)-N-((2′-ethoxy-5-(4-(6-ethoxy-2-(trifluoromethyl)nicotinoyl)-2-ethylpiperazin-1-yl)-[2,3′-bipyridin]-6-yl)methyl)-2-nitrobenzenesulfonamide (200 mg, 1 Eq, 205 μmol). The reaction mixture was stirred at 20° C. for 1 hour. The reaction crude was purified by Flash-HPLC using the following conditions (IntelFlash-1): Column, C18 silica gel; mobile phase, water (0.1% FA)/CH₃CN (CH₃CN: 30-98% in 8 min, 98% in 3 min); Detector, UV 254 &220 nm. This resulted in tri-tert-butyl 2,2′,2″-(10-(1-(2′-ethoxy-5-((R)-4-(6-ethoxy-2-(trifluoromethyl)nicotinoyl)-2-ethylpiperazin-1-yl)-[2,3′-bipyridin]-6-yl)-25,25-dimethyl-2-((2-nitrophenyl)sulfonyl)-19,23-dioxo-6,9,12,15,24-pentaoxa-2,18-diazahexacosan-22-yl)-1,4,7,10-tetraazacyclododecane-1,4,7-triyl)triacetate (150 mg, 90.4 μmol, 44.1%) as a yellow solid. [M+H]=1660.1.

Step 2: Into an 8 mL vial, were placed tri-tert-butyl 2,2′,2″-(10-(1-(2′-ethoxy-5-((R)-4-(6-ethoxy-2-(trifluoromethyl)nicotinoyl)-2-ethylpiperazin-1-yl)-[2,3′-bipyridin]-6-yl)-25,25-dimethyl-2-((2-nitrophenyl)sulfonyl)-19,23-dioxo-6,9,12,15,24-pentaoxa-2,18-diazahexacosan-22-yl)-1,4,7,10-tetraazacyclododecane-1,4,7-triyl)triacetate (140 mg, 1 Eq, 84.3 μmol), ACN (2 mL), K₂CO₃ (50 mg, 4.3 Eq, 0.36 mmol), and 2-chlorobenzenethiol (50 mg, 4.1 Eq, 0.35 mmol). The resulting mixture was stirred at 50° C. for 1 hour. The reaction crude was purified by Flash-HPLC using the following conditions: Column, C18 OBD Column; mobile phase, water (0.1% FA)/CH₃CN (CH₃CN: 30-70% in 13 min); Detector, UV 254&220 nm. This resulted in tri-tert-butyl 2,2′,2″-(10-(1-(2′-ethoxy-5-((R)-4-(6-ethoxy-2-(trifluoromethyl)nicotinoyl)-2-ethylpiperazin-1-yl)-[2,3′-bipyridin]-6-yl)-25,25-dimethyl-19,23-dioxo-6,9,12,15,24-pentaoxa-2,18-diazahexacosan-22-yl)-1,4,7,10-tetraazacyclododecane-1,4,7-triyl)triacetate (130 mg, 79 μmol, 94%, 90% Purity) as a yellow solid. [M+H]=1474.8.

Step 3: Into an 8 mL vial, were added tri-tert-butyl 2,2′,2″-(10-(1-(2′-ethoxy-5-((R)-4-(6-ethoxy-2-(trifluoromethyl)nicotinoyl)-2-ethylpiperazin-1-yl)-(2,3′-bipyridin]-6-yl)-25,25-dimethyl-19,23-dioxo-6,9,12,15,24-pentaoxa-2,18-diazahexacosan-22-yl)-1,4,7,10-tetraazacyclododecane-1,4,7-triyl)triacetate (120 mg, 1 Eq, 81.4 μmol) and TFA (2 mL). The resulting mixture was stirred at 30° C. for 2 hours. The reaction mixture was concentrated under vacuum. The remaining residue was purified by Prep-HPLC using the following conditions (Flash-HPLC-013): Column, SunFire Prep C18 OBD Column, 19×150 mm 5 μm; mobile phase, Water (0.05% TFA) and ACN (20.0% ACN up to 68.0% in 10 min); Total flow rate, 20 mL/min; Detector, UV 220 nm. This resulted in 2,2′,2″-(10-(22-carboxy-1-(2′-ethoxy-5-((R)-4-(6-ethoxy-2-(trifluoromethyl)nicotinoyl)-2-ethylpiperazin-1-yl)-(2,3′-bipyridin]-6-yl)-19-oxo-6,9,12,15-tetraoxa-2,18-diazadocosan-22-yl)-1,4,7,10-tetraazacyclododecane-1,4,7-triyl)triacetic acid-2,2,2-trifluoroacetic acid (1/2) (60 mg, 41 μmol, 50%) as a white solid. [M+H-2TFA]=1250.7.

### Example 127: ^{69/71}Gallium Complex of Compound 18

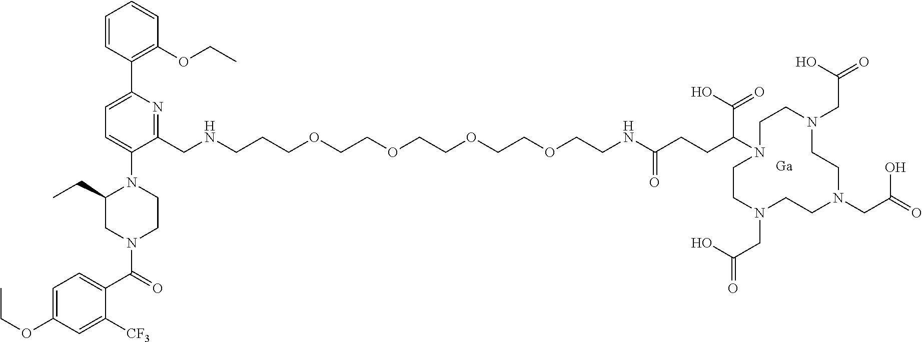

Into an 8 mL flask was added a mixture of 2,2′,2″-(10-(22-carboxy-1-(2′-ethoxy-5-((R)-4-(6-ethoxy-2-(trifluoromethyl)nicotinoyl)-2-ethylpiperazin-1-yl)-(2,3′-bipyridin]-6-yl)-19-oxo-6,9,12,15-tetraoxa-2,18-diazadocosan-22-yl)-1,4,7,10-tetraazacyclododecane-1,4,7-triyl)triacetic acid (10 mg, 1 Eq, 8.0 μmol), ^{69/71}Gallium chloride (5 mg, 2 μL, 4 Eq, 0.03 mmol), Sodium bicarbonate (4 mg, 2 μL, 6 Eq, 0.05 mmol), Water (0.1 mL) and ACN (0.2 mL). The resulting mixture was stirred at 80° C. for 2 hours. The reaction mixture was diluted with 4 mL of DMSO and filtered. The filtrate was purified by Prep-HPLC using the following conditions (Prep-HPLC-007): Column, SunFire Prep C18 OBD Column, 19×150 mm, 5 μm; mobile phase, Water and ACN (30% ACN up to 80% in 15 min); Total flow rate, 20 mL/min; Detector, UV 220 nm. This resulted in gallium(IV) 2,2′,2″-(10-(22-carboxylato-1-(2′-ethoxy-5-((R)-4-(6-ethoxy-2-(trifluoromethyl)nicotinoyl)-2-ethylpiperazin-1-yl)-[2,3′-bipyridin]-6-yl)-19-oxo-6,9,12,15-tetraoxa-2,18-diazadocosan-22-yl)-1,4,7,10-tetraazacyclododecane-1,4,7-triyl)triacetate (6.8 mg, 5.2 μmol, 65%) as a white solid. [M+H]=1316.6.

### Example 128: ¹¹⁵Indium Complex of Compound 18

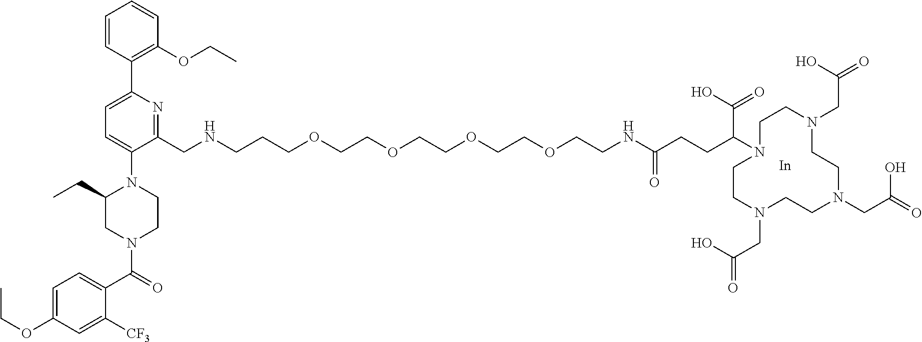

Into an 8 mL flask was added a mixture of 2,2′,2″-(10-(22-carboxy-1-(2′-ethoxy-5-((R)-4-(6-ethoxy-2-(trifluoromethyl)nicotinoyl)-2-ethylpiperazin-1-yl)-[2,3′-bipyridin]-6-yl)-19-oxo-6,9,12,15-tetraoxa-2,18-diazadocosan-22-yl)-1,4,7,10-tetraazacyclododecane-1,4,7-triyl)triacetic acid (10 mg, 1 Eq, 8.0 μmol), ¹¹⁵InCl₃ (6 mg, 2 μL, 3 Eq, 0.03 mmol), Sodium bicarbonate (5 mg, 2 μL, 7 Eq, 0.06 mmol), ACN (0.2 mL) and Water (0.1 mL). The resulting mixture was stirred at 80° C. for 2 hours. The reaction mixture was diluted with 4 mL of DMSO and filtered. The filtrate was purified by Prep-HPLC using the following conditions (Prep-HPLC-007): Column, SunFire Prep C18 OBD Column, 19×150 mm 5 μm 10 nm; mobile phase, Water (0.05% TFA) and ACN (30% ACN up to 75% in 15 min); Total flow rate, 20 mL/min; Detector, UV 220 nm. This resulted in the TFA salt of indium(IV) 2,2′,2″-(10-(22-carboxylato-1-(2′-ethoxy-5-((R)-4-(6-ethoxy-2-(trifluoromethyl)nicotinoyl)-2-ethylpiperazin-1-yl)-[2,3′-bipyridin]-6-yl)-19-oxo-6,9,12,15-tetraoxa-2,18-diazadocosan-22-yl)-1,4,7,10-tetraazacyclododecane-1,4,7-triyl)triacetate (8.9 mg, 5.6 μmol, 70%) as a white solid. [M+H-2TFA]=1362.6.

### Example 129: ¹¹¹In[In] Complex of Compound 1

[¹¹¹In]InCl₃ (56.8 MBq, 89.7 μL, 0.1 M HCl) and Compound 1 (4 nmol, 4 μL, 1.0 mM in DI water) were added to a NH₄OAc solution (9 μL, 1.0 M). The resulting mixture had a pH of 5.0˜5.5, and was heated at 85° C. in a thermal mixer for 30 min. The radiochemical purity was 96.7% determined by radio HPLC. The radiotracer solution for in vivo studies was prepared by dilution with 0.9% saline.

### Example 130: ¹¹¹In[In] Complex of Compound 9

[¹¹¹In]InCl₃ (39 MBq, 105.4 μL, 0.1 M HCl) and Compound 9 (2.7 nmol, 2.7 μL, 1.0 mM in DI water) were added to a NH₄OAc solution (10.8 μL, 2.5 M, 50 mM ascorbic acid, 50 mM gentisic acid). The resulting mixture had a pH of 5.0˜5.5, and was heated at 75° C. in a thermal mixer for 30 min. At the end of labeling, Ca-DTPA (12 μL, 4 mM) was added. The radiochemical purity was 93.5% determined by radio HPLC.

### Example 131: ¹⁷⁷Lu[Lu] Complex of Compound 9

[¹⁷⁷Lu]LuCl₃ (248.7 MBq, 124.4 μL, 0.1 M HCl) and Compound 9 (1.39 nmol, 13.9 μL, 0.1 mM in DI water) were added to a NH₄OAc solution (387.2 μL, 0.4 M, 0.325 M gentisic acid). The resulting mixture had a pH of 3.9˜4.2, and was heated at 80° C. in a thermal mixer for 30 min. At the end of labeling, Ca-DTPA (12.4 μL, 4 mM) was added. The radiochemical purity was 98.9% determined by radio HPLC.

### Example 132: ⁶⁸Ga[Ga] Complex of Compound 9

[⁶⁸Ga]GaCl₃ (9.2 MBq, 50.0 μL, 0.1 M HCl-5 M NaCl fractionated elution), ethanol (5.0 μL) and Compound 9 (1.0 nmol, 1 μL, 1.0 mM in DI water) were added to a NaOAc solution (15.0 μL, 1.0 M). The resulting mixture had a pH of 3.9, and was heated at 90° C. in a thermal mixer for 10 min. At the end of labeling, EDTA (2.5 μL, 50 mM) was added. The radiochemical purity was 99% determined by radio HPLC.

### Example 133: ¹¹¹In[In] Complex of Compound 10

[¹¹¹In]InCl₃ (120 MBq, 191 μL, 0.1 M HCl) and Compound 10 (4.0 nmol, 4.0 μL, 1.0 mM in DI water) were added to a NaOAc solution (19.1 μL, 2.5 M, 50 mM sodium ascorbate, 50 mM gentisic acid). The resulting mixture had a pH of 5.0˜5.5, and was heated at 80° C. in a thermal mixer for 30 min. At the end of labeling, Ca-DTPA (19.1 μL, 4 mM) was added. The radiochemical purity was 95% determined by radio HPLC.

### Example 134: ¹⁷⁷Lu[Lu] Complex of Compound 10

[¹⁷⁷Lu]LuCl₃ (248.3 MBq, 124.4 μL, 0.1 M HCl) and Compound 10 (1.39 nmol, 13.9 μL, 0.1 mM in DI water) were added to a NH₄OAc solution (387.2 μL, 0.4 M, 0.325 M gentisic acid). The resulting mixture had a pH of 3.9˜4.2, and was heated at 80° C. in a thermal mixer for 30 min. At the end of labeling, Ca-DTPA (12.4 μL, 4 mM) was added. The radiochemical purity was 99.0% determined by radio HPLC.

### Example 135: ⁶⁸Ga[Ga] Complex of Compound 10

[⁶⁸Ga]GaCl₃ (15 MBq, 100.0 μL, 0.1 M HCl fractionated elution), ethanol (10.0 μL) and Compound 10 (2 nmol, 2 μL, 1.0 mM in DI water) were added to a NaOAc solution (11.0 μL, 1.0 M). The resulting mixture had a pH of 3.6, and was heated at 90° C. in a thermal mixer for 10 min. At the end of labeling, EDTA (5 μL, 50 mM) was added. The radiochemical purity was 96% determined by radio HPLC.

### Example A-1: Parenteral Pharmaceutical Composition

To prepare a parenteral pharmaceutical composition suitable for administration by injection (subcutaneous, intravenous), 0.001-500 mg of a compound Formula (I), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection

### BIOLOGY EXAMPLES

### Example B-1: Measurement of Antagonist Inhibition of Binding of ACTH in Human MC2R-Containing Membranes

Crude membrane fractions are prepared from CellSensor CRE-blaCHO-K1 cells stably expressing functional hMC2 receptor (ThermoFisher #K1483). The cells are cultured to 85-100% confluency in growth media [GlutaMax DMEM (Gibco #10569-010) supplemented with 10% dialyzed fetal bovine serum (Gemini Bio-Products #100-108), 0.1 mM non-essential amino acids (Gibco #11140-050), 100 U/mL penicillin; 100 μg/mL streptomycin; 2 mM L-glutamine (Gemini Bio-Products #400-110), 5 μg/mL blasticidin (GoldBio #B-800-100), 100 μg/mL zeocin (Invitogen #R25005) and 600 μg/mL hygromycin B (GoldBio #31282-04-9)]. Cells were collected and washed in Dulbecco's phosphate buffered saline (Corning #21-031-CV). The cell pellet is reconstituted in membrane preparation buffer [20 mM HEPES (Biopioneer #C0113), 6 mM MgCl2 (Sigma #M8266) and 1 mM EDTA (JT Baker #4040-01), protease inhibitor tablets (Pierce #A32963); pH7.4)] and homogenized using a Dounce homogenizer. The membrane fraction is separated from cell debris and collected in membrane preparation buffer, flash frozen in liquid nitrogen and stored at −80° C. The inhibition of binding to the human MC2R receptor was measured in a radioactive competition binding assay using the radiolabeled [125I]-TYR23]-ACTH (1-39) ligand (PerkinElmer #NEX083, custom synthesis) as the probe ligand for the receptor. Briefly, membranes from cells expressing the human MC2R were incubated with SPA beads (PerkinElmer #RPNQ0001) in binding assay buffer [50 mM HEPES (Biopioneer #C0113), 5 mM MgCl2 (Sigma #M8266), 1 mM CaCl2) (Fisher Scientific #BP510), 0.2% BSA (Fisher Scientific #BP1600), and protease inhibitors (Pierce #A32963); pH 7.4]. Membranes bound to SPA bead were treated with various dilutions of compounds (final concentrations typically 0-10,000 nM), and 0.2 nM radiolabeled ligand in 96-well isoplates (PerkinElmer #6005040) for 90 minutes at room temperature. The radioactive signal was detected using a MicroBetaTriLux 1450 LSC (PerkinElmer). All data manipulations to determine the Kivalues were performed using GraphPad v8 (GraphPad, San Diego CA)

The metal complexes in Table B comprises nonradioactive gallium, indium, and lutetium.

TABLE B
Representative Binding Activity

| | | hMC2 |
|---|---|---|
| | | membrane |
| Cmpd No. | Compound Structure | binding (nM) |
| 1 | 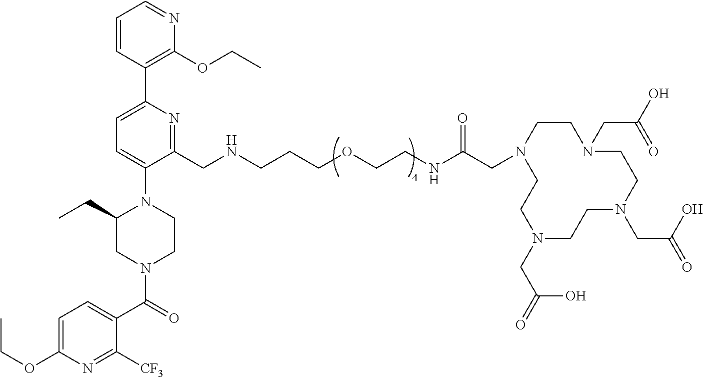 | <10 |
| | ¹¹⁵In complex of Compound 1 | <10 |
| | ¹⁷⁵Lu complex of Compound 1 | <10 |
| 2 | 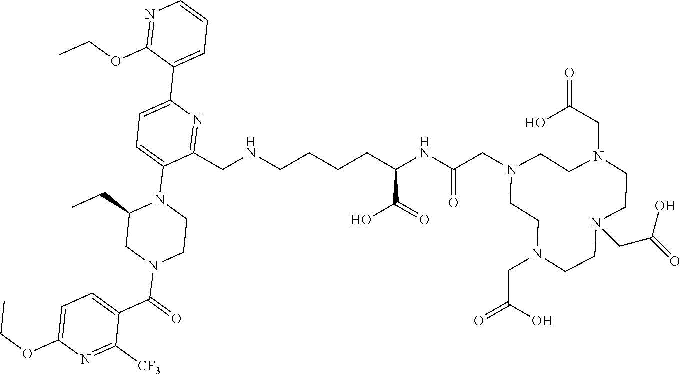 [SMILES: CCOc1ccc(C(=O)N2CCN(c3ccc(-c4cccnc4OCC)nc3CNCCCC[C@@H](NC(=O)CN3CCN(CC(=O)O)CCN(CC(=O)O)CCN(CC(=O)O)CC3)C(=O)O)[C@H](CC)C2)c(C(F)(F)F)n1] | |
| | ¹¹⁵In complex of Compound 2 | <10 |
| 3 | 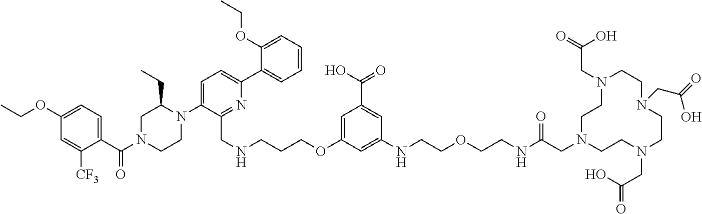 [SMILES: CCOc1ccc(C(=O)N2CCN(c3ccc(-c4ccccc4OCC)nc3CNCCCOc3cc(NCCOCCNC(=O)CN4CCN(CC(=O)O)CCN(CC(=O)O)CCN(CC(=O)O)CC4)cc(C(=O)O)c3)[C@H](CC)C2)c(C(F)(F)F)c1] | |
| | ¹¹⁵In complex of Compound 3 | <10 |
| 4 | 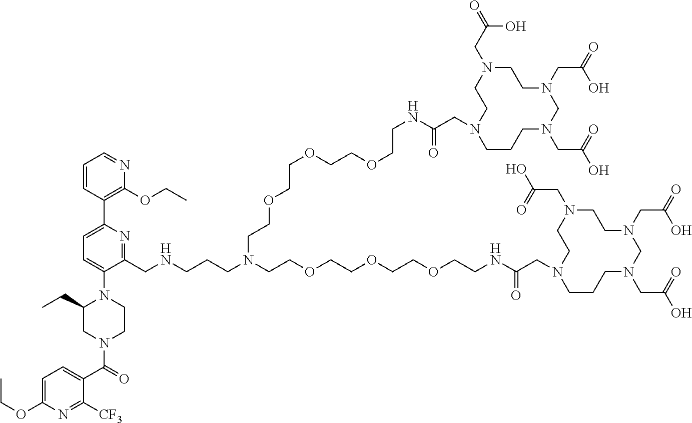 [SMILES: CCOc1ccc(C(=O)N2CCN(c3ccc(-c4cccnc4OCC)nc3CNCCCN(CCOCCOCCOCCNC(=O)CN3CCCN(CC(=O)O)CN(CC(=O)O)CCN(CC(=O)O)CC3)CCOCCOCCOCCNC(=O)CN3CCCN(CC(=O)O)CN(CC(=O)O)CCN(CC(=O)O)CC3)[C@H](CC)C2)c(C(F)(F)F)n1] | |
| | ¹¹⁵In complex of Compound 4 | <10 |
| 5 | 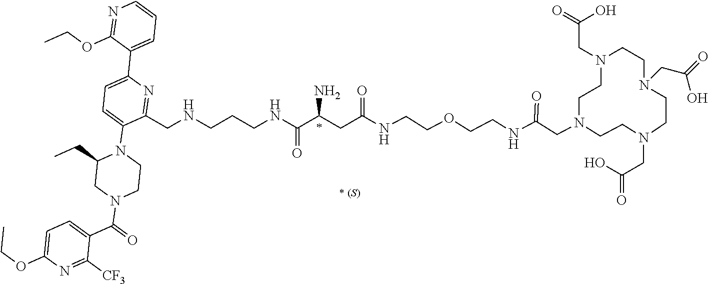 [SMILES: CCOc1ccc(C(=O)N2CCN(c3ccc(-c4cccnc4OCC)nc3CNCCCNC(=O)[C@@H](N)CC(=O)NCCOCCNC(=O)CN3CCN(CC(=O)O)CCN(CC(=O)O)CCN(CC(=O)O)CC3)[C@H](CC)C2)c(C(F)(F)F)n1] | |
| | ¹¹⁵In complex of Compound 5 | <10 |
| 6 | 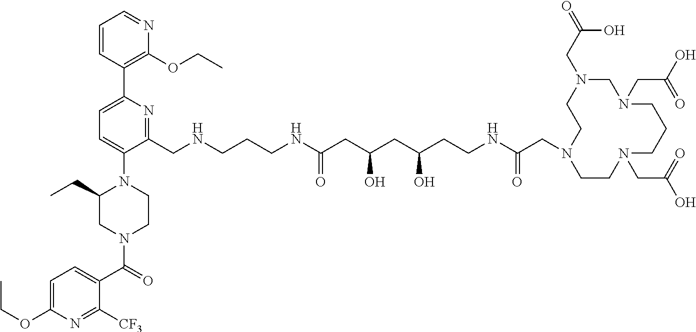 [SMILES: CCOc1ccc(C(=O)N2CCN(c3ccc(-c4cccnc4OCC)nc3CNCCCNC(=O)C[C@H](O)C[C@H](O)CCNC(=O)CN3CCN(CC(=O)O)CCCN(CC(=O)O)CN(CC(=O)O)CC3)[C@H](CC)C2)c(C(F)(F)F)n1] | <10 |
| | ¹¹⁵In complex of Compound 6 | <10 |
| 7 | 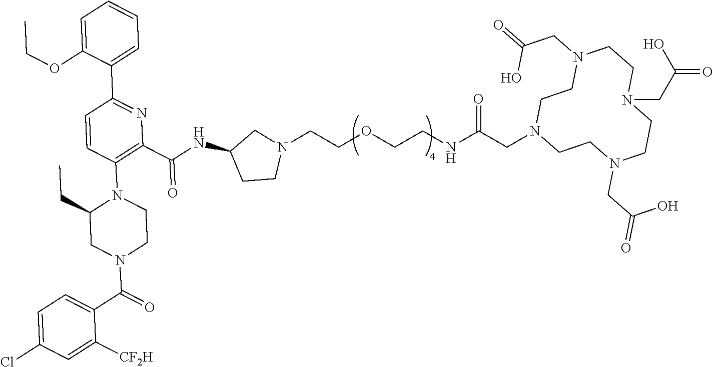 | |
| | ¹¹⁵In complex of Compound 7 | <10 |
| 8 | 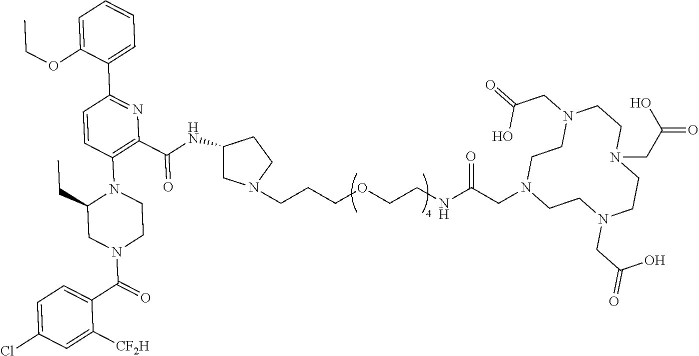 | |
| | ¹¹⁵In complex of Compound 8 | <10 |
| 9 | 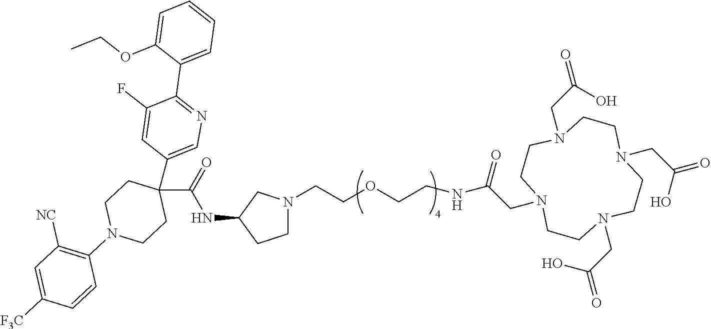 | <10 |
| | ¹¹⁵In complex of Compound 9 | <10 |
| 10 | 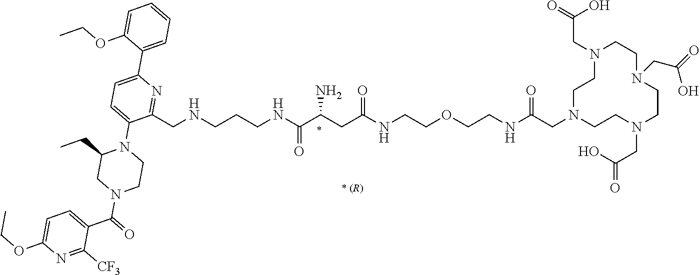 [SMILES: CCOc1ccc(C(=O)N2CCN(c3ccc(-c4ccccc4OCC)nc3CNCCCNC(=O)[C@H](N)CC(=O)NCCOCCNC(=O)CN3CCN(CC(=O)O)CCN(CC(=O)O)CCN(CC(=O)O)CC3)[C@H](CC)C2)c(C(F)(F)F)n1] | <10 |
| | ¹¹⁵In complex of Compound 10 | <10 |
| 11 | 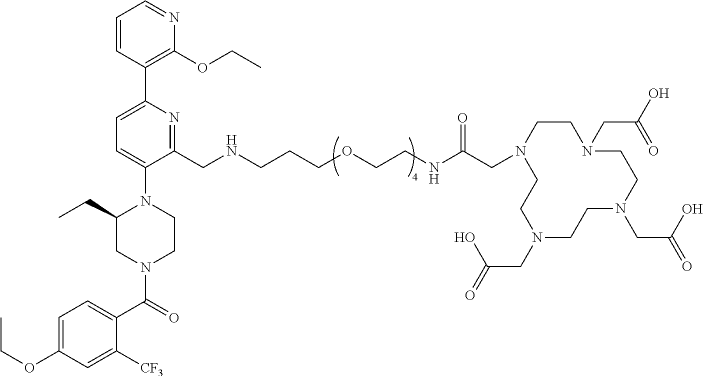 | <10 |
| | ¹¹⁵In complex of Compound 11 | <10 |
| 12 | 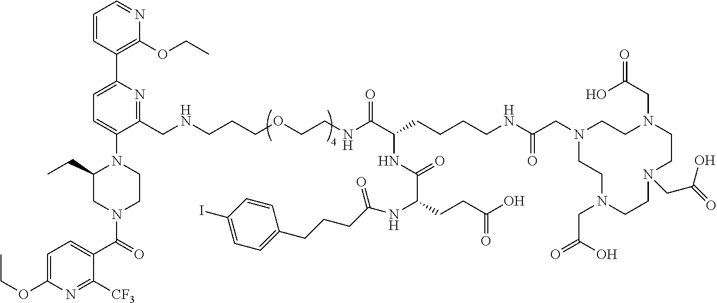 | <10 |
| 13 | 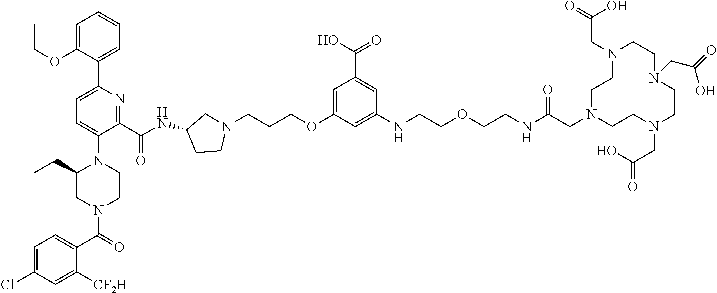 [SMILES: CCOc1ccccc1-c1ccc(N2CCN(C(=O)c3ccc(Cl)cc3C(F)F)C[C@H]2CC)c(C(=O)N[C@H]2CCN(CCCOc3cc(NCCOCCNC(=O)CN4CCN(CC(=O)O)CCN(CC(=O)O)CCN(CC(=O)O)CC4)cc(C(=O)O)c3)C2)n1] | <10 |
| | ¹¹⁵In complex of Compound 13 | <10 |
| 14 | 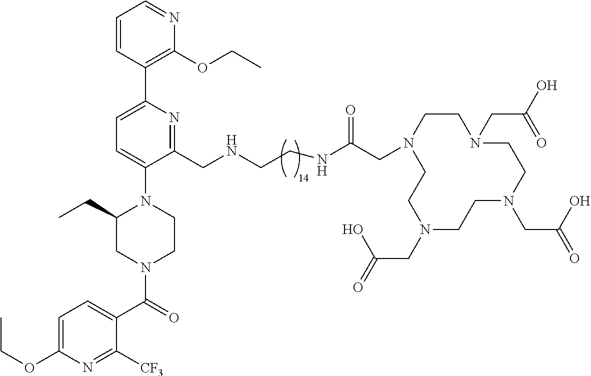 | <10 |
| | ¹¹⁵In complex of Compound 14 | <10 |
| 15 | 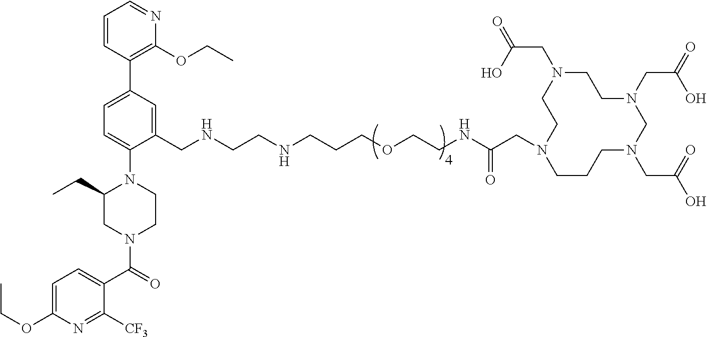 | <10 |
| | ¹¹⁵In complex of Compound 15 | <10 |
| | ¹⁷⁵Lu complex of Compound 15 | <10 |
| | ^{69/71}Ga complex of Compoud 15 | <10 |
| 16 | 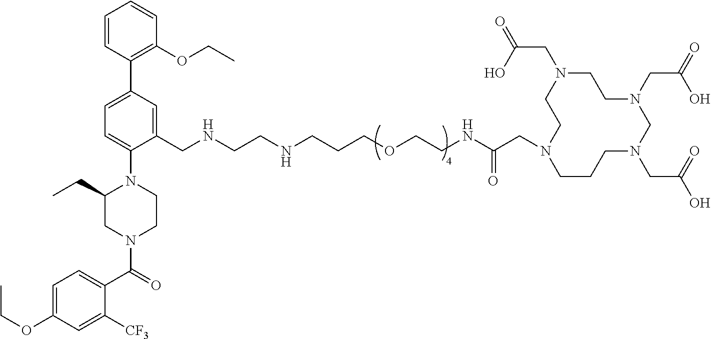 | <10 |
| 17 | 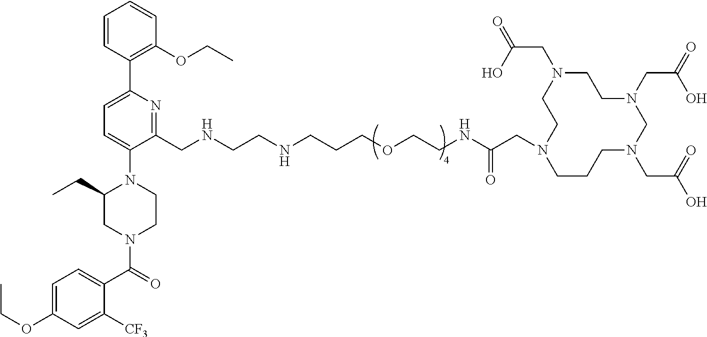 | <10 |
| | ¹¹⁵In complex of Compound 17 | <10 |
| 18 | 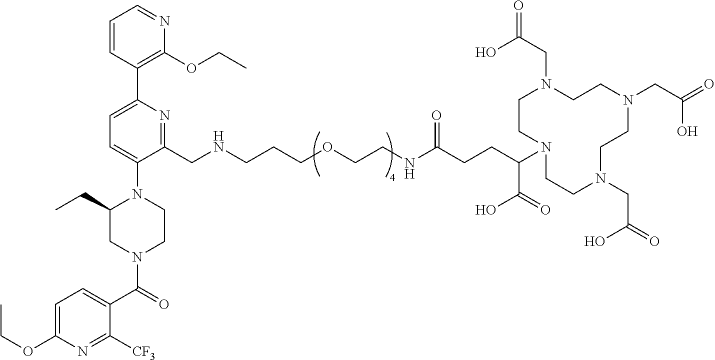 | <10 |
| | ¹¹⁵In complex of Compound 18 | <10 |
| | ¹⁷⁵Lu complex of Compound 18 | <10 |
| | ^{69/71}Ga complex of Compound 18 | <10 |
| 19 | 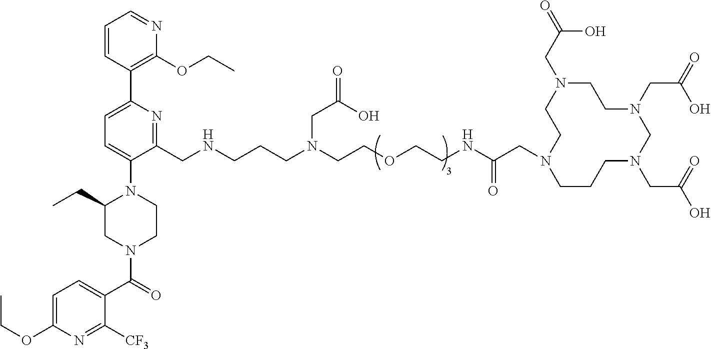 | |
| | ¹¹⁵In complex of Compound 19 | <10 |

### Example B-2: Biodistribution in Healthy Sprague Dawley Rats

Biodistribution study outline: 24 hours prior to the start of the biodistribution study, the study compound (CMPD) was radiolabeled as described herein. On the day of the study animals received a single IV injection of 200 uL into the caudal vein via a catheter with 5 MBq of the ¹¹¹In[In] complex of Compound 1 (1 nmol) per animal.

| Group | # of animals | Readout Time | | MBq/animal | Route/Schedule |
|---|---|---|---|---|---|
| 1 | 3 | 0.5 | hour | 5 | IV/Q1Dx1 |
| 2 | 3 | 5.5 | hours | 5 | IV/Q1Dx1 |
| 3 | 3 | 24 | hours | 5 | IV/Q1Dx1 |

After drug administration, animals were euthanized at timepoints (0.5 hour, 5.5 hours, 24 hours) and organs (Brain, Heart, Femur, Liver, Lungs, Kidneys, Blood, Adrenals, Eyes) were collected, weighed, and radioactivity was assessed in each organ/tissue. Activity was quantitated and expressed as % ID/g (Percentage of Initial Dose/gram of tissue).

TABLE 1
Biodistribution of ¹¹¹In[In] complex of Compound 1. Percentage
of injected dose per mass of whole organ (% ID/g)

| Time Post Inoculation | | |
|---|---|---|
| 0.5 h | 5.5 h | 24 h |

| Region | AVG | SD | AVG | SD | AVG | SD |
|---|---|---|---|---|---|---|
| Brain | 0.027 | 0.007 | 0.002 | 0.001 | 0.001 | 0.000 |
| Heart | 0.145 | 0.047 | 0.013 | 0.001 | 0.013 | 0.001 |
| Femur | 0.091 | 0.021 | 0.019 | 0.002 | 0.020 | 0.002 |
| Liver | 0.650 | 0.200 | 0.245 | 0.053 | 0.297 | 0.063 |
| Lungs | 0.315 | 0.060 | 0.059 | 0.009 | 0.065 | 0.016 |
| Tail | 0.237 | 0.077 | 0.053 | 0.017 | 0.019 | 0.005 |
| Kidneys | 1.156 | 0.196 | 0.312 | 0.026 | 0.339 | 0.020 |
| Blood | 0.350 | 0.102 | 0.006 | 0.001 | 0.004 | 0.002 |
| Adrenals | 8.187 | 2.278 | 1.719 | 0.483 | 0.123 | 0.034 |
| Eyes | 0.066 | 0.018 | 0.012 | 0.001 | 0.010 | 0.001 |

### Example B-3: Biodistribution of ¹¹¹In[In] Complex of Compound 10 in Non Tumor-Bearing Sprague Dawley Rats

Study outline: 24 hours prior to the start of the biodistribution study, the study compound (CMPD) was radiolabeled as described herein. On the day of the study animals received a single IV injection of 200 uL into the caudal vein via a catheter with 5 MBq of ¹¹¹In[In] complex of Compound 10 (1 nmol) per animal. The blocking study (Group 5) combined 1 nmol of ¹¹¹In[In] complex of Compound 10 (5 MBq) with 100 nmol 115In-Compound 10.

| | # of | Readout | | | | Route/ |
|---|---|---|---|---|---|---|
| Group | animals | Time | | MBq/animal | nmol/animal | Schedule |
| 1 | 3 | 0.5 | hours | 5 | 1 | IV/Q1Dx1 |
| 2 | 3 | 3 | hours | 5 | 1 | IV/Q1Dx1 |
| 3 | 3 | 5 | hours | 5 | 1 | IV/Q1Dx1 |
| 4 | 3 | 24 | hours | 5 | 1 | IV/Q1Dx1 |
| 5 | 3 | 3 | hours | 5 + 0 | 1 + 100 | IV/Q1Dx1 |

After drug administration, animals were euthanized at timepoints (0.5 hour, 3 hours, 5 hours, 24 hours) and organs (brain, heart, femur, liver, lungs, kidneys, blood, adrenals, colon, eyes, tail) were collected, weighed, and radioactivity was assessed in each organ/tissue. Activity was quantitated and expressed as % ID/g (Percentage of Initial Dose/gram of tissue).

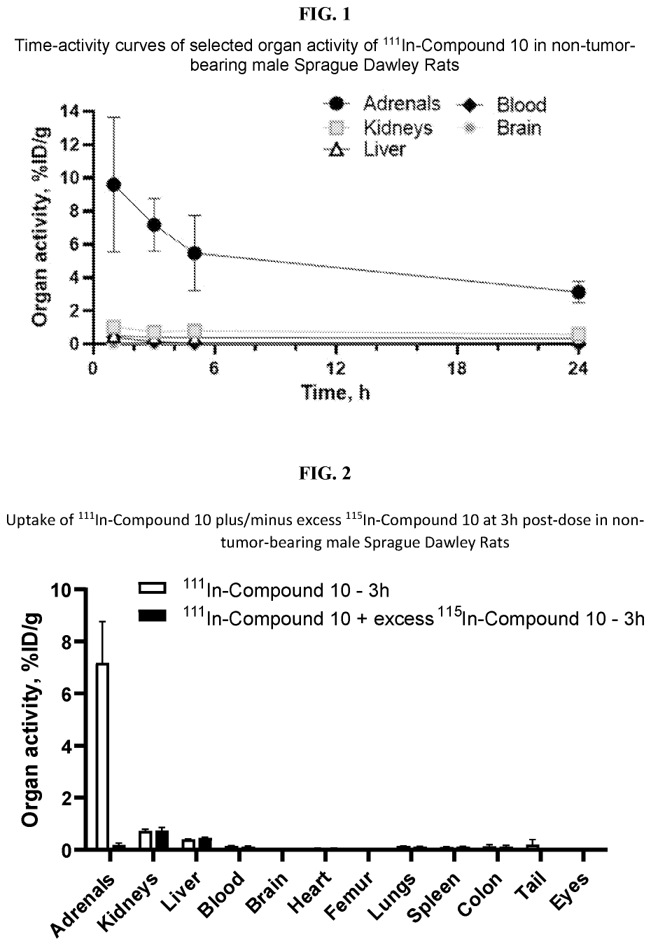

Biodistribution Results: In the non tumor-bearing Sprague Dawley rat ¹¹¹In[In] complex of Compound 10 showed high and sustained MC2R-mediated uptake in the adrenal glands, the only known site of MC2R expression in healthy rodents. The uptake of ¹¹¹In[In] complex of Compound 10 was demonstrated to be MC2R-mediated by selective blocking, as shown in competition studies where a 100-fold molar excess of non-radioactive ¹¹⁵In-Compound 10 was co-administered (FIG. 1.). The studies in rat also demonstrated minimal to no measurable uptake in MC2R-negative tissues.

Uptake in MC2R-expressing adrenals glands with minimal uptake in other non-MC2R expressing organs. Mean±SD n=3 (see FIG. 2). Organ activity, as expressed in % ID/g, at 3 h post-dose. Selective block of ¹¹¹In[In] complex of Compound 10 uptake in the presence of excess ¹¹⁵In-Compound 10 shows MC2R-mediated uptake in adrenals glands, with minimal to no uptake uptake in other non-MC2R positive organs. Mean±SD n=3.

### Example B-4: Biodistribution of ¹⁷⁷Lu[Lu] Complex of Compound 10 in Non Tumor-Bearing Sprague Dawley Rats

Study outline: 24 hours prior to the start of the biodistribution study, the study compound (CMPD) was radiolabeled as described herein. On the day of the study animals received a single IV injection of 200 uL into the caudal vein via a catheter with 5 MBq of ¹⁷⁷Lu[Lu] complex of Compound 10 (1 nmol) per animal. The blocking study (Group 7) combined 1 nmol of ¹⁷⁷Lu[Lu] complex of Compound 10 (5 MBq) with 100 nmol unlabeled/cold-Compound 10.

| | # of | Readout | | | | Route/ |
|---|---|---|---|---|---|---|
| Group | animals | Time | | MBq/animal | nmol/animal | Schedule |
| 1 | 4 | 0.5 | hours | 15 | 1 | IV/Q1Dx1 |
| 2 | 4 | 3 | hours | 15 | 1 | IV/Q1Dx1 |
| 3 | 4 | 6 | hours | 15 | 1 | IV/Q1Dx1 |
| 4 | 4 | 24 | hours | 15 | 1 | IV/Q1Dx1 |
| 5 | 4 | 72 | hours | 15 | 1 | IV/Q1Dx1 |
| 6 | 4 | 168 | hours | 15 | 1 | IV/Q1Dx1 |
| 7 | 4 | 3 | hours | 15 + 0 | 1 + 100 | IV/Q1Dx1 |

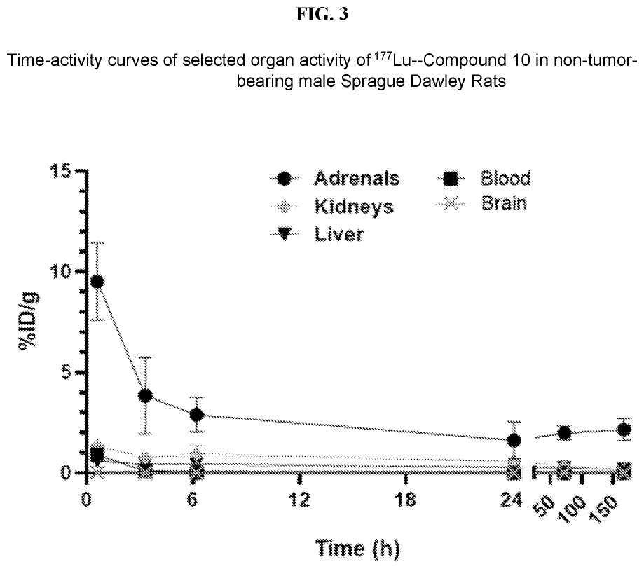

After drug administration, animals were euthanized at timepoints (0.5 hour, 3 hours, 6 hours, 24 hours, 72 hours, 168 hours) and organs (blood, heart, lungs, thymus, thyroid, liver, gallbladder, spleen, pancreas, eyes, stomach, small intestine, large intestine, kidneys, adrenals, brain, bone (right femur), bone marrow (harvested from left femur), muscle, skin, testes, urinary bladder, tail and remaining carcass) were collected, weighed, and radioactivity was assessed in each organ/tissue. Activity was quantitated and expressed as % ID/g (Percentage of Initial Dose/gram of tissue). See FIG. 3.

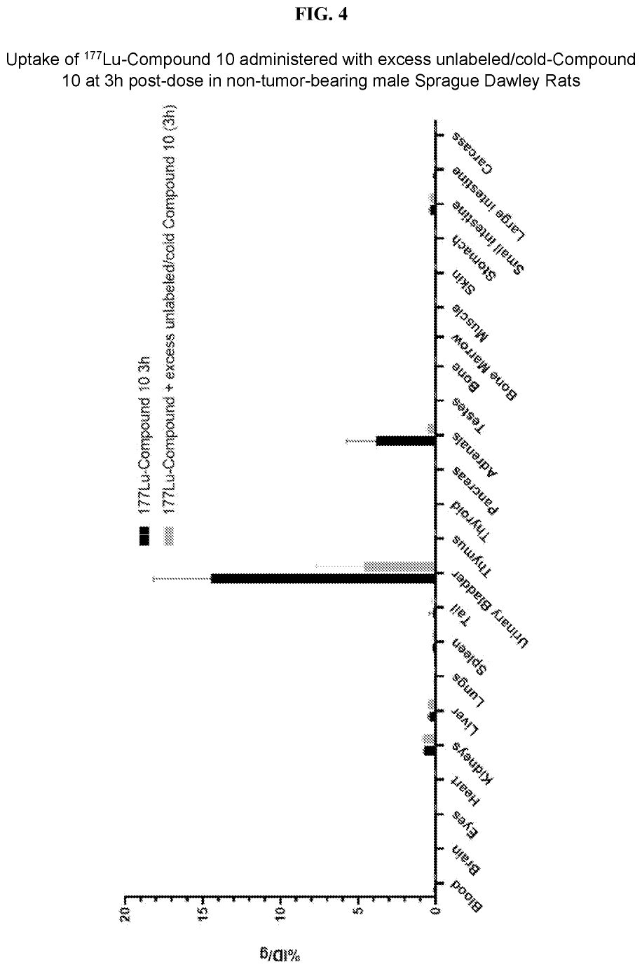

Time course of uptake of ¹⁷⁷Lu[Lu] complex of Compound 10 in MC2R-expressing adrenals glands with minimal uptake in other non-MC2R expressing organs. Mean±SD n=3. See FIG. 4. Organ activity in % ID/g of organ at 3 h post-dose. Selective block of ¹⁷⁷Lu[Lu] complex of Compound 10 uptake in the presence of excess unlabeled/cold-Compound 10 shows MC2R-mediated uptake in adrenals glands, with minimal uptake in other MC2R negative organs. Uptake in bladder shows primary excretion pathway. Mean±SD n=3

### Example B-5: Biodistribution in Human MC2R Tumor-Bearing Female BRGSF Mice Dosed with ¹¹¹In[In] Complex of Compound 10

Study Design: 24 hours prior to the start of the biodistribution study, the study compound (CMPD) was radiolabeled as described herein. On the day of the study animals received a single IV injection of 200 uL into the caudal vein via a catheter with 5 MBq of ¹¹¹In[In] complex of Compound 10 (1 nmol) per animal. The blocking study (Group 5) combined 1 nmol of ¹¹¹In[In] complex of Compound 10 (5 MBq) with 100 nmol 1151n-Compound 10.

| | # of | Readout | | | | Route/ |
|---|---|---|---|---|---|---|
| Group | animals | Time | | MBq/animal | nmol/animal | Schedule |
| 1 | 3 | 0.5 | hours | 5 | 1 | IV/Q1Dx1 |
| 2 | 3 | 2 | hours | 5 | 1 | IV/Q1Dx1 |
| 3 | 3 | 5 | hours | 5 | 1 | IV/Q1Dx1 |
| 4 | 3 | 24 | hours | 5 | 1 | IV/Q1Dx1 |
| 5 | 3 | 2 | hours | 5 + 0 | 1 + 100 | IV/Q1Dx1 |

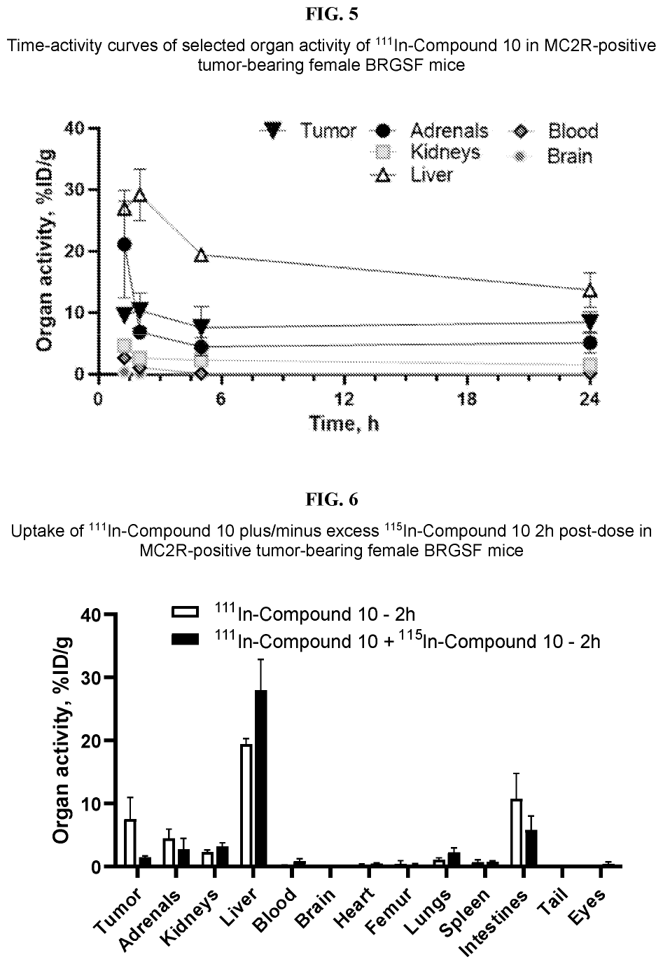

Results; ¹¹¹In[In] complex of Compound 10 demonstrated high uptake in adrenal glands, the only known site of endogenous MC2R expression and in tumors expressing human MC2R. The specificity of ¹¹¹In[In] complex of Compound 10 for MC2R-positive tissues (adrenal glands and tumors) was demonstrated by competition studies where co-administration of excess ¹¹⁵In-Compound 10 blocked ¹¹¹In[In] complex of Compound 10 uptake in MC2R-positive tumors and adrenal glands. The kidney, liver, and intestine transient uptake does not appear to be receptor-mediated and is attributed to the excretion routes. See FIG. 5.

Uptake in MC2R-expressing tumors and adrenal glands. Minimal non-target organ uptake in blood, brain and other tissues (not shown). Uptake in kidney and liver is transient and attributed to excretion routes. Mean±SD n=3. See FIG. 6.

Organ activity, expressed as % ID/g, at 2 h post-dose. Selective blocking of ¹¹¹In[In] complex of Compound 10 uptake in the presence of excess ¹¹⁵In-Compound 10 shows MC2R-mediated uptake in tumor and adrenal glands. Non-target organ uptake is not receptor-mediated with kidney, liver, and intestines transient uptake attributed to excretion routes. Mean±SD n=3.

### Example B-6: Anti-Tumor Efficacy of ¹⁷⁷Lu[Lu] Complex of Compound 10 in Female BRGSF Mice Bearing Human MC2R Expressing Tumors

Study Design: 24 hours prior to the start of the biodistribution study, the study compound (CMPD) was radiolabeled as described herein. When tumors reach approx. 150-250 mm³ animals were randomized, and animals received a IV injection of 100-120 uL into the caudal vein via a catheter with ¹⁷⁷Lu[Lu] complex of Compound 10. Dosing is outlined in the table below.

| | | Dose | Dose | | | |
|---|---|---|---|---|---|---|
| | # of | (MBq/ | (nmol/ | | nmol/ | |
| Group | animals | Mouse) | Mouse) | Schedule | animal | Route |
| 1 | 4 | — | — | Q7Dx3 | — | IV |
| 2 | 4 | 120 | 1 | Q1Dx1 | 1 | IV |
| 3 | 4 | 90 | 1 | Q7Dx2 | 1 | IV |
| 4 | 4 | 60 | 1 | Q7Dx3 | 1 | IV |

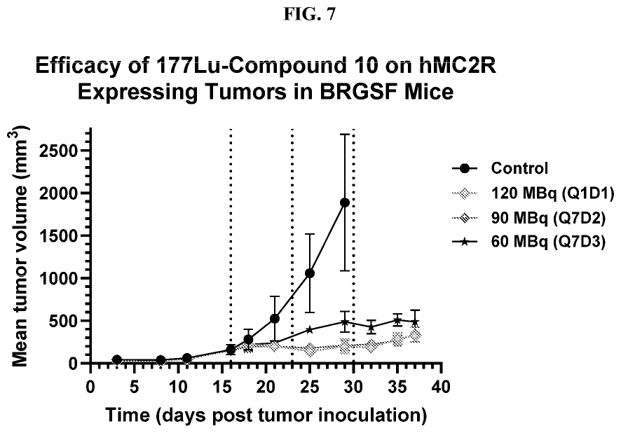

Results: ¹⁷⁷Lu[Lu] complex of Compound 10 demonstrated strong anti-tumor activity in BRGSF mice with hMC2R expressing tumors. ¹⁷⁷Lu[Lu] complex of Compound 10 dosed with a single dose at 120 MBq had a tumor growth inhibition (TGI) of 89% compared to control. Animals that received two doses of ¹⁷⁷Lu[Lu] complex of Compound 10 at 90 MBq also had a TGI of 89%. Animals that received three doses of ¹⁷⁷Lu[Lu] complex of Compound 10 at 60 MBq had a TGI of 74%. See FIG. 7. All doses were well tolerated, with weight loss not exceeding 10% in any treatment group (data not shown).

Animals in vehicle group were terminated on Day 29 due to excessive tumor burden. Tumor growth inhibition was calculated at day 29 to vehicle group. Body weight loss was <10% at all doses on day 29. Tumor volumes were calculated as (Length×width²)×0.5. Data shown in mean±SD. Vertical lines represent day of dosing.

Biodistribution studies in non-tumor-bearing rats demonstrate that ¹¹¹In[In] complex of Compound 10 has high, specific, and prolonged uptake in MC2R-positive adrenals. In MC2R-negative tissues, uptake was transient in organs association with excretion (i.e., kidney and bladder). In xenograft tumor-bearing mouse studies, ¹¹¹In[In] complex of Compound 10 showed sustained uptake in the MC2R-positive tumors and adrenal glands and ¹⁷⁷Lu[Lu] complex of Compound 10 demonstrated good anti-tumor efficacy.

### Example B-7: Safety and Dosimetry Study in Patients with Adrenocortical Carcinoma (ACC) and Healthy Volunteers

A non-limiting example of a phase 1 safety and dosimetry study of ⁶⁸Ga-complex of a compound of Formula (I) in patients with ACC and healthy volunteers is described below.

Purpose: This is an open-label, two-part, first-in-human, Phase 1 study of ⁶⁸Ga-complex of a compound of Formula (I) designed to characterize its safety, tolerability, biodistribution, radiation dosimetry and PET imaging properties in subjects diagnosed with ACC and in adult healthy volunteers. In some embodiments, a ⁶⁸Ga-complex of a compound of Formula (I) is used to localize to MC2R-expressing lesions and identify ACC subjects with MC2R-expressing tumors who may benefit from treatment with MC2R-targeting therapeutic agents, such as ¹⁷⁷Lu-complex of a compound of Formula (I). In healthy humans, MC2R is found primarily in the bilateral adrenal cortices, which are steroid hormone-producing endocrine organs, and is one of the genes that define adrenocortical cell identity. ACC is a rare cancer of the adrenal cortex with limited treatment options, especially in the advanced/recurrent setting in which only one drug, mitotane, is approved in the United States. MC2R is expressed in ACC with highest expression in tumors from subjects with poor clinical outcomes.

This study will enroll approximately 25 evaluable subjects in total, including approximately 21 subjects with a diagnosis of ACC and 4 healthy volunteers. ACC subjects at the time of initial diagnosis and those with recurrent/relapsed disease with at least one CT-measurable target lesion by Response Evaluation Criteria in Solid Tumors (RECIST) v1.1 target lesion criteria are eligible. To further understand the dosimetry of ⁶⁸Ga-complex of a compound of Formula (I), including uptake in healthy adrenal glands, a cohort of healthy volunteers will also be evaluated.

Study Populations: Subjects with ACC; healthy volunteers

Primary Objectives: To evaluate the overall safety and tolerability of ⁶⁸Ga-complex of a compound of Formula (I). To determine the organ and whole-body dosimetry of ⁶⁸Ga-complex of a compound of Formula (I).

Secondary Objectives: To determine tumor uptake of ⁶⁸Ga-complex of a compound of Formula (I) by timepoint and location in subjects with ACC. To compare of ⁶⁸Ga-complex of a compound of Formula (I) positron emission tomography/computed tomography (PET/CT) scans to anatomic imaging (contrast-enhanced diagnostic CT images) in detecting tumor lesions in subjects with ACC. To select a of ⁶⁸Ga-complex of a compound of Formula (I) dose and imaging time for further assessment in ACC. To determine the tumor-to-background ratio of tumors detected by of ⁶⁸Ga-complex of a compound of Formula (I) PET/CT. To assess the pharmacokinetic (PK) profile of ⁶⁸Ga-complex of a compound of Formula (I).

Exploratory Objective: To evaluate the expression of tumor markers in tumor tissue.

Primary Endpoints: Incidence of adverse events (AE) characterized overall and by type, frequency seriousness, relationship to study drug, timing, and severity graded according to the National Cancer Institute (NCI) Common Terminology Criteria for Adverse Events (CTCAE), Version 5.0. Absolute values and changes in clinical laboratory parameters. Absorbed dose coefficients (milliGray [mGy]/megabecquerel [MBq]) in target organs and the effective dose coefficient (milliSievert [mSv]/MBq). Whole-body absorbed dose coefficient (Gy/MBq).

Secondary Endpoints: Number and location of tumors identified by a ⁶⁸Ga-complex of a compound of Formula (I) PET/CT and by anatomic imaging (contrast-enhanced diagnostic CT). Maximum standard update value (SUVmax) of each tumor and of source organs. Ratio of the tumor SUV over reference region SUV. PK parameters and radioactive blood counts from serial blood samples.

Exploratory Endpoint: Expression of tumor markers (e.g., melanocortin 2 receptor [MC2R], other) in the histology samples from biopsy and surgical tumor specimens from subjects with ACC, as measured by immunohistochemistry or other methods.

Study Design: Approximately 13 evaluable subjects in total, including 9 subjects with ACC and 4 healthy volunteers (2 male, 2 female), will be enrolled in Part 1 (dosimetry and dose selection). Each subject enrolled in Part 1 will be assigned to a dose cohort, receive a single injection of ⁶⁸Ga-complex of a compound of Formula (I) at the assigned dose, and be imaged continuously by PET/CT for the first 20 minutes post-dose with the camera centered over a selected target tumor lesion (ACC subjects) or over the adrenal glands (healthy volunteers), followed by whole-body scans at 30-, 60-, 120-, and 180-minutes post-dose. There will be 3 sequential dose cohorts of ACC subjects enrolled using a de-escalation design, as follows: Cohort 1 (8 mCi), Cohort 2a (5 mCi), Cohort 3 (2.5 mCi). Additionally, 4 healthy volunteers will be enrolled in parallel at a single dose level (Cohort 2b; 5 mCi).

Upon completion of Part 1, a dose of ⁶⁸Ga-complex of a compound of Formula (I) and imaging time(s) will be selected to further evaluate safety and tumor uptake of the ⁶⁸Ga-complex of a compound of Formula (I) in approximately 12 ACC subjects in Part 2 (ACC expansion).

All subjects with ACC will undergo a contrast-enhanced diagnostic CT scan of chest, abdomen, and pelvis (or magnetic resonance imaging [MRI] if subject is allergic to CT contrast media) within 14 days of the study drug injection (preferably on the same day as the PET/CT scan).

In Part 1 only, serial blood samples will be collected for PK and dosimetry analyses. A urine sample will be collected after completion of imaging activities for dosimetry analysis.

In order to assess the expression of MC2R and other markers of interest and to compare to SUV, tumor samples (frozen, paraffin blocks or processed slides) will be obtained from subjects with ACC who have had prior tumor biopsies or resections, when available.

All subjects must meet all the inclusion eligibility criteria and none of the exclusion eligibility criteria for either ACC subjects or healthy volunteers, as appropriate and provided below.

Inclusion Criteria for ACC Subjects: Pathologically confirmed ACC. Newly diagnosed or recurrent/relapsed ACC with at least 1 measurable target lesion per RECIST v1.1 criteria. Male or non-pregnant, non-lactating female subjects age ≥18 years. Sexually active must agree to use adequate method(s) of effective contraception during their participation in the study. Eastern Cooperative Oncology Group (ECOG) Performance Status ≤2. Adequate hepatic function as defined by a) serum alanine aminotransaminase (ALT)/aspartate aminotransaminase (AST)≤3× upper limit of normal (ULN) or ≤5×ULN if liver metastases are present or received prior mitotane therapy, and b) serum bilirubin−total≤1.5×ULN (unless due to Gilbert's syndrome or hemolysis in which case total ≤3.0×ULN). Adequate renal function as measured by creatinine clearance calculated by the Cockcroft-Gault formula (≥60 mL/minute). Able to understand and willing to sign a written informed consent form.

Exclusion Criteria for ACC Subjects: Administered a radionuclide within a period of time corresponding to less than 10 physical half-lives of the radionuclide prior to study Day 1. Radiotherapy ≤14 days prior to study Day 1. Major surgery ≤21 days prior to study Day 1 or has not recovered from adverse effects of such procedure. History of cerebrovascular accident within 6 months or that resulted in ongoing neurologic instability. History of other previous or concurrent cancer that would interfere with the determination of safety. Any other condition that in the opinion of the Investigator would place the subject at an unacceptable risk or cause the subject to be unlikely to fully participate or comply with study procedures.

Inclusion Criteria for Healthy Volunteers: Healthy male or non-pregnant, non-lactating female subjects aged between 18 and 59 years (inclusive). Sexually active must agree to use adequate method(s) of effective contraception during their participation in the study. Body mass index between 18.0 and 32.0 kg/m² (inclusive). Adequate renal function as measured by creatinine clearance calculated at ≥60 mL/minute by the Cockcroft-Gault formula. Able to understand and willing to sign a written informed consent form.

Exclusion Criteria for Healthy Volunteers: Prior unilateral or bilateral adrenalectomy. History of significant hypersensitivity, intolerance, or allergy to any drug compound, food, or other substance, unless approved by the Investigator. Subjects diagnosed with adrenal disease, including Cushing's syndrome, adrenal insufficiency, or congenital adrenal hyperplasia. Glucocorticoid steroid use (including topical) within 4 weeks prior to study Day 1 (inhaled steroids are allowed). Administered a radionuclide within a period of time corresponding to less than 10 physical half-lives of the radionuclide prior to study Day 1. Donation of blood or significant blood loss within 3 months prior to screening, donation of plasma within 2 weeks prior to screening, or donation of platelets within 6 weeks prior to screening. Any other condition that in the opinion of the Investigator would place the subject at an unacceptable risk or cause the subject to be unlikely to fully participate or comply with study procedures.

### Study Drug, Dose, and Mode of Administration

Study Drug: A ⁶⁸Ga-complex of a compound of Formula (I). In some embodiments, the ⁶⁸Ga-complex is a ⁶⁸Ga-complex of Compound 1. In some embodiments, the ⁶⁸Ga-complex is a 68G-complex of Compound 2. In some embodiments, the ⁶⁸Ga-complex is a ⁶⁸Ga-complex of Compound 3. In some embodiments, the ⁶⁸Ga-complex is a ⁶⁸Ga-complex of Compound 4. In some embodiments, the ⁶⁸Ga-complex is a ⁶⁸Ga-complex of Compound 5. In some embodiments, the ⁶⁸Ga-complex is a ⁶⁸Ga-complex of Compound 6. In some embodiments, the ⁶⁸Ga-complex is a ⁶⁸Ga-complex of Compound 7. In some embodiments, the ⁶⁸Ga-complex is a ⁶⁸Ga-complex of Compound 8. In some embodiments, the ⁶⁸Ga-complex is a ⁶⁸Ga-complex of Compound 9. In some embodiments, the ⁶⁸Ga-complex is a ⁶⁸Ga-complex of Compound 10. In some embodiments, the ⁶⁸Ga-complex is a 68G-complex of Compound 11. In some embodiments, the ⁶⁸Ga-complex is a ⁶⁸Ga-complex of Compound 12. In some embodiments, the ⁶⁸Ga-complex is a ⁶⁸Ga-complex of Compound 13. In some embodiments, the ⁶⁸Ga-complex is a ⁶⁸Ga-complex of Compound 14. In some embodiments, the ⁶⁸Ga-complex is a ⁶⁸Ga-complex of Compound 15. In some embodiments, the ⁶⁸Ga-complex is a ⁶⁸Ga-complex of Compound 16. In some embodiments, the ⁶⁸Ga-complex is a ⁶⁸Ga-complex of Compound 17. In some embodiments, the ⁶⁸Ga-complex is a ⁶⁸Ga-complex of Compound 18. In some embodiments, the ⁶⁸Ga-complex is a ⁶⁸Ga-complex of Compound 19.

Dose: Part 1 (dosimetry and dose selection): 8.0 mCi (±20%), 5.0 mCi (±20%), or 2.5 mCi (±20%) for subjects with ACC; 5.0 mCi (±20%) for healthy volunteers; Total carrier mass of the compound of Formula (I): not more than (NMT) 90 μg/dose. Part 2 (ACC expansion): Selected dose from Part 1.

Mode of Administration: Intravenous.

Duration of Participation: A 28-day Screening window will be utilized where the subject will undergo study assessments to deem the subject eligible for the study. Once confirmed, subjects will receive the study drug, ⁶⁸Ga-complex of a compound of Formula (I), and PET/CT imaging on Day 1. The subjects will return to the clinical site on Day 2 (+2 days) for a safety evaluation. The final assessment will be a follow up contact to the subject on Day 7 (+2 days).

Study Duration: The start of the study will be the date on which the first subject provides informed consent. The end of the study will be the last subject's last assessment.

The examples and embodiments described herein are for illustrative purposes only and various modifications or changes suggested to persons skilled in the art are to be included within the spirit and purview of this application and scope of the appended claims.

1-78. (canceled)

79. A compound of Formula (I), or a pharmaceutically acceptable salt thereof: 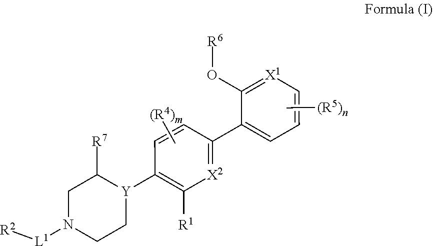
wherein:
R¹ is R^{a}; and Y is N;
or R¹ is R⁴; and Y is C—R^{a};
R^{a} is —CH₂NRs-L²-R^{b}, or —C(═O)NR⁸-L²-R^{b};
L² is absent, -(unsubstituted or substituted C₁-C₆ alkylene)-, -(unsubstituted or substituted C₁-C₆ alkylene)-N(R⁹)—, -(unsubstituted or substituted C₁-C₆ alkylene)_{q}-(unsubstituted or substituted C₃-C₆cycloalkyl), -(unsubstituted or substituted C₁-C₆ alkylene)_{q}-(unsubstituted or substituted aryl), -(unsubstituted or substituted C₁-C₆ alkylene)_{q}-(unsubstituted or substituted C₂-C₆heterocycloalkyl), or -(unsubstituted or substituted C₁-C₆ alkylene)_{q}-(unsubstituted or substituted heteroaryl); q is 0 or 1;
R^{b} is -L³-Q;
L³ is a linker;
Q is a chelating moiety or a radionuclide complex thereof;
L¹ is absent or —C(═O)—;
R² is ring that is selected from the group consisting of: C₃-C₈cycloalkyl, C₂-C₈heterocycloalkyl, aryl, or heteroaryl, wherein R² is unsubstituted or is substituted with R^{3a}, R^{3b}, or R^{3c}, or combinations thereof,
each R^{3a}, R^{3b}, R^{3c}, R⁴, and R⁵ is independently hydrogen, halogen, substituted or unsubstituted C₁-C₄alkyl, substituted or unsubstituted C₁-C₄alkenyl, substituted or unsubstituted C₁-C₄alkynyl, substituted or unsubstituted C₁-C₄fluoroalkyl, substituted or unsubstituted C₁-C₄heteroalkyl, —CN, —N(R⁹)₂, or —OR⁹;
R⁶ is C₁-C₄alkyl;
R⁷ is hydrogen or C₁-C₄alkyl;
R⁸ is hydrogen or C₁-C₄alkyl;
each R⁹ is independently hydrogen, C₁-C₄alkyl, C₁-C₄fluoroalkyl, substituted or unsubstituted C₁-C₄heteroalkyl;
X¹ is CR⁵ or N;
X² is CR⁴ or N;
m is 0, 1, or 2; and n is 0, 1, 2, or 3.

80. The compound of claim 79, or a pharmaceutically acceptable salt thereof, wherein:
R² is

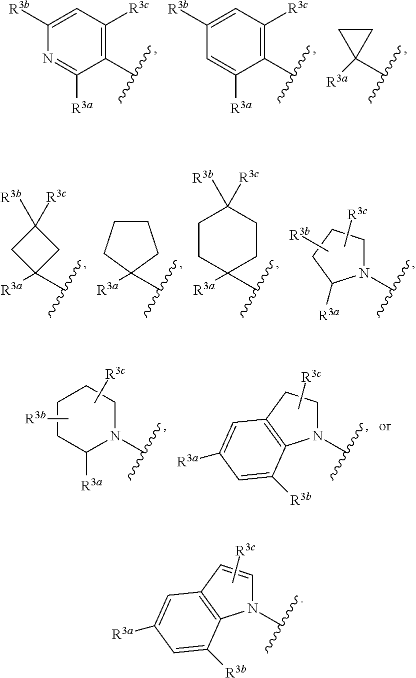

81. The compound of claim 79, or a pharmaceutically acceptable salt thereof, wherein:
each R^{3a}, R^{3b}, R^{3c}, R⁴, and R⁵ is independently hydrogen, F, Cl, Br, —CN, —OH, —OCH₃, —OCH₂CH₃, —CH₃, —CH₂CH₃, —CH₂CH₂CH₃, —CH(CH₃)₂, —C(CH₃)₃, —CH═CH₂, —CH₂OH, —CH₂CN, —CH₂F, —CHF₂, —CF₃, —CH₂CH₂OH, —CH₂CH₂CN, —CH₂CH₂F, —CH₂CHF₂, —CH₂CF₃, —CH₂OCH₃, —CH₂CH₂OCH₃, —CH₂NH₂, —CH₂NHCH₃, —CH₂N(CH₃)₂, —CH₂CH₂NH₂, —CH₂CH₂NHCH₃, or —CH₂CH₂N(CH₃)₂.

82. The compound of claim 79, or a pharmaceutically acceptable salt thereof, wherein:
R⁶ is —CH₂CH₃; and
R⁷ is hydrogen, —CH₃ or —CH₂CH₃.

83. The compound of claim 79, or a pharmaceutically acceptable salt thereof, wherein the compound of Formula (I) has the structure of Formula (VI), or a pharmaceutically acceptable salt thereof: 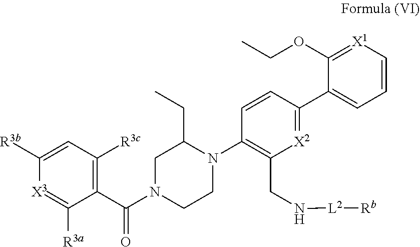
wherein X³ is CH or N.

84. The compound of claim 83, or a pharmaceutically acceptable salt thereof, wherein the compound of Formula (VI) has the structure of Formula (VIa), Formula (VIb), Formula (VIc), or Formula (VId), or a pharmaceutically acceptable salt thereof:

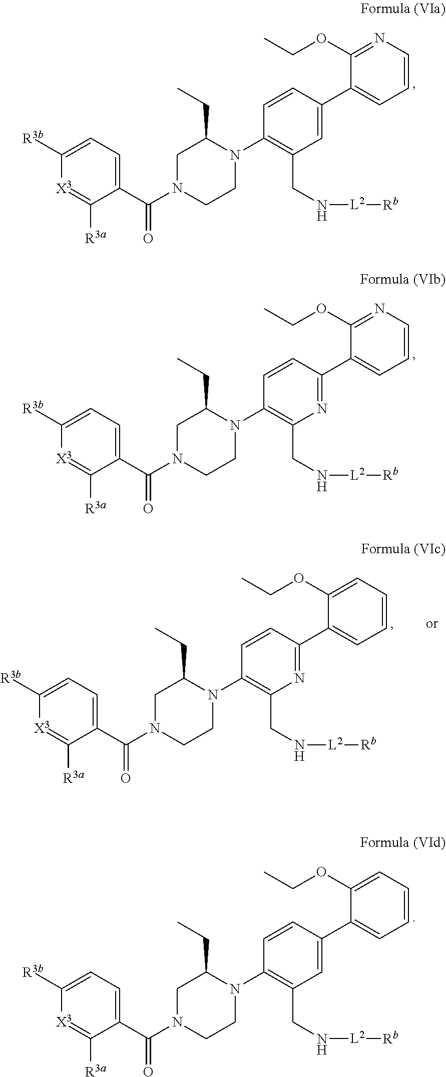

85. The compound of claim 79, or a pharmaceutically acceptable salt thereof, wherein the compound of Formula (I) has the structure of Formula (VIII), or a pharmaceutically acceptable salt thereof: 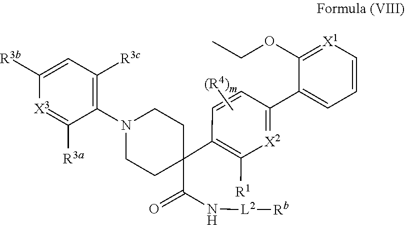
wherein X³ is CH or N.

86. The compound of claim 85, or a pharmaceutically acceptable salt thereof, wherein the compound of Formula (I) has the structure of Formula (VIIIa), Formula (VIIIb), or Formula (VIIIc), or a pharmaceutically acceptable salt thereof:

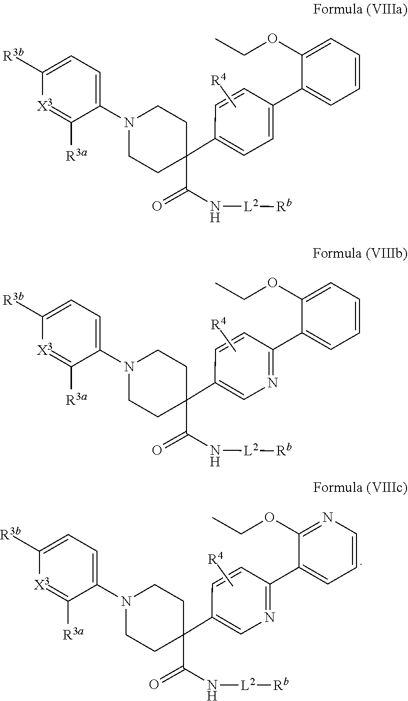

87. The compound of claim 84, or a pharmaceutically acceptable salt thereof, wherein:
each R^{3a}, R^{3b}, and R^{3c} is independently hydrogen, F, Cl, Br, —CN, —OH, —OCH₃, —OCH₂CH₃, —CH₃, —CH₂CH₃, —CH₂CH₂CH₃, —CH(CH₃)₂, —C(CH₃)₃, —CH═CH₂, —CH₂OH, —CH₂CN, —CH₂F, —CHF₂, —CF₃, —CH₂CH₂OH, —CH₂CH₂CN, —CH₂CH₂F, —CH₂CHF₂, —CH₂CF₃, —CH₂OCH₃, —CH₂CH₂OCH₃, —CH₂NH₂, —CH₂NHCH₃, —CH₂N(CH₃)₂, —CH₂CH₂NH₂, —CH₂CH₂NHCH₃, or —CH₂CH₂N(CH₃)₂; and
each R⁴ is independently hydrogen, F, Cl, Br, —CN, —OH, —OCH₃, —CH₃, —CH₂F, —CHF₂, or —CF₃.

88. The compound of claim 79, or a pharmaceutically acceptable salt thereof, wherein the compound of Formula (I) has one of the following structures, or a pharmaceutically acceptable salt thereof:

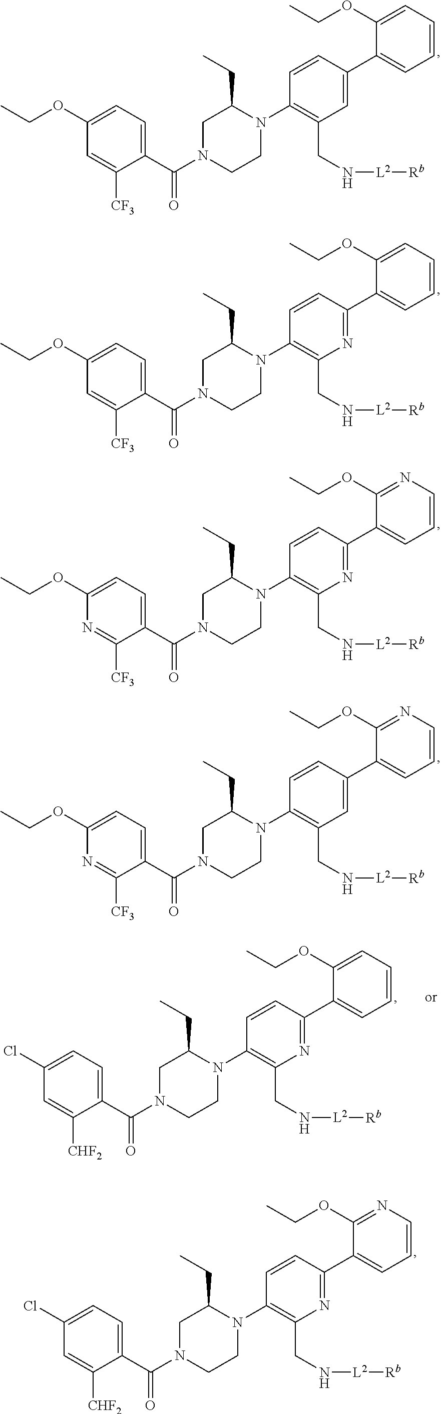

89. The compound of claim 79, or a pharmaceutically acceptable salt thereof, wherein the compound of Formula (I) has one of the following structures, or a pharmaceutically acceptable salt thereof:

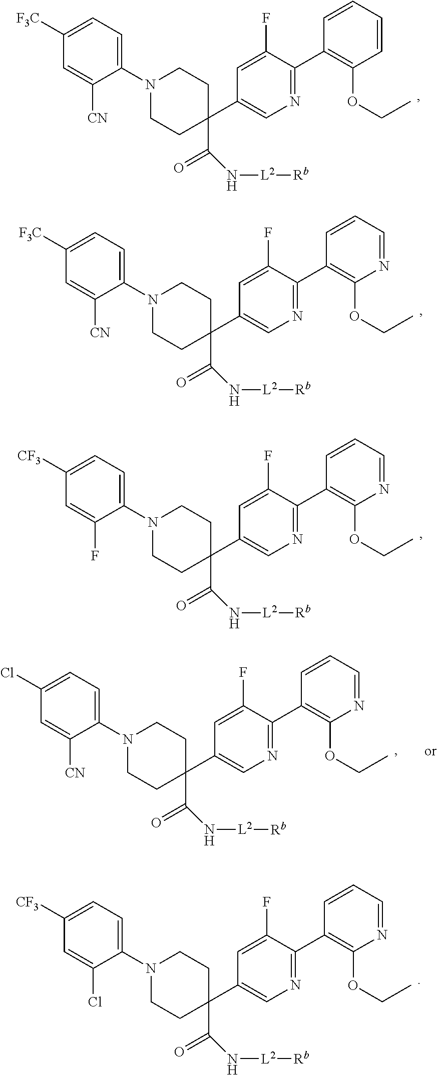

90. The compound of claim 79, or a pharmaceutically acceptable salt thereof, wherein:
L² is absent, —CH₂—, —CH₂CH₂—, —CH₂CH₂CH₂—, —CH₂CH₂CH₂CH₂—, —CH₂NH—, —CH₂CH₂NH—, —CH₂CH₂CH₂NH—, —CH₂(CH₂)₂NH—,

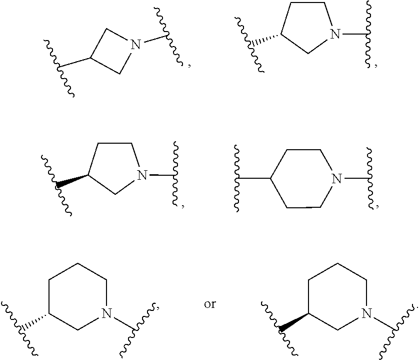 [SMILES: *C1CN(*)C1 ; *[C@@H]1CCN(*)C1 ; *[C@H]1CCN(*)C1 ; *C1CCN(*)CC1 ; *[C@@H]1CCCN(*)C1 ; *[C@H]1CCCN(*)C1]

91. The compound of claim 79, or a pharmaceutically acceptable salt thereof, wherein Q is a chelating moiety selected from the group consisting of:
1,4,7,10-tetraazacyclododecane-1,4,7,10-tetraacetic acid (DOTA);
1,4,7,10-tetraazacyclododecane-1,4,7-triacetic acid (DO3A);
1,4,7,10-tetraazacyclododecane-1,7-diacetic acid (DO2A);
α,α′,α″,α′″-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid;
benzyl-1,4,7,10-tetraazacyclododecane-1,4,7,10-tetraacetic acid (Bn-DOTA);
p-hydroxy-benzyl-1,4,7,10-tetraazacyclododecane-1,4,7,10-tetraacetic acid (p-OH-Bn-DOTA);
p-SCN-benzyl-1,4,7,10-tetraazacyclododecane-1,4,7,10-tetraacetic acid (p-SCN-Bn-DOTA);
6,6′-(((pyridine-2,6-diylbis(methylene))bis((carboxymethyl)azanediyl))bis(methylene))-dipicolinic acid (H₄pypa); H₄pypa-benzyl; H₄pypa-benzyl-NCS;
6,6′,6″,6′″-(((pyridine-2,6-diylbis(methylene))bis(azanetriyl))tetrakis(methylene))-tetrapicolinic acid (H₄py4pa); H₄py4pa-benzyl; H₄py4pa-benzyl-NCS;
6,6′-((ethane-1,2-diylbis((carboxymethyl)azanediyl))bis(methylene))dipicolinic acid (H₄octapa); H₄octapa-benzyl-NCS; H₄octapa-benzyl;
3,6,9,12-tetrakis(carboxymethyl)-3,6,9,12-tetraazatetradecanedioic acid (TTHA);
or a radionuclide complex thereof.

92. The compound of claim 79, or a pharmaceutically acceptable salt thereof, wherein Q is a chelating moiety selected from the group consisting of: 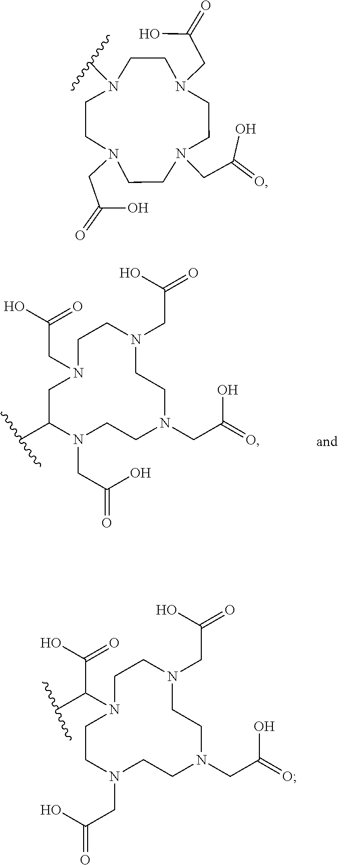 [SMILES: *N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]
or a radionuclide complex thereof.

93. The compound of claim 79, or a pharmaceutically acceptable salt thereof, wherein:
L³ is -L⁴-, -L⁵-, -L⁶-, -L⁷-, -L⁸-, -L⁹-, -L¹⁰-, -L⁴-L⁹-L¹⁰-, -L⁴-L⁵-L⁶-L⁷-L⁸-L⁹-L¹⁰-, -L⁴- L⁶-L⁵-L⁷-L⁸-L⁹-L¹⁰-, -L⁶-L⁵-L⁷-L⁸-L⁹-L¹⁰-, or -L⁵-L⁷-L⁸-L⁹-L¹⁰-, or a combination thereof,
L⁴ is unsubstituted or substituted C₁-C₂₀alkylene, unsubstituted or substituted C₁-C₂₀heteroalkylene, unsubstituted or substituted C₂-C₂₀alkenylene, unsubstituted or substituted C₂-C₂₀alkynylene, C₄-C₂₀polyethylene glycol, —C(═O)—, —C(═O)NH—, —C(═O)-unsubstituted or substituted C₁-C₂₀alkylene, —C(═O)-unsubstituted or substituted C₁-C₂₀heteroalkylene, —C(═O)—C₄-C₂₀polyethylene glycol, —C(═O)NH-unsubstituted or substituted C₁-C₂₀alkylene, —C(═O)NH-unsubstituted or substituted C₁-C₂₀heteroalkylene, —C(═O)NH—C₄-C₂₀polyethylene glycol, —NHC(═O)-unsubstituted or substituted C₁-C₂₀alkylene, —NHC(═O)-unsubstituted or substituted C₁-C₂₀heteroalkylene, or —NHC(═O)—C₄-C₂₀polyethylene glycol;
L⁵ is absent, —S—S—, one or more independently selected natural or unnatural amino acids, wherein any free amine of an amino acid or amide bond linking the 2 or more amino acids is optionally independently substituted with —CH₃, and any peptide that is formed is a linear or branched peptide, and wherein any free amine of the amino acid or peptide is optionally substituted with —C(═O)—(CH₂)₁₋₄-(4-iodophenyl);
L⁶ is absent, —O—, —S—, —S(═O)—, —S(═O)₂—, —NH—, —CH(OH)—, —NHC(═O)—, —C(═O)NH—, —C(═O)O—, —OC(═O)—, —CH(═N)—, —CH(═N—NH)—, —CCH₃ (═N)—, —CCH₃ (═N—NH)—, —OC(═O)NH—, —NHC(═O)NH—, —NHC(═O)O—, —(CH₂)ᵥ—, —C(═O)—(CH₂CH₂X⁴)ᵥ—, or —(CH₂CH₂X⁴)ᵥ—, —C(═O)—(X⁴CH₂CH₂)ᵥ—, or —(X⁴CH₂CH₂)ᵥ—, each instance of v is independently 1, 2, 3, 4, 5, 6, 7, 8, 9, 10, 11, or 12;
each X⁴ is independently selected from O and NR^{X}; and each R^{X} is independently selected from hydrogen, C₁-C₄alkyl and —CH₂CO₂H;
L⁷ is absent, unsubstituted or substituted C₁-C₆alkylene, unsubstituted or substituted C₁-C₆heteroalkylene, unsubstituted or substituted C₂-C₆alkenylene, unsubstituted or substituted C₂-C₆alkynylene, unsubstituted or substituted cycloalkylene, unsubstituted or substituted heterocycloalkylene, unsubstituted or substituted arylene, unsubstituted or substituted heteroarylene,
L⁸ is absent, —[CH(R^{Y})]_{y}—, —(CH₂)_{y}—, —(X⁵CH₂CH₂)_{y}—, or —(CH₂CH₂X⁵)_{y}—, each y is 1, 2, 3, 4, 5, 6, 7, 8, 9, 10, 11, or 12; each R^{Y} is independently selected from hydrogen and —OH; each X⁵ is independently selected from O and NR^{X}; and each R^{X} is independently selected from hydrogen, C₁-C₄alkyl and —CH₂CO₂H;
L⁹ is absent, —(CH₂)—, —O—, —S—, —S(O)—, —S(O)₂—, —NH—, —CH(OH)—, —C(═O)—, —C(═O)NH—, —NHC(═O)—, —C(═S)NH—, —NHC(═S)—, —C(═O)O—, —OC(═O)—, —OC(═O)NH—, —NHC(═O)NH—, or —NHC(═O)O—;
L¹⁰ is absent, unsubstituted or substituted C₁-C₆alkylene, or unsubstituted or substituted C₁-C₆heteroalkylene, or unsubstituted or substituted benzyl.

94. The compound of claim 93, or a pharmaceutically acceptable salt thereof, wherein L³ is -L⁴-L⁹-L¹⁰-;
L⁴ is unsubstituted or substituted C₁-C₂₀alkylene, C₄-C₂₀polyethylene glycol, —C(═O)-unsubstituted or substituted C₁-C₂₀alkylene, —C(═O)—C₄-C₂₀polyethylene glycol, —C(═O)NH-unsubstituted or substituted C₁-C₂₀alkylene, —C(═O)NH—C₄-C₂₀polyethylene glycol, —NHC(═O)-unsubstituted or substituted C₁-C₂₀alkylene, or —NHC(═O)—C₄-C₂₀polyethylene glycol;
L⁹ is —C(═O)NH—, —NHC(═O)—, —C(═S)NH—, or —NHC(═S)—; and
L¹⁰ is C₁-C₂alkylene or benzyl.

95. The compound of claim 93, or a pharmaceutically acceptable salt thereof, wherein L³ comprises -L⁹-L¹⁰-, and -L⁹-L¹⁰- is —NHC(═O)—CH₂— or —NHC(═O)—CH₂CH₂—.

96. The compound of claim 93, or a pharmaceutically acceptable salt thereof, wherein L³ is -L⁴-L⁶-L⁵-L⁷-L⁸-L⁹-L¹⁰-, -L⁶-L⁵-L⁷-L⁸-L⁹-L¹⁰-, or -L⁵-L⁷-L⁸-L⁹-L¹⁰-;
L⁴ is unsubstituted or substituted C₁-C₂₀alkylene, or C₄-C₂₀polyethylene glycol;
L⁶ is absent, —O—, —NH—, —NHC(═O)—, —C(═O)NH—, —(CH₂)ᵥ—, —C(═O)—(CH₂CH₂O)ᵥ—, —C(═O)—(CH₂CH₂O)ᵥ—(CH₂CH₂NR^{X})—, —(CH₂CH₂O)ᵥ—, —(CH₂CH₂NR^{X})—(CH₂CH₂O)ᵥ—, —(OCH₂CH₂)ᵥ—, —(NR^{X}CH₂CH₂)—(OCH₂CH₂)ᵥ—, or —C(═O)—(NR^{X}CH₂CH₂)(OCH₂CH₂)ᵥ—, each instance of v is independently 1, 2, 3, 4, 5, 6, 7, or 8;
each R^{X} is independently selected from hydrogen, C₁-C₄alkyl and —CH₂CO₂H;
L⁵ is absent, one or more independently selected natural or unnatural amino acids, wherein any free amine of an amino acid or amide bond linking the 2 or more amino acids is optionally independently substituted with —CH₃, and any peptide that is formed is a linear or branched peptide, and wherein any free amine of the amino acid or peptide is optionally substituted with —C(═O)—(CH₂)₁₋₄-(4-iodophenyl);
L⁷ is absent, unsubstituted or substituted C₁-C₆alkylene, unsubstituted or substituted C₁-C₆heteroalkylene, unsubstituted or substituted cycloalkylene, unsubstituted or substituted heterocycloalkylene, unsubstituted or substituted phenylene, unsubstituted or substituted heteroarylene,
L⁸ is absent, —[CH(R^{Y})]_{y}—, —(CH₂)_{y}—, —(NR^{X}CH₂CH₂)—(OCH₂CH₂)_{y}—, each y is 1, 2, 3, 4, 5, 6, 7 or 8; each R^{Y} is independently selected from hydrogen and —OH; R^{X} is selected from hydrogen, C₁-C₄alkyl and —CH₂CO₂H;
L⁹-L¹⁰- is —NHC(═O)—C₁-C₂alkylene.

97. The compound of claim 93, or a pharmaceutically acceptable salt thereof, wherein L³ is -L⁴-L⁵-L⁶-L⁷-L⁸-L⁹-L¹⁰-;
L⁴ is absent;
L⁵ is one or more independently selected natural or unnatural amino acids, wherein any free amine of an amino acid or amide bond linking the 2 or more amino acids is optionally independently substituted with —CH₃, and any peptide that is formed is a linear or branched peptide, and wherein any free amine of the amino acid or peptide is optionally substituted with —C(═O)—(CH₂)₁₋₄-(4-iodophenyl);
L⁶ is —C(═O)—(CH₂CH₂X⁴)ᵥ—, or —(CH₂CH₂X⁴)ᵥ—, —C(═O)—(X⁴CH₂CH₂)ᵥ—, or —(X⁴CH₂CH₂)ᵥ—; v is independently 1, 2, 3, or 4;
L⁷ is absent or unsubstituted or substituted C₁-C₆alkylene;
L⁸ is —(X⁵CH₂CH₂)_{y}— or —(CH₂CH₂X⁵)_{y}—;
y is 1, 2, 3, or 4; and
L⁹-L¹⁰- is —NHC(═O)—C₁-C₂alkylene.

98. The compound of claim 93, or a pharmaceutically acceptable salt thereof, wherein L³ is -L⁴-L⁵-L⁶-L⁷-L⁸-L⁹-L¹⁰;
L⁴ is C₄-C₂₀polyethylene glycol, —C(═O)—C₄-C₂₀polyethylene glycol, —C(═O)NH—C₄-C₂₀polyethylene glycol, or —NHC(═O)—C₄-C₂₀polyethylene glycol;
L⁵ is absent;
L⁶ is absent;
L⁷ is unsubstituted or substituted C₁-C₆alkylene;
L⁸ is absent; and
L⁹-L¹⁰- is —NHC(═O)—C₁-C₂alkylene.

99. The compound of claim 93, or a pharmaceutically acceptable salt thereof, wherein L⁵ is

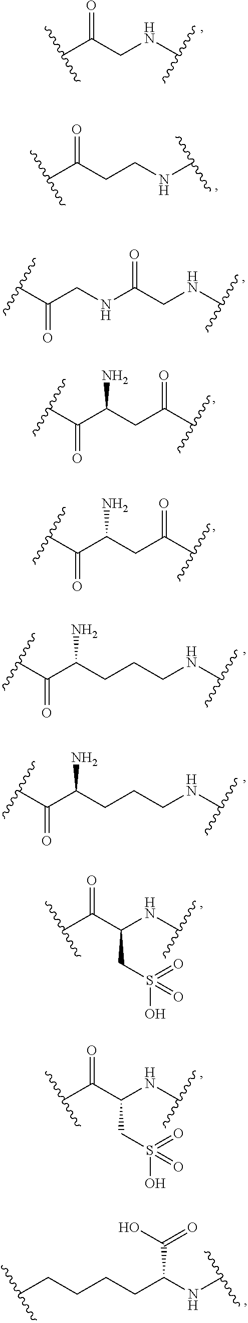

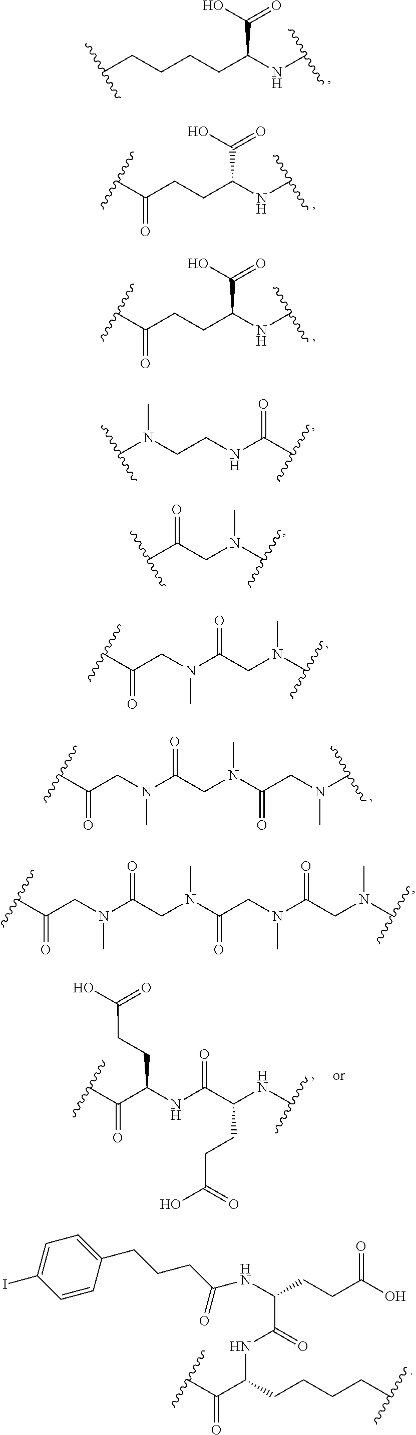

100. The compound of claim 79, or a pharmaceutically acceptable salt thereof, wherein L³-Q is: 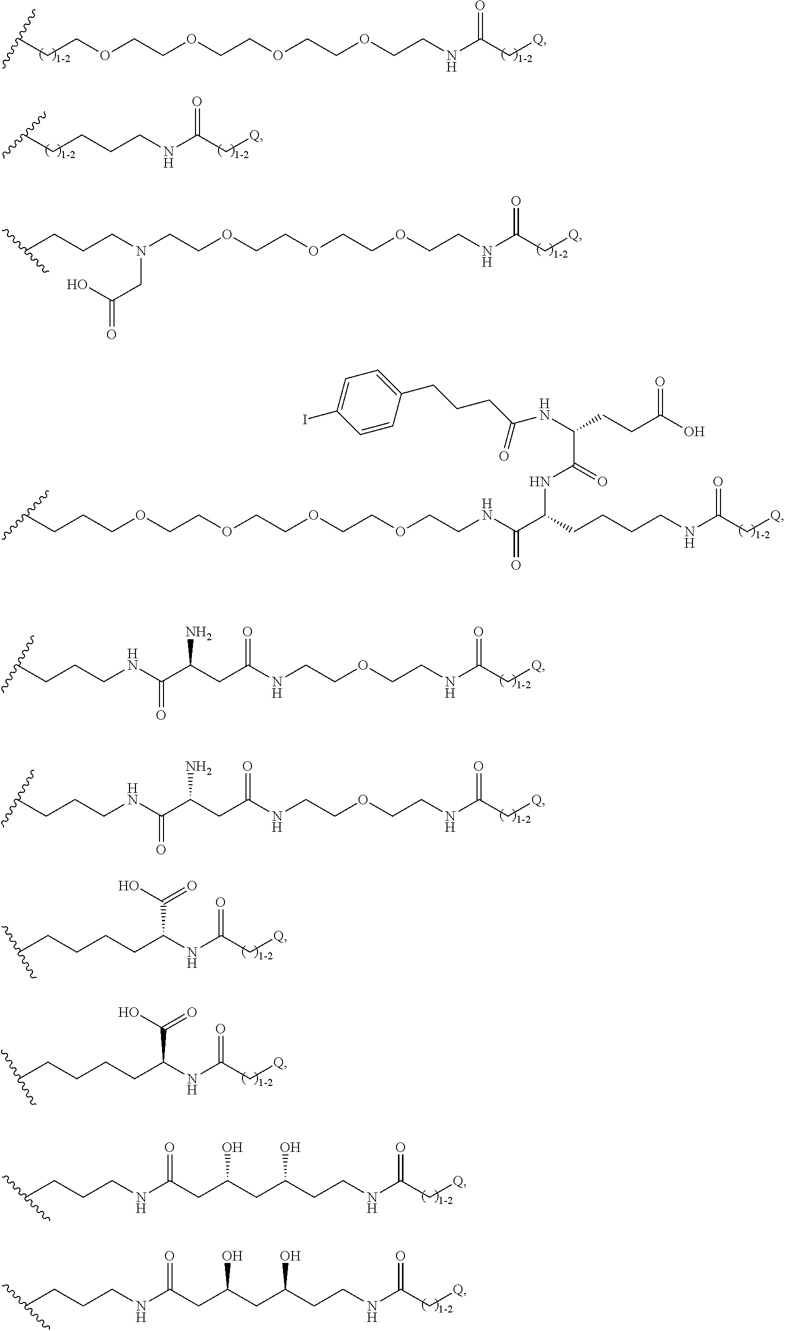 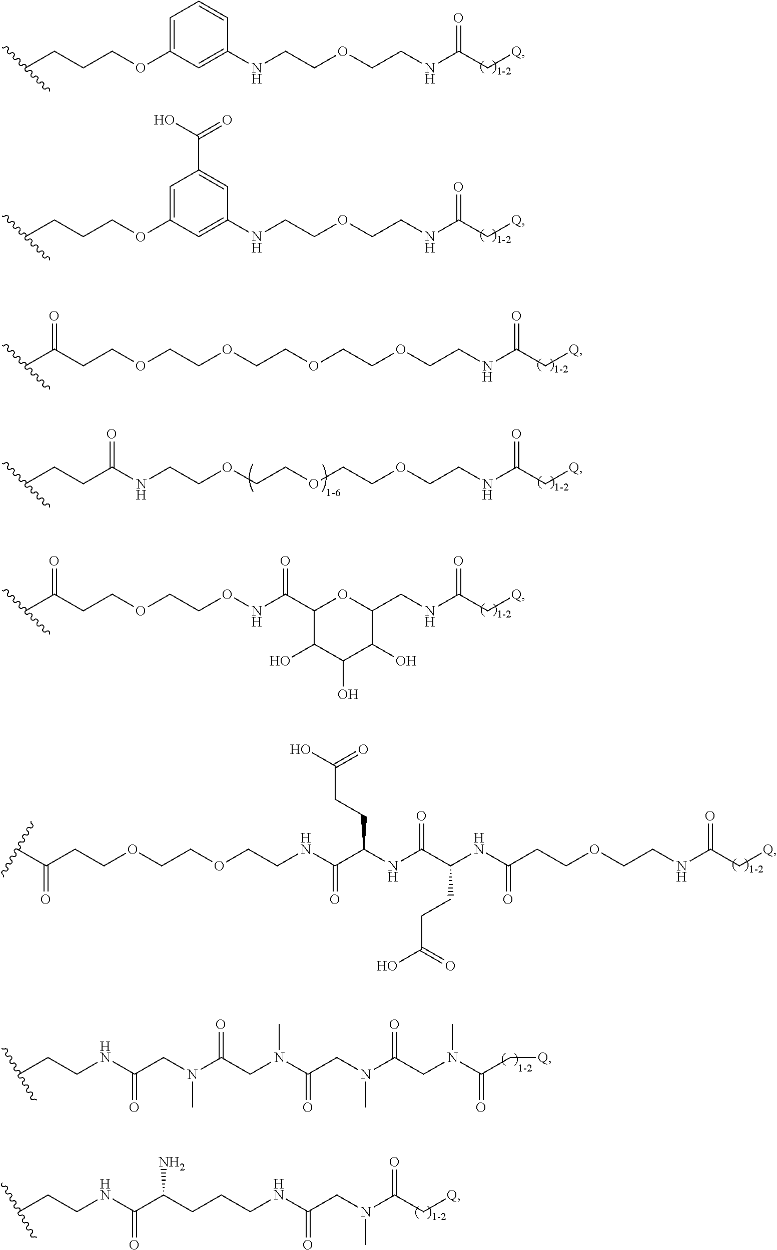 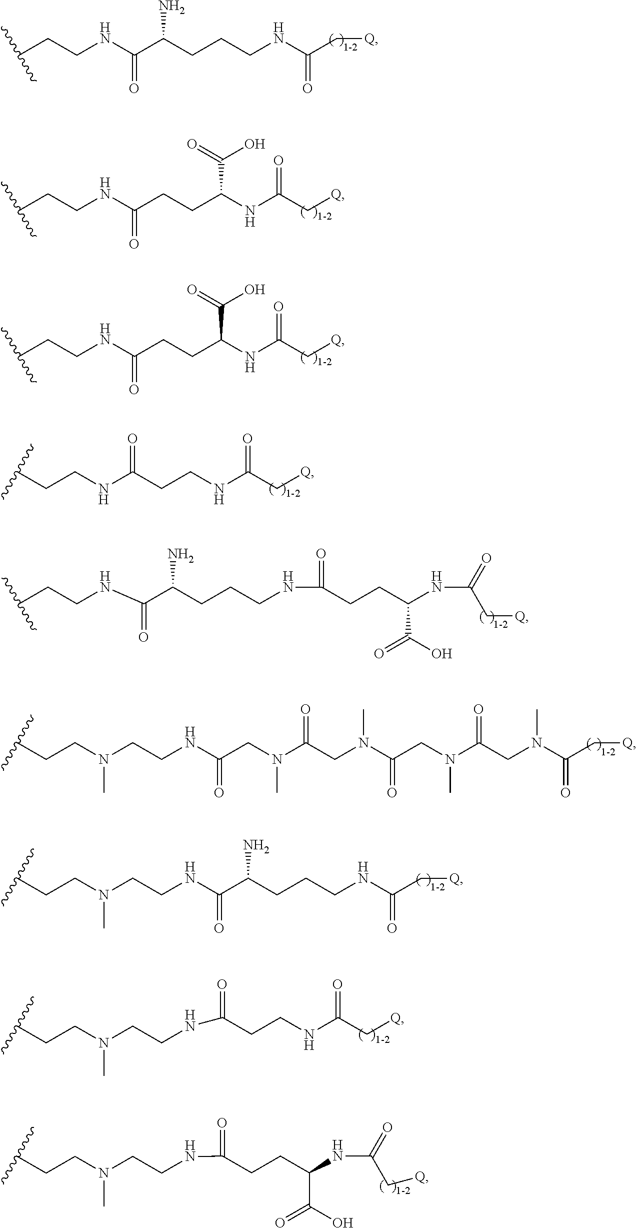 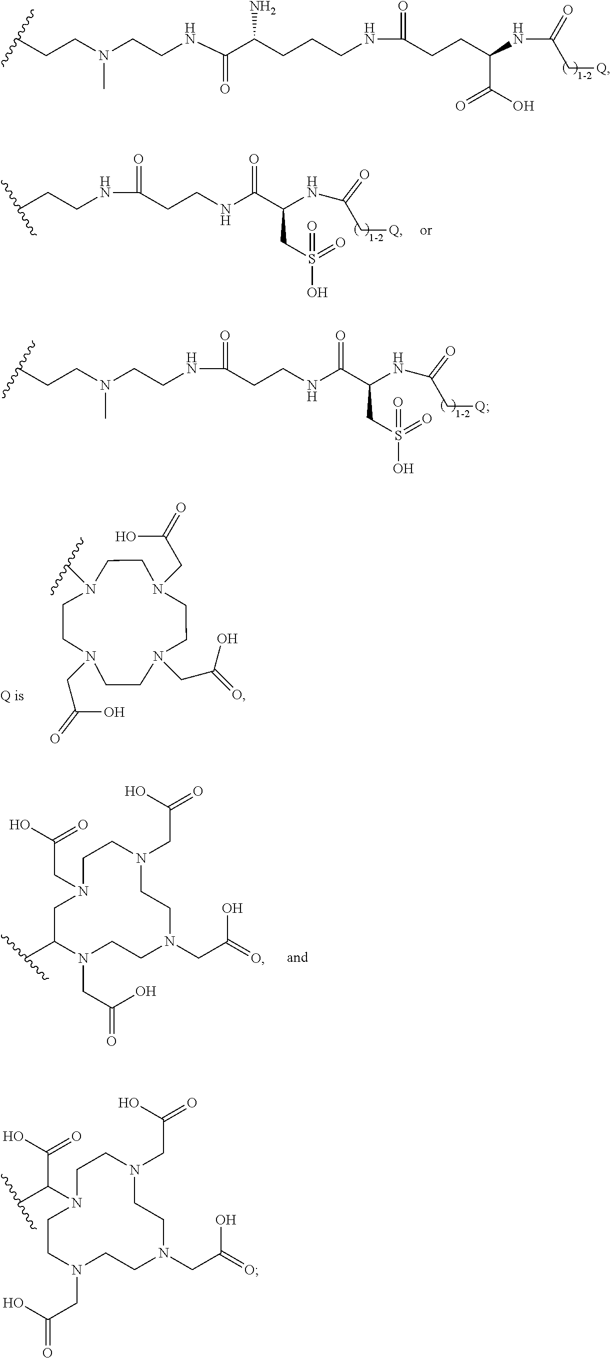
or a radionuclide complex thereof.

101. The compound of claim 79, or a pharmaceutically acceptable salt thereof, wherein
L² is absent, —CH₂CH₂NH—, —CH₂CH₂CH₂NH—, 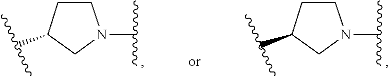 [SMILES: *[C@@H]1CCN(*)C1 ; *[C@H]1CCN(*)C1]
L³-Q is: 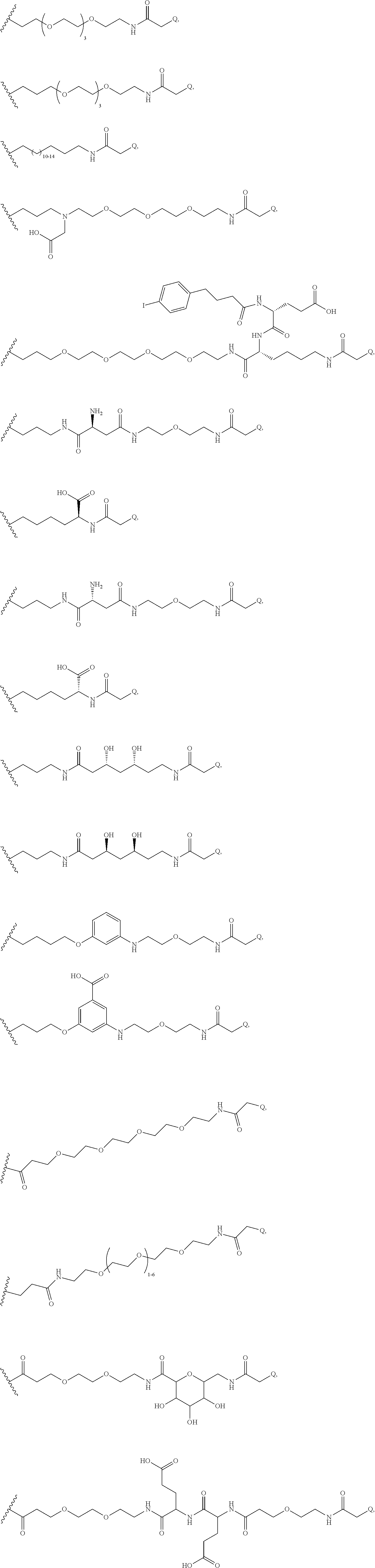 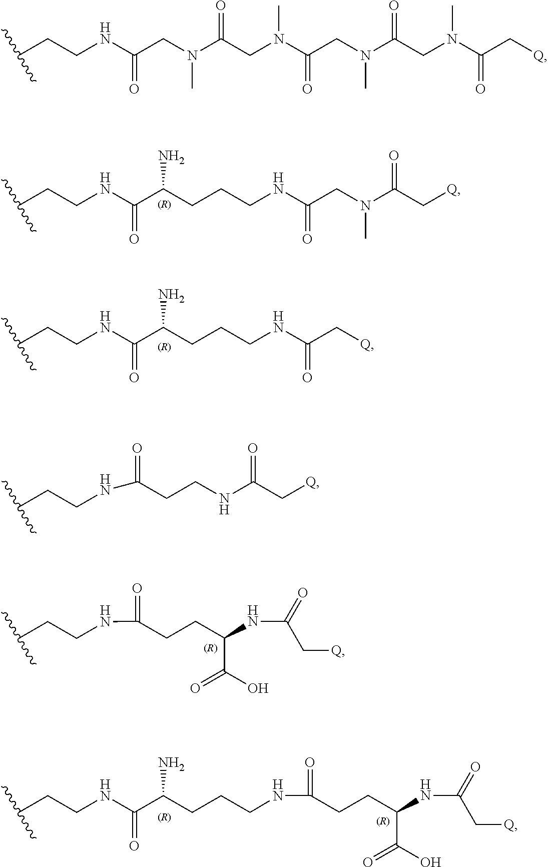 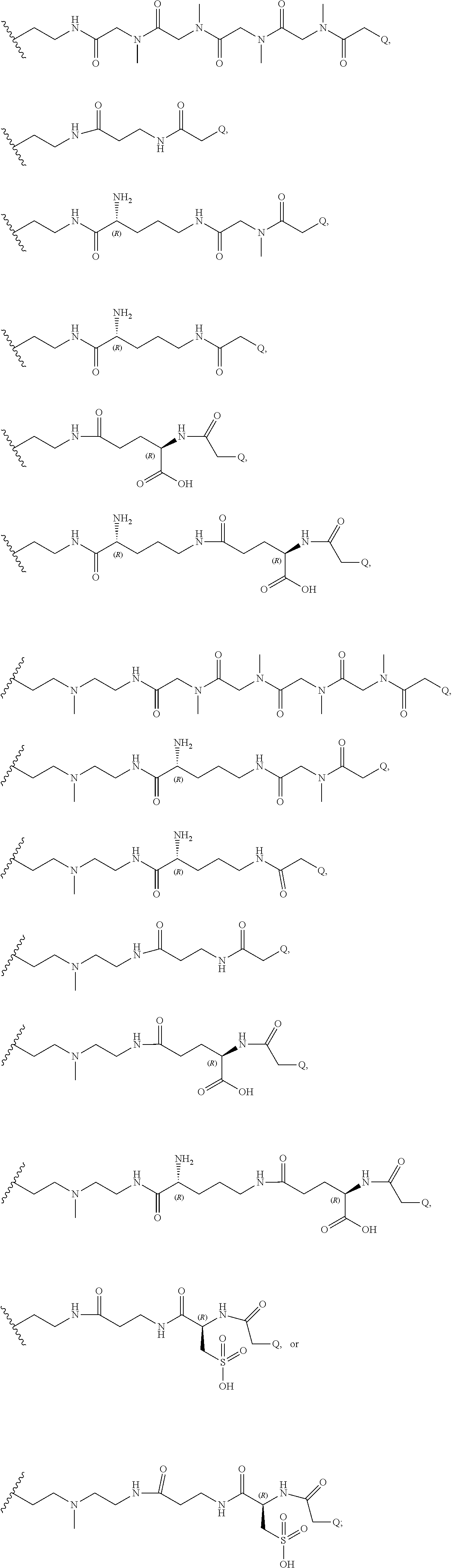
Q is 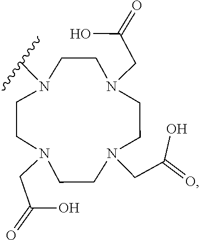 [SMILES: *N1CCN(CC(=O)O)CCN(CC(=O)O)CCN(CC(=O)O)CC1]
or a radionuclide complex thereof

102. The compound of claim 79, or a pharmaceutically acceptable salt thereof, wherein the compound of Formula (I) has one of the following structures, or a pharmaceutically acceptable salt thereof: 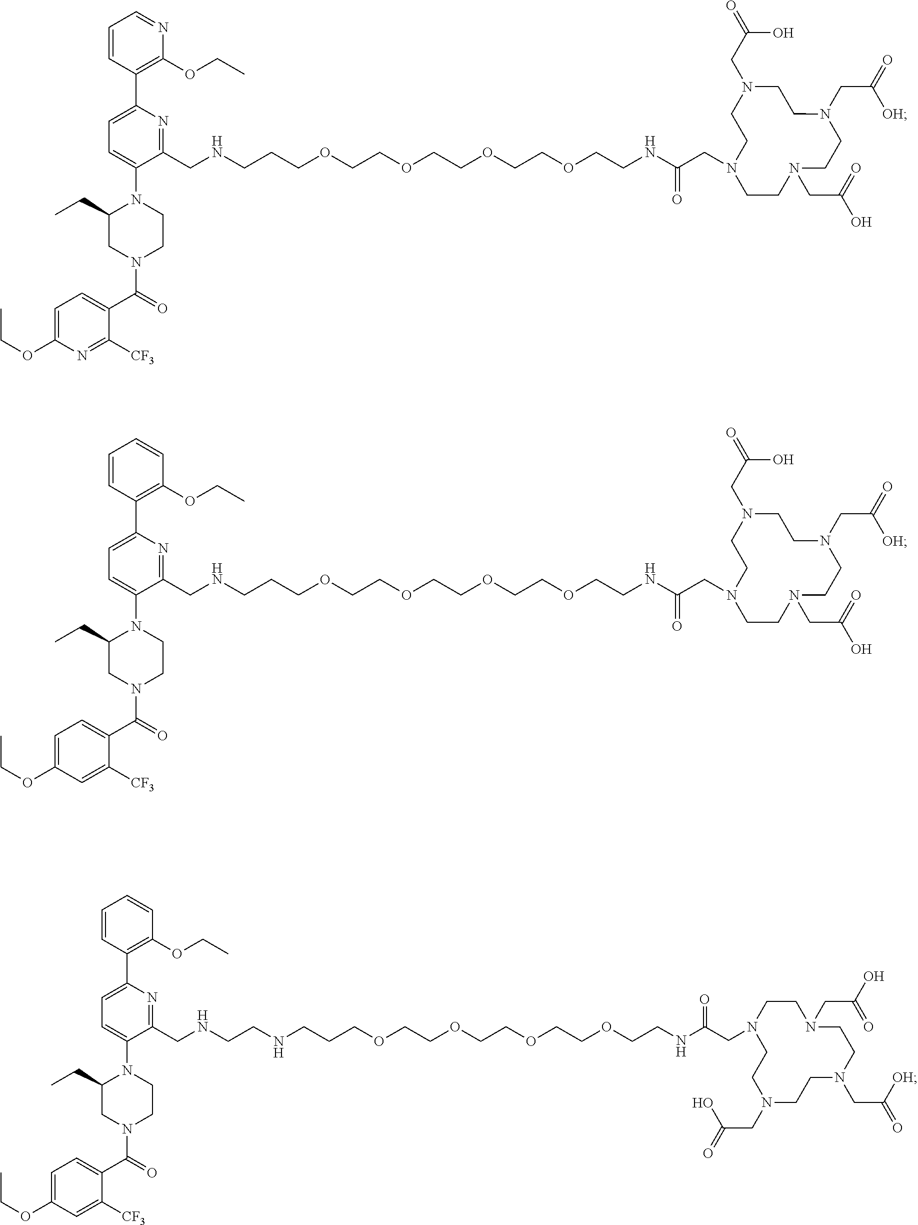 [SMILES: CCOc1ccc(C(=O)N2CCN(c3ccc(-c4cccnc4OCC)nc3CNCCCOCCOCCOCCOCCNC(=O)CN3CCN(CC(=O)O)CCN(CC(=O)O)CCN(CC(=O)O)CC3)[C@H](CC)C2)c(C(F)(F)F)n1 ; CCOc1ccc(C(=O)N2CCN(c3ccc(-c4ccccc4OCC)nc3CNCCCOCCOCCOCCOCCNC(=O)CN3CCN(CC(=O)O)CCN(CC(=O)O)CCN(CC(=O)O)CC3)[C@H](CC)C2)c(C(F)(F)F)c1 ; CCOc1ccc(C(=O)N2CCN(c3ccc(-c4ccccc4OCC)nc3CNCCNCCCOCCOCCOCCOCCNC(=O)CN3CCN(CC(=O)O)CCN(CC(=O)O)CCN(CC(=O)O)CC3)[C@H](CC)C2)c(C(F)(F)F)c1] 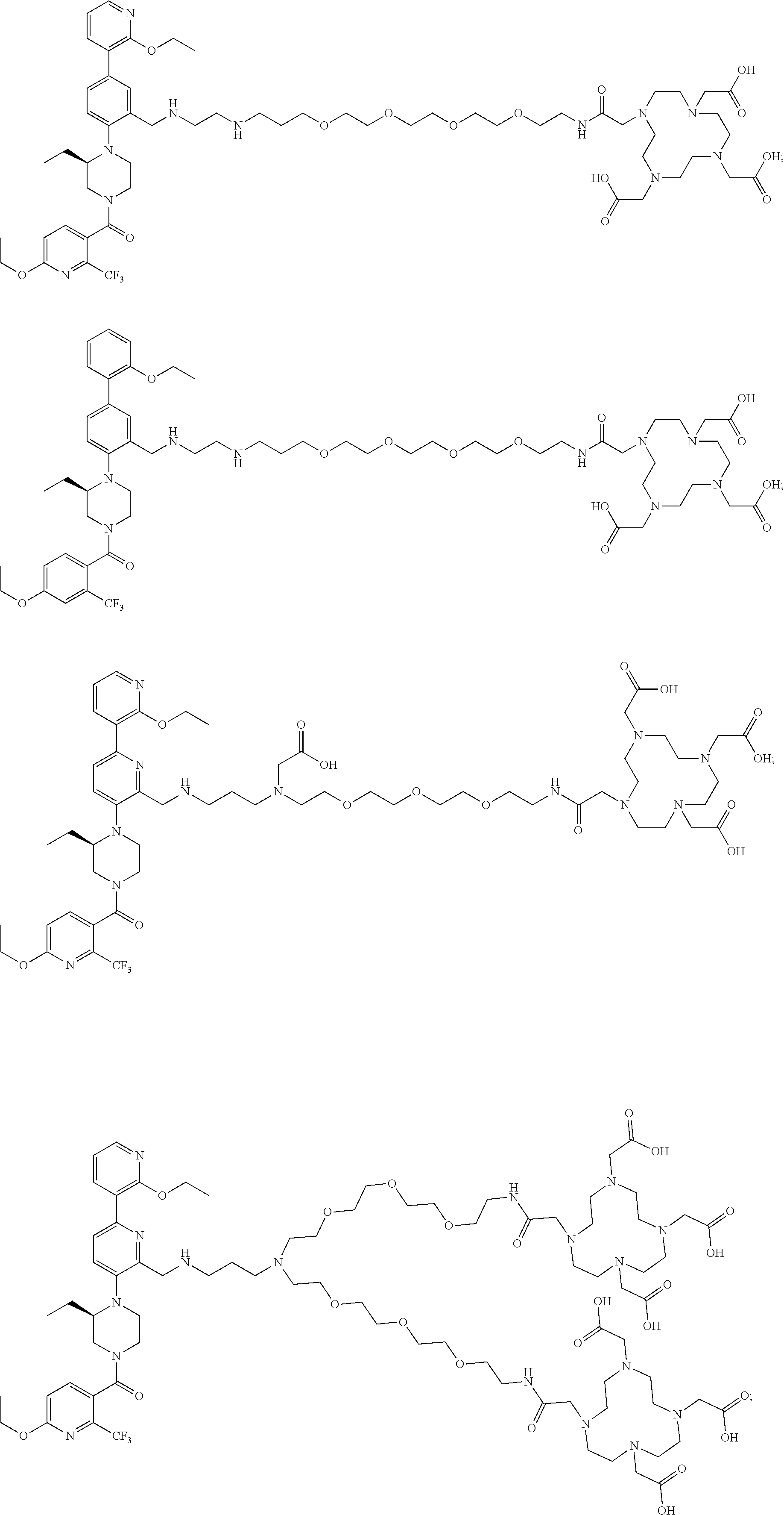 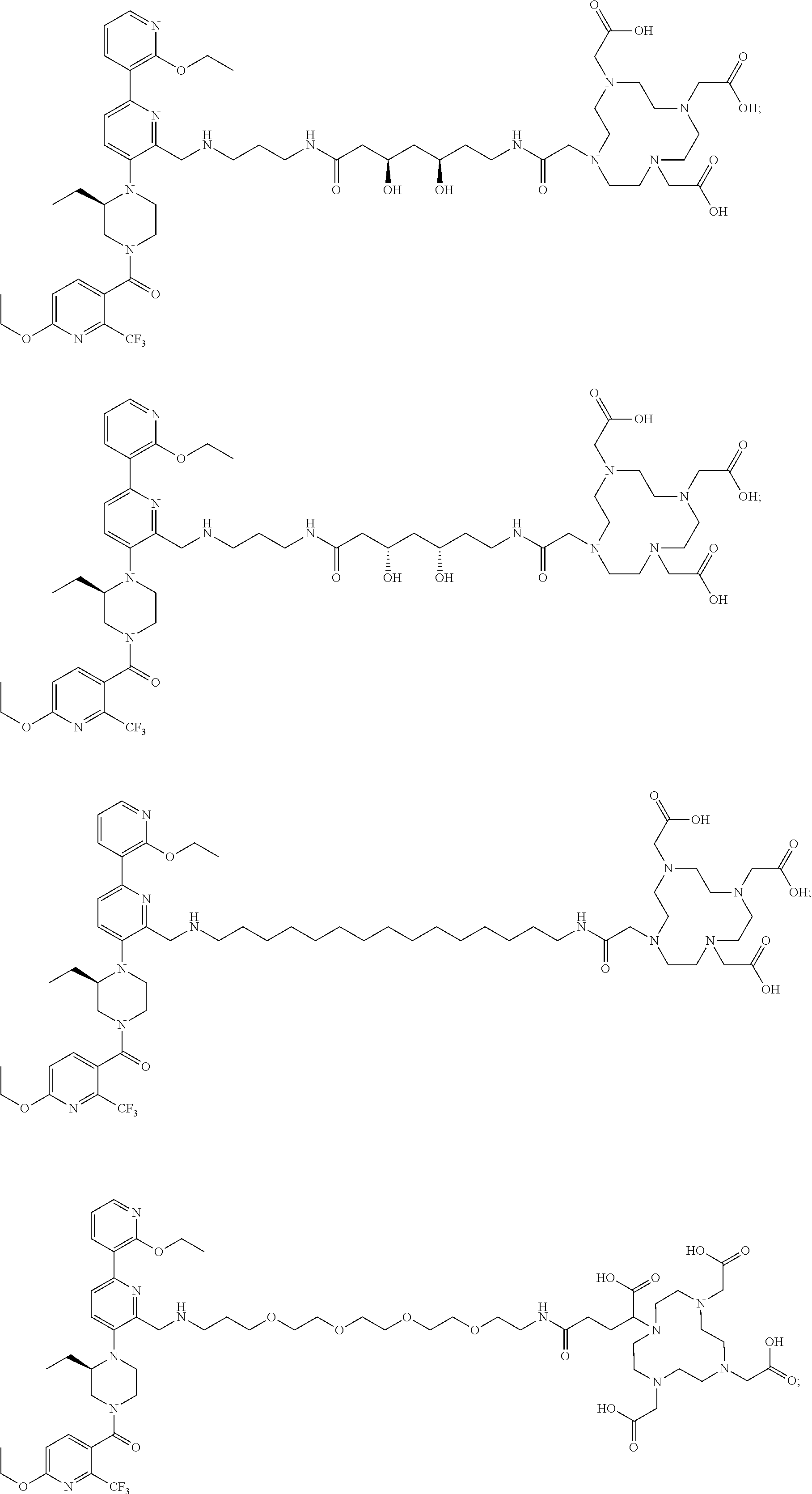 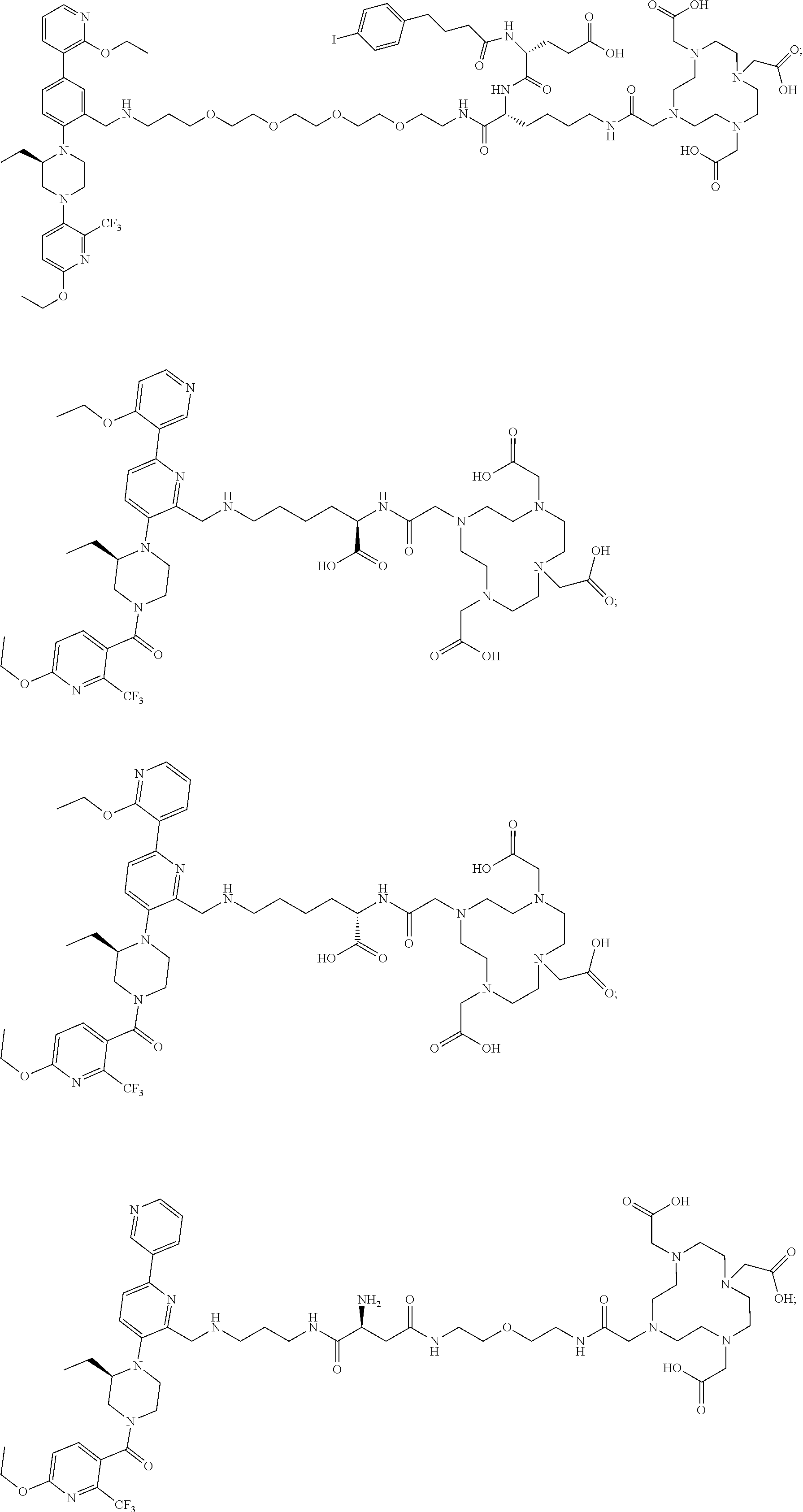 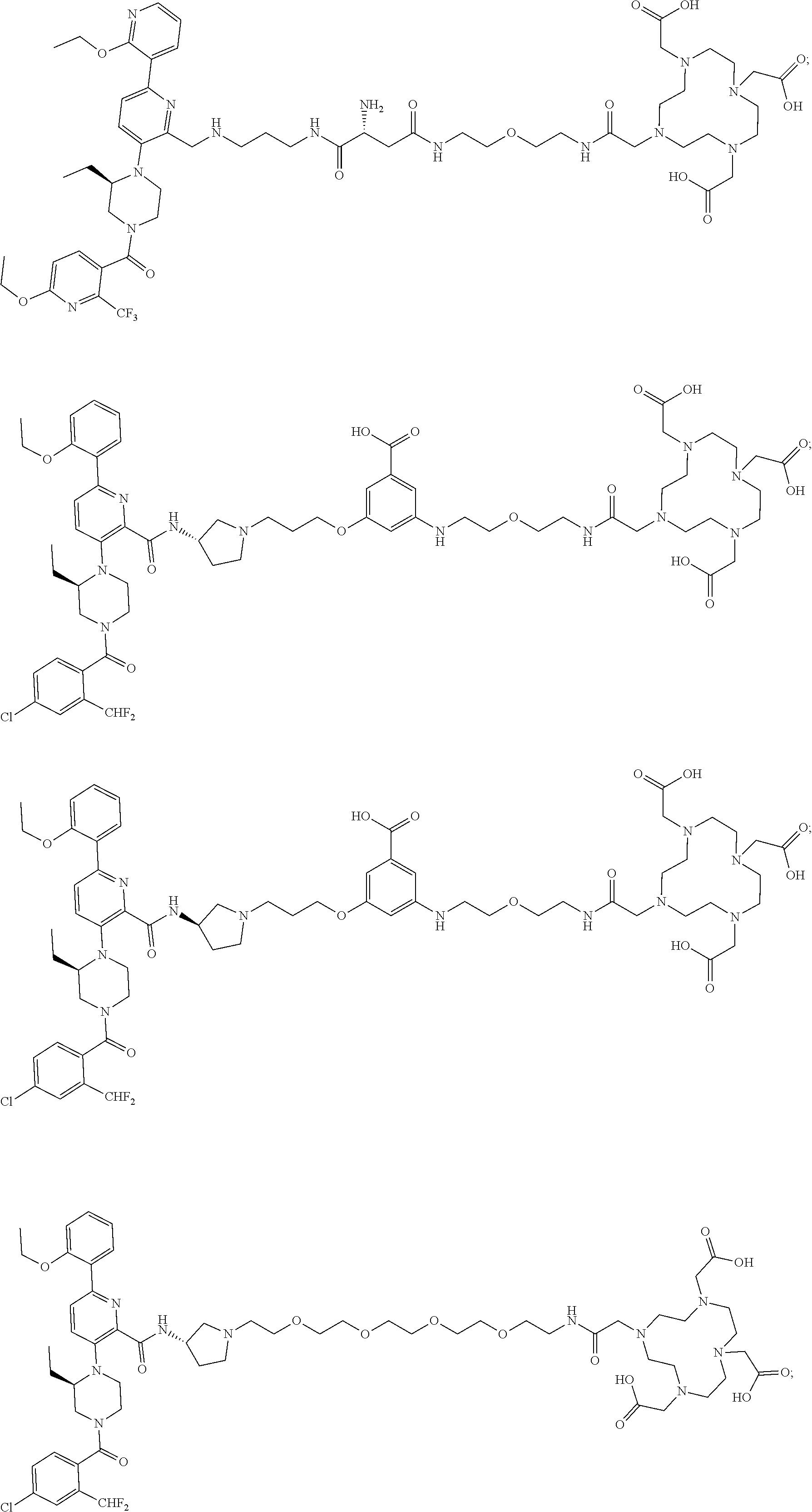 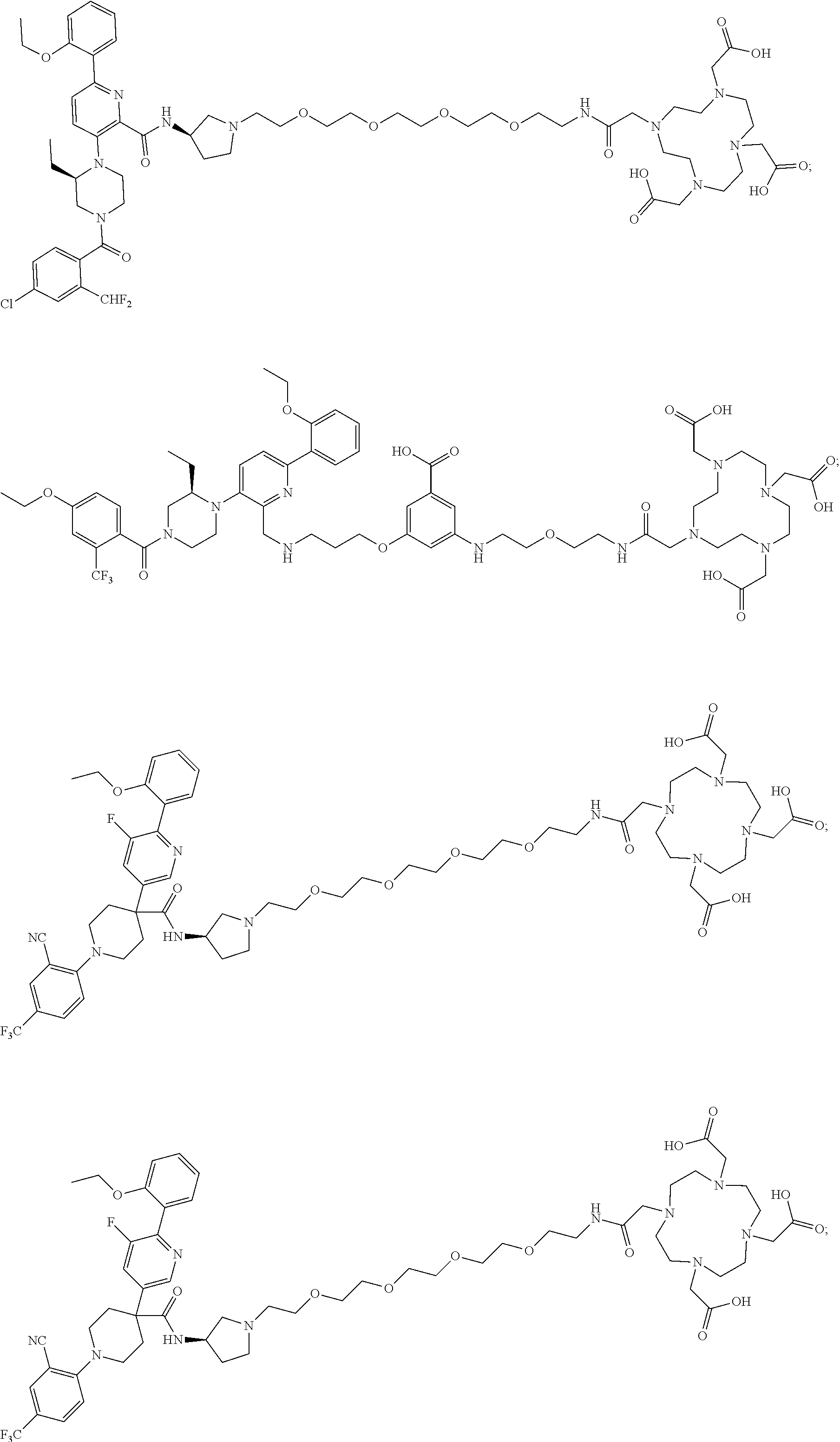 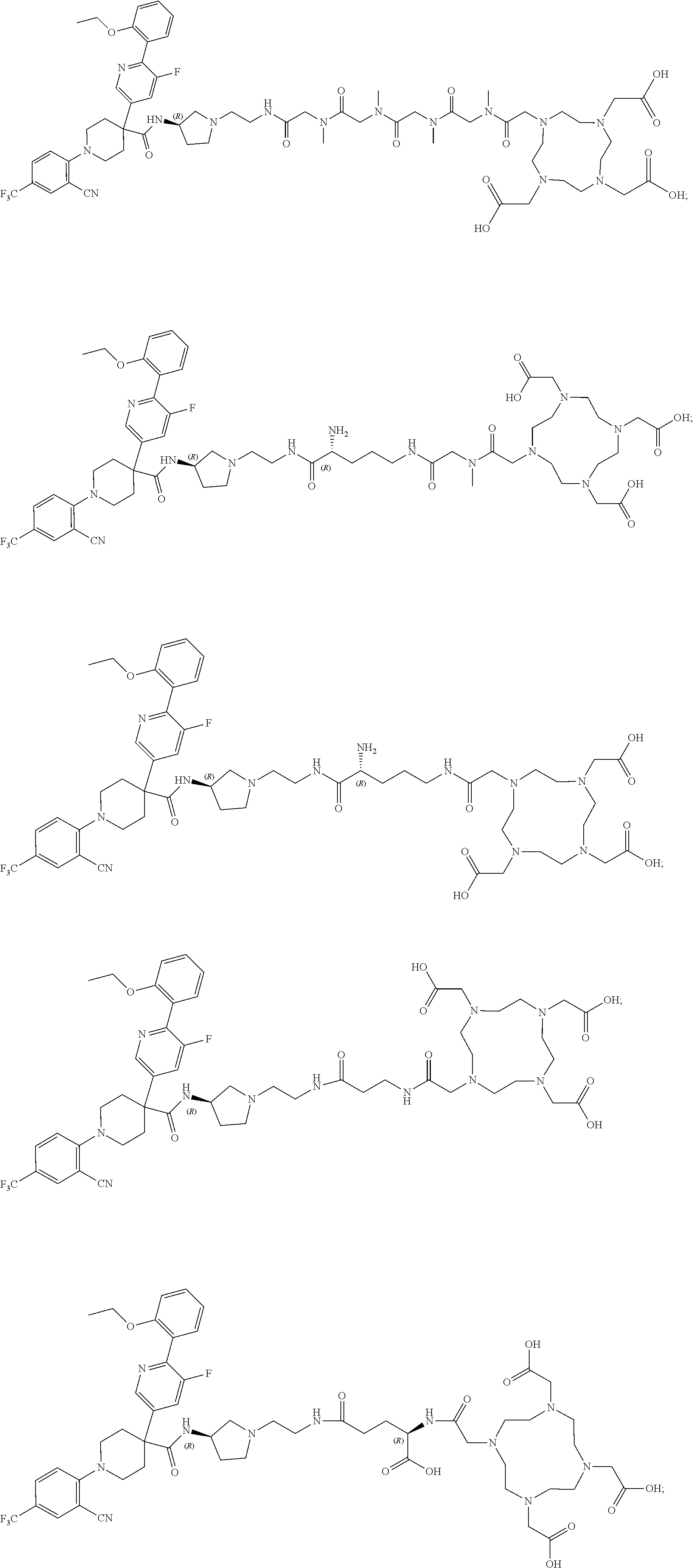 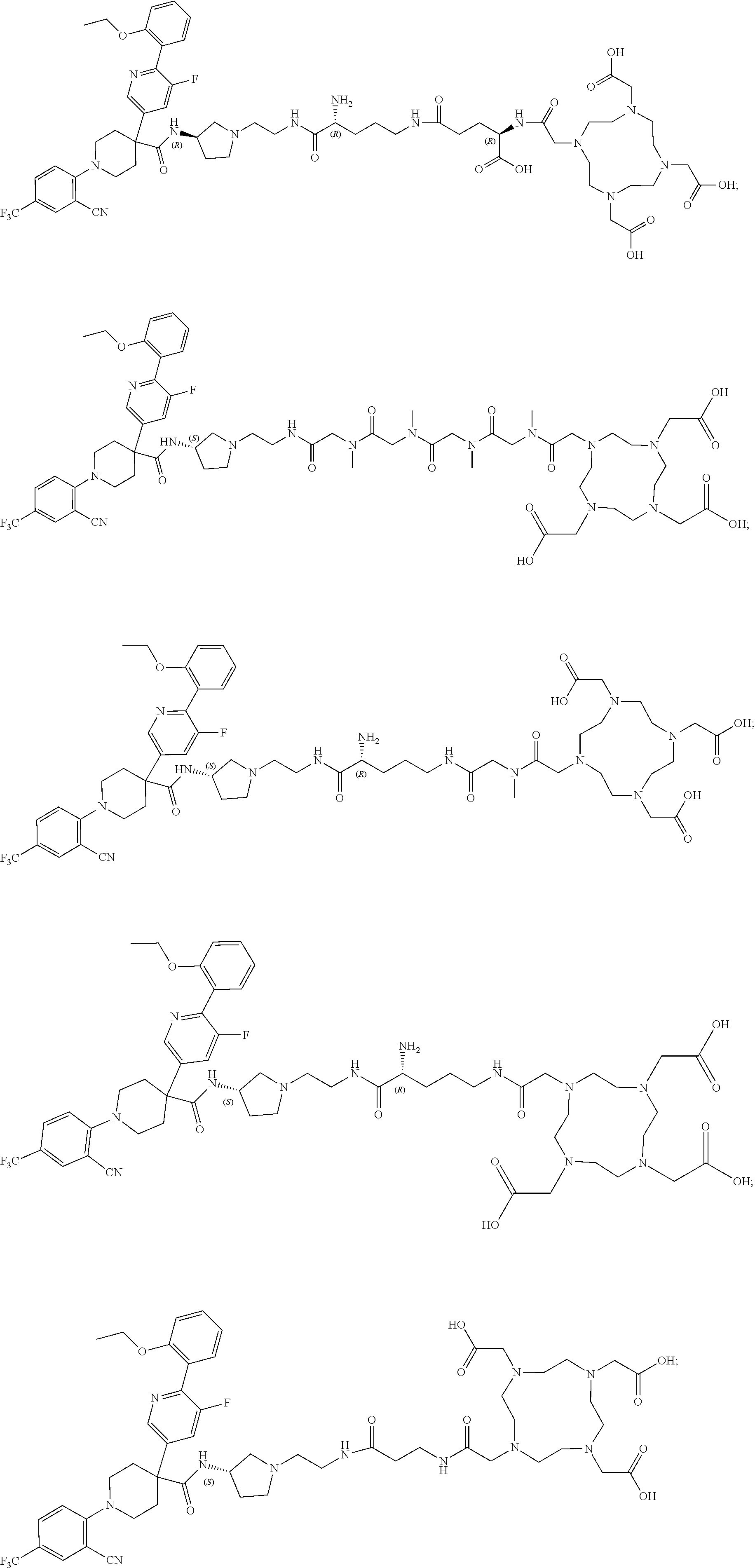 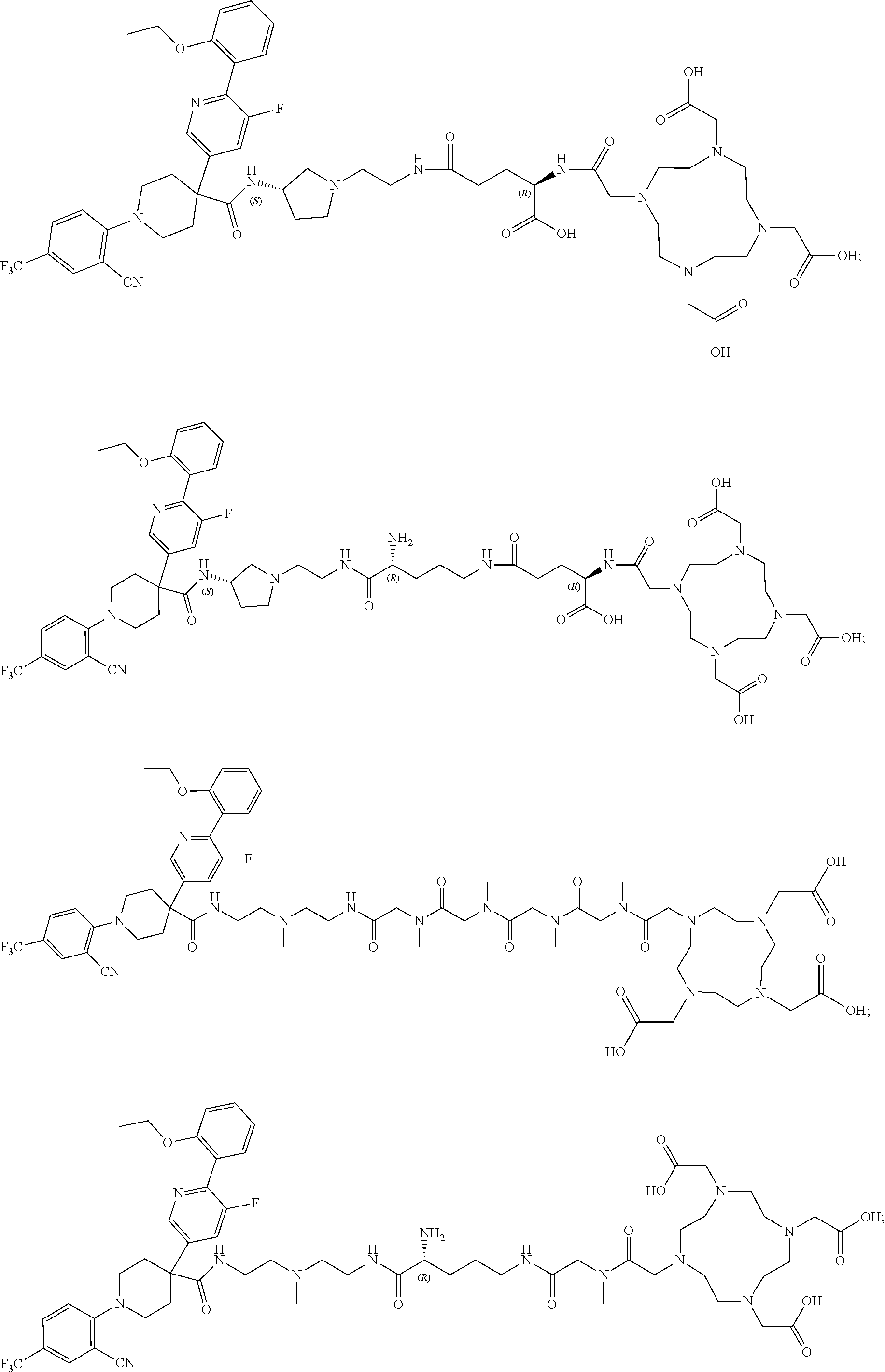 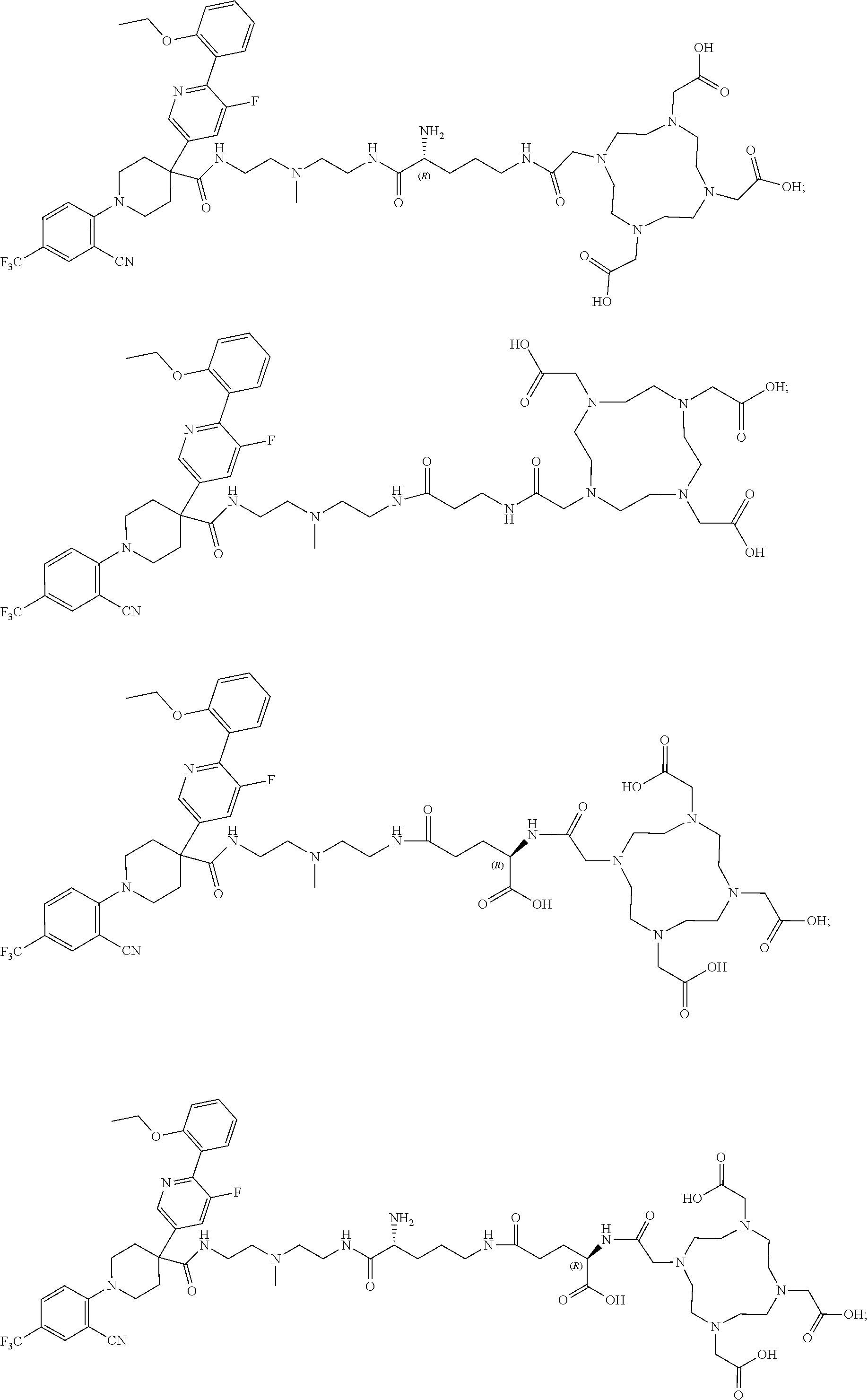 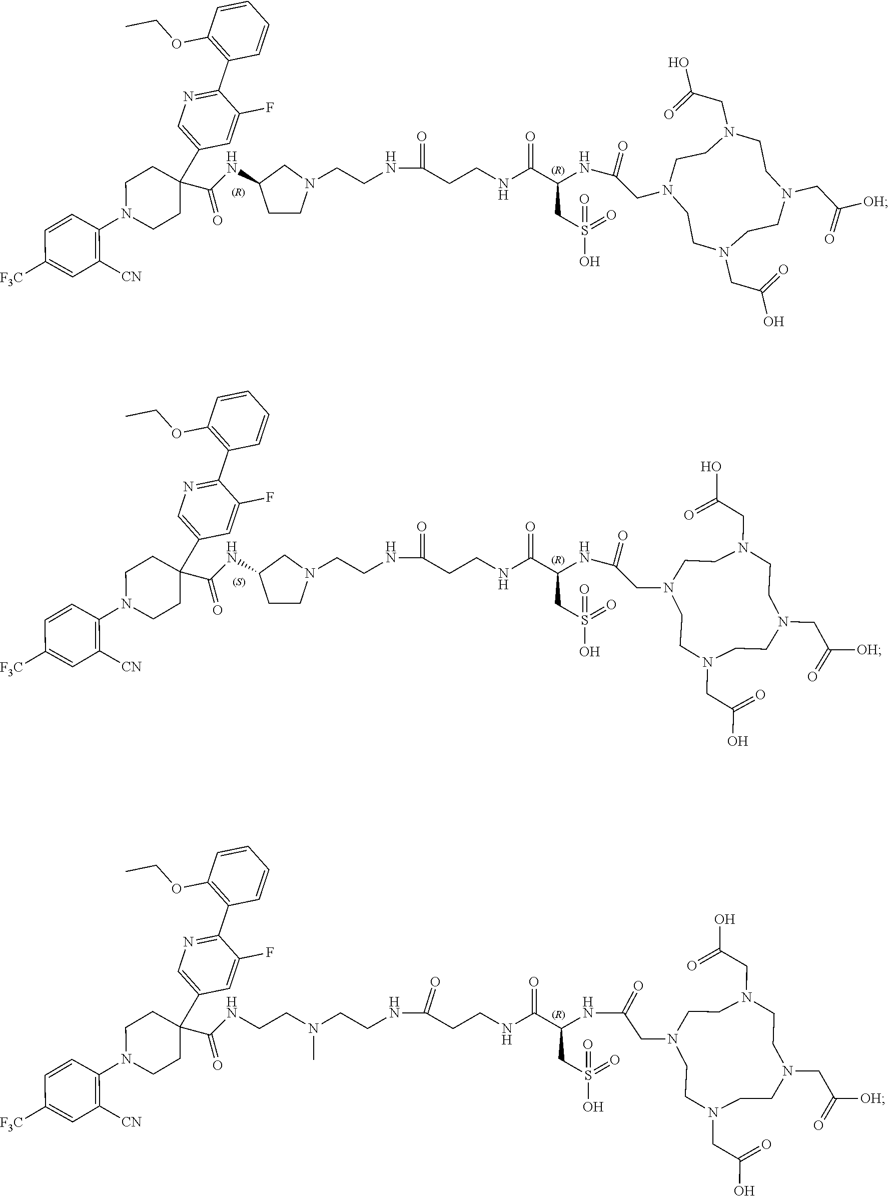
or radionuclide complex thereof.

103. The compound of claim 79, or a pharmaceutically acceptable salt thereof, wherein the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

104. The compound of claim 79, or a pharmaceutically acceptable salt thereof, wherein the radionuclide of the radionuclide complex is 111-indium (¹¹¹In), 115-indium (¹¹⁵In), 67-gallium (⁶⁷Ga), 68-gallium (⁶⁸Ga), 70-gallium (⁷⁰Ga), 225-actinium (²²⁵Ac), 175-lutetium (¹⁷⁵Lu) or 177-lutetium (¹⁷⁷Lu).

105. A pharmaceutical composition comprising a compound of claim 79, or a pharmaceutically acceptable salt thereof, and at least one pharmaceutically acceptable excipient.

106. A method for the treatment of cancer comprising administering to a mammal with cancer an effective amount of a compound of claim 79, or a pharmaceutically acceptable salt thereof, wherein the cancer comprises tumors and the tumor overexpress the melanocortin subtype-2 receptor (MC2R).

107. The method of claim 106, wherein the cancer is an endocrine cancer or adrenocortical carcinoma.

108. A method of killing tumors in a mammal that overexpress the melanocortin subtype-2 receptor (MC2R) comprising administering to the mammal a compound of claim 79, or a pharmaceutically acceptable salt thereof, wherein the compound of claim 1, or a pharmaceutically acceptable salt thereof, comprises a therapeutic radionuclide.

109. The method of claim 108, wherein the mammal has been diagnosed with adrenocortical carcinoma.
